SEQUENCE LISTING (230 sequences)

SEQ ID NO: 1 - DNA, 1053 bp, Aquifex aeolicus
gtggaaaccacaatattccagttccagaaaacttttttcacaaaacctccgaaggagagggtcttcgtccttcatggagaagagcagtatctcataagaacctttttgtctaagctgaaggaaaagtacggggagaattacacggttctgtggggggatgagataagcgaggaggaattctacactgccctttccgagaccagtatattcggcggttcaaaggaaaaagcggtggtcatttacaacttcggggatttcctgaagaagctcggaaggaagaaaaaggaaaaagaaaggcttataaaagtcctcagaaacgtaaagagtaactacgtatttatagtgtacgatgcgaaactccagaaacaggaactttcttcggaacctctgaaatccgtagcgtctttcggcggtatagtggtagcaaacaggctgagcaaggagaggataaaacagctcgtccttaagaagttcaaagaaaaagggataaacgtagaaaacgatgcccttgaataccttctccagctcacgggttacaacttgatggagctcaaacttgaggttgaaaaactgatagattacgcaagtgaaaagaaaattttaacactcgatgaggtaaagagagtagccttctcagtctcagaaaacgtaaacgtatttgagttcgttgatttactcctcttaaaagattacgaaaaggctcttaaagttttggactccctcatttccttcggaatacaccccctccagattatgaaaatcctgtcctcctatgctctaaaactttacaccctcaagaggcttgaagagaagggagaggacctgaataaggcgatggaaagcgtgggaataaagaacaactttctcaagatgaagttcaaatcttacttaaaggcaaactctaaagaggacttgaagaacctaatcctctccctccagaggatagacgctttttctaaactttactttcaggacacagtgcagttgctgagggatttcttgacctcaagactggagagggaagttgtgaaaaatacttctcatggtggataa

SEQ ID NO: 2 - DNA, 1053 bp, Aquifex aeolicus; identical to SEQ ID NO: 1
  CDS (1)..(1053)

SEQ ID NO: 3 - protein, 350 aa, Aquifex aeolicus
VETTIFQFQKTFFTKPPKERVFVLHGEEQYLIRTFLSKLKEKYGENYTVLWGDEISEEEFYTALSETSIFGGSKEKAVVIYNFGDFLKKLGRKKKEKERLIKVLRNVKSNYVFIVYDAKLQKQELSSEPLKSVASFGGIVVANRLSKERIKQLVLKKFKEKGINVENDALEYLLQLTGYNLMELKLEVEKLIDYASEKKILTLDEVKRVAFSVSENVNVFEFVDLLLLKDYEKALKVLDSLISFGIHPLQIMKILSSYALKLYTLKRLEEKGEDLNKAMESVGIKNNFLKMKFKSYLKANSKEDLKNLILSLQRIDAFSKLYFQDTVQLLRDFLTSRLEREVVKNTSHGG

SEQ ID NO: 4 - DNA, 1029 bp, Bacillus halodurans
atgaactatttaaaattaaagcaagatgtccaagcgggacgagtggcccctgtatattttatgtatgggaccgagttgtttttaatggaagacctcatacaggacattttgtccgtaacgttatcggatgaggaaagggatatgaatgtttcatcttattcccttactgatgttccgattgaggcggcgctcgaagaagcagaaacggttcctttttttggctcgaagcgggtcgtgatcttaaaagatgctgccctatttacaagccaaaagcttgatgttgagcacgatgtgaaacgattggagcaatacatattgaacccagttccagagacggttttgctgatcatggccccttatgaaaagctcgatgaacgaaaaaaaatcacgaagcttatcaaaaaagagtcgcttgttcttgaggcaaagcctttggacgaaggagaggcaaaagcatggctatcctcactggcaagcgagcttcaggtggagatggacgagaaagctatcgagacgttgttagggatgacgggtcttcgcttgacccaattagcatctgaaatgaataaattagcgttgtatgtaggggaaggaggcatcatccgatcagaggacgtaaccttgctcgttgctaaaacgctcgatcaaaacatatttgatctgattgattttgccatcaatcagaggactcatcaagcgctctctctttaccatgagctcctgaaacaaaaagaggagccattgaagctgttggccttattaacacggcagtttcgcatcatgtaccaagtcaaagagcttggacgaagaggatacaccccgaatcagatggcgaagccgttaaaaattcatccctatgttgccaaacttgcgggaaaaaaagcagccagcatgtcagaccaactgttatattccctaatcgaaaaagctgcggatacagagtttgcgattaaaagtggaaaggttgataaagtgctggcattagaattgtttttactcacaatgggttctagggagagcgtaggttag

SEQ ID NO: 5 - DNA, 1029 bp, Bacillus halodurans; identical to SEQ ID NO: 4
  CDS (1)..(1029)

SEQ ID NO: 6 - protein, 342 aa, Bacillus halodurans
MNYLKLKQDVQAGRVAPVYFMYGTELFLMEDLIQDILSVTLSDEERDMNVSSYSLTDVPIEAALEEAETVPFFGSKRVVILKDAALFTSQKLDVEHDVKRLEQYILNPVPETVLLIMAPYEKLDERKKITKLIKKESLVLEAKPLDEGEAKAWLSSLASELQVEMDEKAIETLLGMTGLRLTQLASEMNKLALYVGEGGIIRSEDVTLLVAKTLDQNIFDLIDFAINQRTHQALSLYHELLKQKEEPLKLLALLTRQFRIMYQVKELGRRGYTPNQMAKPLKIHPYVAKLAGKKAASMSDQLLYSLIEKAADTEFAIKSGKVDKVLALELFLLTMGSRESVG

SEQ ID NO: 7 - DNA, 1044 bp, Bacillus subtilis
atggtatttgatgtgtggaaaagcctgaaaaaaggagaggtccatccggtttattgtttatacggaaaagagacatatctgctgcaagaaaccgtcagcaggatcagacagacggtcgttgatcaggagacaaaggatttcaatctgtccgtttttgatctggaagaggacccgctggatcaagcgattgcagatgctgaaacgtttccgtttatgggggagcggcgtcttgtcattgtgaaaaatccatattttttaacaggtgaaaagaaaaaagaaaaaattgagcacaacgtcagtgcgcttgaatcatatatacaatcacctgcgccttacacagtctttgtcctgcttgctccgtatgaaaagcttgatgagcgaaaaaagctgacgaaagcgttaaagaagcacgcgtttatgatggaggcgaaggaattaaatgcaaaggaaacgacagactttactgtcaaccttgcaaaaacagagcagaaaacaatcggcacggaagcggcggagcatttggttctgcttgtgaacggtcatctgtcatcgatttttcaggagattcaaaagctctgcacgtttattggagatcgtgaagaaattacgctggatgatgtaaaaatgcttgttgctagaagccttgaacaaaatatttttgagctgatcaataaaatcgtcaaccgaaaacgaacagagagtctgcaaattttttatgatttgctaaaacaaaatgaagaaccgatcaaaattatggcgcttatttcgaatcagttccggctgattctgcagacgaagtacttcgcggaacagggatacggacaaaaacaaatcgcttctaatctaaaagttcacccatttcgggtaaagctggcgatggatcaagcaaggcttttttcagaggaggagctccgtttaattattgaacagcttgccgtcatggactatgagatgaaaaccgggaaaaaggacaagcagctcctgctcgaactgttccttttacagctgttaaaaagaaatgaaaaaaacgatccccattattga

SEQ ID NO: 8 - DNA, 1044 bp, Bacillus subtilis; identical to SEQ ID NO: 7
  CDS (1)..(1044)

SEQ ID NO: 9 - protein, 347 aa, Bacillus subtilis
MVFDVWKSLKKGEVHPVYCLYGKETYLLQETVSRIRQTVVDQETKDFNLSVFDLEEDPLDQAIADAETFPFMGERRLVIVKNPYFLTGEKKKEKIEHNVSALESYIQSPAPYTVFVLLAPYEKLDERKKLTKALKKHAFMMEAKELNAKETTDFTVNLAKTEQKTIGTEAAEHLVLLVNGHLSSIFQEIQKLCTFIGDREEITLDDVKMLVARSLEQNIFELINKIVNRKRTESLQIFYDLLKQNEEPIKIMALISNQFRLILQTKYFAEQGYGQKQIASNLKVHPFRVKLAMDQARLFSEEELRLIIEQLAVMDYEMKTGKKDKQLLLELFLLQLLKRNEKNDPHY

SEQ ID NO: 10 - DNA, 996 bp, Buchnera
atgaaaaatatatatccaaatgaactaaaaaaaagtttaatgcaaaaattgcattatttttatatttttttaggagaagatttttttttattagaaaaaaatcaagatatgatattgaattttgcatataaaaaaggattcctagaaaaaattataattgatgtagaaaaaaatcaagattggaaaaaaattattttattttataaaactaataatttattttttaaaaaaactactttagtaatcaactttcttataaaaaagttaaatgttattttaattcaaaatcttaataaaatgttttctttattgcatccagatatattaataatattaaaattcaatcatttatcacgttttattcaaaaaaataaatcattaaaagaatttaaaaactacaatatagtttcttgttttacgccgtataatttaaattttattaattggattaagtatgaaatacaagaaaaaaaaataaatattgaggaaaaagcattttttttattatgtaaatattatgaaggtaatactttatttatatataaaattttagatatgctatttataatatggcccgatacatgtattacagaaaaaaaaataaaaaaaattatcattgagttttttgatgtttctccatcatattggatcaattctatatttcaaggtaaaacagaaaaatccttttatatattaaatattttttttaaaaaaaaatataatcctcttattttagtacgttctttgcaaaaagatttgttacaattgattcatatgaagcgtgaaaaaaaaataagtatttatgtaatgttagaaaaatataatatttttgtaacaagacgtaagttttttatcaaggcatttaataaaattaacaacgatagtttattaaaagctattcaaattcttgtaaaaatagaagtaaatatcaaaaaaaaatataataattatgtttggaatcaactacaagaattaattttaatattatgtaactaa

SEQ ID NO: 11 - DNA, 996 bp, Buchnera; identical to SEQ ID NO: 10
  CDS (1)..(996)

SEQ ID NO: 12 - protein, 331 aa, Buchnera
MKNIYPNELKKSLMQKLHYFYIFLGEDFFLLEKNQDMILNFAYKKGFLEKIIIDVEKNQDWKKIILFYKTNNLFFKKTTLVINFLIKKLNVILIQNLNKMFSLLHPDILIILKFNHLSRFIQKNKSLKEFKNYNIVSCFTPYNLNFINWIKYEIQEKKINIEEKAFFLLCKYYEGNTLFIYKILDMLFIIWPDTCITEKKIKKIIIEFFDVSPSYWINSIFQGKTEKSFYILNIFFKKKYNPLILVRSLQKDLLQLIHMKREKKISIYVMLEKYNIFVTRRKFFIKAFNKINNDSLLKAIQILVKIEVNIKKKYNNYVWNQLQELILILCN

SEQ ID NO: 13 - DNA, 990 bp, Borrelia burgdorferi
atgcaagcggtttatttattgttgggtaatgagcaaggtttaaaagaagcctatttaaaagagcttttaattaaaatggatgcttttaaatccgaagtttcagttactaaaatttttttgtcagaactctcagctgtaggatttgctgagaaattattttccaattcttttttttcaaaaaaagaaatttttattgtttatgagtctgaacttttaaaagcaggaaaagatttagagctagtgtgtaattcaatcttaaagtctaacaataaaactgttatttttgtttccaatagcaatacatgtaacattgattttaagaataagcttaagtttataaaaaaagttttttatgagattcctgatgatgataaatttacatttgtaaaaagaaatttttttaatcttaatattaaaattacagattctgcaataaatttaatgcttttaatgttaaattcagatactaaaattttgaaattttatatagattcttttgcgctttttgccaagaataataccattgaggaggaagatatagcttcttggattagttttattcgctttgagaacaccttttccttatttaattcaattttgagaaaagatatgactcagtctttgatcaagattaagtctattttggatcagggagaagatttgcttaatattttaatgagtcttatttggcaatttaaaagattattaaaggtgcaaatagattataatgcatatgggagcttgcagagtgcattgaataaaaataaaatctttttttcattaaataaaatttatagagtaggaattaaaaattattcaattccagaaattaaaattgttttgaaaattttatataagtttgatttatatttgagaatttattctaaaaatattcatcaaaatttgtcatattttttaatattctcaattttaaagcttaataataattttttaatgcattattctacagaatcaaaatttaatttttaa

SEQ ID NO: 14 - DNA, 990 bp, Borrelia burgdorferi; identical to SEQ ID NO: 13
  CDS (1)..(990)

SEQ ID NO: 15 - protein, 329 aa, Borrelia burgdorferi
MQAVYLLLGNEQGLKEAYLKELLIKMDAFKSEVSVTKIFLSELSAVGFAEKLFSNSFFSKKEIFIVYESELLKAGKDLELVCNSILKSNNKTVIFVSNSNTCNIDFKNKLKFIKKVFYEIPDDDKFTFVKRNFFNLNIKITDSAINLMLLMLNSDTKILKFYIDSFALFAKNNTIEEEDIASWISFIRFENTFSLFNSILRKDMTQSLIKIKSILDQGEDLLNILMSLIWQFKRLLKVQIDYNAYGSLQSALNKNKIFFSLNKIYRVGIKNYSIPEIKIVLKILYKFDLYLRIYSKNIHQNLSYFLIFSILKLNNNFLMHYSTESKFNF

SEQ ID NO: 16 - DNA, 966 bp, Campylobacter jejuni
atgtatagaaaagagcttcaaatcttactttctaaagattcaatacctaattttttctttctttatggagcagataattttcaaagcgaactttatgctgaatttataaaagaaaaatataagcccgatgaaactttgaaacttttttttgaagaatacaacttcactcgtgcgagtgattttttaagcgcaggatcgctttttagtgaaaaaaaattactcgaaattaaaacttctaaaaaaattccaacaaaagatcttaaagttttagtcgaactttgtaaaaacaatacggacaatttctttcttttagagctttatgatgaaagttctaaacaaagcgatatagaaaaaattttctctcctcattttgtaagattttttaaagctaacggagctaaagaaggcgtggagcttttaagcataaaagcaaaacaactaggggttgaaatcactcaaaatgctttatttactctttttacaagttttgatgaaaacttatatcttgctgcgagtgagcttaataaatttagtggtttaagagtagatgaaaaaaccatagaacaatactgctatagtttaaatacaggaagttttgaaagcttttttgataaaattttaaaaaaacaagactttaaaagcgaacttgaaaaaattttagataattttaatgaaattgctttaatcaactctttatataactctttttatcgtttatttaaaattgcactttatgctaaaatcaatggtaaaattgattttaaagagcttttaggctacactccaccccctcaagtgggacaaaatttaagctctcaagcctttagtttaaaaatcgaacaatataaagaaattttcactcttttgcttaaaagcgaatacgaacttaaaaccaatcctaaactcgtaaaaaaagaatttttgatttcaaatttacttaaacttgcaaggattttaaaaaattaa

SEQ ID NO: 17 - DNA, 966 bp, Campylobacter jejuni; identical to SEQ ID NO: 16
  CDS (1)..(966)

SEQ ID NO: 18 - protein, 321 aa, Campylobacter jejuni
MYRKELQILLSKDSIPNFFFLYGADNFQSELYAEFIKEKYKPDETLKLFFEEYNFTRASDFLSAGSLFSEKKLLEIKTSKKIPTKDLKVLVELCKNNTDNFFLLELYDESSKQSDIEKIFSPHFVRFFKANGAKEGVELLSIKAKQLGVEITQNALFTLFTSFDENLYLAASELNKFSGLRVDEKTIEQYCYSLNTGSFESFFDKILKKQDFKSELEKILDNFNEIALINSLYNSFYRLFKIALYAKINGKIDFKELLGYTPPPQVGQNLSSQAFSLKIEQYKEIFTLLLKSEYELKTNPKLVKKEFLISNLLKLARILKN

SEQ ID NO: 19 - DNA, 1053 bp, Caulobacter crescentus
atgatcctgtccaagcgtccggacgtcgaacgcttcctcaaggagccgaccccggacatccgggccgtagtgatctacggcaaggatcggggcgtcgtgcgggaccgggccaatgcgctggcccggcgcgtggtcgagcgcccggatgatcccttcgatacggcgcaactgaccgacggcgatctcgacgccgacccggccaagcttgaggacgaactgtcggcgatgtcgctgatgggggggcggcgcctggtgcgcctgcgactgtccagcgacaaggctggccccgacaagcaggccgccgaagccctgacccgccatgtcgagggtcaactcaatccggacgccttctttctggtcgaggccggcgcgctgggcagagactcacagcttcgcaaggtcggcgagaaggccaacggctgcgccgtcatcccctgttacgaggacgaggccggggacctagcccgcctgacgcgcgacaccctggccaacgacaaggtcagcgtgaacagcgaggcgctggaactcttcgtttcgcgcctacccaaggaacggggcgtcgcccgcgccgagatcgaacggctggccctctacctcggcccgggcagcggtatcaacgccaccgccgcagacctgaccgacttcctgggcgtcgaacccgaggcctcccttagtgacgccgccgccgatgcgttcggcggaaaggtcggcgcggcccagcaggcgcttcgccgcgccgccgccgagggggaaggcggaccggccgccgtcagggcgatgggctactatctgggtcgcctgcgtcgcgtcctgaccctgcacaagaacggcgtcgacctgcaagccgccgccaaggccgccggcgtgttctggaagcaggagcgagaattcctccgtcaggcgcgagcctggaacctggaggccattctcgagatccagggcgagatcctgaccgccgaccgagcctgcaagacgtcgggctcccccgatcaactgatcgccgagcgtctggccctgatgatcgccggccgcgcccgccgactgggcctgtag

SEQ ID NO: 20 - DNA, 1053 bp, Caulobacter crescentus; identical to SEQ ID NO: 19
  CDS (1)..(1053)

SEQ ID NO: 21 - protein, 350 aa, Caulobacter crescentus
MILSKRPDVERFLKEPTPDIRAVVIYGKDRGVVRDRANALARRVVERPDDPFDTAQLTDGDLDADPAKLEDELSAMSLMGGRRLVRLRLSSDKAGPDKQAAEALTRHVEGQLNPDAFFLVEAGALGRDSQLRKVGEKANGCAVIPCYEDEAGDLARLTRDTLANDKVSVNSEALELFVSRLPKERGVARAEIERLALYLGPGSGINATAADLTDFLGVEPEASLSDAAADAFGGKVGAAQQALRRAAAEGEGGPAAVRAMGYYLGRLRRVLTLHKNGVDLQAAAKAAGVFWKQEREFLRQARAWNLEAILEIQGEILTADRACKTSGSPDQLIAERLALMIAGRARRLGL

SEQ ID NO: 22 - DNA, 945 bp, Chlamydia muridarum
atggaaacctgccaaaatagtgtccgcatagagtctataaaagactttattcatcattttgaaaatcaacgctttaaagtgtttataattggctcttcatttcccgaagacaaggatatttttctggaactttatgtttccggaagagggcgactatttgatggacaacgtttagttaaacaagagcttttatcttggactgataattttgggttgttcacttcaaaggaaactattggtatttatcaagttgagaagatgagctcctctttacaagagtttatcataagctatgctcatcatcctaatccgcatctcacattatttcttttcacaagtaaagcagagttcttctcagctttttcatcgaagctccctaatgccctttgtttatctctctttggtgagtattttgcagaacgcgactctcgtatagcacaggtattgatcaagcgttcaatggatgttcaactgtcttgttctttaggggtcgctaagcttttcgtaagtaagttcccgcaggttggagtttttgaaatttttagtgaatttcaaaagttgatttgtcaaattgggggcaagagttgtttagaagcttcagacgttcaatcgtttgtagaaaaaagagaatctgcttctttatggaaattacgggatgctcttcttcgtagagatctctcaacagggcaaagtttgatgcaatctctggtatctgatttgggagaagatcctcttgctattctcaattttttacgtagccaatatttatctgggcttcgagcaatagcggaacagtcgaaagatcgcaaaacacaaatctttttcgcagcaggagagcaggctcttttgaatggattaaacttattttttcatgcggagagtttgattaagaataacgtgcaagatcccattctttctttagaaaccatgattgcaagactgtaa

SEQ ID NO: 23 - DNA, 945 bp, Chlamydia muridarum; identical to SEQ ID NO: 22
  CDS (1)..(945)

SEQ ID NO: 24 - protein, 314 aa, Chlamydia muridarum
METCQNSVRIESIKDFIHHFENQRFKVFIIGSSFPEDKDIFLELYVSGRGRLFDGQRLVKQELLSWTDNFGLFTSKETIGIYQVEKMSSSLQEFIISYAHHPNPHLTLFLFTSKAEFFSAFSSKLPNALCLSLFGEYFAERDSRIAQVLIKRSMDVQLSCSLGVAKLFVSKFPQVGVFEIFSEFQKLICQIGGKSCLEASDVQSFVEKRESASLWKLRDALLRRDLSTGQSLMQSLVSDLGEDPLAILNFLRSQYLSGLRAIAEQSKDRKTQIFFAAGEQALLNGLNLFFHAESLIKNNVQDPILSLETMIARL

SEQ ID NO: 25 - DNA, 945 bp, Chlamydia trachomatis
atgggaaacagccagaatagtattcacataacgtctactaaagattttgttcagtatattgaacgtgaacgatttagagtgattgtgattggctcttcatctcttgaggacaaagatatcttttcggaactttatatttctggaaggaagagcttttttgacggacagcgtttgttgcaacaagagctcttatcttggacggatcattttggtttgttcgcttcccaagagactataggcatttatcaagcagagaaaatgagctcttctctacaagagtttattatagattacactcggcatcctaacccgaatcttacgttgttccttttcacaaataaagcagagctgttctcttctttatcttcgaagctttctaatgctctttgtttatctttatttggagaatattttgctgagcgagatgctcgcatagctcaggttttgataaaacgttctaaagagtcgcagctttcttgctctttaggagttgcaaagatttttgtgagtaagtttcctcagacaggtttatttgaaattcttagtgaatttcaaaagttaatttgtcagatgggagacaaggaatctatagaagcttcggatattcaatcatttgtagagaaaaaagaggcgatctctctgtggaagttgcgagatgctcttcttcgtaaagaccgtgtcgctgcacacagtctgatgagatctttggtgtctgatatgggagaggagcctctggcgattctcaactttttacgtagtcagtacctcttaggactgagggcagtggcagagcagtcgaaagagcgtaaaacacaaatatttattgctgcaggagagccggctcttttaaatggtttgaacctgtttttccatacggaaagtttaattaagaataacttccaagatcccatgctttcattagaaatgctagtttcacgactttag

SEQ ID NO: 26 - DNA, 945 bp, Chlamydia trachomatis; identical to SEQ ID NO: 25
  CDS (1)..(945)

SEQ ID NO: 27 - protein, 314 aa, Chlamydia trachomatis
MGNSQNSIHITSTKDFVQYIERERFRVIVIGSSSLEDKDIFSELYISGRKSFFDGQRLLQQELLSWTDHFGLFASQETIGIYQAEKMSSSLQEFIIDYTRHPNPNLTLFLFTNKAELFSSLSSKLSNALCLSLFGEYFAERDARIAQVLIKRSKESQLSCSLGVAKIFVSKFPQTGLFEILSEFQKLICQMGDKESIEASDIQSFVEKKEAISLWKLRDALLRKDRVAAHSLMRSLVSDMGEEPLAILNFLRSQYLLGLRAVAEQSKERKTQIFIAAGEPALLNGLNLFFHTESLIKNNFQDPMLSLEMLVSRL

SEQ ID NO: 28 - DNA, 948 bp, Chlamydophila pneumonia
atgactctacctatgcaaaaatccttaacgagttttgatgacttttcccaggcgtatgcagagaaagtgcccgctatagctcttatagggagtgctttggaagacgataaagatgcgctgattgaattattagtctctgagagcttcaaagagctcggtggtcagggactcatgccagcaaccctcatgtcttggaccgagacgtttgcactctttcaagagcatgaaactttggggattattcatgcagagaaattccctctagcaactaaggaatttctaagccgctatgctcggaatcctcaacctcaccttacgattttgatcttcaccacaaaacaagaatgctttcgagaactgtcaaaagccttgccatcggctctttctttgagtttatttggtgagtggcccgcagatcgtcagaaaaggatcatacgcctcctgttgcaaagagctgagcgtgtggggatttcttgctctcaatcattggcatctttgtttttgcgtgcacttgcttcaacctctcttcctgatattctcagtgaattcgataagctactgtgctctgttggcaagaaaacgtccttggatcactctgatattaaagagctcgttgtcaaaaaagaaaaggcttccctatggaaatttcgagactctctattgaagagggatccggtagaaggtcaccagcagttgcattttctactcgaggatggtgaagatcccttggggattattactttccttcgtacccaatgtctctatggtttacgtagtattgaagagggatcgaaagaaaataaacaccgaatgttcgtcctttatggaaaggagagactacaccaagccttaaattctttattttatgcagaaaccttaattaaaaataatgtccaagatcctatagtagctgtggaaactttagtgattagaatggttaacctgtga

SEQ ID NO: 29 - DNA, 948 bp, Chlamydophila pneumonia; identical to SEQ ID NO: 28
  CDS (1)..(948)

SEQ ID NO: 30 - protein, 315 aa, Chlamydophila pneumonia
MTLPMQKSLTSFDDFSQAYAEKVPAIALIGSALEDDKDALIELLVSESFKELGGQGLMPATLMSWTETFALFQEHETLGIIHAEKFPLATKEFLSRYARNPQPHLTILIFTTKQECFRELSKALPSALSLSLFGEWPADRQKRIIRLLLQRAERVGISCSQSLASLFLRALASTSLPDILSEFDKLLCSVGKKTSLDHSDIKELVVKKEKASLWKFRDSLLKRDPVEGHQQLHFLLEDGEDPLGIITFLRTQCLYGLRSIEEGSKENKHRMFVLYGKERLHQALNSLFYAETLIKNNVQDPIVAVETLVIRMVNL

SEQ ID NO: 31 - DNA, 903 bp, Deinococcus radiodurans
atgccagtcctcgcctttaccggcaaccgttttctggctgacgaaaccctgcgcgacacgctgagtgcgcgtgggctcaacgcccgtgacctgccgcgcttttccggcgaggacgtgagcgccgagacgctggggccgcacctcgcgccgagtctctttggcgacggcggcgtagtcgtcgatttcgagggcctcaagcccgacaaggccctgctggagctgctttcctcggcgcccgtcacggtggccgtgctcgacgaggcgccgcccgcaacccggctcaagctctatcagaaggcgggcgaggttatcccctcggcggccccgagtaagcccggcgacgtgaccggctgggtggtcacgcgggccaaaaaaatgggcctgcggttggagcgcgacgcggcgagctatctggccgaggtgttcggtgccgacctggcgggcatcgcgggcgagttgaacaagctggagctgctcggcggcgcgctgaatcgagaacgggtgcagggcatcgtgggccgcgatccgcccggcgactcgtttgccatgctcggcgcggcgacggcgggccgccccggtgaagcggtgctgcaactgcgccgcctgctcggctcgggcgaagaccccttccggctgctcggcgcggtggtgtggcagtacagcctgattgcccgcacggtcggcctgctccaggaggaaggccgcgtgaatgacgcggtggccgctcagcgcctgggcgtcaagccctttccggcgaaaaaggcgctggaagtcgcgcggcgcctgagtgaaagccggattcgcagccacctgggcgccattctcgatgccgacctcgccatgaagcgcgggctggacccgcaagtcgcccttgagcggctgctggtcaagctgagcgtatag

SEQ ID NO: 32 - DNA, 903 bp, Deinococcus radiodurans; identical to SEQ ID NO: 31
  CDS (1)..(903)

SEQ ID NO: 33 - protein, 300 aa, Deinococcus radiodurans
MPVLAFTGNRFLADETLRDTLSARGLNARDLPRFSGEDVSAETLGPHLAPSLFGDGGVVVDFEGLKPDKALLELLSSAPVTVAVLDEAPPATRLKLYQKAGEVIPSAAPSKPGDVTGWVVTRAKKMGLRLERDAASYLAEVFGADLAGIAGELNKLELLGGALNRERVQGIVGRDPPGDSFAMLGAATAGRPGEAVLQLRRLLGSGEDPFRLLGAVVWQYSLIARTVGLLQEEGRVNDAVAAQRLGVKPFPAKKALEVARRLSESRIRSHLGAILDADLAMKRGLDPQVALERLLVKLSV

SEQ ID NO: 34 - DNA, 1035 bp, Haemophilus influenzae
atgaaccgcatttttccagagcaactaaatcatcatcttgcgcaaggcttggctagagtttatctcttgcaagggcaagatcctttattacttagtgaaactgaggatactatttgtcaagtggcaaacctgcaaggttttgatgaaaaaaatactattcaggttgatagccaaactgattgggcgcaacttatagaatcttgtcaatctataggattgttttttagtaaacaaatactaagcttgaatttacctgaaaatttcaccgcacttttgcagaaaaatctgcaagaacttatatccgtattgcataaagatgtcttgcttattttacaggtagcaaaattggctaaagggattgaaaagcaaacgtggtttattacacttaatcaatatgaaccaaatacaatactgataaattgtcaaacaccgacggtagaaaacttaccacgttgggtaaaaaatcgtaccaaagcaatgggattagacgctgacaacgaagcaatccaacaactttgttatagttatgaaaataacctgctcgcactcaaacaagctctgcagcttttagatttgctctatcctgaccataaacttaactacaatcgtgttattagcgtggtggaacaatcctcaatttttacaccatttcaatggattgatgcgttgcttgttggaaaggcaaatcgtgctaaacgtattttaaaaggtttacaagctgaagatgtgcaacctgttattttattacgcacactacagcgagaattatttacgttgcttgaactcacaaaaccacagcaacgtattgtgacaacggaaaaacttccaattcaacaaattaagactgaatttgaccgtctaaaaatctggcaaaatcgccgaccgctctttttaagtgcgattcaacgtttaacttaccagactctctacgaaattatccaagaactcgctaatattgaacgccttgctaaacaagaatttagtgatgaagtgtggatcaaactcgctgatttatctgtaaaaatttgtttgtag

SEQ ID NO: 35 - DNA, 1035 bp, Haemophilus influenzae; identical to SEQ ID NO: 34
  CDS (1)..(1035)

SEQ ID NO: 36 - protein, 344 aa, Haemophilus influenzae
MNRIFPEQLNHHLAQGLARVYLLQGQDPLLLSETEDTICQVANLQGFDEKNTIQVDSQTDWAQLIESCQSIGLFFSKQILSLNLPENFTALLQKNLQELISVLHKDVLLILQVAKLAKGIEKQTWFITLNQYEPNTILINCQTPTVENLPRWVKNRTKAMGLDADNEAIQQLCYSYENNLLALKQALQLLDLLYPDHKLNYNRVISVVEQSSIFTPFQWIDALLVGKANRAKRILKGLQAEDVQPVILLRTLQRELFTLLELTKPQQRIVTTEKLPIQQIKTEFDRLKIWQNRRPLFLSAIQRLTYQTLYEIIQELANIERLAKQEFSDEVWIKLADLSVKICL

SEQ ID NO: 37 - DNA, 1023 bp, Helicobacter pylori 26695
atgtatcgtaaagatttggaccattatttaaaacaacgcctccctaaagcggtgtttttgtatggggagtttgatttttttatccattattatattcaaacgattagcgcgctttttaaatgcgataaccctgacatagaaacttcgcttttttatgcgagcgattatgaaaaaagccagattgcgacccttttagagcaggattctttatttggagggagcagtttagtcgttttaaaactggattttgccctgcataagaaatttaaggaaaatgatatcaatctttttttaaaagctttagagcggcctagccataacaggcttatcatagggctttataatgctaaaagcgacaccacaaaatacaaatacactagcgatgctatcgttaaatttttccaaaaaagccccttgaaagatgaagccatttgcgcgcgcttttttatccctaaaacttgggagagtttgaaattcttgcaagaaagggctaattttttgcatttagacatcagcggccatcttttaaacgctctttttgaaattaataacgaagatttaggcgtttcgtttaacgatttagacaagctagcggttttaaacgcacccatcactttagaagacattcaagaattaagctccaatgcgggggatatggatttgcaaaagctcattttagggctttttttaaaaaaaagtgcgcttgatatttatgattatttgttaaaagagggcaaaaaagatgcggatattttaagggggttagagcgctatttttaccaactttttttatttttcgctcatattaaaacgaccggtttaatggacgctaaagaggttttaggctacgctccccctaaagaaattgccgaaaattacgctaaaaacgccttgcgtttgaaagaagccggctataagagggtttttgaaatttttaggttatggcacattcaaagcatgcaagggcaaaaggaattgggctttttgtatttgacctccattcaaaaaatcattaacccctaa

SEQ ID NO: 38 - DNA, 1023 bp, Helicobacter pylori 26695; identical to SEQ ID NO: 37
  CDS (1)..(1023)

SEQ ID NO: 39 - protein, 340 aa, Helicobacter pylori 26695
MYRKDLDHYLKQRLPKAVFLYGEFDFFIHYYIQTISALFKCDNPDIETSLFYASDYEKSQIATLLEQDSLFGGSSLVVLKLDFALHKKFKENDINLFLKALERPSHNRLIIGLYNAKSDTTKYKYTSDAIVKFFQKSPLKDEAICARFFIPKTWESLKFLQERANFLHLDISGHLLNALFEINNEDLGVSFNDLDKLAVLNAPITLEDIQELSSNAGDMDLQKLILGLFLKKSALDIYDYLLKEGKKDADILRGLERYFYQLFLFFAHIKTTGLMDAKEVLGYAPPKEIAENYAKNALRLKEAGYKRVFEIFRLWHIQSMQGQKELGFLYLTSIQKIINP

SEQ ID NO: 40 - DNA, 1023 bp, Helicobacter pylori J99
atgtatcgtaaagatttggataattacttaaaacagcgcctccctaaagcggtgtttttgtatggggagtttgattttttcatccattattatattcaaacgattagcgcgctttttaaaggcaataaccctgacacagaaacttcgcttttttatgcgagcgattatgaaaaaagccagattgcgacccttttagagcaggattctttatttggagggagcagtttagttattttaaaactggattttgcattgcataagaaatttaaggaaaatgatatcaatccttttttaaaagctttagagcggcctagccataataggcttatcatagggctttataatgctaaaagcgacaccacaaaatacaaatacactagcgaaattatcgttaaatttttccaaaaaagccccttgaaagatgaagccatttgcgtgcgcttttttacccctaaagcgtgggagagtttgaaattcttgcaagaaagggctaattttttgcatttagacatcagcggccatcttttaaacgctctttttgaaattaataacgaagatttaagcgtttcgtttaacgatttagacaagctagcggttttaaacgcgcccatcactttagaagacattcaagaattaagctccaatgcgggggatatggatttgcaaaagctcattttagggctttttttgaaaaaaagcgtccttgatatttatgattatttgttaaaagagggcaaaaaggatgcggatattttaagggggttagagcgctatttttaccagctttttttatttttcgcccacattaaaacgaccggtttaatggacgctaaagaggtcttaggctacgctcctcctaaagagattgtagaaaattacgctaaaaacgccctgcgtttgaaagaagccggctataagagggtttttgaaatttttaggttatggcaccttcaaagcatgcaagggcaaaaggaattgggctttttgtatttgacccccattcaaaaaatcattaacccttga

SEQ ID NO: 41 - DNA, 1023 bp, Helicobacter pylori J99; identical to SEQ ID NO: 40
  CDS (1)..(1023)

SEQ ID NO: 42 - protein, 340 aa, Helicobacter pylori J99
MYRKDLDNYLKQRLPKAVFLYGEFDFFIHYYIQTISALFKGNNPDTETSLFYASDYEKSQIATLLEQDSLFGGSSLVILKLDFALHKKFKENDINPFLKALERPSHNRLIIGLYNAKSDTTKYKYTSEIIVKFFQKSPLKDEAICVRFFTPKAWESLKFLQERANFLHLDISGHLLNALFEINNEDLSVSFNDLDKLAVLNAPITLEDIQELSSNAGDMDLQKLILGLFLKKSVLDIYDYLLKEGKKDADILRGLERYFYQLFLFFAHIKTTGLMDAKEVLGYAPPKEIVENYAKNALRLKEAGYKRVFEIFRLWHLQSMQGQKELGFLYLTPIQKIINP

SEQ ID NO: 43 - DNA, 1005 bp, Lactococcus lactis
atgacagtatttgatgaatttaatgaaatcaaaagaaaagggctccctcaaattttagtaatttttggtgagtcaagggaactagttggagaattgaaaaatcaactcttaacagaggttaatttcgagccaacggatttgagccaggcttattatgacttaactcctagtaatagcgatttagcccttgaagatttggaatctctcccttttttctcagattctcggttggtaatcctggaaaacttggttaatttaacaacggctaaaaaaagtgttttagatgaaaaacagttgaagcgctttgaaaattttttagatgatccgccggaaactacgcaacttgttttaatcctttatggaaaattggacagtcgtcttaaagttgttaaaaaattaaaagctaaagcaactttacttgaagcacaagaattaaagagtcaagaattaattcaatattttagtcgaataagtcctttaccaaaggtagttttgagtttaattgccgctaaatcaaatgataatttttcagtaatgaagcaaaatattgacctcgttcaaacttatgcactagggcgagaagtgacacttgatgatgtggaaaaagtcgtcccaaaatcgctacaagataatatttttgccttaactgatttaatttttaaaggaaaaattgatgaggcgcgtgatttagtccatgatttgaccttgcaaggggaagatttaattaaaatattagctattttgacaaattcttatcgtttgtatttacaagtcaaacttttccaagccaagggttggcaagaaaatcaacaagttgcctttttaaaaatgcacccttatcctgtaaaattggccaatcaattggttagaaaattaaacgttaaatccttaaaaaatggactttctgatttaattaaacttgattttgatataaaaacaagtgcagctgataaatcctatctttttgatatcactttaatcaggttgaccttgaaaaaaaactga

SEQ ID NO: 44 - DNA, 1005 bp, Lactococcus lactis; identical to SEQ ID NO: 43
  CDS (1)..(1005)

SEQ ID NO: 45 - protein, 334 aa, Lactococcus lactis
MTVFDEFNEIKRKGLPQILVIFGESRELVGELKNQLLTEVNFEPTDLSQAYYDLTPSNSDLALEDLESLPFFSDSRLVILENLVNLTTAKKSVLDEKQLKRFENFLDDPPETTQLVLILYGKLDSRLKVVKKLKAKATLLEAQELKSQELIQYFSRISPLPKVVLSLIAAKSNDNFSVMKQNIDLVQTYALGREVTLDDVEKVVPKSLQDNIFALTDLIFKGKIDEARDLVHDLTLQGEDLIKILAILTNSYRLYLQVKLFQAKGWQENQQVAFLKMHPYPVKLANQLVRKLNVKSLKNGLSDLIKLDFDIKTSAADKSYLFDITLIRLTLKKN

SEQ ID NO: 46 - DNA, 1170 bp, Mycobacterium leprae
gtgcctgcggcaatcgttcccggtccggctagtgccctcggcgtgctgctgctggtgttggtcctgtggcggtgctttcgggtggcgatgatttcagcactgatggtcgccgttgcgtgcttgcctgattggttgtcagggttcctgaccggcgggcttatcgcgggctcgtcggctcgtcgtgccaccatctacggagtgagtaataagttttcgtcgctgcacttggtcctaggaaatgaggaactgctggttgagcgggccgtgggggaggtgttgcggtcggcgcggcagcgggcaggcacccaggacgtccccgttagccgaatgcgagccggtgacgtcggcacctatgagctcaccgaactgctgagcccgtcattgttcgccgacgagcggattgtcgtgctagaggccgccgctgaagcgggtaaggaggccgctgcgctgatcgtgtcggctgccgctgacataccgcaaggcacggtgctggtggtggtgcattcaggtggtggtcgagccaaagcgctggctaacgagctgcagtcgttgggtgctacggtgcatccctgcgcgcggatcactaagcttagcgagcgtactgattttgttcgaaaagaattgcgttcgttgcgggtcaaggtcgacgagggggcagtgaccgccctgctgaacgctgttggttctgacgtgcgcgaactcgcttcggcctgttcgcaattggtagccgacaccgccggtgatgtcgacgccgacgcggttcagcgttatcacagcggcaaagcggaagtgaagggctttgacattgccgacaaggcggtcggtggcgatgtctcaggagcagtggaggcgttgcggtgggcgatgatgcgcggcgagccactggtggtgttggctgatgcgctggccgaagcagtgcacacgatcggccgggttgggccgctgtccggtgattcctaccgcctggcgtcgcggttgggaatgccgccctggcgggtgcagaaggcacaacaacaggctcggcgttggtcgcgtgacacagtggcagccgcgatgagagtggtggctgcgctcaacgccgacgtgaaaggggccgccgcggacgcctactacgctctggaatccgcggtcaggaaagttgctgagctggccgccgatgggagccgatga

SEQ ID NO: 47 - DNA, 1170 bp, Mycobacterium leprae; identical to SEQ ID NO: 46
  CDS (1)..(1170)

SEQ ID NO: 48 - protein, 389 aa, Mycobacterium leprae
VPAAIVPGPASALGVLLLVLVLWRCFRVAMISALMVAVACLPDWLSGFLTGGLIAGSSARRATIYGVSNKFSSLHLVLGNEELLVERAVGEVLRSARQRAGTQDVPVSRMRAGDVGTYELTELLSPSLFADERIVVLEAAAEAGKEAAALIVSAAADIPQGTVLVVVHSGGGRAKALANELQSLGATVHPCARITKLSERTDFVRKELRSLRVKVDEGAVTALLNAVGSDVRELASACSQLVADTAGDVDADAVQRYHSGKAEVKGFDIADKAVGGDVSGAVEALRWAMMRGEPLVVLADALAEAVHTIGRVGPLSGDSYRLASRLGMPPWRVQKAQQQARRWSRDTVAAAMRVVAALNADVKGAAADAYYALESAVRKVAELAADGSR

SEQ ID NO: 49 - DNA, 969 bp, Mycobacterium tuberculosis
gtgagcgaggctaagccgttgcacctggtcctgggagacgaagaactgctggtcgaaagggcggtggccgacgtgttgcgctcggctcggcagcgggcaggtacagccgacgtcccggtgagccgaatgcgcgcgggtgacgtcggtgcctatgagctcgccgaactgctgagcccgtcactgttcgccgaggagcggatcgttgtgctgggggccgctgcggaggcgggcaaggacgctgccgcggtaatcgagtcggccgccgccgatcttccggccggcaccgtgctggtagtggtccactcgggtggcgggcgcgccaaatcgctggccaaccagctgcggtcgatgggtgcgcaggttcatccgtgcgcgcggatcaccaaggtcagtgagcgcgccgacttcatccgtagcgagttcgcgtcgctgcgggtcaaggtcgacgacgagaccgtgaccgccctgctggacgccgtcggctccgacgtgcgcgaactcgcctcggcctgttcacagctggtcgccgataccggaggagccgtcgacgccgccgctgtacggcgctatcacagcggcaaagccgaggtgaggggcttcgacatcgccgacaaggcggtagccggcgacgtggcgggagctgccgaagcgttgcggtgggcgatgatgcgcggtgagccgctagtggtgttggccgatgcgctcgccgaagccgtgcacaccatcggccgggtcgggccgcagtccggcgacccgtaccgcctggccgcacaactggggatgccgccctggcgggtgcagaaagcccagaagcaggctcggcggtggtcgcgtgacacggtggcgaccgcgatgaggttggtggccgaactcaatgctaacgtcaagggcgccgtcgcggatgcggactacgcgctggaatccgcggtccggcaggtcgccgagttggtggccgaccgcggccgatga

SEQ ID NO: 50 - DNA, 969 bp, Mycobacterium tuberculosis; identical to SEQ ID NO: 49
  CDS (1)..(969)

SEQ ID NO: 51 - protein, 322 aa, Mycobacterium tuberculosis
VSEAKPLHLVLGDEELLVERAVADVLRSARQRAGTADVPVSRMRAGDVGAYELAELLSPSLFAEERIVVLGAAAEAGKDAAAVIESAAADLPAGTVLVVVHSGGGRAKSLANQLRSMGAQVHPCARITKVSERADFIRSEFASLRVKVDDETVTALLDAVGSDVRELASACSQLVADTGGAVDAAAVRRYHSGKAEVRGFDIADKAVAGDVAGAAEALRWAMMRGEPLVVLADALAEAVHTIGRVGPQSGDPYRLAAQLGMPPWRVQKAQKQARRWSRDTVATAMRLVAELNANVKGAVADADYALESAVRQVAELVADRGR

SEQ ID NO: 52 - DNA, 1044 bp, Mesorhizobium loti
atggcacagaagaaagctttcgaggtcgattcctggctcgcgcggcctgacgcgcgcatctccatcgtcctgctctacggccccgatcgcggcctcgtctccgagcgtgcaaaggcctttgccgaaaagaccggcctgccgctcgacgatccgttttccgtggtccggctcgaggggtcggaggtcgaccgcgacgaaggccggctgctcgacgaagcccgcaccgtgccgatgttttccgaccgccgcctgctctgggtgcgcaatgccaccggccagaaggcgctggccgaggacgtcaaggcgctgacagcggagcccgcgcgcgatgccatcatcctcatcgaggccggcgacctcaagaagggcgtcggcttgcgcgccgtggtcgagggcgccgacaatgccatggcgctgccttgctacgccgacgaggcccgcgacatcgacacggtcatcgacgatgaattgcgcaaggccggcatgtcgatgacgctggaggcgaggcaggcgctgcgccgcaatctcggcggcgacaggctcgcttcgcgcggcgagatcgaaaaactcatcctctacgcccacggccagaaggagatcggcctggaggatgtcagggcgatgtcgggcgacgtgtccggcacctcgttcgacgacgccgtcgacgcgctgctggaaggcaaggtcggcgacttcgacaccgccttcacccgccactgccagtccggcggcccacccttcctggtgctgtcctcggcgatgcgccagctgcagcagatccaaatcatgcgcggccagatggactctggcgggcgcaatgccgcgtcggtggtcgccgccgcgcgcccgccggttttcttttcgcggcgcaagctggtggagaaggcgctcgagcgctggagcagcgatgcgctgagccgcgcgctaacccggctgcagaccgcggtgctgcagacccgcaaacggcccgatctgtcagaggcgctggcgcgccaggcgctgctcggcattgcggtggagagcgcgaggctggggcagcgttag

SEQ ID NO: 53 - DNA, 1044 bp, Mesorhizobium loti; identical to SEQ ID NO: 52
  CDS (1)..(1044)

SEQ ID NO: 54 - protein, 347 aa, Mesorhizobium loti
MAQKKAFEVDSWLARPDARISIVLLYGPDRGLVSERAKAFAEKTGLPLDDPFSVVRLEGSEVDRDEGRLLDEARTVPMFSDRRLLWVRNATGQKALAEDVKALTAEPARDAIILIEAGDLKKGVGLRAVVEGADNAMALPCYADEARDIDTVIDDELRKAGMSMTLEARQALRRNLGGDRLASRGEIEKLILYAHGQKEIGLEDVRAMSGDVSGTSFDDAVDALLEGKVGDFDTAFTRHCQSGGPPFLVLSSAMRQLQQIQIMRGQMDSGGRNAASVVAAARPPVFFSRRKLVEKALERWSSDALSRALTRLQTAVLQTRKRPDLSEALARQALLGIAVESARLGQR

SEQ ID NO: 55 - DNA, 894 bp, Mycoplasma genitalium
atgacaataatttatggtcaagatattgggctaattcaccaaaaattaagtcaaatcaaaagcaattctccatataaaactatttggtttaaagatcttaagcaactttatgatctcttttcacaaccattatttggttctaataatgaaaagtttattgtcaataattgcagttttttggaaaaaacaagcttaactagacaagaaaacttgtgtttagaaaaactaaaaacaactgatgtagttttaactgtttatactgacaatccttttagtgggattaaaaccattaaatcaatcacaactgttttttgtgataaattggattggaaatcaatgcataaagcaattggtgaagtttgtagagaacttaacttaaaacttgatttggaaataattgattggttagcgaatgctttacctttaaatatgggggtaatttaccaagaaattaacaaacttagcttgttaggtaaaaatgaaatcaaagataacaaattagttgaaacagttatttgtgattatcaacctgttcaaatttacaaattaaccaaagctctaacaaattcacaaattgtaaaggcatttaaatatattgatgaattggcaagtacaaaaccaaattttgccacccaatttttagaattttttagtggggagttgttattagcattgatggttaaaagttgtaatccaaagcaactaactaacattaatctaaatgttaatcagtttcgcttaattgctattcaaagtcagtaccataactttactagcaaagttttagtaaatataattaatgcaattcaaaaattggatattaaattaaagcacaatgatggctttgccattccgttgttaaaaaactttgctttaagcttttttactaactaa

SEQ ID NO: 56 - DNA, 894 bp, Mycoplasma genitalium; identical to SEQ ID NO: 55
  CDS (1)..(894)

SEQ ID NO: 57 - protein, 297 aa, Mycoplasma genitalium
MTIIYGQDIGLIHQKLSQIKSNSPYKTIWFKDLKQLYDLFSQPLFGSNNEKFIVNNCSFLEKTSLTRQENLCLEKLKTTDVVLTVYTDNPFSGIKTIKSITTVFCDKLDWKSMHKAIGEVCRELNLKLDLEIIDWLANALPLNMGVIYQEINKLSLLGKNEIKDNKLVETVICDYQPVQIYKLTKALTNSQIVKAFKYIDELASTKPNFATQFLEFFSGELLLALMVKSCNPKQLTNINLNVNQFRLIAIQSQYHNFTSKVLVNIINAIQKLDIKLKHNDGFAIPLLKNFALSFFTN

SEQ ID NO: 58 - DNA, 945 bp, Mycoplasma pneumoniae
atgggctgtggggcttgttgtatactgtgggtacagtcttttactatgaccgtagtttatggtgccgatattgggttaattcaccagcagttaaaccaactgctcaacccagccgcttgtaaacaggtctggtttcaagatgttaataagctgtatgatgtggtgttaaaccagaatctgtttgctgaggacactaaaccgatcctcattcacaactgcagttttttggaaaagaacaacttaactaaggctgaattacactgcttaaaaacacttaaagatactgatgtggtggtcaccatttacagcgacagtcccgctaatgccctcattaatgaccgggcaattaccaagtatgcttgtaaaccagtcacagctaaaaccatccaccaggtgatcagtaaggccgctaagacactcaaactcaacttaaaccctgatttaattgaccacttagccacgattctaccctttaacttaggggtcattgaacaggagttgcgtaagttaactctacttagcccggcagaattacaggacaaaaaaatgttagaagccgttttgtgtgattaccaaaccagtcaaatcttacaattaactgacgccatggtgcgtttgcaaacagctaaagccttaaagttgattgaacgcttgtttttaccaaagcagctcacaccaccgcaattcctagaatttttagcaaatgaactacttttggccttgatggtcaaaggcgttaaaccacaagctgttttccaactacagtggaatgtcaatccctttcgtttaaaggcgattcaaagccagtaccgcttttggtccaccaaccaattaacggccttaattaatgcgatttggcagctggatattaaaattaagcgcaatgatggcttagcaatccatttgctaaagcacttcgtgttgcgattctttgcgcaaaagtaa

SEQ ID NO: 59 - DNA, 945 bp, Mycoplasma pneumoniae; identical to SEQ ID NO: 58
  CDS (1)..(945)

SEQ ID NO: 60 - protein, 314 aa, Mycoplasma pneumoniae
MGCGACCILWVQSFTMTVVYGADIGLIHQQLNQLLNPAACKQVWFQDVNKLYDVVLNQNLFAEDTKPILIHNCSFLEKNNLTKAELHCLKTLKDTDVVVTIYSDSPANALINDRAITKYACKPVTAKTIHQVISKAAKTLKLNLNPDLIDHLATILPFNLGVIEQELRKLTLLSPAELQDKKMLEAVLCDYQTSQILQLTDAMVRLQTAKALKLIERLFLPKQLTPPQFLEFLANELLLALMVKGVKPQAVFQLQWNVNPFRLKAIQSQYRFWSTNQLTALINAIWQLDIKIKRNDGLAIHLLKHFVLRFFAQK

SEQ ID NO: 61 - DNA, 999 bp, Neisseria meningitidis MC58
gtggcggcacatatcggacgcattgatacggacgcgcctttgaaacccctgtacgtcatccacggcgaggaagaactgttgcgtatcgaggcattggacgcattgagggcggcggcgaagaaacaaggttaccttaatcgggaagtttatacggcagacaatgccttcgattggaacgagctgctgcaaaccgcaggcagtgcgggtctgtttgccgatttgaagctgttggaactgcatatccctaacggcaagcccggcaaaaccggcggcgaggcgttgcaggattttgccgcccgattgccggaagatacggtaacgctggttttgctgcccaaactggagaaaacccagctccagtccaaatggtttgccgcattggcggcaaagggggaagtgtgggaagccaaaccggtcggcgcggcggctttgccccaatggatacgcggacggctggacaaaatcggtttgggtatcgaggcagacgcattggcactgtttgctgagcgcgtggaaggcaatctgttggcggcgcgtcaggaaatcgacaagctcgggctgctgtatccgaaagggcataccgtcaatatcgatgaggcgcaaaccgccgttgccaacgtcgcccgcttcgacgcgttccaactggcaggcgcgtggatgaagggcgatgtcctgcgcgtatgcaggcttttggacggattgcgggaagagggcgaagaaccggtgctgttgctgtgggcggttgccgaagacgtgcggacgctgatccggcttgctgccgccctgaagcaggggcagagcatccaatccgtccgcaacagcctcaggctttggggcgacaagcagacgctcgcaccgcttgcggtcaagcggatttccgtcgtccgcctgcttgacgcgctcaaaacctgcgcccaaatcgaccgaatcatcaaaggtgcggaagacggcgacgcatggacggtattcaaacggcttgtcgtgtcgctggcggaataa

SEQ ID NO: 62 - DNA, 999 bp, Neisseria meningitidis MC58; identical to SEQ ID NO: 61
  CDS (1)..(999)

SEQ ID NO: 63 - protein, 332 aa, Neisseria meningitidis MC58
VAAHIGRIDTDAPLKPLYVIHGEEELLRIEALDALRAAAKKQGYLNREVYTADNAFDWNELLQTAGSAGLFADLKLLELHIPNGKPGKTGGEALQDFAARLPEDTVTLVLLPKLEKTQLQSKWFAALAAKGEVWEAKPVGAAALPQWIRGRLDKIGLGIEADALALFAERVEGNLLAARQEIDKLGLLYPKGHTVNIDEAQTAVANVARFDAFQLAGAWMKGDVLRVCRLLDGLREEGEEPVLLLWAVAEDVRTLIRLAAALKQGQSIQSVRNSLRLWGDKQTLAPLAVKRISVVRLLDALKTCAQIDRIIKGAEDGDAWTVFKRLVVSLAE

SEQ ID NO: 64 - DNA, 999 bp, Neisseria meningitidis Z2491
gtggcggcacatatcggacgcattgatacggacgcgcctttgaaacctctgtacgtcatccacggcgaggaagaactgttgcgtatcgaggcattggacgcattgagggcggcggcgaagaaacaaggttaccttaatcgggaagtttatacggcagacaatgccttcgattggaacgaactgttgcaaaccgcaggcagtgcgggtctgtttgccgatttgaagctgttggaactgcatatccccaacggaaaacccggcaaaaccggcggcgaggcgttgcaggattttgccgcccgattgccggaagatacggtaacgctggttttgctgcccaaactggagaaaacccagctccagtccaaatggtttgccgcattggcggcaaagggggaagtgtgggaagccaaaccggtcggcgcggcggctttgccccaatggatacgcggacggctggacaaaatcggtttgggtatcgaggcagacgcattggcactgtttgctgagcgcgtggaaggcaatctgttggcggcgcgtcaggaaatcgacaagctcgggctgctgtatccgaaagggcataccgtcaatatcgatgaggcgcaaaccgccgttgccaacgtcgcccgcttcgacgcgttccaactggcaggcgcgtggatgaagggcgatgtcctgcgcgtatgcaggcttttggacggattgcgggaagagggcgaagaaccggtgctgttgctgtgggcggttgccgaagacgtgcggacgctgatccggcttgctgccgccctgaagcaggggcagagcatccaatccgtccgcaacagcctcaggctttggggcgacaagcagacgctcgcaccgcttgcggtcaagcggatttccgtcgtccgcctgcttgacgcgctcaaaacctgcgcccaaatcgaccgaatcatcaaaggcgcggaagagggcgacgcatggacggtattcaaacggcttgtcgtgtcgctggcggaataa

SEQ ID NO: 65 - DNA, 999 bp, Neisseria meningitidis Z2491; identical to SEQ ID NO: 64
  CDS (1)..(999)

SEQ ID NO: 66 - protein, 332 aa, Neisseria meningitidis Z2491
VAAHIGRIDTDAPLKPLYVIHGEEELLRIEALDALRAAAKKQGYLNREVYTADNAFDWNELLQTAGSAGLFADLKLLELHIPNGKPGKTGGEALQDFAARLPEDTVTLVLLPKLEKTQLQSKWFAALAAKGEVWEAKPVGAAALPQWIRGRLDKIGLGIEADALALFAERVEGNLLAARQEIDKLGLLYPKGHTVNIDEAQTAVANVARFDAFQLAGAWMKGDVLRVCRLLDGLREEGEEPVLLLWAVAEDVRTLIRLAAALKQGQSIQSVRNSLRLWGDKQTLAPLAVKRISVVRLLDALKTCAQIDRIIKGAEEGDAWTVFKRLVVSLAE

SEQ ID NO: 67 - DNA, 1080 bp, Pasteurella multocida
atgcaacggattttcacagaacaattaggatctgctctacaaaacaaactagccaagctctattatttcgtagggcaagatccgttactgttaagtgaaaatcaagatcgcataaatcaatatgcattcaagcaaggttttgacgaaaaaaatgtcatcacaattgatcagaatactaattggtctgatctctttgaacgctgccaatccattggcttgtttttcaataaacagattttgtttcttaatttaccggaaaacctcagtgcaagtttacaaactcgtttacaagaactgatctcactcctcaacgatgacatcctgttaatactacaattgcctaaactgaacaaagcgactgaaaaacaaacatggtttatccaagctaatcagtatgaaccggaagccgtactcgtgcattgccaaaccccaaccattgaacaattgcctcgttggatcgcgttgcgtgcgaaaagtatgggactacagattgaagagcaagcaatccaacttctttgttatagccatgaaaataacttattagccttaaaacaaagtttgcagctccttgctttactctatcctgacaacaaaatcagttatgcacgagtacaatccactatcgctcagacctcagtgttcacaccatttcaatggctcgatgcgctattagaaggcaaaagtaaacgcgcccaacgtattttagaaggattaaaagcagaggaaatggagcccattattttattgcggacgttacaacgagatttgctcaccttattaggattggcgaagccatcacatcctgtcacccttaccacgcccctcccagctcatcaactgcgtgaacaatttgatcaattaaaagtgtggcaaaatcgacgccctttctttactcaagcctttaaacgcttgaattacaaacagctttatttgattattcaagcgctagcagagatcgaacgtttagcgaaacacacatttagccaagatatttgggatcgtttagccgaactttctgctttcatttgtttaccagatgatcccaatcacctactatctctaccaactttctctcgttga

SEQ ID NO: 68 - DNA, 1080 bp, Pasteurella multocida; identical to SEQ ID NO: 67
  CDS (1)..(1080)

SEQ ID NO: 69 - protein, 359 aa, Pasteurella multocida
MQRIFTEQLGSALQNKLAKLYYFVGQDPLLLSENQDRINQYAFKQGFDEKNVITIDQNTNWSDLFERCQSIGLFFNKQILFLNLPENLSASLQTRLQELISLLNDDILLILQLPKLNKATEKQTWFIQANQYEPEAVLVHCQTPTIEQLPRWIALRAKSMGLQIEEQAIQLLCYSHENNLLALKQSLQLLALLYPDNKISYARVQSTIAQTSVFTPFQWLDALLEGKSKRAQRILEGLKAEEMEPIILLRTLQRDLLTLLGLAKPSHPVTLTTPLPAHQLREQFDQLKVWQNRRPFFTQAFKRLNYKQLYLIIQALAEIERLAKHTFSQDIWDRLAELSAFICLPDDPNHLLSLPTFSR

SEQ ID NO: 70 - DNA, 1038 bp, Pseudomonas aeruginosa
atgaagctgactcccgcgcaactcgccaagcacctccagggccccctggcgcccgtctatgtcgtcagcggcgatgaaccgctgctctgccaggaagcctgcgatgccattcgccaggcctgccgcgagcgcgacttcggcgagcgccaggtattcaatgccgaagccaacttcgattggggcttgctgctggaggccggcgccagcctgtcgctgttcgccgagaagcgcctgatcgaactgcgcctgccctccggcaagcccggcgacaaaggcgcggcgattctccaggaatacctgcaacggccgccggaagacaccgtgctcctgctcggtctgcccaagctcgatggcagcacgcagaagaccaagtgggccaaggccctgatcgatggcaacgccgcgcagttcatccaggtctggccggtcgacgtccaccagttgccgcagtggattcgccagcgcctgtcccaggctggcctgtccgccagccccgaagccctggagctgatcgccgcgcgcgtggaaggcaacctgctcgccgccgcccaggaaatcgaaaagctcaagctgctcgccgagggcaaccagatcgacgccgccaccgtacaggccgcggtcgccgacagcgcacgcttcgatgtcttcggcctgatcgacgctgcccttggcggtgaagcggcccacgccctgcgcatcctggaaggactgcgcggcgaaggcatcgagccgccggtgatcctctggggcctggcccgcgagatccgcctgctggctggcctgtcccagcaatacggccagggcatcccgctggagaaagccttcgcccaggcgcgtccgccggtctgggacaagcgccgtccgctgctgacccgtgccctgcaacgccactccagcagccgctggaaccagatgctgagggatgcccagttgatcgacgcgcagatcaagggacaggcacccggcagcccctggagcggtctctcgcttctcgccctgagcctcgccggccagcgcctcggcctgccggcagcctga

SEQ ID NO: 71 - DNA, 1038 bp, Pseudomonas aeruginosa; identical to SEQ ID NO: 70
  CDS (1)..(1038)

SEQ ID NO: 72 - protein, 345 aa, Pseudomonas aeruginosa
MKLTPAQLAKHLQGPLAPVYVVSGDEPLLCQEACDAIRQACRERDFGERQVFNAEANFDWGLLLEAGASLSLFAEKRLIELRLPSGKPGDKGAAILQEYLQRPPEDTVLLLGLPKLDGSTQKTKWAKALIDGNAAQFIQVWPVDVHQLPQWIRQRLSQAGLSASPEALELIAARVEGNLLAAAQEIEKLKLLAEGNQIDAATVQAAVADSARFDVFGLIDAALGGEAAHALRILEGLRGEGIEPPVILWGLAREIRLLAGLSQQYGQGIPLEKAFAQARPPVWDKRRPLLTRALQRHSSSRWNQMLRDAQLIDAQIKGQAPGSPWSGLSLLALSLAGQRLGLPAA

SEQ ID NO: 73 - DNA, 966 bp, Rickettsia prowazekii
atgcaattttatttttcgcaaataggtagattatttgaattaatagcagccgggaagattagggctcttttactatatggtcctgataaaggctatatagaaaagatttgtacatatttaatcaaaaacttgaatatgctacaatcttctatagaatatgaagatcttaatattttatctttagatatattacttaattcacctaatttttttggtcaaaaagaattaattaaagtgagatcaattggaaattcactcgataagaatttaaaaacaattctaagcagtgattatataaatttccctgtatttataggggaagatatgaattctagtgggagtgttaaaaagttttttgaaacagaagaatatttagcggtagttgcttgttatcatgacgatgaagcaaaaattgaacgaattattttaggtaaattagcaaaaactaataaagttataagtaaagaagctatcacttacttaaaaactcatttaaaaggtgatcatgctttaatttgtagcgaaatcaataaattgatattttttgctcatgatgtacatgaaattactttaaaccatgttttagaagttataagtagtgaaattacagcaaatggtagtaatttggcaatatatttttcaaaaaaagattatagtaattttctgcaagagcttgatattttaaaaaaacaaaatattaatgaagttttaattattagagctttaataaggcattatttaaatttatatatagttttatcgaaagtaaaaaatggtgaatgtttggaaattgcaataaaatcattatcaccgccgattttctatcataatattaatgattttacaaagatagtacatgctctcagcctaactgaatgtataaagactttgaaactactacaacaggcagaagtggattataagttaaatcctgctagtttcgatttatttcaaagtatactctaa

SEQ ID NO: 74 - DNA, 966 bp, Rickettsia prowazekii; identical to SEQ ID NO: 73
  CDS (1)..(966)

SEQ ID NO: 75 - protein, 321 aa, Rickettsia prowazekii
MQFYFSQIGRLFELIAAGKIRALLLYGPDKGYIEKICTYLIKNLNMLQSSIEYEDLNILSLDILLNSPNFFGQKELIKVRSIGNSLDKNLKTILSSDYINFPVFIGEDMNSSGSVKKFFETEEYLAVVACYHDDEAKIERIILGKLAKTNKVISKEAITYLKTHLKGDHALICSEINKLIFFAHDVHEITLNHVLEVISSEITANGSNLAIYFSKKDYSNFLQELDILKKQNINEVLIIRALIRHYLNLYIVLSKVKNGECLEIAIKSLSPPIFYHNINDFTKIVHALSLTECIKTLKLLQQAEVDYKLNPASFDLFQSIL

SEQ ID NO: 76 - DNA, 1002 bp, Synechocystis
atgcccgtatatttctattggggcgaagaccaatttaccctgcaccaagcagtcaaacaactgcagaaacgttgtttagatccccaatgggaggcctttaattttgaaaaaattccgggggaacaagctgatgctacccaacggggtctggagcaagccttgacacccccttttggtagtggcgatcgcctggtgtgggtggtggactccaccttaggacaaagttgcgacgatggtttgctggctaggttgcaaaaaagtttgcccgccattcccaccgactgccatttactattcacatccagtaaaaagctcgatcgccgactgaaatcgaccaaatatttggaaggtaatgccactatccgggaatttgccctcatttcaccctggaatgtggacgctctcatccatcaaattcaggcgatcgcccaggatctgcaacttcccttagcaacggaaacggaaggttttttggcagaagcgttgggtaatgacacgaggctaatttggaatgagttgggcaagctcaaactctatagcgagagtcaaacgggccccttaacggtggcccaggtagaacagttggtcaataccagcacccaaaatagtttgcaattagcccaggcgatccgggatggagaaaccccccaggcattgcaattggtggcagacttattgctgacccacaacgaaccggctttaaaaatattggctaccctcaccggccagtttcgtacttggacattgatcaagcttgctttggaggcaggggaaaaagatgacaaagcgatcgccgcccaggccgatctcagtaatcccaaacgtttgtattttttaaagaaggaattgcggggcattaccggcgggcaattgctcaaaaccttacccctgttgttggaattggaatatcaactaaagcgaggagcggacccctgttcgagcctacaaactaaggtgattgaactatcatcattattccagcccatacaccgtcgccgttga

SEQ ID NO: 77 - DNA, 1002 bp, Synechocystis; identical to SEQ ID NO: 76
  CDS (1)..(1002)

SEQ ID NO: 78 - protein, 333 aa, Synechocystis
MPVYFYWGEDQFTLHQAVKQLQKRCLDPQWEAFNFEKIPGEQADATQRGLEQALTPPFGSGDRLVWVVDSTLGQSCDDGLLARLQKSLPAIPTDCHLLFTSSKKLDRRLKSTKYLEGNATIREFALISPWNVDALIHQIQAIAQDLQLPLATETEGFLAEALGNDTRLIWNELGKLKLYSESQTGPLTVAQVEQLVNTSTQNSLQLAQAIRDGETPQALQLVADLLLTHNEPALKILATLTGQFRTWTLIKLALEAGEKDDKAIAAQADLSNPKRLYFLKKELRGITGGQLLKTLPLLLELEYQLKRGADPCSSLQTKVIELSSLFQPIHRRR

SEQ ID NO: 79 - DNA, 990 bp, Thermotoga maritima
atgagaggtgattgtatgccagtcacgtttctcacaggtactgcagaaactcagaaggaagaattgataaagaaactcctgaaggatggtaacgtggagtacataaggatccatccggaggatcccgacaagatcgatttcataaggtctttactcaggacaaagacgatcttttccaacaagacgatcattgacatcgtcaatttcgatgagtggaaagcacaggagcagaagcgtctcgttgaacttttgaaaaacgtaccggaagacgttcatatcttcatccgttctcaaaaaacaggtggaaagggagtagcgctggagcttccgaagccatgggaaacggacaagtggcttgagtggatagaaaagcgcttcagggagaatggtttgctcatcgataaagatgcccttcagctgtttttctccaaggttggaacgaacgacctgatcatagaaagggagattgaaaaactgaaagcttattccgaggacagaaagataacggtagaagacgtggaagaggtcgtttttacctatcagactccgggatacgatgatttttgctttgctgtttccgaaggaaaaaggaagctcgctcactctcttctgtcgcagctgtggaaaaccacagagtccgtggtgattgccactgtccttgcgaatcacttcttggatctcttcaaaatcctcgttcttgtgacaaagaaaagatactacacctggcctgatgtgtccagggtgtccaaagagctgggaattcccgttcctcgtgtggctcgtttcctcggtttctcctttaagacctggaaattcaaggtgatgaaccacctcctctactacgatgtgaagaaggttagaaagatactgagggatctctacgatctggacagagccgtgaaaagcgaagaagatccaaaaccgttcttccacgagttcatagaagaggtggcactggatgtatattctcttcagagagatgaagaataa

SEQ ID NO: 80 - DNA, 990 bp, Thermotoga maritima; identical to SEQ ID NO: 79
  CDS (1)..(990)

SEQ ID NO: 81 - protein, 329 aa, Thermotoga maritima
MRGDCMPVTFLTGTAETQKEELIKKLLKDGNVEYIRIHPEDPDKIDFIRSLLRTKTIFSNKTIIDIVNFDEWKAQEQKRLVELLKNVPEDVHIFIRSQKTGGKGVALELPKPWETDKWLEWIEKRFRENGLLIDKDALQLFFSKVGTNDLIIEREIEKLKAYSEDRKITVEDVEEVVFTYQTPGYDDFCFAVSEGKRKLAHSLLSQLWKTTESVVIATVLANHFLDLFKILVLVTKKRYYTWPDVSRVSKELGIPVPRVARFLGFSFKTWKFKVMNHLLYYDVKKVRKILRDLYDLDRAVKSEEDPKPFFHEFIEEVALDVYSLQRDEE

SEQ ID NO: 82 - DNA, 765 bp, Treponema pallidum
atgtccgtctggctttttaccggacctgaaataggggagcgagatagtgcagttcaggaggtgtgcgcgcgtgcacaagcgcaagggacggtggacgtacatcggctctatgcacacgaaacgcctgtggcagatcttgtagatctcctgagaactcgtgcgctgtttgcagatgcagtctgcgtggtactgtacaacgcagaggttattaagaagtgtgacgaagtgcacgtgcttacagaatggataaaggatggcggctcgcgcgctgacgtgtttttggtactgatttccgatagtgtcagtatacataagcgtatagaacagaatatttctcccgtgcataagcgtgttttttgggagttgtttgaaaataaaaaacacgcgtgggtgcagcgatttttttttcagcatgaaatgcgcattgaacaggaggctatcgaatctcttcttgagttggtggagaacaacactcgtgcgctcaaaactgtttgtacgcagctttctcttttttttgaaaaaggacgccgcatcactgcgcacgacattagttcgttgctggtgcacacaaaagaagagacatcgtttaccttattcgatgcgctgtccaaacgagatcttgagcattcacttatgattctcaacacgctgctttgttcgaaagacgttgcccctgtacaaatccttgcaggactgcttacgcgttccgtcgccttgcgcattggcaccatataccggaggacaaaacgcaccctgcaacgctaa

SEQ ID NO: 83 - DNA, 765 bp, Treponema pallidum; identical to SEQ ID NO: 82
  CDS (1)..(765)

SEQ ID NO: 84 - protein, 254 aa, Treponema pallidum
MSVWLFTGPEIGERDSAVQEVCARAQAQGTVDVHRLYAHETPVADLVDLLRTRALFADAVCVVLYNAEVIKKCDEVHVLTEWIKDGGSRADVFLVLISDSVSIHKRIEQNISPVHKRVFWELFENKKHAWVQRFFFQHEMRIEQEAIESLLELVENNTRALKTVCTQLSLFFEKGRRITAHDISSLLVHTKEETSFTLFDALSKRDLEHSLMILNTLLCSKDVAPVQILAGLLTRSVALRIGTIYRRTKRTLQR

SEQ ID NO: 85 - DNA, 945 bp, Ureaplasma urealyticum
atgaacttaataaataaagaactttttttaattactggtgacaatcgtttaaaaatggatcaaaaaattaatgaaattgcgacctcttttgatgaattagttaaaattaatgatcaagaaatttcattaatttcttttaaaaatcttattgaacaagatgatttattcaatagtaataagatctacttatttaaaaacgttaactggttcgaaaatttagaacatttaaaaaatgttagtgatttaattgattatttttttaataatcatgttgctattattattacaattgaatctacaaaaatttctacagcaaaaaaaattcaagaaacaattgcaaaatttaatcatcaaattacttttatgacatatacaaatgaaaatgctatggctttcttaaaagaagagttaaatcagcgtaatttatcattaaataaatctataatgcaaacgattattcaaaaaacaaattttaatattaattttcttaataatgaattagataaaatagaattaattaatgattttttaaaaaatcatggtatgtttgatattaataattttatatgtgattatggtgaatatcaaatttttagtttattaaatcttttgtatcaacataaaataaatgaatcaataaatttaattaataaaatgctcattgacaaaattgatgaactcacaataattaatatgttagcgacaataatgagtacacattacttaattaaattattacatgaaaaaaaatataacaaaaacgttatatctagctctttaaatcaaaaaccttatattattgatttaaatttaaaattaattagaaattattcagctaaatttttacttaaaaaaatgtatgatttattacaattagaaattaaagtcaaggaaaataaaattgataaatattttggattaattaattgggttttgaatttttaa

SEQ ID NO: 86 - DNA, 945 bp, Ureaplasma urealyticum; identical to SEQ ID NO: 85
  CDS (1)..(945)

SEQ ID NO: 87 - protein, 314 aa, Ureaplasma urealyticum
MNLINKELFLITGDNRLKMDQKINEIATSFDELVKINDQEISLISFKNLIEQDDLFNSNKIYLFKNVNWFENLEHLKNVSDLIDYFFNNHVAIIITIESTKISTAKKIQETIAKFNHQITFMTYTNENAMAFLKEELNQRNLSLNKSIMQTIIQKTNFNINFLNNELDKIELINDFLKNHGMFDINNFICDYGEYQIFSLLNLLYQHKINESINLINKMLIDKIDELTIINMLATIMSTHYLIKLLHEKKYNKNVISSSLNQKPYIIDLNLKLIRNYSAKFLLKKMYDLLQLEIKVKENKIDKYFGLINWVLNF

SEQ ID NO: 88 - DNA, 1029 bp, Vibrio cholerae
atgcgcatttacgctgaaaaactggctgaatcactgcacaaaacgctctatccgatttacctcgtattcggtaatgagccactgctgctccaagaagccaaaaccgccatcgaaaaaacggcacaagcgcaaggttttttggaaaaacaccgatttagcgcggatgccgggctcgattggaacgcggtgtatgactgttgccaagcactgagtctgttttcatctcgccaacttatcgaaattgagatcccagaatctggggtcaatgctcaaacggcaaaggaacttagcgcactggtaggccaacttcaccaagacattctcttgctggtgattggccccaaactgactaaggcgcaagagaacgcagcttggttcaaaacgcttgcgcagcaagcctgttgggttaactgcctcacgccagaattgagccgtttacctcagtttgttcagcagcgctgttttgccctcggtttaaaaccggatgccgaagcggtacaaatgttggcgcagtggcatgaaggtaatctctttgcattagcccagagtctcgaaaaattagcgttactctaccccgatggtctactgactttagtgcgcttggaagagtcgctgagccgccataaccatttcacgccttatcactggatggatgcgctattagaaggcaaagccaaccgtgctcagcgtattttgcgccagcttatgctcgaagagagtgagccgattattctgatccgcaccgcgcaaaaagagctgacccaactgcttaaatggcaacaagagcgccaacagctgggtaatttggggagtctttttgatcgctatcgtgtctggcagaatcgaagaccactctactctgccgctttacagcgcttaccaagtcgcgccctacttcggttggttggaatattaactcaggctgaattactggcaaaaacgcaatacgaacagccagtatggccaatcttgcagcaactaagccttgaatgctgcaatcctcaggcaaacctacctttgcctcactaa

SEQ ID NO: 89 - DNA, 1029 bp, Vibrio cholerae; identical to SEQ ID NO: 88
  CDS (1)..(1029)

SEQ ID NO: 90 - protein, 342 aa, Vibrio cholerae
MRIYAEKLAESLHKTLYPIYLVFGNEPLLLQEAKTAIEKTAQAQGFLEKHRFSADAGLDWNAVYDCCQALSLFSSRQLIEIEIPESGVNAQTAKELSALVGQLHQDILLLVIGPKLTKAQENAAWFKTLAQQACWVNCLTPELSRLPQFVQQRCFALGLKPDAEAVQMLAQWHEGNLFALAQSLEKLALLYPDGLLTLVRLEESLSRHNHFTPYHWMDALLEGKANRAQRILRQLMLEESEPIILIRTAQKELTQLLKWQQERQQLGNLGSLFDRYRVWQNRRPLYSAALQRLPSRALLRLVGILTQAELLAKTQYEQPVWPILQQLSLECCNPQANLPLPH

SEQ ID NO: 91 - DNA, 1032 bp, Xylella fastidiosa
atggaacttcgcccagagcaactcgccaacctgcctgccaatcaacccttagagcctgcctacctcattgccggccccgaaaccctacgcttagtcgaagctgccgatgcgatacgtacccatgctcgtgccgacggcatcaccgaacgcgaaatattcgacgtcgaaagccgtgacttcgactggcaacaacttgcctccagcttcaacgcccccagcctcttcagctcacgtcgcttaatcgaaatacgcctcccaggaggtaaacctggtaaagaaggcagtgaagtcatcacaaggttctgcgccaccccctcatcagatgttatcttgctgatcacctgcaacgaatggagcaaagcacaccatggcaagtggaccgatgccatcgctcgcatcggtaaaatcgcaatcgcctggaccatcaagcctcacgaactacccgactggattacacgccgcctacatagcaaaggcttacgtgccaacacagatgtgatacaccaattaagcacacgcctagaaggcaacctactcgccgccgcccaagaaattgacaaattggcgttattgaaccaaggccctctcctcgacctagagaccatggaatcctttgttgccgacacagcgcgttacgacgtattccgactgaccgaagcgatgctctccggccaacccaccgcaacgatacgcatgctcggcaaactgcgggccgaaggtgaagcagtggcagcactgctgcccattctgaccaaagaactcatccgtaccacgaccctgtcatgggtcaaacatgacaacggcaatctagctgccgaaatgaaagcccagggcatctgggaaacacgtcaagcaccgttcaagcgcgcattacaacgtcatacctcaccgtacaaatgggaacacttcgttgccgaagtcgggcgcatagaccgcatcaccaaaggacgcaacgacggcaatgcatggctatcactcgaacgcctcatggtggcagtggcagaagcagatgcaacacgtctgctagcataa

SEQ ID NO: 92 - DNA, 1032 bp, Xylella fastidiosa; identical to SEQ ID NO: 91
  CDS (1)..(1032)

SEQ ID NO: 93 - protein, 343 aa, Xylella fastidiosa
MELRPEQLANLPANQPLEPAYLIAGPETLRLVEAADAIRTHARADGITEREIFDVESRDFDWQQLASSFNAPSLFSSRRLIEIRLPGGKPGKEGSEVITRFCATPSSDVILLITCNEWSKAHHGKWTDAIARIGKIAIAWTIKPHELPDWITRRLHSKGLRANTDVIHQLSTRLEGNLLAAAQEIDKLALLNQGPLLDLETMESFVADTARYDVFRLTEAMLSGQPTATIRMLGKLRAEGEAVAALLPILTKELIRTTTLSWVKHDNGNLAAEMKAQGIWETRQAPFKRALQRHTSPYKWEHFVAEVGRIDRITKGRNDGNAWLSLERLMVAVAEADATRLLA

SEQ ID NO: 94 - DNA, 945 bp, Mycoplasma pulmonis
atgcatttaatttatggacaagaagaatttttaattgaatcaaaaacaaaaaacataattgagaatcaaaaaaaagattcgatagtttttacattttatgaaaactttgaaattgatgatcttttaaatgatattgttcaaaaagatctttttagtcctaataaaataattcatattaaaaactggaaattacttgaaaatccaagaccttcaaaagtaattttaaacaaactaaaattgcttagcgaaataatagaaaatgataaaagtttaatttttattttttctcacattggtgaaattgaaaaaaattttttaagtgattttttattaaaaaaagcttcagtaaaacactataaaaaacttgagttaaatgaaaaaattaaattcattgaaagctatgttactaaaaacaaaattaaaatttcaacagtgcttatttcttatttattaatcaatgtttcatcatctatttcaattgttataaatgaaattaataaactatttttagaaaaccaaaacatcactagagaaatgattgaaaagtcagttggtttttatactaaagaacctaactttgaatttatcaattcaatcaaatctagaaattcaaattcaatttggaaaacatttttagaacaaaaaaattcaggaattgaacctttagatcttttaaaacaaatttcatcagcttttattttaggaaataaaattaacaacatcatcaaattagatcactcacttgatcaagttgctaaaattttaaatgtgcacatttatagagcaaaagaaattttttctttaattaaaggttttagtcaaaactcaattaatgaaattatcgaattattagagttaactgataaaagaatcaaaactacatatatggatcctgagatggctttagatttttttattctaaaattaattagacactataattaa

SEQ ID NO: 95 - DNA, 945 bp, Mycoplasma pulmonis; identical to SEQ ID NO: 94
  CDS (1)..(945)

SEQ ID NO: 96 - protein, 314 aa, Mycoplasma pulmonis
MHLIYGQEEFLIESKTKNIIENQKKDSIVFTFYENFEIDDLLNDIVQKDLFSPNKIIHIKNWKLLENPRPSKVILNKLKLLSEIIENDKSLIFIFSHIGEIEKNFLSDFLLKKASVKHYKKLELNEKIKFIESYVTKNKIKISTVLISYLLINVSSSISIVINEINKLFLENQNITREMIEKSVGFYTKEPNFEFINSIKSRNSNSIWKTFLEQKNSGIEPLDLLKQISSAFILGNKINNIIKLDHSLDQVAKILNVHIYRAKEIFSLIKGFSQNSINEIIELLELTDKRIKTTYMDPEMALDFFILKLIRHYN

SEQ ID NO: 97 - DNA, 975 bp, Staphylococcus aureus
atgagcgacaatattgtagctatttatggagatgtgcctgaattggttgaaaaacaaagtgcagaaatcatatcacaatttttgaaaagtgatagagatgactttaactttgtgaaatataatttatacgaaaaagagattgcaccaattgttgaagaaacattaacattgcctttcttttcagataaaaaagcaattttggttaaaaatgcatatatatttacaggtgaaaaagcgccaaaagatatggctcataatgtagatcaattaatagaatttattgaaaaatatgatggcgaaaatttgattgtctttgagatatatcaaaataaacttgatgaaagaaaaaagttaactaaaactctaaaaaagcatgcaaggcttaaaaaaatagagcaaatgtctgaagaagaaataaaaaaatggattcaaagtaaattaaatgagaatttcaaagatatcaaaagagatgcattagatttatttattgagttgacaggtattaactttaatattgtctcacaagagatagaaaagttgattttatttttaggcgatagaccaacaattaataagcaggatgttaaccaaattattaatagaagtttagagcaaaatgtatttttactgactgaatacattcagaaaagaaagaaagaacaagcaattcatttagtaaaagatttaataactatgaaagaagaaccaattaaattacttgcactaattacaagtaattaccgattattttatcaatgtaagattctgagtcaaaaaggatatagtggacagcaaattgctaaaacaataggagtgcatccatacagagtaaaattagcgttaggacaagtaagacattatcaacttgatgaattattgaatattgtagatgcttgtgcggaaactgattataaacttaaatcatcatatatggataaacagttaatactggaattattcattctatctttataa

SEQ ID NO: 98 - DNA, 975 bp, Staphylococcus aureus; identical to SEQ ID NO: 97
  CDS (1)..(975)

SEQ ID NO: 99 - protein, 324 aa, Staphylococcus aureus
MSDNIVAIYGDVPELVEKQSAEIISQFLKSDRDDFNFVKYNLYEKEIAPIVEETLTLPFFSDKKAILVKNAYIFTGEKAPKDMAHNVDQLIEFIEKYDGENLIVFEIYQNKLDERKKLTKTLKKHARLKKIEQMSEEEIKKWIQSKLNENFKDIKRDALDLFIELTGINFNIVSQEIEKLILFLGDRPTINKQDVNQIINRSLEQNVFLLTEYIQKRKKEQAIHLVKDLITMKEEPIKLLALITSNYRLFYQCKILSQKGYSGQQIAKTIGVHPYRVKLALGQVRHYQLDELLNIVDACAETDYKLKSSYMDKQLILELFILSL

SEQ ID NO: 100 - DNA, 1011 bp, Streptomyces coelicolor
gtgcgtgcgatgcttgtcgcgatggccaggaagactgcgaacgacgaccctctcgccccggtgacccttgccgtgggccaggaggacctcctgctcgaccgtgccgtgcaggaggtggtggccgccgcgaaggccgccgacgccgacacggacgtacgcgacctcacccccgaccagctgcagcccggcaccctcgccgagctgaccagcccctcgctcttcgccgagcgcaaagtcgtggtcgtacgcaatgcgcaggacctgtccgccgacacggtcaaggacgtcaaggcctacctcggcgcccccgccgaggagatcaccctcgtcctgctgcacgcgggcggcgccaagggcaagggcgtcctcgacgccggccgcaaggcgggggcgcgcgaggtggcctgcccgaagatgaccaagcccgccgaccggctggccttcgtgcgcgcggagttccgcacggccgggcggtcggccacacccgaggcctgccaggccctggtcgacgcgatcggcagcgacctcagggagctggcctcggcggtgtcccagctgaccgccgacgtcgaggggaccgtcgacgaggcggtcgtcgggcggtactacaccgggcgggcggaggcctccagcttcacggtcgccgaccgggcggtcgagggacgcgcggcggaggcgctggaggcgctgcgctggtcgctggcgaccggagtggcgccggtgctgatcaccagcgcgctcgcccagggcgtgcgggccatcggcaagctgtcctccgcccgcggcggacgccccgccgacctcgcccgcgagctggggatgccgccctggaagatcgaccgcgtccggcagcagatgcgcggctggaccccggacggcgtctccgtggccctgcgcgcggtcgccgaggccgacgcgggggtgaagggcggcggggacgatcccgagtacgccctggagaaggccgtggtgatcatcgcgcgggcggcgcgttcacggggccggacctga

SEQ ID NO: 101 - DNA, 1011 bp, Streptomyces coelicolor; identical to SEQ ID NO: 100
  CDS (1)..(1011)

SEQ ID NO: 102 - protein, 336 aa, Streptomyces coelicolor
VRAMLVAMARKTANDDPLAPVTLAVGQEDLLLDRAVQEVVAAAKAADADTDVRDLTPDQLQPGTLAELTSPSLFAERKVVVVRNAQDLSADTVKDVKAYLGAPAEEITLVLLHAGGAKGKGVLDAGRKAGAREVACPKMTKPADRLAFVRAEFRTAGRSATPEACQALVDAIGSDLRELASAVSQLTADVEGTVDEAVVGRYYTGRAEASSFTVADRAVEGRAAEALEALRWSLATGVAPVLITSALAQGVRAIGKLSSARGGRPADLARELGMPPWKIDRVRQQMRGWTPDGVSVALRAVAEADAGVKGGGDDPEYALEKAVVIIARAARSRGRT

SEQ ID NO: 103 - DNA, 1041 bp, Streptococcus pyogenes
atgattgcgatagaaaagattgaaaaactgagtaaagaaaatttgggtcttataacccttgtcacaggagatgacattggtcagtatagccagttgaaatcccgcttaatggagcagattgcttttgataaggatgatttggcctattcttactttgatatgtctgaggccgcttatcaggatgcagaaatggatctagtgagcctacccttctttgctgagcagaaggtggttatttttgaccatttgttagatatcacgaccaataaaaaaagtttcttaaaagaaaaagacctaaaggcctttgaagcctatttagaaaatcccttagagactactcgactaattatctttgctccaggtaaattggatagtaagagacggcttgttaagcttttgaaacgtgatgcccttgttttagaagccaaccctctgaaagaagcagagctaagaacttattttcaaaaatacagtcatcaactgggtttaggtttcgagagtggtgcctttgaccaattacttttgaaatcaaacgatgattttagtcaaatcatgaaaaacatggcctttttaaaagcctataaaaaaacgggaaatattagcctaactgatattgagcaagccattcctaaaagtttacaagataatattttcgatctgactagacttgtcctaggaggtaaaattgatgcggctagagatttgattcatgatttacggttatctggagaagatgacattaaattaatcgctatcatgctaggccaatttcgcttatttttgcagctgactattcttgctagagatgtaaaaaacgagcaacaactagtgattagtttatcagatattcttgggcggcgggttaatccttaccaggtcaagtatgcgttaaaggattctaggaccttatctcttgcctttctaacaggagcggtgaaaaccttgattgagacagattaccagataaaaacaggactttatgagaagagttatctagttgatattgctctcttaaaaatcatgactcactctcaaaaatag

SEQ ID NO: 104 - DNA, 1041 bp, Streptococcus pyogenes; identical to SEQ ID NO: 103
  CDS (1)..(1041)

SEQ ID NO: 105 - protein, 346 aa, Streptococcus pyogenes
MIAIEKIEKLSKENLGLITLVTGDDIGQYSQLKSRLMEQIAFDKDDLAYSYFDMSEAAYQDAEMDLVSLPFFAEQKVVIFDHLLDITTNKKSFLKEKDLKAFEAYLENPLETTRLIIFAPGKLDSKRRLVKLLKRDALVLEANPLKEAELRTYFQKYSHQLGLGFESGAFDQLLLKSNDDFSQIMKNMAFLKAYKKTGNISLTDIEQAIPKSLQDNIFDLTRLVLGGKIDAARDLIHDLRLSGEDDIKLIAIMLGQFRLFLQLTILARDVKNEQQLVISLSDILGRRVNPYQVKYALKDSRTLSLAFLTGAVKTLIETDYQIKTGLYEKSYLVDIALLKIMTHSQK

SEQ ID NO: 106 - DNA, 879 bp, Thermus thermophilus
atggtcatcgccttcaccggggatcccttcctggcgcgggaggccctcttagaggaggcaaggcttaggggcctttcccgcttcaccgagcccaccccggaggccctggcccaggccctcgccccggggcttttcgggggcgggggggcgatgctggacctgagggaggtgggggaggcggagtggaaggccctaaagcccctcctggaaagcgtgcccgagggcgtccccgtcctcctcctggaccctaagccaagcccctcccgggcggccttctaccggaaccgggaaaggcgggacttccccacccccaagggaaaggacctggtgcggcacctggaaaaccgggccaagcgcctggggctcaggctcccgggcggggtggcccagtacctggcctccctggagggggacctcgaggccctggaacgggagctggagaagcttgccctcctctcccctcccctcaccctggagaaggtggagaaggtggtggccctgaggccccccctcacgggctttgacctggtgcgctccgtcctggagaaggaccccaaggaggccctcctgcgcctcaggcgcctcaaggaggagggggaggagcccctcaggctcctcggggccctctcctggcagttcgccctcctcgcccgggccttcttcctcctccgggaaaaccccaggcccaaggaggaggacctcgcccgcctcgaggcccacccctacgccgccaaaaaggccctggaggcggcgaggcgccttacggaagaagccctcaaggaggccctggacgccctcatggaggcggaaaagagggccaagggggggaaagacccatggcttgccctggaggcggcggtcctccgcctcgcccgttga

SEQ ID NO: 107 - DNA, 879 bp, Thermus thermophilus; identical to SEQ ID NO: 106
  CDS (1)..(879)

SEQ ID NO: 108 - protein, 292 aa, Thermus thermophilus
MVIAFTGDPFLAREALLEEARLRGLSRFTEPTPEALAQALAPGLFGGGGAMLDLREVGEAEWKALKPLLESVPEGVPVLLLDPKPSPSRAAFYRNRERRDFPTPKGKDLVRHLENRAKRLGLRLPGGVAQYLASLEGDLEALERELEKLALLSPPLTLEKVEKVVALRPPLTGFDLVRSVLEKDPKEALLRLRRLKEEGEEPLRLLGALSWQFALLARAFFLLRENPRPKEEDLARLEAHPYAAKKALEAARRLTEEALKEALDALMEAEKRAKGGKDPWLALEAAVLRLAR

SEQ ID NO: 109 - DNA, 1035 bp, Yersinia pestis
atgatccggatttaccctgaacaacttgtcgcgcagctccatgaggggctacgcgcttgttatctattgtgtggcaatgaacccttgttgctacaggaaagtcaggaccacattcgtcgggtggcatcgcagcacgatttcactgagcatttcagctttgcgttggacgcgcacaccgaatgggagcatatttttagcctttgccaggcactgagtttgttcgccagccgccagacactgctgctgagtttcccagacagtgggctgaccgcgccaattagcgagcagttggttaaattatctggcctgttacacccagacattttattgatattgcgggcgaataagttgaccaaagcgcaagagaacagcgcctggtttaaagctctcagtaaaaatggggtatttgtcagttgccagacaccagaacaggcccagcttccccgctgggtgagcgctcgcgccaagagcctcaacttgaacgtagacgatgccgccattcaattactttgctactgctatgaaggtaacttgctggccttatctcaggcgttagaacggctctcgctgctctaccccgacggtaagttaaccctgcctaaagtagaacaagccgtcaacgacgctgcgcatttcacaccataccattggcttgatgccttgttgatggggaagagcaagcgggcttggcatattttgcagcaattacagcaagaagacagtgaacccgttattttactgcgtaccgtacaacgtgaactgctcctgctgttagcgctcaaacgtcagatggaacaagttccattgcgcgcattattcgatcaacacaaaatttggcagaaccggcggcccatgatgactcaggcactgcaacggctctcccttcagcaactgcaacaggcggttcatctgctcacacagatggaaatccggctgaaacaggattatggtcaatcaatctggcctgagttagaaacgctatctatgctgatgtgtggaaaaacattacctgagagcttttttgatgcccattaa

SEQ ID NO: 110 - DNA, 1035 bp, Yersinia pestis; identical to SEQ ID NO: 109
  CDS (1)..(1035)

SEQ ID NO: 111 - protein, 344 aa, Yersinia pestis
MIRIYPEQLVAQLHEGLRACYLLCGNEPLLLQESQDHIRRVASQHDFTEHFSFALDAHTEWEHIFSLCQALSLFASRQTLLLSFPDSGLTAPISEQLVKLSGLLHPDILLILRANKLTKAQENSAWFKALSKNGVFVSCQTPEQAQLPRWVSARAKSLNLNVDDAAIQLLCYCYEGNLLALSQALERLSLLYPDGKLTLPKVEQAVNDAAHFTPYHWLDALLMGKSKRAWHILQQLQQEDSEPVILLRTVQRELLLLLALKRQMEQVPLRALFDQHKIWQNRRPMMTQALQRLSLQQLQQAVHLLTQMEIRLKQDYGQSIWPELETLSMLMCGKTLPESFFDAH

SEQ ID NO: 112 - DNA, 1038 bp, Streptococcus agalactiae
atgattgcgattgaagaaattgggagaattaccccagataatttagggttagtaactgttttagcaggagaagatttagggcaatatgctcagatgaaagagaagctatttcaggtaattggattcaataaagacgatttagcctattcttattttgatttaagtgaagaagactatcaaaatgcggaattagacttagaaagtttaccttttttatcagactacaaggttgttatttttgatcagtttcaagatattacaactgataaaaaaacttatttagatgaacaagcaatgaagcgcttggaagcctacttgcagaatcctgtagatactacgagacttgttatttgtgctccaggaaaacttgatggaaaacgtcgacttgtaaagttattgaaacgtgatgctcgtgttttagaggctaatactttaaaagaatcagatttaaagacgtattttcaaaaatatgctcaccaagaaggattagtttttgaagcaggtgtttttgatgagttattgattaagtctaactatgattttagtgatactttaacgaatatcgctttcttaaagtcatataaaacagacggtcatatttcgtccaatgatgttagagaggctatccctaaaagcctgcaagataatatatttgatttgactcaagatgtcttacttggcaggatagatttaccgcgtgatttagtaagagatttacgtctacaaggcgaagacgaaatcaaattgattgcaatcatgttaggacagtttcggatgtttttgcaagttaaaatattggctagtaaagggaagtcagagagtcaaattgtttctgaactatcccattacataggacgtaaaatcaacccttatcaagtaaaatttgctgtcagagattctagaaaccttccccttgcttttttaaaagaagcgattaggattttaatcgagactgactatgctattaaaagagggacttatgataaggattatctctttgatttagccttattaaaaattgctcataaaaagccctaa

SEQ ID NO: 113 - DNA, 1038 bp, Streptococcus agalactiae; identical to SEQ ID NO: 112
  CDS (1)..(1038)

SEQ ID NO: 114 - protein, 345 aa, Streptococcus agalactiae
MIAIEEIGRITPDNLGLVTVLAGEDLGQYAQMKEKLFQVIGFNKDDLAYSYFDLSEEDYQNAELDLESLPFLSDYKVVIFDQFQDITTDKKTYLDEQAMKRLEAYLQNPVDTTRLVICAPGKLDGKRRLVKLLKRDARVLEANTLKESDLKTYFQKYAHQEGLVFEAGVFDELLIKSNYDFSDTLTNIAFLKSYKTDGHISSNDVREAIPKSLQDNIFDLTQDVLLGRIDLPRDLVRDLRLQGEDEIKLIAIMLGQFRMFLQVKILASKGKSESQIVSELSHYIGRKINPYQVKFAVRDSRNLPLAFLKEAIRILIETDYAIKRGTYDKDYLFDLALLKIAHKKP

SEQ ID NO: 115 - DNA, 1038 bp, Streptococcus pneumonia
atgctagccattgaagaaagtcagaagttgactttatcaaatttaccgagcctgagcctatttacagggacagatcagggtcagtttgaagtgatgaagagtcaaatgttgaaacagattgggtatgattctgctgacctcaactttgcctactttgatatgaaagaagtagtttacaaggatgtggaactggagttggtcagccttcctttctttgcggatgagaaaatcgtgatattagattattttatggatatcacgactgctaagaaacgctttttgacagatgatgagcttaagtcatttgaggaataccttgacaatccttctccaacaaccaagttgataatctttgcagaaggaaagctggatagcaaaagacggttagtcaaattacttaagcgtgatgccaaggccttcgatgcagtagaagtaaaagaacaagaattgcgccagtacttccaaaagtggagtcagaaacaaggtctgcagtttaccaatcattcttttgaaaatctcctcatcaagtcggggtttcaatttagcgaaatccagaaaaatcttctctttttacagtcctataaggcgaattctgttattgaggaagaggatattgttaacgcaattcccaagaccttgcaggacaatatttttgatttaactcagtttattctgactaaaaagatggatcaggcgcgcgatttggtgagagacttgaccttgcaaggggaagatgaaatcaaactgattgcagtcatgctgggacaatttcggacttttactcaggtgaagattttggcggagtctggccaaacagaatcgcagattgcaagtagtttaggtagttatctgggacgtaacccaaatccttatcaaatcaagtttgcattaagagattcgagaggactttctttgagctttttgaagcaagctatttcctatttgattgagacagactatcagattaagacaggtctttatgaaaaaggtttcctttttgaaaaggcactcttacagattgctagtcaggtcaattga

SEQ ID NO: 116 - DNA, 1038 bp, Streptococcus pneumonia; identical to SEQ ID NO: 115
  CDS (1)..(1038)

SEQ ID NO: 117 - protein, 345 aa, Streptococcus pneumonia
MLAIEESQKLTLSNLPSLSLFTGTDQGQFEVMKSQMLKQIGYDSADLNFAYFDMKEVVYKDVELELVSLPFFADEKIVILDYFMDITTAKKRFLTDDELKSFEEYLDNPSPTTKLIIFAEGKLDSKRRLVKLLKRDAKAFDAVEVKEQELRQYFQKWSQKQGLQFTNHSFENLLIKSGFQFSEIQKNLLFLQSYKANSVIEEEDIVNAIPKTLQDNIFDLTQFILTKKMDQARDLVRDLTLQGEDEIKLIAVMLGQFRTFTQVKILAESGQTESQIASSLGSYLGRNPNPYQIKFALRDSRGLSLSFLKQAISYLIETDYQIKTGLYEKGFLFEKALLQIASQVN

SEQ ID NO: 118 - DNA, 1035 bp, Actinobacillus actinomycetemcomitans
atgaatcggctatttcctgaacagcttgcttcgtcactggaacggcatttagcccatgtgtattttctggtgggcgaggatccgttgttgctcaatgaaagcgccaatggtatttaccaaacggcgcttcagcggggattcgatgaaaaagttgaactggacattaatgcctccaccgactggaatgatttattcgagcgggtgcaatccatgggcttatttttcaataaacagctcattattttagatttacctgaaaatgcgaccgcacttttacagaaaaacttatccgaattcatttcgttgcttcagcccgatgtgctaccgattttccgtttagccaaactcactaaagccgctgaaaagcaggcgtggtttatggcggcgaatcagtatgaaccgcaggcggtattggtaaactgtcaaacccccaatgccgaacaattgccccgctgggtggcgaatcgggcgaagatgttggggttaagcattgaacaagaagcggtgcagttgttgtgttacagctacgaaaacaacctgttggcactcaagcaaacactgcaattactggatttgctttatcctgatcgtaaactcacttttgcccgcgtgaatagcgtggtggaacagtcctcggtgtttacgccgttccaatgggttgatgcgattttaggcggaaaagggaatcgcgcccgccgaattctcaccggtttaaaagatgaagatgttcagccgattattctcctgcgcacgttgcagcgcgatttaatgactttactggagatcagtaaaccggaacaaccccaatcccttgactcgcctttacccaccgatcaactgcgtgaacagtttgatcgcctgaaagtatggcaaaaccgccgttcattgtttactcaagcggtgcaacgtttaacctaccgtaaactctatcttttttttcagcaactcgccgatgtggagcgctgtgcgaaacaggaatttagcgatgatatttggcaacaactggaagatttatcggtaagatttgccggttag

SEQ ID NO: 119 - DNA, 1035 bp, Actinobacillus actinomycetemcomitans; identical to SEQ ID NO: 118
  CDS (1)..(1035)

SEQ ID NO: 120 - protein, 344 aa, Actinobacillus actinomycetemcomitans
MNRLFPEQLASSLERHLAHVYFLVGEDPLLLNESANGIYQTALQRGFDEKVELDINASTDWNDLFERVQSMGLFFNKQLIILDLPENATALLQKNLSEFISLLQPDVLPIFRLAKLTKAAEKQAWFMAANQYEPQAVLVNCQTPNAEQLPRWVANRAKMLGLSIEQEAVQLLCYSYENNLLALKQTLQLLDLLYPDRKLTFARVNSVVEQSSVFTPFQWVDAILGGKGNRARRILTGLKDEDVQPIILLRTLQRDLMTLLEISKPEQPQSLDSPLPTDQLREQFDRLKVWQNRRSLFTQAVQRLTYRKLYLFFQQLADVERCAKQEFSDDIWQQLEDLSVRFAG

SEQ ID NO: 121 - DNA, 1056 bp, Bordetella pertussis
atggctgcgcccctggacagcgacgccctggccagccacctcgcacgcgcggggggccggctggcgccgctgtacacggtatcgggcgacgagccgctgctggtcaccgaggccgccgacgccatccgcgcggcggcgcgcgcggccggctacaccgaccgcacctccatggtcatggacgcgcgcagcgactggagcgcggtggcggccgccacgcaaagcgtctcgctgttcggcgaccggcgcctgctggaactgaaaatcccgaccggcaagcccggcaagagcggcggcgagatgctggcccggctggccgaccaggcgcgcgaccaggccgacgccgacacgctcgtggtcgtggccctgccccgcctggacaaggccacgcgcgaaagcaaatgggcccaggcgctggcgcgcggcggcgtcatggccgacatcgccaacgtcgagcgcggccgcctgccggcatggattggcatgcgcctggggcgccagaaccagcgcgccgacaccgccacgctgcaatggatggccgacaaggtcgagggcaacctgctggcggcccaccaggaaatccagaagctggggctgctttatccggaaggccagctggccgccgaggacgtcgagcgcgccgtgctcaacgtggcgcgctacgacgtcttcgggctgcgcgacgccatgctggccggcgacacggcgcgcacggtgcgcatgctcgacggcttgcgcgccgagggcgaagccctgccgctggtgctgtgggcggtcggcgaggaaatccgcctgctggcgcgcgtagcgcaggcgcgccagcaaggccaggacgccggcgcactgatgcgccgtctgcgcattttcggcgcccacgaacggcttgcgctgcaagcgctgggccgggtcgcacccggcgcatggccggcagccgtgcagcacgcccatgaagtcgatcgcctgatcaaaggcctgagcgtaccgggccggctggccgatccctgggaagaaatgacccggctcgcgctgcgcgtggccgcggccggcgcccgaccgtga

SEQ ID NO: 122 - DNA, 1056 bp, Bordetella pertussis; identical to SEQ ID NO: 121
  CDS (1)..(1056)

SEQ ID NO: 123 - protein, 351 aa, Bordetella pertussis
MAAPLDSDALASHLARAGGRLAPLYTVSGDEPLLVTEAADAIRAAARAAGYTDRTSMVMDARSDWSAVAAATQSVSLFGDRRLLELKIPTGKPGKSGGEMLARLADQARDQADADTLVVVALPRLDKATRESKWAQALARGGVMADIANVERGRLPAWIGMRLGRQNQRADTATLQWMADKVEGNLLAAHQEIQKLGLLYPEGQLAAEDVERAVLNVARYDVFGLRDAMLAGDTARTVRMLDGLRAEGEALPLVLWAVGEEIRLLARVAQARQQGQDAGALMRRLRIFGAHERLALQALGRVAPGAWPAAVQHAHEVDRLIKGLSVPGRLADPWEEMTRLALRVAAAGARP

SEQ ID NO: 124 - DNA, 1011 bp, Bacillus anthracis
atgagtgatatacataaaaagattaaaaaaaagcagtttgctccgttgtatttactgtatggaacggaagcgttttttataaatgaaacgataaagcttattacaacagaagcgcttgaagaggaagatcgcgagtttaatgttgtgacatatgatttagaagaagcgtatttagaagatgtggttgaggatgcacgtacacttccttttttcggagaacgtaaagtgttattaataaaatcaccattatttttaacgtcacaaaaagaaaagttagaacaaaatataaaaattttagaagaatatatcggggaaccttctccattttctattcttgtttttgttgcgccttacgaaaagcttgatgaacgaaaaaaaattacaaaactattaaagaaaacagcagatatagtagaagcgaatgcgatgcaagtgcaggatgttcaaaagtggattgtagctcgtgcagaggaagggcatgtacatattgataatgcagctgttagtttattgttagagcttgtgggaagtaatgtgacgatgttggcgaaggaaatggacaagttaacgttatacgtcggtatgggcggagagattacgccgaaacttgttgcagagcttgtgccaaaatcagttgaacaaaatgtatttgctttaacagaaaaagtggtaaaaaaagatatcgcaggtgcgatgcaaattttggatggattatttacgcagcaggaggaaccgattaaattgcttgcgttgttagtaagtcaattccgcttgctgcatcaagtaaaagagttacagcagcgtggttatggacaaaatcaaattgcttctcatattggtgtacatccatatcgtgtaaagttagcgatgaatcaaacgaagtttttctcttttgaagaactaaaaaaagtgattatagaattggcggaagctgattatagtatgaagactggaaaaatggataagaaacttgtgcttgagtttttcttaatgcgattaaatcatatgtga

SEQ ID NO: 125 - DNA, 1011 bp, Bacillus anthracis; identical to SEQ ID NO: 124
  CDS (1)..(1011)

SEQ ID NO: 126 - protein, 336 aa, Bacillus anthracis
MSDIHKKIKKKQFAPLYLLYGTEAFFINETIKLITTEALEEEDREFNVVTYDLEEAYLEDVVEDARTLPFFGERKVLLIKSPLFLTSQKEKLEQNIKILEEYIGEPSPFSILVFVAPYEKLDERKKITKLLKKTADIVEANAMQVQDVQKWIVARAEEGHVHIDNAAVSLLLELVGSNVTMLAKEMDKLTLYVGMGGEITPKLVAELVPKSVEQNVFALTEKVVKKDIAGAMQILDGLFTQQEEPIKLLALLVSQFRLLHQVKELQQRGYGQNQIASHIGVHPYRVKLAMNQTKFFSFEELKKVIIELAEADYSMKTGKMDKKLVLEFFLMRLNHM

SEQ ID NO: 127 - DNA, 1089 bp, Burkholderia pseudomallei
atgcaactgcgacttgacgcgctggagccgcatctctccaagggcctggccggactgtacgtcgtctacggcgacgaaccgctgctcgcgcaggaggcgtgcgacaagatccgcgccgccgcgcgcgcggcgggcttcaccgagcgttcggtgcacaccgtcgagcgcggcttcgactggagcacgctgatcggcgcgagccaggcgatgtcgctgttcggcgagcggcagctcgtcgagctgcggattccgtccggcaagcccggcaaggacggcgccgacgcgctgaagacgctcgccggcgcggccaacccggacgtgctgatgctcgtcacgctgccgcggctcgatgcggcgacgcagaagtccgcgtggttcacggcgctcgcgaacggcggcgtcgcgctgaagatcgaccccgtcgagcgcgcgcagttgccgaactggatcggtcagcggctcgcgctgcaggggcagcgggtcgccccgggcgacgacggacggcgcgcgctcgcgttcgtcgccgagcgggtcgaaggcaatctgctcgccgcgcatcaggaaatccagaagctcggcctgctgtatccggccggcgcgctgacgttcgagcagatccacgacgcggtgctgaacgtcgcgcgctacgacgtgttcaagctcaacgaggcgatgctcgcgggcgacgccgcgcggctttcgcggatgatcgacggcctgaagggcgagggcgaggcgctcgtgctcgtgctgtgggcggtcgtcgaggagttgcgcacgctgttgcggatcaagcgcggcgtcgcggccggcaagccgcttgccgtgctcgtgcgcgagaaccgcgtgtgggggccgcgcgagcggctcgtcgggcccgcgctttcgcgcgtatcggaagcggcgctcgagcacgcgctcgcgttcgccgcgcggctcgaccggcaggtcaaggggttgtcggccgtgtcgcgcggcgcggcgcgcggcgagccgccgcccgatccgtgggacggcctgttccagctcgcgatgaccgtcgcgcgcgcggccgggccaggcggcgacgcgccgcgccggcccgcctga

SEQ ID NO: 128 - DNA, 1089 bp, Burkholderia pseudomallei; identical to SEQ ID NO: 127
  CDS (1)..(1089)

SEQ ID NO: 129 - protein, 362 aa, Burkholderia pseudomallei
MQLRLDALEPHLSKGLAGLYVVYGDEPLLAQEACDKIRAAARAAGFTERSVHTVERGFDWSTLIGASQAMSLFGERQLVELRIPSGKPGKDGADALKTLAGAANPDVLMLVTLPRLDAATQKSAWFTALANGGVALKIDPVERAQLPNWIGQRLALQGQRVAPGDDGRRALAFVAERVEGNLLAAHQEIQKLGLLYPAGALTFEQIHDAVLNVARYDVFKLNEAMLAGDAARLSRMIDGLKGEGEALVLVLWAVVEELRTLLRIKRGVAAGKPLAVLVRENRVWGPRERLVGPALSRVSEAALEHALAFAARLDRQVKGLSAVSRGAARGEPPPDPWDGLFQLAMTVARAAGPGGDAPRRPA

SEQ ID NO: 130 - DNA, 1041 bp, Chlorobium tepidum
atggctgacctgaaaaaacagattgcctcaggcaaaatcgcccctgtctatttttttcagggtccggaaagctggctcaaggaggagattgaaacgcaactgaaggcggccattttcccttcggaaaacgaggctgccctcaacacgcttgtcctgtacgggccagacctcaccctcggccagatcgtttcagcggcctcagaatacccgatgttcaccgaaaagaagctggtcgtcgtccgccagttcgacaagctgaaaaaggtgaccgacaaaacggaaagccagcggcaggagaagtcgctcctgagctatctttccgatccgccgtcatttaccgtgctggtgctcgatgcagacgagatcgaaaaaacggagcttgaaaaagccccgtacaaacatctcaaagcgtttcgccatgacttcggggcgatcaaaaatgcggagatcttcgccgctgaaagagcccgggaagcgggatgggagttcgagccggaagccctgaaaacattctcgggatacatcgagccatcatcgaggctgatctgccaggagatcgagaagctcacgctctacgcctcgaacaaacgccccgaacggcgcatcaccctcgacgacgtctgcgactgcgtcggcatctctcgaaaatataatgtcttcgagcttgaaaaagcactggtcagcaagaacctccggcagtgtagcggcatcgctttgatgatcatggagcaggaggggcagaaagaggggctcatgaacatcgtgcgctacctgaccacctttttcctgcggatatggaagctccagacaccgggcgcacagcaactctcccttcaggaaacagcagcgatgctcggcatgtacggacgccaggagtatttcgccaaaaacttcatcggctacgcccgccagttcagcgccgccgaaaccgaaaatgccattctcgccctgcgtgacgccgatgccgccctcaaaggcattattccctcgcccgacgacagattcaccctgctcaaactgatgcagcaactcttcaattga

SEQ ID NO: 131 - DNA, 1041 bp, Chlorobium tepidum; identical to SEQ ID NO: 130
  CDS (1)..(1041)

SEQ ID NO: 132 - protein, 346 aa, Chlorobium tepidum
MADLKKQIASGKIAPVYFFQGPESWLKEEIETQLKAAIFPSENEAALNTLVLYGPDLTLGQIVSAASEYPMFTEKKLVVVRQFDKLKKVTDKTESQRQEKSLLSYLSDPPSFTVLVLDADEIEKTELEKAPYKHLKAFRHDFGAIKNAEIFAAERAREAGWEFEPEALKTFSGYIEPSSRLICQEIEKLTLYASNKRPERRITLDDVCDCVGISRKYNVFELEKALVSKNLRQCSGIALMIMEQEGQKEGLMNIVRYLTTFFLRIWKLQTPGAQQLSLQETAAMLGMYGRQEYFAKNFIGYARQFSAAETENAILALRDADAALKGIIPSPDDRFTLLKLMQQLFN

SEQ ID NO: 133 - DNA, 840 bp, Chloroflexus aurantiacus
caactggttgcggcatgtgaggcacacccgtttcttgccgagcggcgactggtgatcgtctacgatgcgttgaagcatagtaaggcagggaaggggcgcgaagaattacgtagttattgcgagcgtgtgccggcaacctgtgatctggtgtttgtggagcaagaagaggttgatcgccgacatctgcttttcacatacctcagcaagcatggtgtcgttcgtgaattcccgttgctccagggagcagagttactccgctggattgaacagcgggcgaagatgctgcatgcgacgattgaatctgccgccgcccaacgtctggtcgaactggcagggaatgatagccggttactggctaacgagctggccaaactggccagttatgtgggtcgcaatgggaagatcgatctgcgggctgtagatcgcctggttaccgatcaacaagagcaaaatctgtttgcctttctcgatgaattaagttcgcgacggttagggccggcattacgcggcgcacgagcactggttgaagatggccaggcaccgccctacgtgctctttatgctggctcgtcagatcagaattctcttgcaggtcaggcaactgctcagtcaacggcgtcgcgctgatgagattgctgccgagctgaacctgaaaccattcgtggcccgaaaggcaactgcacaggcgcgtaacttcagccttcccgaactgatacacgcccatgatcgtctgctcgaacttgatcacgcgatcaagaccggacgtatccaggccgaaacagcacttgaactatttgttgtggaagtgtgtcagatgccgcaagaggtggggaagcgttag

SEQ ID NO: 134 - DNA, 840 bp, Chloroflexus aurantiacus; identical to SEQ ID NO: 133
  CDS (1)..(840)

SEQ ID NO: 135 - protein, 279 aa, Chloroflexus aurantiacus
QLVAACEAHPFLAERRLVIVYDALKHSKAGKGREELRSYCERVPATCDLVFVEQEEVDRRHLLFTYLSKHGVVREFPLLQGAELLRWIEQRAKMLHATIESAAAQRLVELAGNDSRLLANELAKLASYVGRNGKIDLRAVDRLVTDQQEQNLFAFLDELSSRRLGPALRGARALVEDGQAPPYVLFMLARQIRILLQVRQLLSQRRRADEIAAELNLKPFVARKATAQARNFSLPELIHAHDRLLELDHAIKTGRIQAETALELFVVEVCQMPQEVGKR

SEQ ID NO: 136 - DNA, 1044 bp, Clostridium difficile
atgaatcacaaggatattattaggaatgtaaaagagaaaaattttgaaaaagtgtatcttttttatggaagagagtattatttaattgagaatgcaataaaagcatttaaagatagtctaaacgagggaatgcttgattttaatttagatataatagatgggaaggaaatagtcttaaatcagcttataagctctatagaaacacttccatttatggatgataggaagatagtaataataaaagactttgaactattaaaaggaaagaaaaagaattttacagatagtgatgagaaatacttaatagagcatttagacaatattccagacaccacaactatagtttttgttgtatatggagatgtagacaaaagaaaatcattagttaagaaaataggaaataatggaattgtttttgattgtgataagctttctgatatggatttgtttaaatggataaaaaaatcttttgctttaaatgatgttataatagataattctcaaatcatgtattttattgaacaagaaggatacagagataagagtagtgaaaagacattatctgatttagaaaatgaaataaataaaataagttcatttgttggtaaagggaataatgtaaccaatgatgtaattgataaattatctcaaaaaaaagttgagaatgatatattcaaattaatcgattatattggagaacaaaatgcttctaatgcaatgaaaatattaaatgatatgatacaagaaggcgaatctgtattaggtatattttctatgatagcaagacaatttaaaataattatgcaagtaagacagttacaacttgatggatattcaacaaaacttatagctgataaattaaaaatgcatcaatttgttgtaggaaaagcattaaagcaaactaaaaatttttctgatgatataattgtggaaatattaaactatatattagaaagtgattataaaataaagacaggtcttataagagatactttagcagttgaaatgttggttagtagatactgtaaaagagaagcaatttaa

SEQ ID NO: 137 - DNA, 1044 bp, Clostridium difficile; identical to SEQ ID NO: 136
  CDS (1)..(1044)

SEQ ID NO: 138 - protein, 347 aa, Clostridium difficile
MNHKDIIRNVKEKNFEKVYLFYGREYYLIENAIKAFKDSLNEGMLDFNLDIIDGKEIVLNQLISSIETLPFMDDRKIVIIKDFELLKGKKKNFTDSDEKYLIEHLDNIPDTTTIVFVVYGDVDKRKSLVKKIGNNGIVFDCDKLSDMDLFKWIKKSFALNDVIIDNSQIMYFIEQEGYRDKSSEKTLSDLENEINKISSFVGKGNNVTNDVIDKLSQKKVENDIFKLIDYIGEQNASNAMKILNDMIQEGESVLGIFSMIARQFKIIMQVRQLQLDGYSTKLIADKLKMHQFVVGKALKQTKNFSDDIIVEILNYILESDYKIKTGLIRDTLAVEMLVSRYCKREAI

SEQ ID NO: 139 - DNA, 975 bp, Corynebacterium diphtheriae
atgggtcagcaacttctaccagtgcacctcgtagttggcgacgatgaattcctagccgaacgcgcacgacacagcatcatcgcagccatcaacaaaaccattggcagcgacgtccccgtgaccacccttcgggcaggcgaagtcaacgcctccgaactcatccaactcaccagcccgtcgctcttcggcgaagaccgcatcattgtgctgaccaacatggaagatgctggcaaagaacccgcggaactcgtcctcaacgttgccgtcgacccagcaccaggaatctacctcatcatcgtgcactccggcggcggacggcaaaaagcactagtaccaaaactcagcaaaatcagccaagtccacgaagccaacaaactcaaacctaaagaccgcgtgggctgggtcaccaacgaattcaaacgccacggagtacgccccaccccagacgtggtccacgccctgctcgaaggcgtaggatccgacctccgcgaactcgcctccgccatcggccagcttgttgctgacaacaatggcgacgtcaccctcgccaccgtccgcgactactacgtgggagtagcagaagtctccggtttcgacgtggcagatcttgcctgctcaggacaaacccaacgtgccgtcgccagcgcgcgccgggcactgcaactaggagtctcaccggtggcacttgccgccgcattgagcatgaaagttagcgctatcgcgcggttgtattccacacgagggcgtattgactcccgtaaactcgcaggagagctgggcatgcaccccttcgttgttgaaaaaaccgccaaagttgcgcgcatgtggacgggtaatgctgtgagccaagcggtgatcttgatggcggatgtggattccagtcttaagggccacggcctcgatggcgaactcggcggtgaccccgactacgaaatcgaggatgctatccgcaggatctctgagctcgcaggctaa

SEQ ID NO: 140 - DNA, 975 bp, Corynebacterium diphtheriae; identical to SEQ ID NO: 139
  CDS (1)..(975)

SEQ ID NO: 141 - protein, 324 aa, Corynebacterium diphtheriae
MGQQLLPVHLVVGDDEFLAERARHSIIAAINKTIGSDVPVTTLRAGEVNASELIQLTSPSLFGEDRIIVLTNMEDAGKEPAELVLNVAVDPAPGIYLIIVHSGGGRQKALVPKLSKISQVHEANKLKPKDRVGWVTNEFKRHGVRPTPDVVHALLEGVGSDLRELASAIGQLVADNNGDVTLATVRDYYVGVAEVSGFDVADLACSGQTQRAVASARRALQLGVSPVALAAALSMKVSAIARLYSTRGRIDSRKLAGELGMHPFVVEKTAKVARMWTGNAVSQAVILMADVDSSLKGHGLDGELGGDPDYEIEDAIRRISELAG

SEQ ID NO: 142 - DNA, 1035 bp, Cytophaga hutchinsonii
atggctgaaaccagttctgctccggataaagtattaaaagaccttaaggaaggtaaatacgctcctgtatattttttgcagggagaggaaccctattttatagatctaatatccagttatatagaaaataactgtttgccggaatcggagagaggatttaatcagacgatcctgtatggcaaagataccaatgtttctaccattcttcagaatgcccgcaagtatccgatgttttcggaaaggcaggtagtaatggttaaggaagcacaggatgtttcggatctgaccaaagattcaacagagaaatttttaatggcatatctcgaaaatccattgcctacaaccattctggtattttgccacaagtataaaaaactggatgcccgtaaaaaaataaccaagcacctgcagaagtattcggtgtttgttgaatctgccaaactttacgataataaattgcctgaatggattaatggctatttcagggaaaagaattacaggattgatccgaaggcggtcgttctattggccgaatctattggtacggatctttcccgaatggcaaatgaaattgataagctgctcatcaacgttaaagatcctgctataaccatcaccgaagatttaatcgaaaaatatgttggcgtgagcaaagaatacaatgtctttgaattacaaaaagctatcggtgtacgggatataataaaagcgaataaaatcgttaatcattttgaatccaatccaaaagcgaatccgattgtaatggtgctgtcaagtttgtttacttactatcttaaaatactggcgattcatgccgccacagataagagtgaagcaagcctggcaaaggttgttggtgtacatccattctttgtaaaagaatacatcaatgcgtcgcgcatcaatgatattaataaatgcatctcttgtgtgcgcgccattcgtgaagcggacaaaatggtgaaagggtatacggttgttaatgttaccgatggctttattttaaaagaattattgtttaaattattacattga

SEQ ID NO: 143 - DNA, 1035 bp, Cytophaga hutchinsonii; identical to SEQ ID NO: 142
  CDS (1)..(1035)

SEQ ID NO: 144 - protein, 344 aa, Cytophaga hutchinsonii
MAETSSAPDKVLKDLKEGKYAPVYFLQGEEPYFIDLISSYIENNCLPESERGFNQTILYGKDTNVSTILQNARKYPMFSERQVVMVKEAQDVSDLTKDSTEKFLMAYLENPLPTTILVFCHKYKKLDARKKITKHLQKYSVFVESAKLYDNKLPEWINGYFREKNYRIDPKAVVLLAESIGTDLSRMANEIDKLLINVKDPAITITEDLIEKYVGVSKEYNVFELQKAIGVRDIIKANKIVNHFESNPKANPIVMVLSSLFTYYLKILAIHAATDKSEASLAKVVGVHPFFVKEYINASRINDINKCISCVRAIREADKMVKGYTVVNVTDGFILKELLFKLLH

SEQ ID NO: 145 - DNA, 1200 bp, Dehalococcoides ethenogenes
atgtttgcggagttcgtagggcattacaacttcttcgggcattgtttcaaatcctccaagtgcagcttacgggcagattgtaacatactgccaggcatttttgcactaaacttcaaaggttacgcgcccgtttcgttatgctacaatatcagccgaaggtggctgttaaatttgctgtatatttttaccggcagtgatgatttttccctgaaagaacgcctgaagcagataaaactggctttgggtgatgagtctttgtcctctgccaataccaccatgctggagggcaaaaacctgacggctgccgaacttcaaaactatgtccagactattccctttttagcccctgcccggctggttatggtaaacggcctgctggagaggtttgaaggctcaaaaggtaaagccgccgccaaagataaggcagacaaaaacagcccggatgaatttgcccgggcgctgtctaatctgcctcccacaaccctggcggtgcttctggactataccaccgctaaagacggtgattaccgtgaaaaagaaaaagtatccgctttggcctcaaaccccctttttaagctggtaagccccggtgctgaggtaaagcactttgcacccctttctgcccagaacctgcccggctgggtaatgcagcgtgccaaacagtcttctatccagatgtctgttccggcagcccgtctgctggcccgttttgtgggggcggacttgtgggcgctttcctccgagattgagaaactgggcttgtatgcccaggggcgggcggtggaggaagccgatgttgaaaatatggtgggttattcccaggaagtaaatatattcggtttggtggatgccgtactggaccgcaaggttaaagacgcccagcagtctctggcggcttttwctgycggcggcgggtyacccggttttttgctgtttatgctggtacgccagctgcggctgatggtgcgttataccgcccttaaaaagcgggggctgaaagaggccgagatttttaaagctatggkcggctctgagtacgcccttaggaaaacagcctctcaggcggcctgccagaacatggacagccttaaagctatttattccaaactgctggaaacagattttaatataaaaaccggccgctttgaaccggagctggccatgggtttgctggttgccgaactttgcgggagaaaatag

SEQ ID NO: 146 - DNA, 1200 bp, Dehalococcoides ethenogenes
  CDS (1)..(1200)
atgtttgcggagttcgtagggcattacaacttcttcgggcattgtttcaaatcctccaagtgcagcttacgggcagattgtaacatactgccaggcatttttgcactaaacttcaaaggttacgcgcccgtttcgttatgctacaatatcagccgaaggtggctgttaaatttgctgtatatttttaccggcagtgatgatttttccctgaaagaacgcctgaagcagataaaactggctttgggtgatgagtctttgtcctctgccaataccaccatgctggagggcaaaaacctgacggctgccgaacttcaaaactatgtccagactattccctttttagcccctgcccggctggttatggtaaacggcctgctggagaggtttgaaggctcaaaaggtaaagccgccgccaaagataaggcagacaaaaacagcccggatgaatttgcccgggcgctgtctaatctgcctcccacaaccctggcggtgcttctggactataccaccgctaaagacggtgattaccgtgaaaaagaaaaagtatccgctttggcctcaaaccccctttttaagctggtaagccccggtgctgaggtaaagcactttgcacccctttctgcccagaacctgcccggctgggtaatgcagcgtgccaaacagtcttctatccagatgtctgttccggcagcccgtctgctggcccgttttgtgggggcggacttgtgggcgctttcctccgagattgagaaactgggcttgtatgcccaggggcgggcggtggaggaagccgatgttgaaaatatggtgggttattcccaggaagtaaatatattcggtttggtggatgccgtactggaccgcaaggttaaagacgcccagcagtctctggcggcttttactgccggcggcgggtcacccggttttttgctgtttatgctggtacgccagctgcggctgatggtgcgttataccgcccttaaaaagcgggggctgaaagaggccgagatttttaaagctatgggcggctctgagtacgcccttaggaaaacagcctctcaggcggcctgccagaacatggacagccttaaagctatttattccaaactgctggaaacagattttaatataaaaaccggccgctttgaaccggagctggccatgggtttgctggttgccgaactttgcgggagaaaatag

SEQ ID NO: 147 - protein, 399 aa, Dehalococcoides ethenogenes
MFAEFVGHYNFFGHCFKSSKCSLRADCNILPGIFALNFKGYAPVSLCYNISRRWLLNLLYIFTGSDDFSLKERLKQIKLALGDESLSSANTTMLEGKNLTAAELQNYVQTIPFLAPARLVMVNGLLERFEGSKGKAAAKDKADKNSPDEFARALSNLPPTTLAVLLDYTTAKDGDYREKEKVSALASNPLFKLVSPGAEVKHFAPLSAQNLPGWVMQRAKQSSIQMSVPAARLLARFVGADLWALSSEIEKLGLYAQGRAVEEADVENMVGYSQEVNIFGLVDAVLDRKVKDAQQSLAAFTAGGGSPGFLLFMLVRQLRLMVRYTALKKRGLKEAEIFKAMGGSEYALRKTASQAACQNMDSLKAIYSKLLETDFNIKTGRFEPELAMGLLVAELCGRK

SEQ ID NO: 148 - DNA, 1008 bp, Porphyromonas gingivalis
atggcctcctattccgatattgtcgattctgtacgtaagtgccaattctcgccactctatctgctggccggcgaagaacccttctatatagacgagttggccactctgctggagacgcacgtggtgccggtagatgaatgggacttcaacagggttatcctctacggcgacaaaacctccgtagccgatatagcgaacgaagcgcgccgtttcccgatgatgggccggaggcagttgatcgtcgttcgggaggcgcagttagtggacaatatcgacttgctcgaagcacattatggcactttccccgatactacgattctggtcattgcctataagaagaaacccgataagcgaaaggctttctatacgaaggctgaaaagttcgggaaagtattcgtgtccgagaccatacccgactataagatgccggacttcatcctgtcggctgctgccggcaagaaactgagcgtatcgcccgaagtggcatatatgctggccgattacctcggcaatgatctggagaagttgatgaatgagctggacaagctcattctcataacacaagatagtaggggagtggtcacgtccgagatcgtggagcagcatatcggagtaagcaaatccttcaataatttcgaactccttcgtgccatagtcaatcgcgaaacgggcaaaacctttcgcatcgctcactactttgccagaaatgagaaggagcaccccatacaggccactctacccgtactcttcaactatttcagcaacctgatgatcgtttgctacttgccacagaaaaaacccgatgctatcatgaaagcattgtcgatacgcaatttccaagtgcgcgactatatgacagggctgaagatgtattcgacgaggaaggttttcgacattatacacgagatccggatgacggatgcccgcagtaagggggtggatacgaccggcacatttgccggcagtggggatttgcttcgggagctactccattttatattccattga

SEQ ID NO: 149 - DNA, 1008 bp, Porphyromonas gingivalis; identical to SEQ ID NO: 148
  CDS (1)..(1008)

SEQ ID NO: 150 - protein, 335 aa, Porphyromonas gingivalis
MASYSDIVDSVRKCQFSPLYLLAGEEPFYIDELATLLETHVVPVDEWDFNRVILYGDKTSVADIANEARRFPMMGRRQLIVVREAQLVDNIDLLEAHYGTFPDTTILVIAYKKKPDKRKAFYTKAEKFGKVFVSETIPDYKMPDFILSAAAGKKLSVSPEVAYMLADYLGNDLEKLMNELDKLILITQDSRGVVTSEIVEQHIGVSKSFNNFELLRAIVNRETGKTFRIAHYFARNEKEHPIQATLPVLFNYFSNLMIVCYLPQKKPDAIMKALSIRNFQVRDYMTGLKMYSTRKVFDIIHEIRMTDARSKGVDTTGTFAGSGDLLRELLHFIFH

SEQ ID NO: 151 - DNA, 1005 bp, Prochlorococcus marinus
atgccaatacaaataatatggggaaatgatttaaatgcaagtaataagtttataaaacaaataattgatgaaaaagtatccaaaacatggggagaaataaatattagttacttaaatggtgatgatgataatcaaataaaacaagcatttgatgaaatactaacacctcctttgggggatggatctcgagtagttgtattaaaaaataacccattattcacaaataaaaatgaggaaataaggctcaaatttgaaaaaatttatcaaaacattccaagtaacacttactttcttttacaaaatacaaaaaaaccagattcaagactaaagagtacaaaatttattcaaaatttaataaaaaaagatttagcaattgaaacctcattttcattgcccgatatctgggattttgaaggccagaaaagatacttagaattcacagcaaattcgatgaatattcatttaggtaaagggaccgctgatttaattattgattcagtaggaaatgatagttttaacttagctagcgaattatctaaagcaaagatatatctatctgcaataaataataacgaaaacaaaggacttattctagaaagtgatgttgtaaaaaaaatattcaatgatcaacagtcaaatatctttaaaataatcgatcatctattacaaaatagtattagtgatggtcttatagaaatctattatttattaaaaaaaggggaacctgctttaaggctcaatgcgggattaataagtcaaatcagaatgcataccatagtaaaattacttcataaatctggtgagcaagatttatcaaaaatatgcaaacttgcaaatatatctaacccaaaaagaattttttttatccgtaaaaaagtaaacaatatatcctcagatttcttaattaaattgatgagtaatttattagatattgaatcatcactaaaaaaaggcaataatcctattaatgtatttgcggaaaatctcgttaacttaagttaataa

SEQ ID NO: 152 - DNA, 1005 bp, Prochlorococcus marinus; identical to SEQ ID NO: 151
  CDS (1)..(1005)

SEQ ID NO: 153 - protein, 333 aa, Prochlorococcus marinus
MPIQIIWGNDLNASNKFIKQIIDEKVSKTWGEINISYLNGDDDNQIKQAFDEILTPPLGDGSRVVVLKNNPLFTNKNEEIRLKFEKIYQNIPSNTYFLLQNTKKPDSRLKSTKFIQNLIKKDLAIETSFSLPDIWDFEGQKRYLEFTANSMNIHLGKGTADLIIDSVGNDSFNLASELSKAKIYLSAINNNENKGLILESDVVKKIFNDQQSNIFKIIDHLLQNSISDGLIEIYYLLKKGEPALRLNAGLISQIRMHTIVKLLHKSGEQDLSKICKLANISNPKRIFFIRKKVNNISSDFLIKLMSNLLDIESSLKKGNNPINVFAENLVNLS

SEQ ID NO: 154 - DNA, 1032 bp, Salmonella typhi
atgatcaggttgtatcctgaacaactccgcgcgcagctcaacgaagggctgcgtgcggcatatctattattgggcaacgatccgctgctgctgcaggaaagtcaggacgctattcgtctggcggctgcgtcgcaaggcttcgaagagcaccacgcttttactctcgatcccagtaccgactggggatcgcttttctcactctgccaggcgatgagcctgttcgccagccgccaaacgctggtgctgcaactgccggaaaacgggcctaacgcggcaatgaatgagcaactcgccaccctgagcgagctactgcatgacgacctgttattaatagtacgcggaaacaagctcaccaaagcgcaggaaaacgccgcctggtataccgcgctggcagatcgtagcgttcaggtcagttgtcagacgccggagcaggcgcagcttccccgctgggtggccgccagagcgaaagcacagaatttgcaactggatgacgccgcgaaccaactgctgtgctattgctatgagggcaatctgttggcgctggcgcaggcgctggaacggctgtcgctgctttggcctgacggtaagttaacgcttcctcgcgtggagcaggccgtcaatgacgccgcccactttacccctttccactgggtggatgcgttactgatggggaaaagcaaacgcgcgctgcatatcctgcaacagctacgccttgaaggcagcgaaccggttattctgctgcggaccctccagcgagagctgctgctactggtaaatctcaaacgtcagtccgcgcatacgccgttacgcgcgctgtttgataaacaccgggtctggcaaaaccgtcgccccatgatcggcgacgcgttgcagcgtctccatcctgcacagcttcgccaggccgtgcagttactgacgcgtacagaaattacgctcaaacaggattatggtcagtcggtatgggcagatcttgaagggctgtcgctcctgctctgccataaggcgctggcagacgtatttattgatgggtga

SEQ ID NO: 155 - DNA, 1032 bp, Salmonella typhi; identical to SEQ ID NO: 154
  CDS (1)..(1032)

SEQ ID NO: 156 - protein, 343 aa, Salmonella typhi
MIRLYPEQLRAQLNEGLRAAYLLLGNDPLLLQESQDAIRLAAASQGFEEHHAFTLDPSTDWGSLFSLCQAMSLFASRQTLVLQLPENGPNAAMNEQLATLSELLHDDLLLIVRGNKLTKAQENAAWYTALADRSVQVSCQTPEQAQLPRWVAARAKAQNLQLDDAANQLLCYCYEGNLLALAQALERLSLLWPDGKLTLPRVEQAVNDAAHFTPFHWVDALLMGKSKRALHILQQLRLEGSEPVILLRTLQRELLLLVNLKRQSAHTPLRALFDKHRVWQNRRPMIGDALQRLHPAQLRQAVQLLTRTEITLKQDYGQSVWADLEGLSLLLCHKALADVFIDG

SEQ ID NO: 157 - DNA, 32 bp, Artificial Sequence
  Description of Artificial Sequence Primer
gatcctgcaggaaaccacaatattccagttcc

SEQ ID NO: 158 - DNA, 40 bp, Artificial Sequence
  Description of Artificial Sequence Primer
gatcaagcttttattatccaccatgagaagtatttttcac

SEQ ID NO: 159 - DNA, 32 bp, Artificial Sequence
  Description of Artificial Sequence Primer
gatccatatggaaaccacaatattccagttcc

SEQ ID NO: 160 - DNA, 40 bp, Artificial Sequence
  Description of Artificial Sequence Primer
gatcggtaccttattatccaccatgagaagtatttttcac

SEQ ID NO: 161 - DNA, 32 bp, Artificial Sequence
  Description of Artificial Sequence Primer
gatcggtacccagaggtgattgtatgccagtc

SEQ ID NO: 162 - DNA, 34 bp, Artificial Sequence
  Description of Artificial Sequence Primer
gatcactagtttattattcttcatctctctgaag

SEQ ID NO: 163 - DNA, 29 bp, Artificial Sequence
  Description of Artificial Sequence Primer
gatccatatgagaggtgattgtatgccag

SEQ ID NO: 164 - DNA, 35 bp, Artificial Sequence
  Description of Artificial Sequence Primer
gatcctgcaggaaaaagtttttttggaaaaactcc

SEQ ID NO: 165 - DNA, 36 bp, Artificial Sequence
  Description of Artificial Sequence Primer
gatcggtaccttattaatccgcctgaacggctaacg

SEQ ID NO: 166 - DNA, 35 bp, Artificial Sequence
  Description of Artificial Sequence Primer
gatccatatggaaaaagtttttttggaaaaactcc

SEQ ID NO: 167 - DNA, 31 bp, Artificial Sequence
  Description of Artificial Sequence Primer
gatcctgcagagtatttcagaaaaacccttg

SEQ ID NO: 168 - DNA, 35 bp, Artificial Sequence
  Description of Artificial Sequence Primer
gatcggtacctaatcaaacgctgaacttttcttcg

SEQ ID NO: 169 - DNA, 27 bp, Artificial Sequence
  Description of Artificial Sequence Primer
gatctcatgagtatttcagaaaaaccc

SEQ ID NO: 170 - DNA, 33 bp, Artificial Sequence
  Description of Artificial Sequence Primer
gatcctgcagaacgatttgatcagaaagtacgc

SEQ ID NO: 171 - DNA, 34 bp, Artificial Sequence
  Description of Artificial Sequence Primer
gatcggtaccttatcagctccaagcgttgacacc

SEQ ID NO: 172 - DNA, 33 bp, Artificial Sequence
  Description of Artificial Sequence Primer
gatccatatgaacgatttgatcagaaagtacgc

SEQ ID NO: 173 - protein, 1161 aa, Aquifex aeolicus
MSKDFVHLHLHTQFSLLDGAIKIDELVKKAKEYGYKAVGMSDHGNLFGSYKFYKALKAEGIKPIIGMEAYFTTGSRFDRKTKTSEDNITDKYNHHLILIAKDDKGLKNLMKLSTLAYKEGFYYKPRIDYELLEKYGEGLIALTACLKGVPTYYASINEVKKAEEWVKKFKDIFGDDLYLELQANNIPEQEVANRNLIEIAKKYDVKLIATQDAHYLNPEDRYAHTVLMALQMKKTIHELSSGNFKCSNEDLHFAPPEYMWKKFEGKFEGWEKALLNTLEVMEKTADSFEIFENSTYLLPKYDVPPDKTLEEYLRELAYKGLRQRIERGQAKDTKEYWERLEYELEVINKMGFAGYFLIVQDFINWAKKNDIPVGPGRGSAGGSLVAYAIGITDVDPIKHGFLFERFLNPERVSMPDIDVDFCQDNREKVIEYVRNKYGHDNVAQIITYNVMKAKQTLRDVARAMGLPYSTADKLAKLIPQGDVQGTWLSLEEMYKTPVEELLQKYGEHRTDIEDNVKKFRQICEESPEIKQLVETALKLEGLTRHTSLHAAGVVIAPKPLSELVPLYYDKEGEVATQYDMVQLEELGLLKMDFLGLKTLTELKLMKELIKERHGVDINFLELPLDDPKVYKLLQEGKTTGVFQLESRGMKELLKKLKPDSFDDIVAVLALYRPGPLKSGLVDTYIKRKHGKEPVEYPFPELEPVLKETYGVIVYQEQVMKMSQILSGFTPGEADTLRKAIGKKKADLMAQMKDKFIQGAVERGYPEEKIRKLWEDIEKFASYSFNKSHSVAYGYISYWTAYVKAHYPAEFFAVKLTTEKNDNKFLNLIKDAKLFGFEILPPDINKSDVGFTIEGENRIRFGLARIKGVGEETAKIIVEARKKYKQFKGLADFINKTKNRKINKKVVEALVKAGAFDFTKKKRKELLAKVANSEKALMATQNSLFGAPKEEVEELDPLKLEKEVLGFYISGHPLDNYEKLLKNRYTPIEDLEEWDKESEAVLTGVITELKVKKTKNGDYMAVFNLVDKTGLIECVVFPGVYEEAKELIEEDRVVVVKGFLDEDLETENVKFVVKEVFSPEEFAKEMRNTLYIFLKREQALNGVAEKLKGIIENNRTEDGYNLVLTVDLGDYFVDLALPQDMKLKADRKVVEEIEKLGVKVII

SEQ ID NO: 174 - protein, 363 aa, Aquifex aeolicus
MRVKVDREELEEVLKKARESTEKKAALPILANFLLSAKEENLIVRATDLENYLVVSVKGEVEEEGEVCVHSQKLYDIVKNLNSAYVYLHTEGEKLVITGGKSTYKLPTAPAEDFPEFPEIVEGGETLSGNLLVNGIEKVEYAIAKEEANIALQGMYLRGYEDRIHFVGSDGHRLALYEPLGEFSKELLIPRKSLKVLKKLITGIEDVNIEKSEDESFAYFSTPEWKLAVRLLEGEFPDYMSVIPEEFSAEVLFETEEVLKVLKRLKALSEGKVFPVKITLSENLAIFEFADPEFGEAREEIEVEYTGEPFEIGFNGKYLMEALDAYDSERVWFKFTTPDTATLLEAEDYEKEPYKCIIMPMRV

SEQ ID NO: 175 - protein, 202 aa, Aquifex aeolicus
MKSSPKSLKMRDNLLDGTFVVIDLEATGFDVEKSEVIDLAAVRVEGGIITEKFSTLVYPGYFIPERIKKLTGITNAMLVGQPTIEEVLPEFLEFVGDNIVVGHFVEQDIKFINKYTKQYRGKKFRNPSLCTLKLARKVFPGLKKYSLKEIAENFGFETNGVHRALKDATLTAEIFIKILEELWFKYGIGDYYSLKRLEKGKF

SEQ ID NO: 176 - protein, 473 aa, Aquifex aeolicus
MNYVPFARKYRPKFFREVIGQEAPVRILKNAIKNDRVAHAYLFAGPRGVGKTTIARILAKALNCKNPSKGEPCGECENCREIDRGVFPDLIEMDAASNRGIDDVRALKEAVNYKPIKGKYKVYIIDEAHMLTKEAFNALLKTLEEPPPRTVFVLCTTEYDKILPTILSRCQRIIFSKVRKEKVIEYLKKICEKEGIECEEGALEVLAHASEGCMRDAASLLDQASVYGEGRVTKEVVENFLGILSQESVRSFLKLLLNSEVDEAIKFLRELSEKGYNLTKFWEMLEEEVRNAILVKSLKNPESVVQNWQDYEDFKDYPLEALLYVENLINRGKVEARTREPLRAFELAVIKSLIVKDIIPVSQLGSVVKETKKEEKKVEVKEEPKVKEEKPKEQEEDRFQKVLNAVDGKILKRILEGAKREERDGKIVLKIEASYLRTMKKEFDSLKETFPFLEFEPVEDKKKPQKSSGTRLF

SEQ ID NO: 177 - protein, 147 aa, Aquifex aeolicus
MLNKVFIIGRLTGDPVITYLPSGTPVVEFTLAYNRRYKNQNGEFQEESHFFDVKAYGKMAEDWATRFSKGYLVLVEGRLSQEKWEKEGKKFSKVRIIAENVRLINRPKGAELQAEEEEEVPPIEEEIEKLGKEEEKPFTDEEDEIPF

SEQ ID NO: 178 - protein, 478 aa, Thermotoga maritima
MEVLYRKYRPKTFSEVVNQDHVKKAIIGAIQKNSVAHGYIFAGPRGTGKTTLARILAKSLNCENRKGVEPCNSCRACREIDEGTFMDVIELDAASNRGIDEIRRIRDAVGYRPMEGKYKVYIIDEVHMLTKEAFNALLKTLEEPPSHVVFVLATTNLEKVPPTIISRCQVFEFRNIPDELIEKRLQEVAEAEGIEIDREALSFIAKRASGGLRDALTMLEQVWKFSEGKIDLETVHRALGLIPIQVVRDYVNAIFSGDVKRVFTVLDDVYYSGKDYEVLIQEAVEDLVEDLERERGVYQVSANDIVQVSRQLLNLLREIKFAEEKRLVCKVGSAYIATRFSTTNVQENDVREKNDNSNVQQKEEKKETVKAKEEKQEDSEFEKRFKELMEELKEKGDLSIFVALSLSEVQFDGEKVIISFDSSKAMHYELMKKKLPELENIFSRKLGKKVEVELRLMGKEETIEKVSQKILRLFEQEG

SEQ ID NO: 179 - protein, 366 aa, Thermotoga maritima
MKVTVTTLELKDKITIASKALAKKSVKPILAGFLFEVKDGNFYICATDLETGVKATVNAAEISGEARFVVPGDVIQKMVKVLPDEITELSLEGDALVISSGSTVFRITTMPADEFPEITPAESGITFEVDTSLLEEMVEKVIFAAAKDEFMRNLNGVFWELHKNLLRLVASDGFRLALAEEQIENEEEASFLLSLKSMKEVQNVLDNTTEPTITVRYDGRRVSLSTNDVETVMRVVDAEFPDYKRVIPETFKTKVVVSRKELRESLKRVMVIASKGSESVKFEIEENVMRLVSKSPDYGEVVDEVEVQKEGEDLVIAFNPKFIEDVLKHIETEEIEMNFVDSTSPCQINPLDISGYLYIVMPIRLA

SEQ ID NO: 180 - protein, 189 aa, Thermotoga maritima
MLAMIWNDTVFCVVDTETTGTDPFAGDRIVEIAAVPVFKGKIYRNKAFHSLVNPRIRIPALIQKVHGISNMDIVEAPDMDTVYDLFRDYVKGTVLVFHNANFDLTFLDMMAKETGNFPITNPYIDTLDLSEEIFGRPHSLKWLSERLGIKTTIRHRALPDALVTARVFVKLVEFLGENRVNEFIRGKRG

SEQ ID NO: 181 - protein, 1367 aa, Thermotoga maritima
MKKIENLKWKNVSFKSLEIDPDAGVVLVSVEKFSEEIEDLVRLLEKKTRFRVIVNGVQKSNGDLRGKILSLLNGNVPYIKDVVFEGNRLILKVLGDFARDRIASKLRSTKKQLDELLPPGTEIMLEVVEPPEDLLKKEVPQPEKREEPKGEELKIEDENHIFGQKPRKIVFTPSKIFEYNKKTSVKGKIFKIEKIEGKRTVLLIYLTDGEDSLICKVFNDVEKVEGKVSVGDVIVATGDLLLENGEPTLYVKGITKLPEAKRMDKSPVKRVELHAHTKFSDQDAITDVNEYVKRAKEWGFPAIALTDHGNVQAIPYFYDAAKEAGIKPIFGIEAYLVSDVEPVIRNLSDDSTFGDATFVVLDFETTGLDPQVDEIIEIGAVKIQGGQIVDEYHTLIKPSREISRKSSEITGITQEMLENKRSIEEVLPEFLGFLEDSIIVAHNANFDYRFLRLWIKKVMGLDWERPYIDTLALAKSLLKLRSYSLDSVVEKLGLGPFRHHRALDDARVTAQVFLRFVEMMKKIGITKLSEMEKLKDTIDYTALKPFHCTILVQNKKGLKNLYKLVSDSYIKYFYGVPRILKSELIENREGLLVGSACISGELGRAALEGASDSELEEIAKFYDYIEVMPLDVIAEDEEDLDRERLKEVYRKLYRIAKKLNKFVVMTGDVHFLDPEDARGRAALLAPQGNRNFENQPALYLRTTEEMLEKAIEIFEDEEIAREVVIENPNRIADMIEEVQPLEKKLHPPIIENADEIVRNLTMKRAYEIYGDPLPEIVQKRVEKELNAIINHGYAVLYLIAQELVQKSMSDGYVVGSRGSVGSSLVANLLGITEVNPLPPHYRCPECKYFEVVEDDRYGAGYDLPNKNCPRCGAPLRKDGHGIPFETFMGFEGDKVPDIDLNFSGEYQERAHRFVEELFGKDHVYRAGTINTIAERSAVGYVRSYEEKTGKKLRKAEMERLVSMITGVKRTTGQHPGGLMIIPKDKEVYDFTPIQYPANDRNAGVFTTHFAYETIHDDLVKIDALGHDDPTFIKMLKDLTGIDPMTIPMDDPDTLAIFSSVKPLGVDPVELESDVGTYGIPEFGTEFVRGMLVETRPKSFAELVRISGLSHGTDVWLNNARDWINLGYAKLSEVISCRDDIMNFLIHKGMEPSLAFKIMENVRKGKGITEEMESEMRRLKVPEWFIESCKRIKYLFPKAHAVAYVSMAFRIAYFKVHYPLQFYAAYFTIKGDQFDPVLVLRGKEAIKRRLRELKAMPAKDAQKKNEVSVLEVALEMILRGFSFLPPDIFKSDAKKFLIEGNSLRIPFNKLPGLGDSVAESIIRAREEKPFTSVEDLMKRTKVNKNHIELMKSLGVLGDLPETEQFTLF

SEQ ID NO: 182 - protein, 842 aa, Thermotoga maritima
MIPWVISPYSFDGSVVRFEKLALLLKRKGLKSVILADRNFHAAVKFNTIMRKHGLIPVHGLWKDGRIFVARNREEFDSLVRYYNGETHEIEDIPVFQESELTPVRYLDASEKKASIFMRKIFGLDEDVQGFPEKCEDVADILNAEAYDLRVNHRFPTPPKNWNELLIKKAEPLGEEYISRLKRELEVIKRKGFTPYIYTVEKVVEIAKKMGIKVGPGRGSAVGSLVAYLCGITEVDPIKYDLLFERFLNEERQEPPDIDVDVEDRRRKDLIKELSKSFQVYQVSTFGNLTEKSLKNLINSVLPDASLEEKNEIYKTVYGLPHHPSVHAAGVVISENPLPLPTRTEEDIPITDYDMYDLQEIGVVKIDILGLKTLSFIKDFKKEIFDYSDEKTYHLISKGKTLGVFQLEGLQARKLCRRISPRNMDELSILLALNRPGPLRSGLDVMFSNSKNVPAFFRKMFPETRGVLIYQEQIMRLAMFAGLSGTEADILRRAIAKKEREKMEPLLEKMKKGLLEKGMENAEQILEILLNFSSYAFNKSHSVAYAHITYQTAYLKAHHLEEFFKLYFAYNSSDAGKIFLAVQELRNEGYRVHPPDINISGKDLVFHGKDVYLPLTVVKGVGVTLVEQIEKIRPVSSVRELQERVTGVPRNVVESLITAGAFDKLYENRKLALEELNKRVEKDILEIRSLFGEKVEQESSNIKIGDITELEEKSMGFPLTPVHEVPTGLFARIDDVFTYGRILPVLVKRVSRNIVTDGLSVCRVRTDVPDGVHLVLLSPLQKIIKIWPFNENTRFVYRVDFTATLEKAGQNEITEVLKNGAVVRYEGYRPLTDEYRYRVVPR

SEQ ID NO: 183 - protein, 141 aa, Thermotoga maritima
MSFFNKIILIGRLVRDPEERYTLSGTPVTTFTIAVDRVPRKNAPDDAQTTDFFRIVTFGRLAEFARTYLTKGRLVLVEGEMRMRRWETPTGEKRVSPEVVANVVRFMDRKPAETVSETEEELEIPEEDFSSDTFSEDEPPF

SEQ ID NO: 184 - DNA, 250 bp, Artificial Sequence
  Description of Artificial Sequence PCR product
gaattcggatcccgggtctagaggaggttaattaacatatgaaaaaaaaaaccaggttgctagcggtaccactagtggtggtggtggtctggttccgcgtggttcccaccaccaccacctgcaccacatcctggacgctcagaaaatggtttggaaccactgccgttgatgagcagttagagaaagaacccatagtaggagcagaaactttctaggtcgacagcttggctgttttggcggatgagagaag

SEQ ID NO: 185 - DNA, 250 bp, Artificial Sequence
  Description of Artificial Sequence PCR product
cttctctcatccgccaaaacagccaagctgtcgacctagaaagtttctgctcctactatgggttctttctctaactgctcatcaacggcagtggttccaaaccattttctgagcgtccaggatgtggtgcaggtggtggtggtgggaaccacgcggaaccagaccaccaccaccactagtggtaccgctagcaacctggttttttttttcatatgttaattaacctcctctagacccgggatccgaattc

SEQ ID NO: 186 - DNA, 200 bp, Artificial Sequence
  Description of Artificial Sequence PCR product
gaattcggatcccgggtctagaggaggtaataacatatggctggttgcctgaacgacatcttcgaagctcagaaaatcgaatggcaccatcaccatcaccatctggttccgcgtggttccggcggcggcggcctgcaggtacccaaaaatgcatgagctcgctagcaagcttaaaaaaaaaactagtgtcgacagcttgg

SEQ ID NO: 187 - DNA, 200 bp, Artificial Sequence
  Description of Artificial Sequence PCR product
ccaagctgtcgacactagttttttttttaagcttgctagcgagctcatgcatttttgggtacctgcaggccgccgccgccggaaccacgcggaaccagatggtgatggtgatggtgccattcgattttctgagcttcgaagatgtcgttcaggcaaccagccatatgttattacctcctctagacccgggatccgaattc

SEQ ID NO: 188 - DNA, 200 bp, Artificial Sequence
  Description of Artificial Sequence PCR product
gaattcggatcccgggtctagaggaggtaataacatatggctggttgcctgaacgacatcttcgaagctcagaaaatcgaatggcaccatcaccatcaccatctggttccgcgtggttccggcggcggcggcctgcagaacctaggaaaaaaaaaggtaccaaaaaaaaaggccggccactagtgtcgacagcttggctg

SEQ ID NO: 189 - DNA, 200 bp, Artificial Sequence
  Description of Artificial Sequence PCR product
cagccaagctgtcgacactagtggccggcctttttttttggtacctttttttttcctaggttctgcaggccgccgccgccggaaccacgcggaaccagatggtgatggtgatggtgccattcgattttctgagcttcgaagatgtcgttcaggcaaccagccatatgttattacctcctctagacccgggatccgaattc

SEQ ID NO: 190 - DNA, 250 bp, Artificial Sequence
  Description of Artificial Sequence PCR product
acacagaattcggatcccgggtctagaggaggttaattaaccatggaaaaaaaaaggtaccaaaaaaaaaggccggccactagtggtggtggtggtctggttccgcgtggttcccaccaccaccacctgcaccacatcctggacgctcagaaaatggtttggaaccactgccgttgatgagcagttagagaaagaacccatagtaggagcagaaactttctaggtcgacagcttggctgttttggcggat

SEQ ID NO: 191 - DNA, 250 bp, Artificial Sequence
  Description of Artificial Sequence PCR product
atccgccaaaacagccaagctgtcgacctagaaagtttctgctcctactatgggttctttctctaactgctcatcaacggcagtggttccaaaccattttctgagcgtccaggatgtggtgcaggtggtggtggtgggaaccacgcggaaccagaccaccaccaccactagtggccggcctttttttttggtacctttttttttccatggttaattaacctcctctagacccgggatccgaattctgtgt

SEQ ID NO: 192 - DNA, 150 bp, Artificial Sequence
  Description of Artificial Sequence PCR product
acacagaattcggatcccgggtctagaggaggttaattaaccatgagtatttcagaaaaacccttgagtgaactgctcaggccgaaagacttcgaggatttcgtcggtcaggatcatatattcggtaataaagggattctccggcgaact

SEQ ID NO: 193 - DNA, 150 bp, Artificial Sequence
  Description of Artificial Sequence PCR product
agttcgccggagaatccctttattaccgaatatatgatcctgaccgacgaaatcctcgaagtctttcggcctgagcagttcactcaagggtttttctgaaatactcatggttaattaacctcctctagacccgggatccgaattctgtgt

SEQ ID NO: 194 - protein, 22 aa, Artificial Sequence
  Description of Artificial Sequence Consensus sequence
XXXXYXXXGXXXXXXXXXXXXX

SEQ ID NO: 195 - protein, 19 aa, Artificial Sequence
  Description of Artificial Sequence Consensus sequence
XXXXXXXXXXFXXXXXXXX

SEQ ID NO: 196 - protein, 21 aa, Artificial Sequence
  Description of Artificial Sequence Consensus sequence
XXXXXXXXXXXXXXXXXXXKX

SEQ ID NO: 197 - protein, 38 aa, Artificial Sequence
  Description of Artificial Sequence Consensus sequence
XXXXXXXXXXXXXXXXXXXXXXXNXXXXXXXXXXXXXX

SEQ ID NO: 198 - protein, 46 aa, Artificial Sequence
  Description of Artificial Sequence Consensus sequence
FXXXXXXXXGXXXXXXXXXXXXXXXGXXPXXXXXXXXXXXXXXXXX

SEQ ID NO: 199 - protein, 34 aa, Artificial Sequence
  Description of Artificial Sequence Consensus sequence
XXXXXXXXXXXXXXXXXXXXXXXXXXXXXXXXXX

SEQ ID NO: 200 - protein, 643 aa, Escherichia coli
MSYQVLARKWRPQTFADVVGQEHVLTALANGLSLGRIHHAYLFSGTRGVGKTSIARLLAKGLNCETGITATPCGVCDNCREIEQGRFVDLIEIDAASRTKVEDTRDLLDNVQYAPARGRFKVYLIDEVHMLSRHSFNALLKTLEEPPEHVKFLLATTDPQKLPVTILSRCLQFHLKALDVEQIRHQLEHILNEEHIAHEPRALQLLARAAEGSLRDALSLTDQAIASGDGQVSTQAVSAMLGTLDDDQALSLVEAMVEANGERVMALINEAAARGIEWEALLVEMLGLLHRIAMVQLSPAALGNDMAAIELRMRELARTIPPTDIQLYYQTLLIGRKELPYAPDRRMGVEMTLLRALAFHPRMPLPEPEVPRQSFAPVAPTAVMTPTQVPPQPQSAPQQAPTVPLPETTSQVLAARQQLQRVQGATKAKKSEPAAATRARPVNNAALERLASVTDRVQARPVPSALEKAPAKKEAYRWKATTPVMQQKEVVATPKALKKALEHEKTPELAAKLAAEAIERDPWAAQVSQLSLPKLVEQVALNAWKEESDNAVCLHLRSSQRHLNNRGAQQKLAEALSMLKGSTVELTIVEDDNPAVRTPLEWRQAIYEEKLAQARESIIADNNIQTLRRFFDAELDEESIRPI

SEQ ID NO: 201 - protein, 15 aa, Artificial Sequence
  Description of Artificial Sequence Consensus sequence
GXXXXGKXXXXXXXX

SEQ ID NO: 202 - protein, 14 aa, Artificial Sequence
  Description of Artificial Sequence Consensus sequence
XXXXGXXXLXXXXD

SEQ ID NO: 203 - protein, 14 aa, Artificial Sequence
  Description of Artificial Sequence Consensus sequence
HAYLFSGXXGXGKX

SEQ ID NO: 204 - protein, 10 aa, Artificial Sequence
  Description of Artificial Sequence Consensus sequence
XDXXEXDXAS

SEQ ID NO: 205 - protein, 12 aa, Artificial Sequence
  Description of Artificial Sequence Consensus sequence
XXXXXDEXHMXX

SEQ ID NO: 206 - protein, 334 aa, Artificial Sequence
  Description of Artificial Sequence Consensus sequence
MRWYPWLRPDFEKLVASYQAGRGHHALLIQALPGMGDDALIYALSRYLLCQQPQGHKSCGHCRGCQLMQAGTHPDYYTLAPEKGKNTLGVDAVREVTEKLNEHARLGGAKVVWVTDAALLTDAAANALLKTLEEPPAETWFFLATREPERLLATLRSRCRLHYLAPPPEQYAVTWLSREVTMSQDALLAALRLSAGSPGAALALFQGDNWQARETLCQALAYSVPSGDWYSLLAALNHEQAPARLHWLATLLMDALKRHHGAAQVTNVDVPGLVAELANHLSPSRLQAILGDVCHIREQLMSVTGINRELLITDLLLRIEHYLQPGVVLPVPHL

SEQ ID NO: 207 - protein, 13 aa, Artificial Sequence
  Description of Artificial Sequence Consensus sequence
XANXXXXXXEXPP

SEQ ID NO: 208 - protein, 8 aa, Artificial Sequence
  Description of Artificial Sequence Consensus sequence
XXXTXXSR

SEQ ID NO: 209 - protein, 343 aa, Artificial Sequence
  Description of Artificial Sequence Consensus sequence
MIRLYPEQLRAQLNEGLRAAYLLLGNDPLLLQESQDAVRQVAAAQGFEEHHTFSIDPNTDWNAIFSLCQAMSLFASRQTLLLLLPENGPNAAINEQLLTLTGLLHDDLLLIVRGNKLSKAQENAAWFTALANRSVQVTCQTPEQAQLPRWVAARAKQLNLELDDAANQVLCYCYEGNLLALAQALERLSLLWPDGKLTLPRVEQAVNDAAHFTPFHWVDALLMGKSKRALHILQQLRLEGSEPVILLRTLQRELLLLVNLKRQSAHTPLRALFDKHRVWQNRRGMMGEALNRLSQTQLRQAVQLLTRTELTLKQDYGQSVWAELEGLSLLLCHKPLADVFIDG

SEQ ID NO: 210 - protein, 324 aa, Thermotoga maritima
MPVTFLTGTAETQKEELIKKLLKDGNVEYIRIHPEDPDKIDFIRSLLRTKTIFSNKTIIDIVNFDEWKAQEQKRLVELLKNVPEDVHIFIRSQKTGGKGVALELPKPWETDKWLEWIEKRFRENGLLIDKDALQLFFSKVGTNDLIIEREIEKLKAYSEDRKITVEDVEEVVFTYQTPGYDDFCFAVSEGKRKLAHSLLSQLWKTTESVVIATVLANHFLDLFKILVLVTKKRYYTWPDVSRVSKELGIPVPRVARFLGFSFKTWKFKVMNHLLYYDVKKVRKILRDLYDLDRAVKSEEDPKPFFHEFIEEVALDVYSLQRDEE

SEQ ID NO: 211 - DNA, 1115 bp, Thermotoga maritima
cactttgaaattgtttctcaaagcgtgttaaaatcaccatgagaggtgattgtatgccagtcacgtttctcacaggtactgcagaaactcagaaggaagaattgataaagaaactcctgaaggatggtaacgtggagtacataaggatccatccggaggatcccgacaagatcgatttcataaggtctttactcaggacaaagacgatcttttccaacaagacgatcattgacatcgtcaatttcgatgagtggaaagcacaggagcagaagcgtctcgttgaacttttgaaaaacgtaccggaagacgttcatatcttcatccgttctcaaaaaacaggtggaaagggagtagcgctggagcttccgaagccatgggaaacggacaagtggcttgagtggatagaaaagcgcttcagggagaatggtttgctcatcgataaagatgcccttcagctgtttttctccaaggttggaacgaacgacctgatcatagaaagggagattgaaaaactgaaagcttattccgaggacagaaagataacggtagaagacgtggaagaggtcgtttttacctatcagactccgggatacgatgatttttgctttgctgtttccgaaggaaaaaggaagctcgctcactctcttctgtcgcagctgtggaaaaccacagagtccgtggtgattgccactgtccttgcgaatcacttcttggatctcttcaaaatcctcgttcttgtgacaaagaaaagatactacacctggcctgatgtgtccagggtgtccaaagagctgggaattcccgttcctcgtgtggctcgtttcctcggtttctcctttaagacctggaaattcaaggtgatgaaccacctcctctactacgatgtgaagaaggttagaaagatactgagggatctctacgatctggacagagccgtgaaaagcgaagaagatccaaaaccgttcttccacgagttcatagaagaggtggcactggatgtatattctcttcagagagatgaagaataactggtacagcctcgcggcccttctctcaaccatatacagcaggcacctcgatgtggaagcaagacctgtgaagtttgaggaaataaa

SEQ ID NO: 212 - DNA, 1200 bp, Aquifex aeolicus
tggcttttacctctttgtagagttaagtattaaatttaatttaactgtggaaaccacaatattccagttccagaaaacttttttcacaaaacctccgaaggagagggtcttcgtccttcatggagaagagcagtatctcataagaacctttttgtctaagctgaaggaaaagtacggggagaattacacggttctgtggggggatgagataagcgaggaggaattctacactgccctttccgagaccagtatattcggcggttcaaaggaaaaagcggtggtcatttacaacttcggggatttcctgaagaagctcggaaggaagaaaaaggaaaaagaaaggcttataaaagtcctcagaaacgtaaagagtaactacgtatttatagtgtacgatgcgaaactccagaaacaggaactttcttcggaacctctgaaatccgtagcgtctttcggcggtatagtggtagcaaacaggctgagcaaggagaggataaaacagctcgtccttaagaagttcaaagaaaaagggataaacgtagaaaacgatgcccttgaataccttctccagctcacgggttacaacttgatggagctcaaacttgaggttgaaaaactgatagattacgcaagtgaaaagaaaattttaacactcgatgaggtaaagagagtagccttctcagtctcagaaaacgtaaacgtatttgagttcgttgatttactcctcttaaaagattacgaaaaggctcttaaagttttggactccctcatttccttcggaatacaccccctccagattatgaaaatcctgtcctcctatgctctaaaactttacaccctcaagaggcttgaagagaagggagaggacctgaataaggcgatggaaagcgtgggaataaagaacaactttctcaagatgaagttcaaatcttacttaaaggcaaactctaaagaggacttgaagaacctaatcctctccctccagaggatagacgctttttctaaactttactttcaggacacagtgcagttgctgagggatttcttgacctcaagactggagagggaagttgtgaaaaatacttctcatggtggataatcttttttatgaagtttgcggtttgcgtttttcccggttctaactgcgattacgacacctactacgttataagggacattcttgaaaaggacgtggaattc

SEQ ID NO: 213 - protein, 350 aa, Aquifex aeolicus
METTIFQFQKTFFTKPPKERVFVLHGEEQYLIRTFLSKLKEKYGENYTVLWGDEISEEEFYTALSETSIFGGSKEKAVVIYNFGDFLKKLGRKKKEKERLIKVLRNVKSNYVFIVYDAKLQKQELSSEPLKSVASFGGIVVANRLSKERIKQLVLKKFKEKGINVENDALEYLLQLTGYNLMELKLEVEKLIDYASEKKILTLDEVKRVAFSVSENVNVFEFVDLLLLKDYEKALKVLDSLISFGIHPLQIMKILSSYALKLYTLKRLEEKGEDLNKAMESVGIKNNFLKMKFKSYLKANSKEDLKNLILSLQRIDAFSKLYFQDTVQLLRDFLTSRLEREVVKNTSHGG

SEQ ID NO: 214 - protein, 32 aa, Escherichia coli
MAGCLNDIFEAQKIEWHHHHHHLVPRGSGGGG

SEQ ID NO: 215 - protein, 305 aa, Aquifex aeolicus
MEKVFLEKLQKTLHIPGGLLFYGKEGSGKTKTAFEFAKGILCKENVPWGCGSCPSCKHVNELEEAFFKGEIEDFKVYKDKDGKKHFVYLMGEHPDFVVIIPSGHYIKIEQIREVKNFAYVKPALSRRKVIIIDDAHAMTSQAANALLKVLEEPPADTTFILTTNRRSAILPTILSRTFQVEFKGFSVKEVMEIAKVDEEIAKLSGGSLKRAILLKENKDILNKVKEFLENEPLKVYKLASEFEKWEPEKQKLFLEIMEELVSQKLTEEKKDNYTYLLDTIRLFKDGLARGVNEPLWLFTLAVQAD

SEQ ID NO: 216 - DNA, 1540 bp, Aquifex aeolicus
actctctttcagacttaaagaaaagatacgggaaattctcggcgagaaatttgagcatttaataaaagttttaggggatataatttccgataatacacttgaaacacctttaataacactcggtgaaggtaaaaacctcaagaaaaagtttaacgaggaattcatagaaacccttgagaatgcttcctattcaaacgaggaggccggaaaattaagggaatttttaaagacactttacgctttggcactctgtaatagtagggaagaaatcactctggacctttgtgtaatattactccactccgaacagcacgcaactttagaaaaacaactcctttctctcgtacaaccgctactaaaaattgataaaaaaccgaagaaagaactgaacctatagtccgcgaagttgcaaaggttataaacgaaacctgtttaaattaaatttgtatggaaaaagtttttttggaaaaactccagaaaaccttgcacatacccggaggactccttttttacggcaaagaaggaagcggaaagacgaaaacagcttttgaatttgcaaaaggtattttatgtaaggaaaacgtaccgtggggatgcggaagttgtccctcctgcaaacacgtaaacgagctggaggaagccttctttaaaggagaaatagaagactttaaagtttataaagacaaggacggtaaaaagcacttcgtttaccttatgggcgaacatcccgactttgtggtaataatcccgagcggacattacataaagatagaacagataagggaagttaagaactttgcctatgtgaagcccgcactaagcaggagaaaagtaattataatagacgacgcccacgcgatgacctctcaggcggcaaacgctcttttaaaggtattggaagagccacctgcggacaccacctttatcttgaccacgaacaggcgttctgcaatcctgccgactatcctctccagaacttttcaagtggagttcaagggcttttcagtaaaagaggttatggaaatagcgaaagtagacgaggaaatagcgaaactctctggaggcagtctaaaaagggctatcttactaaaggaaaacaaagatatcctaaacaaagtaaaggaattcttggaaaacgagccgttaaaagtttacaagcttgcaagtgaattcgaaaagtgggaacctgaaaagcaaaaactcttccttgaaattatggaagaattggtatctcaaaaattgaccgaagagaaaaaagacaattacacctaccttcttgatacgatcagactctttaaagacggactcgcaaggggtgtaaacgaacctctgtggctgtttacgttagccgttcaggcggattaataaaccgttattgattccgtaacatttaaaccttaatctaaattatgagagcctttgaaggaggtctggtatggaaaatttgaagattagatatatagatacgaggaagataggaaccgtgagcggtgtaaaagttccggtaaaaaagggctacaaaatagtcgtggaagacgag

SEQ ID NO: 217 - protein, 599 aa, Thermotoga maritima
MSISEKPLSELLRPKDFEDFVGQDHIFGNKGILRRTLKTGNMFSSILYGPPGSGKTSVFSLLKRYFNGEVVYLSSTVHGVSEIKNVLKRGEQLRKYGKKLLLFLDEIHRLNKNQQMVLVSHVERGDIVLVATTTENPSFVIVPALLSRCRILYFKKLSDEDLMKILKKATEVLNIDLEESVEKAIVRHSEGDARKLLNTLEIVHQAFKNKRVTLEDLETLLGNVSGYTKESHYDFASAFIKSMRGSDPNAAVYYLVKMIEMGEDPRFIARRMIIFASEDVGLADPNALHIAVSTSIAVEHVGLPECLMNLVECAVYLSLAPKSNSVYLAMKKVQELPVEDVPLFLRNPVTEEMKKRGYGEGYLYPHDFGGFVKTNYLPEKLKNEVIFQPKRVGFEEELFERLRKLWPEKYGGESMAEVRKELEYKGKKIRIVKGDITREEVDAIVNAANEYLKHGGGVAGAIVRAGGSVIQEESDRIVQERGRVPTGEAVVTSAGKLKAKYVIHTVGPVWRGGSHGEDELLYKAVYNALLRAHELKLKSISMPAISTGIFGFPKERAVGIFSKAIRDFIDQHPDTTLEEIRICNIDEETTKIFEEKFSV

SEQ ID NO: 218 - DNA, 2640 bp, Thermotoga maritima
caagacaggatccgtacggtgaagcgggcgtggtggccatcttcactgaaaggatgctcaggggagaggaagttcacatattcggtgatggagagtacgtcagagactacgtttacgttgatgacgtggtcagagcgaatcttctcgccatggagaaaggcgacaacgaagtcttcaacataggaacgggaaggggcacaaccgtgaaccagctcttcaaactgctgaaagagatcactggctacgataaagagcctgtctataaaccaccgcgtaagggcgacgtgagaaagagcattctcgattacacgaaggcgaaggaaaagctcggctgggaacccaaagtttcccttgaggagggattgaagctcaccgttgagtatttcagaaaaacccttgagtgaactgctcaggccgaaagacttcgaggatttcgtcggtcaggatcatatattcggtaataaagggattctccggcgaactttaaagacaggcaacatgttctcttctatcctctatggaccaccggggtctggtaagacctcggttttttcactgctgaaaaggtatttcaacggcgaggtagtttatctgagctccaccgttcacggggtttctgaaataaagaacgttctcaaaagaggagaacagttgagaaaatatggaaaaaaattgcttctctttctcgatgaaatacaccgcctgaacaaaaaccagcagatggttctggtttcccatgttgaacgaggagacatcgtactggttgcgacaacaactgaaaatccgagttttgtcatcgtacctgcacttctctcaaggtgcaggattctttatttcaaaaaactctctgacgaggacctgatgaagatcttgaaaaaagcaacagaggttctcaacatcgatctggaagaatcggtagagaaagccatagtaaggcattccgaaggagacgccagaaaactcttgaacaccctggagatcgtccaccaggcgttcaaaaacaagagagtaactcttgaggacctggaaactctgctgggaaacgtgagcgggtacacgaaggaatcacactacgatttcgcttcggccttcataaagagtatgagaggtagcgatccaaacgccgctgtttactacctcgtcaaaatgatagagatgggagaagacccgcgattcatagcacgaagaatgatcatattcgccagcgaagacgttggactcgccgacccaaacgctctacatatcgccgtttcgacctccatcgctgtcgaacacgtgggacttcccgaatgtttgatgaaccttgtagagtgtgccgtctatctttccctcgctcccaagagcaactctgtttatctcgcaatgaaaaaagtccaggaacttcctgtggaggacgtaccgctttttctgagaaatcccgtcaccgaagagatgaaaaagcgtggatacggagaagggtatctttatccccatgatttcggtggtttcgtgaaaacgaactatcttccagaaaagttgaaaaatgaggtcatcttccagccaaagagagtaggttttgaggaagaactctttgaaaggctcagaaaactctggcccgagaagtacgggggtgaaagtatggccgaagtgagaaaagaactggaatacaaagggaaaaagatcagaatcgtaaagggagacatcacaagagaagaagtggacgccatagtgaacgcggcgaacgaatatctgaaacacggaggaggagtagcgggagcgatcgtgagagcaggtggaagcgttatccaggaagaaagcgacaggatcgttcaagaacgaggaagagtcccgacaggtgaagcggtggtaaccagcgctggaaagctgaaggcaaaatacgtgatccacaccgtggggcccgtttggagaggaggcagtcatggagaggacgaacttctctacaaagcagtttacaacgctcttcttcgagctcacgaactgaaattgaagagcatctcaatgcctgctatcagtacaggaatattcggatttccgaaagaaagagcggtgggaatcttttcaaaagcaataagagatttcatcgatcaacatcctgataccactctggaagagatccgcatatgcaacatagatgaggaaacaacgaagattttcgaagaaaagttcagcgtttgatgaaaccaaaagaaggggcaaacgccccttcttttttaagacctctgaggaagtattgaatcaactcaaacgagttcttggacctcttccgcaacagatttccatacctgaaaggaactattgatattttcatcataccacaaaaactaaaaaagtaaagggggccaagggccccctttatttccatcagaattcttcaggcatgctcggtgtttctttcttctcctcgggtttttcaacgatgagcacttccgttgtcaggagcattccagcgatggatgcagcattctgcagggcgctccttgtaacctttgctgggtcaatgattcctctttcaaacatgttgcagtattctcctctgagtgcgtcgaatccataggctggatcgtcattcgaaagtatcttctcaatgatcaccgctccgtcgtatcccgcgttttcagcaatctgcttaataggggctga

SEQ ID NO: 219 - protein, 312 aa, Thermotoga maritima
MNDLIRKYAKDQLETLKRIIEKSEGISILINGEDLSYPREVSLELPEYVEKFPPKASDVLEIDPEGENIGIDDIRTIKDFLNYSPELYTRKYVIVHDCERMTQQAANAFLKALEEPPEYAVIVLNTRRWHYLLPTIKSRVFRVVVNVPKEFRDLVKEKIGDLWEELPLLERDFKTALEAYKLGAEKLSGLMESLKVLETEKLLKKVLSKGLEGYLACRELLERFSKVESKEFFALFDQVTNTITGKDAFLLIQRLTRIILHENTWESVEDQKSVSFLDSILRVKIANLNNKLTLMNILAIHRERKRGVNAWS

SEQ ID NO: 220 - DNA, 1980 bp, Thermotoga maritima
ggaagatagctccgctccgcccaaacgtccggttgctcgttcagaatgtggtacagtatctcaagaccgtggtttgaagtacctacctcgtaagtgtcgggaaaaacgagggctatccggagggaaatttcctctggatctttcacaacgctgttgatttctcttcctatgtatctacttggttttctaacccagggaagtgtttcattcaagaattttagaatcatcctttcacctcagttctgattttatcattggttgttataattagaaggcgaggtgagatcatgaacgatttgatcagaaagtacgctaaagatcaactggaaactttgaaaaggatcatagaaaagtctgaaggaatatccatcctcataaatggagaagatctctcgtatccgagagaagtatcccttgaacttcccgagtacgtggagaaatttcccccgaaggcctcggatgttctggagatagatcccgagggggagaacataggcatagacgacatcagaacgataaaggacttcctgaactacagccccgagctctacacgagaaagtacgtgatagtccacgactgtgaaagaatgacccagcaggcggcgaacgcgtttctgaaggcccttgaagaaccaccagaatacgctgtgatcgttctgaacactcgccgctggcattatctactgccgacgataaagagccgagtgttcagagtggttgtgaacgttccaaaggagttcagagatctcgtgaaagagaaaataggagatctctgggaggaacttccacttcttgagagagacttcaaaacggctctcgaagcctacaaacttggtgcggaaaaactttctggattgatggaaagtctcaaagttttggagacggaaaaactcttgaaaaaggtcctttcaaaaggcctcgaaggttatctcgcatgtagggagctcctggagagattttcaaaggtggaatcgaaggaattctttgcgctttttgatcaggtgactaacacgataacaggaaaagacgcgtttcttttgatccagagactgacaagaatcattctccacgaaaacacatgggaaagcgttgaagatcaaaaaagcgtgtctttcctcgattcaattctcagggtgaagatagcgaatctgaacaacaaactcactctgatgaacatcctcgcgatacacagagagagaaagagaggtgtcaacgcttggagctgaaggcaagggcagtgggagtggagataataccaaagggtaagataatttactactctgtacccaacggtgatgagtacgagagaggagacctcgttctggtactgggagattttggacttgaagtgggaaaggttctgatacctcccagagaggtggtcatagacgaggtggggtacgaactcaaggccgtgatcagaaagttgacggatgaagatctggaacaatacaggaagaatgtagaggatgcgttgaaggccttccagatatgtaagcagaaaataaaagaacacggtcttcccatgaagcttctctacgccaagtacaccttcgatagaactaggctcatctttttcttcagtgcggaaggaagggttgatttcagagagcttgtaagagacctcgcaaagatcttcaaaacaaggatagagttgaggcaagttggtgtgagagatgagatgaagttctttggaggccttggattgtgcggacttccgacatgctgttccacatttctcagagagttcaccagcgtcacactgaagcacgcaaagaagcagcagatgatgatcaatccagcgaagatatccggaccatgcagaaggcttctgtgctgtcttacctacgaatacgatttctatgaaaaggagctggagggaattcctgacgaagggagcacgatcacctacgaaggaaagcgctacaaagtcgtgaacgtgaacgtgtttctcagaacggtgactctcttctcggaaca

SEQ ID NO: 221 - protein, 6 aa, Artificial Sequence
  Description of Artificial Sequence Consensus sequence
GXGKTX

SEQ ID NO: 222 - protein, 11 aa, Artificial Sequence
  Description of Artificial Sequence Consensus sequence
NALLKTLEEPP

SEQ ID NO: 223 - DNA, 996 bp, Mycobacterium tuberculosis
gtgagcgaggctaagccgttgcacctggtgagcgaggctaagccgttgcacctggtcctgggagacgaagaactgctggtcgaaagggcggtggccgacgtgttgcgctcggctcggcagcgggcaggtacagccgacgtcccggtgagccgaatgcgcgcgggtgacgtcggtgcctatgagctcgccgaactgctgagcccgtcactgttcgccgaggagcggatcgttgtgctgggggccgctgcggaggcgggcaaggacgctgccgcggtaatcgagtcggccgccgccgatcttccggccggcaccgtgctggtagtggtccactcgggtggcgggcgcgccaaatcgctggccaaccagctgcggtcgatgggtgcgcaggttcatccgtgcgcgcggatcaccaaggtcagtgagcgcgccgacttcatccgtagcgagttcgcgtcgctgcgggtcaaggtcgacgacgagaccgtgaccgccctgctggacgccgtcggctccgacgtgcgcgaactcgcctcggcctgttcacagctggtcgccgataccggaggagccgtcgacgccgccgctgtacggcgctatcacagcggcaaagccgaggtgaggggcttcgacatcgccgacaaggcggtagccggcgacgtggcgggagctgccgaagcgttgcggtgggcgatgatgcgcggtgagccgctagtggtgttggccgatgcgctcgccgaagccgtgcacaccatcggccgggtcgggccgcagtccggcgacccgtaccgcctggccgcacaactggggatgccgccctggcgggtgcagaaagcccagaagcaggctcggcggtggtcgcgtgacacggtggcgaccgcgatgaggttggtggccgaactcaatgctaacgtcaagggcgccgtcgcggatgcggactacgcgctggaatccgcggtccggcaggtcgccgagttggtggccgaccgcggccgatga

SEQ ID NO: 224 - DNA, 996 bp, Mycobacterium tuberculosis; identical to SEQ ID NO: 223
  CDS (1)..(996)

SEQ ID NO: 225 - protein, 331 aa, Mycobacterium tuberculosis
VSEAKPLHLVSEAKPLHLVLGDEELLVERAVADVLRSARQRAGTADVPVSRMRAGDVGAYELAELLSPSLFAEERIVVLGAAAEAGKDAAAVIESAAADLPAGTVLVVVHSGGGRAKSLANQLRSMGAQVHPCARITKVSERADFIRSEFASLRVKVDDETVTALLDAVGSDVRELASACSQLVADTGGAVDAAAVRRYHSGKAEVRGFDIADKAVAGDVAGAAEALRWAMMRGEPLVVLADALAEAVHTIGRVGPQSGDPYRLAAQLGMPPWRVQKAQKQARRWSRDTVATAMRLVAELNANVKGAVADADYALESAVRQVAELVADRGR

SEQ ID NO: 226 - protein, 350 aa, Aquifex aeolicus; identical to SEQ ID NO: 213

SEQ ID NO: 227 - protein, 316 aa, Mycobacterium tuberculosis
MHLVLGDEELLVERAVADVLRSARQRAGTADVPVSRMRAGDVGAYELAELLSPSLFAEERIVVLGAAAEAGKDAAAVIESAAADLPAGTVLVVVHSGGGRAKSLANQLRSMGAQVHPCARITKVSERADFIRSEFASLRVKVDDETVTALLDAVGSDVRELASACSQLVADTGGAVDAAAVRRYHSGKAEVRGFDIADKAVAGDVAGAAEALRWAMMRGEPLVVLADALAEAVHTIGRVGPQSGDPYRLAAQLGMPPWRVQKAQKQARRWSRDTVATAMRLVAELNANVKGAVADADYALESAVRQVAELVADRGR

SEQ ID NO: 228 - protein, 322 aa, Mycobacterium tuberculosis
MSEAKPLHLVLGDEELLVERAVADVLRSARQRAGTADVPVSRMRAGDVGAYELAELLSPSLFAEERIVVLGAAAEAGKDAAAVIESAAADLPAGTVLVVVHSGGGRAKSLANQLRSMGAQVHPCARITKVSERADFIRSEFASLRVKVDDETVTALLDAVGSDVRELASACSQLVADTGGAVDAAAVRRYHSGKAEVRGFDIADKAVAGDVAGAAEALRWAMMRGEPLVVLADALAEAVHTIGRVGPQSGDPYRLAAQLGMPPWRVQKAQKQARRWSRDTVATAMRLVAELNANVKGAVADADYALESAVRQVAELVADRGR

SEQ ID NO: 229 - protein, 336 aa, Streptomyces coelicolor
MRAMLVAMARKTANDDPLAPVTLAVGQEDLLLDRAVQEVVAAAKAADADTDVRDLTPDQLQPGTLAELTSPSLFAERKVVVVRNAQDLSADTVKDVKAYLGAPAEEITLVLLHAGGAKGKGVLDAGRKAGAREVACPKMTKPADRLAFVRAEFRTAGRSATPEACQALVDAIGSDLRELASAVSQLTADVEGTVDEAVVGRYYTGRAEASSFTVADRAVEGRAAEALEALRWSLATGVAPVLITSALAQGVRAIGKLSSARGGRPADLARELGMPPWKIDRVRQQMRGWTPDGVSVALRAVAEADAGVKGGGDDPEYALEKAVVIIARAARSRGRT

SEQ ID NO: 230 - protein, 478 aa, Thermotoga maritima; identical to SEQ ID NO: 178

(19) United States
(12) Patent Application Publication
(10) Pub. No.: US 2003/0219737 A1
(43) Pub. Date: Nov. 27, 2003

(54) Novel DNA polymerase III holoenzyme delta subunit nucleic acid molecules and proteins

(76) Inventors: James M. Bullard, Longmont, CO (US); Nebojsa Janjic, Boulder, CO (US); Charles S. McHenry, Denver, CO (US)

Correspondence Address: SWANSON & BRATSCHUN L.L.C., 1745 SHEA CENTER DRIVE, SUITE 330, HIGHLANDS RANCH, CO, 80129, US

(21) Appl. No.: 09/906,179

(22) Filed: Jul. 16, 2001

Related U.S. Application Data
(63) Continuation-in-part of application No. 09/818,780, filed on Mar. 28, 2001.
(60) Provisional application No. 60/218,246, filed on Jul. 14, 2000.
(60) Provisional application No. 60/192,736, filed on Mar. 28, 2000.

(51) Int. Cl. C12Q 1/68; G06F 19/00; G01N 33/48; G01N 33/50; C12N 9/22

(52) U.S. Cl. 435/6; 435/199; 702/20

(57) ABSTRACT

Gene and amino acid sequences encoding DNA polymerase III holoenzyme δ subunits and structural genes from bacteria are provided. Also provided are antibodies and other reagents useful to identify DNA polymerase III δ subunit molecules. Also provided are methods to identify DNA polymerase III δ subunit molecules. The use of DNA polymerase III δ subunit molecules in assays to identify candidate antibiotics are also provided.

### CROSS REFERENCE TO RELATED APPLICATIONS

This application claims the benefit of U.S. Provisional Patent Application Serial No. 60/218,246, filed Jun. 14, 2000, entitled “Methods for Identification of Delta and Delta Prime Subunits of Thermophilic Replicases and for Reconstitution of DNA Polymerase III Holoenzyme from Thermophilic Bacteria.”

### FIELD OF THE INVENTION

The present invention relates to gene and amino acid sequences encoding DNA polymerase III holoenzyme δ subunits and structural genes from bacteria. The present invention also provides antibodies and other reagents useful to identify DNA polymerase III δ subunit molecules. The present invention also provides methods for various uses of δ subunit molecules, including their use in DNA replication and in the identification of modulators of DNA replication. The present invention also provides δ proteins of thermophilic organisms for use in for DNA synthesis and amplification.

### BACKGROUND OF THE INVENTION

Like many other complex mechanisms of macromolecular synthesis, the fundamental mechanisms of DNA replication have been conserved throughout biology. The chemistry and direction of synthesis, the requirement for RNA primers, the mechanisms of semi-discontinuous replication with Okazaki fragments on the lagging strand and the need for well-defined origins are shared (Kornberg, A. and Baker, T. A. DNA Replication, W H Freeman and Company, New York (1992)). The basic features of the replicative apparatus are also shared. All replicases consist of a replicative polymerase that is distinguished from others primarily by its capability of participating in specific protein-protein interactions with the rest of the replicative apparatus. All prokaryotic cells have at least three polymerases; eukaryotes have at least five. Yet, only a subset of these polymerases can function as the replicase catalytic subunit (Table 1). In eukaryotes, it has been proposed that the δ polymerase is the leading strand polymerase and ε is the lagging (Burgers, P. M. J., J. Biol. Chem. 266:22698-22706 (1991); Nethanel, T. and Kaufmann, G., J. Virol. 64:5912-5918 (1990)), whereas, in E. coli, the α subunit of DNA polymerase III serves as the sole polymerization subunit. Another key replicase component is the so-called β sliding clamp that confers high processivity on the replicase. Processivity is defined as the number of nucleotides inserted per template association-catalysis-dissociation event. β consists of a bracelet-shaped molecule that clamps around DNA, permitting it to rapidly slide down DNA, but not to dissociate. The clamp contacts the polymerase by protein-protein interactions and prevents it from falling off of the template, ensuring high processivity. Two representative prokaryotic and eukaryotic sliding clamps are β and PCNA, respectively. The crystal structures of yeast PCNA and E. coli β are nearly superimposable (Kong, X. P., et al., Cell 79:1233-1243 (1994); Krishna, T. S., et al., Cell 79:1233-1243 (1994)). In both bacteria and eukaryotes, a 5-protein complex is responsible for transferring the sliding clamp onto a primer-terminus in an ATP-dependent reaction (Lee, S. H., et al., J. Biol. Chem. 266:594-602 (1991); Bunz, F., et al., Proc. Natl. Acad. Sci. U.S.A. 90:11014-11018 (1993)). Some of the subunits exhibit recognizable sequence homology between eukaryotes and both gram-negative and -positive prokaryotes, and would be expected to act by a similar mechanism (Carter, J. R., et al., J. Bacteriol. 175:3812-3822 (1993); O'Donnell, M., et al., Nucleic Acids Res. 21:1-3 (1993)).

The DNA polymerase III holoenzyme is the replicative polymerase of E. coli, responsible for synthesis of the majority of the chromosome (for a review, see Kelman, Z. and O'Donnell, M., Annu. Rev. Biochem. 64:171-200 (1995)). The replicative role of the enzyme has been established both by biochemical and genetic criteria. Holoenzyme was biochemically defined and purified using natural chromosomal assays. Only the holoenzyme form of DNA polymerase III efficiently replicates single-stranded bacteriophages in vitro in the presence of other known replicative proteins (Wickner, W. and Kornberg, A., Proc. Natl. Acad. Sci. U.S.A. 70:3679-3683 (1973); Hurwitz, J. and Wickner, S., Proc. Natl. Acad. Sci. U.S.A. 71:6-10 (1974); McHenry, C. S. and Kornberg, A., J. Biol. Chem. 252:6478-6484 (1977)). Only the holoenzyme functions in the replication of bacteriophage λ plasmids and molecules containing the E. coli replicative origin, oriC. The holoenzyme contains 10 subunits: α, τ, γ, β, δ, δ′, ε, ψ, χ and θ of 129,900, 71,000, 47,400, 40,600, 38,700, 36,900, 26,900, 16,600, 15,000 and 8,800 daltons, respectively.

TABLE 1
Relationships of the Components of Replicative Enzymes

| Component/ | | | |
|---|---|---|---|
| Function | E. coli | Phage T4 | Eukaryotes |
| DNA polymerase | α | Gene 43 | polymerase δ (and |
| | | | ε?) |
| Sliding Clamp | β | Gene 45 | PCNA |
| | | protein | |
| Clamp loading | DnaX, δ, δ′, χ, ψ | Gene 44/62 | Activator 1 (RFC) |
| complex | | complex | |
| Single-stranded | DNA SSB | Gene 32 | RFA |
| binding protein | | protein | |
| Primer generation | DnaG primase | Gene 61 | primase-α |
| | | protein | polymerase complex |

Genetic studies also support assignment of the major replicative role to the pol III holoenzyme. Temperature-sensitive mutations in dnaE, the structural gene for the α catalytic subunit, are conditionally lethal (Gefter, M. L., et al., Proc. Natl. Acad. Sci. U.S.A. 68:3150-3153 (1971)). Similarly, temperature-sensitive, conditionally lethal mutations have been isolated for the dnaN, dnaX and dnaQ genes that encode the β processivity factor, the DnaX protein and the ε proofreading exonuclease, respectively (Sakakibara, Y. and Mizukami, T., Mol. Gen. Genet. 178:541-553 (1980); Chu, H., et al., J. Bacteriol. 132:151-158 (1997); Henson, J. M., et al., Genetics 92:1041-1059 (1979); Horiuchi, T., et al., Mol. Gen. Genet. 163:277-283 (1978)). The structural genes for the final five holoenzyme subunits were identified by a reverse genetics approach (Carter, J. R., et al., (1993), ibid.; Carter, J. R., et al., J. Bacteriol. 174:7013-7025 (1992); Carter, J. R., et al., Mol. Gen. Genet. 241:399-408 (1993); Carter, J. R., et al., Nucleic Acids Res. 21:3281-3286 (1993); Carter, J. R., et al., J. Bacteriol. 175:5604-5610 (1993); Dong, Z., et al., J. Biol. Chem. 268:11758-11765 (1993); Xiao, H., et al., J. Biol. Chem. 268:11773-11778 (1993)). These were named these holA, holB, holC, holD, and hole for the δ, δ′, χ ψ and θ genes, respectively.

There are at least three distinct polymerases in E. coli, yet only the pol III holoenzyme appears to play a major replicative role. What are the special features of the pol III holoenzyme that confer its unique role in replication? Work to date suggests that the rapid elongation rate, high processivity, ability to utilize a long single-stranded template coated with the single-stranded DNA binding protein, resistance to physiological levels of salt and ability to interact with other proteins of the replicative apparatus are all critical to its unique functions. In contrast, other non-replicative polymerases such as DNA polymerase I cannot substitute effectively for replicases either in vitro or in vivo (Kornberg, and Baker, ibid.). This is because of their low processivity, their corresponding slow reaction rate, and their inability to interact with other replication proteins to enable catalysis of a coordinated reaction at the replication fork.

Multiple DNA polymerase III forms—The DNA polymerase III holoenzyme can be biochemically resolved into a series of successively simpler forms. DNA polymerase III core contains the α catalytic subunit complexed tightly to ε (proofreading subunit) and θ. DNA polymerase III′ contains core+τ (DnaX). The DnaX protein is the designation used for the dnaX gene product in this document. The DnaX protein appears in E. coli as two related proteins γ (47.4 kDa) and τ (71 kDa) (Tsuchihashi, Z. and Kornberg, A., J. Biol. Chem. 264:17790-17795 (1989)). γ represents the amino-terminal 5/7 of τ. The two proteins are nearly functionally interchangable for most purposes and are not distinguished for most purposes in this document. DNA polymerase III* contains pol III′+γ, δ-δ′, χ-ψ. Holoenzyme is composed of pol III*+β.

Processivity—Studies of the processivities of the multiple polymerase III forms have revealed individual contributions of subunits (Fay, P. J., et al., J. Biol. Chem. 256:976-983 (1981); Fay, P. J., et al., J. Biol. Chem. 257:5692-5699 (1982)). The multiple forms of DNA polymerase III exhibit strikingly different processivities. The core pol III has a low processivity (ca. 10 bases) in low ionic strength that decreases to being completely distributive (processivity=1) under more physiological conditions. Processivity is enhanced by addition of the τ DnaX subunit to form pol III′. Pol III′ achieves maximum processivity in the presence of physiological concentrations of spermidine, an agent that inhibits the core pol III. Addition of the γ, δδ′ and χψ to pol III′ to form pol III* further increases processivity in the presence of single-stranded DNA binding protein (SSB). The holoenzyme exhibits a processivity orders of magnitude greater than any of its subassemblies. In carefully controlled experiments with single-stranded phage, the entire template (up to 8000 nucleotides) is synthesized in a single processive event in under 15 s at 30° C. (Johanson, K. O. and McHenry, C. S., (1982) ibid.) (1 min at 22° C. (Fay, P. J., et al., (1981), ibid.). For coupled replication fork systems where the holoenzyme acts with primosomal components, processivities of 150-500 kb have been directly observed (Wu, C. A., et al., J. Biol. Chem. 267:4064-4073 (1992)). These products are synthesized at rates of 500-700 nt/s, permitting synthesis of 100,000 bases in ca. 3 min. Thus, a progression in processivities that parallels the structural complexity of the corresponding enzyme form is observed. For comparative purposes, the processivities of “repair-type” polymerases of the bacterial DNA polymerase I class are typically 15-50 bases (Bambara, R. A., et al., J. Biol. Chem. 253:413-423 (1978)).

Initiation Complex Formation—To achieve high processivity, the holoenzyme requires ATP (or dATP) and primed DNA to form a stable initiation complex (Fay, P. J., et al., (1981) ibid.). Initiation complexes can be isolated by gel filtration and, upon addition of dNTPs, form a complete RFII in 10-15 seconds without ever dissociating (Wickner, W. and Kornberg, A., ibid.; Hurwitz, J. and Wickner, S., ibid.; Johanson, K. O. and McHenry, C. S., J. Biol. Chem. 255:10984-10990 (1980)). Initiation complex formation can be monitored experimentally as a conversion of replicative activity to anti-β IgG resistance (Johanson, K. O. and McHenry, C. S., (1982) ibid.; Johanson, K. O. and McHenry, C. S., (1980) ibid.). β participates in elongation; antibody resistance arises from β's immersion in the complex, sterically precluding antibody attachment. Consistent with this observation, M. O'Donnell's lab has found that a kinase recognition peptide fused to the carboxyl-terminus of β can be readily phosphorylated in solution but not in initiation complexes (Stukenberg, P. T., et al., Cell 78:877-887 (1994)).

Structure of the β Sliding Clamp—The X-ray crystallographic structure of β, solved by Kuriyan, O'Donnell and coworkers (Kong, X. P. et al., (1994) ibid.), provides a simple and elegant explanation for its function. The β dimer forms a bracelet-like structure, presumably with DNA passing through the central hole, permitting it to slide down DNA rapidly but preventing it from readily dissociating. Protein-protein contacts between β and other components of the replicative complex tether the polymerase to the DNA, increasing its processivity. A tightly clasped bracelet would not be expected to readily associate with DNA. This explains the need for an energy-dependent clamp-setting complex, the DnaX-complex, to recognize the primer terminus and open and close the β-bracelet around DNA.

DnaX Complex: The Apparatus that Sets the β Sliding Clamp onto Primed DNA—The DnaX protein contains a consensus ATP binding site near its amino-terminus (Yin, K. C., et al., Nucleic Acids Res. 14:6541-6549 (1986)) that is used to bind and hydrolyze ATP, in concert with δ-δ′ and χ-ψ, setting the β processivity clamp on the primer-terminus. DnaX binds ATP with a dissociation constant of ca. 2 μM (Tsuchihashi, Z. and Kornberg, A. (1989) ibid.) and is a DNA-dependent ATPase (ibid.; Lee, S. H. and Walker, J. R., Proc. Natl. Acad. Sci. U.S.A. 84:2713-2717 (1987)). The primer-terminus appears to be the most active effector of the ATPase (Onrust, R., et al., J. Biol. Chem. 266:21681-21686 (1991)). DnaX (τ) binds the α subunit DNA polymerase III core and causes it to dimerize, forming the dimeric scaffold upon which other auxiliary proteins can assemble to form a dimeric replicative complex. In vitro, the τ DnaX subunit can readily form a “τ-complex” (τ-ψ-χ-δ-δ′) that functions to load β onto primed DNA (Dallmann, H. G. and McHenry, C. S., J. Biol. Chem. 270:29563-29569 (1995); Onrust, R., et al., J. Biol. Chem. 270:13348-13357 (1995); Dallmann, H. G., et al., J. Biol. Chem. 270:29555-29562 (1995)). The stoichiometry of the DnaX complexes and has been found to have 3 copies of the DnaX protein and 1 each of the ancillary subunits (DnaX₃δ₁δ′₁χ₁ψ₁) (Pritchard, A., Dallmann, G., Glover, B. and McHenry, C. (2000) EMBO J. 19:6536-6545).

Structure of the DNA polymerase III holoenzyme—FIG. 1 illustrates the current working hypothesis for holoenzyme subunit-subunit interactions. α and ε form an isolable complex upon mixing (Maki, H. and Kornberg, A., Proc. Natl. Acad. Sci. U.S.A. 84:4389-4392 (1987)). Mutations in the structural gene for E have also been found that suppress dnaE (α) mutations (Maurer, R., et al., Genetics 108:25-38 (1984)). Suppressor mutations most likely arise through modification of a subunit that interacts directly with the suppressed mutant gene product. Pol III core (αεθ) is isolable (McHenry, C. S. and Crow, W., J. Biol. Chem. 254:1748-1753 (1979)). DnaX (τ can be isolated in a complex with pol III core (McHenry, C. S., J. Biol. Chem. 257:2657-2663 (1982)). Suppressor data suggest an interaction between DnaX and β (Engstrom, J., et al., Genetics 113:499-516 (1986)), although a direct interaction has not yet been demonstrated biochemically. DnaX in the presence of δ and δ can transfer β to primed DNA to form a preinitiation complex (O'Donnell, M. and Studwell, P. S. (1990) J. Biol. Chem. 265:1179-1187). Suppressor data indicate an interaction between β and α (Kuwabara, N. and Uchida, H., Proc. Natl. Acad. Sci. U.S.A. 78:5764-5767 (1981)), a notion supported by the ability of to interact with and increase the processivity of core pol III (Laduca, R. J., et al., J. Biol. Chem. 261:7550-7557 (1986)) and the observation of an interaction between β and the carboxyl-terminal domain of the α catalytic subunit (Kim, D. R. and McHenry, C. S., J. Biol. Chem. 271:20699-20704 (1996)). Genetic evidence for α-α interaction and for the dimeric nature of pol III holoenzyme was obtained through interallelic complementation between dnaE₁₀₂₆ and dnaE₄₈₆ or dna E₅₁₁ (Bryan, S., et al., “DNA Polymerase III is Required for Mutagenesis,” in DNA Replication and Mutagenesis, Moses, R. & Summers, W., eds., American Society for Microbiology, Washington, DC)—if this interaction occurs, it must be weak and require the presence of other subunits, since no α-α interaction is seen in vitro. χ and ψ have been isolated in a complex with DnaX (Olson, M. W., et al., J. Biol. Chem. 270:29570-29577 (1995); O'Donnell, M. and Studwell, P. S., J. Biol. Chem. 265:1179-1187 (1990)). δ and δ′ can interact weakly by themselves in solution and together can interact with DnaX (Dallmann, H. G. and McHenry, C. S., J. Biol. Chem. 270:29563-29569 (1995); Olson, M. W., et al., ibid.; Onrust, R. and O'Donnell, M., J. Biol. Chem. 268:11766-11772 (1993)). ψ and δ′ are apparently the subunits that interact directly with DnaX (Onrust, R., et al., J. Biol. Chem. 270:13348-13357 (1995)). A direct δ-β interaction has been detected (Naktinis, V., et al., J. Biol. Chem. 270:13358-13365 (1995)). There are three copies of DnaX protein in the holoenzyme (Pritchard, A., et al., (2000) ibid.).

The Polymerase Chain Reaction—The polymerase chain reaction (PCR) has made enormous contributions to molecular biology. It is useful for direct DNA amplification from complex genome preparations, for direct sequence analysis, for detection or diagnosis of mutations, for pathogen diagnosis and forensic analysis, and for a variety of manipulations used in molecular biology research (Arnheim, N. and Erlich, H., Annu. Rev. Biochem. 61:131-156 (1992)). In its simplest form, the reaction involves three steps: (1) Target DNA is thermally denatured to make it single-stranded. (2) Oligonucleotides flanking the sequence to be amplified are annealed. Sequences of ca. 20 nucleotides normally provide the required specificity for amplification of a specific gene. If the genome sequence is known, sequences can be selected to increase specificity and avoid partial regions of complementarity elsewhere on the genome. Solution conditions can also be altered to influence stringency. (3) A thermophilic DNA polymerase extends the primers until replication occurs past the second primer binding site. The three steps are repeated until the product is sufficiently abundant to permit isolation and analysis, typically 25-30 cycles.

A major limitation of the method is the requirement that replication must occur over the entire length of the DNA separating the primers. This introduces serious limitations with natural sequence DNA in the range over 10 kb and, for practical purposes, often over shorter regions. Innovations have enabled PCR amplification to 30-40 kb by well-set up labs. Although a large number of solution conditions must be optimized for each sequence amplified, the major contribution comes from inclusion of a proofreading polymerase in the thermophilic polymerase mixture (Barnes, W. M., Proc. Natl. Acad. Sci. U.S.A. 91:2216-2220 (1994); Cheng, S., et al., Proc. Natl. Acad. Sci. U.S.A. 91:5695-5699 (1994)). Presumably, the 3′→5′ proofreading exonuclease removes misincorporated bases that are not extended by most DNA polymerases. Many labs have found this method difficult to adapt to practice (Hengen, P. N., Trends Biochem. Sci. 19:341-342 (1994)) and even in expert labs, not all sequences over 20 kb are amplifiable (Cheng, S., et al., Proc. Natl. Acad. Sci. U.S.A. 91:5695-5699 (1994)). The thermophilic eubacterial DNA polymerases used in these systems are of the DNA polymerase I class (Lawyer, F. C., et al., J. Biol. Chem. 264:6427-6437 (1989)) that would be expected to have limited processivity and limited efficiency for amplification of very long sequences. Archaebacterial DNA polymerases are less defined. However, the polymerases used are the most abundant in cells and by analogy to both eukaryotic and prokaryotic systems, would be expected to be abundant repair class polymerases of limited processivity. A robust processive thermophilic DNA polymerase III holoenzyme should enable one to develop practical applications for amplification of high molecular weight DNA.

DNA Polymerase III Holoenzyme from Thermophilic Organisms

A subassembly of a thermophilic DNA polymerase III holoenzyme has been isolated, indicating that a thermophilic replicase, which had long-eluded previous isolation attempts, was generally used for bacterial DNA replication and was not limited to mesophilic organisms (McHenry, C. S., et al., J. Mol. Biol. 272:178-189 (1997)). Using protein sequence obtained from the isolated replicase, the identity of γ and τ-like proteins in T. thermophilus were directly demonstrated (McHenry, C. S., et al., (1997) ibid.). In a parallel approach, the O'Donnell laboratory directly isolated a dnaX gene from T. thermophilus chromosomal DNA using PCR and oligonucleotides corresponding to conserved regions (Yurieva, O., et al., J. Biol. Chem. 272:27131-27139 (1997)). Expression of T. thermophilus dnaX in E. coli leads to synthesis of γ- and τ-like proteins (Yurieva, O., et al., ibid.; PCT WO99/13060; PCT WO9953074), apparently by a transcriptional slippage mechanism (Larsen, B., et al., Proc. Natl. Acad. Sci. U.S.A. 97:1683-1688 (2000)). Partial protein sequence from the isolated T. thermophilus α subunit has led to the identification of the structural gene (PCT WO99/13060; PCT WO9953074) and expression of active native and tagged T. thermophilus α subunit (PCT WO99/13060). Taking advantage of the close genetic linkage between the chromosomal initiation protein dnaA and the structural gene for the sliding clamp processivity factor β (dnaN), the T. thermophilus dnaN gene was isolated, sequenced and the information used to direct the synthesis and subsequent purification of active T. thermophilus β (PCT WO99/13060; PCT WO9953074). The Thermus thermophilus DnaX complex subunits δ and δ′ have also been expressed in active form and used to reconstitute an active T. thermophilus DNA polymerase III holoenzyme (U.S. Utility patent application Ser. No. 09/818,780).

From the sequence of the Aquifex aeolicus and Thermotoga maritima genomes, it is apparent that a DNA polymerase III holoenzyme exists in these organisms too (Deckert, G., et al., Nature 392:353-358 (1998); Huang, Y. P. and Ito, J., Nucleic Acids Res. 26:5300-5309 (1998); Nelson, K. E., et al., Nature 399:323-329 (1999)). The sequence of Aquifex aeolicus was annotated, at the time of publication, to reveal homologs of the α (dnaE) and ε (dnaQ) components of the DNA polymerase III core, a β sliding clamp processivity factor (dnaN), a single stranded DNA binding protein (ssb) and the central DnaX protein of the DnaX complex (Deckert, G., et al., (1998) ibid.) (FIGS. 2A-D). Missing however, was the analog of the δ′, δ, χ and ψ DNA polymerase III holoenzyme subunits.

In Thermotoga maritima the sequence of two distinct DNA polymerase III-like catalytic subunits was published (Huang, Y. P. and Ito, J., (1998) ibid.), a situation in parallel with gram-positive (firmicute phylum) mesophilic bacteria (Koonin, E. V., and Bork, P., Trends Biochem. Sci. 21:128-129 (1996)). One DNA polymerase III contained the 3′→5′ exonuclease as part of the same polypeptide while a second α subunit and ε subunit are apparently encoded by an E. coli-like dnaE and dnaQ gene respectively. The homologs for dnaN, ssb, dnaX are annotated (FIGS. 3A-F). An additional “γ-like” gene (TM0771) is annotated that is likely holB; however, because of poor sequence conservation, the structural genes for the homologs of the DNA polymerase III holoenzyme δ, χ and ψ subunits are not apparent. The lack of a structural gene for a δ subunit is particularly problematic because of its expected essentiality for reconstitution of a processive DNA polymerase III holoenzyme from Aquifex aeolicus and Thermotoga maritima.

Replication in Gram-Positive (Firmicute Phylum) Organisms

DNA replication has not been as extensively studied in the prototype for gram-positive (firmicute phylum) organisms, B. subtilis, as in E. coli. Of the elongation components, only the basic DNA polymerase III catalytic subunit has been purified (Low R. L., Rashbaum S. A., and Cozzarelli N. R. J. Biol. Chem. 251:1311-1325 (1976); Hammond, R. A., Barnes, M. H., Mack, S. L., Mitchener, J. A., Brown, N. C., Gene 98:29-36 (1991)). This enzyme shows sequence similarity to the E. coli DNA polymerase III, but differs in that the 3′→5′ proofreading activity is contained within the same polypeptide chain. Examination of the sequence of B. subtilis pol III reveals sequence with close similarity with the E. coli ε proofreading subunit inserted near the amino-terminus (Barnes, M., Hammond, R., Kennedy, C., Mack, S. and Brown, N. C., Gene 111:43-49 (1992)). With the completion of several genomic sequences from gram-positive organisms, it became apparent that a second DNA polymerase III α subunit exists with the sequence more closely resembling the E. coli-like enzyme (Koonin, E. V., and Bork, P., Trends Biochem. Sci. 21:128-129 (1996)). Recently, a similar situation has been observed in the more distant Thermotoga maritima (Huang, Y. P. & Ito, J. (1998) Nucleic Acids Res. 26:5300-5309 (1998)). Pritchard (Pritchard, A. & McHenry, C. S., J. Mol. Biol. 286:1067-1080 (1999)) and Ito (Huang, Y. P., and Ito, J. (1998) ibid.) have referred to the prototypical E. coli-like and B. subtilis-like polymerases type I and II, respectively, a convention followed in this application. A type II pol III, where it exists, always is present as the second pol III-like enzyme. This raises important questions regarding the mechanistic contributions of each polymerase; an issue that these proposed studies should resolve. In B. subtilis, the prototypical gram-positive type II pol III is clearly essential for cell viability. Hydroxyphenylazopyrimidines inhibit this enzyme and drug-resistance maps to the structural gene for the type II enzyme (Love, E., Dambrosio, J., Brown, N., and Dubnau, D., Mol. Gen. Genet. 144:313-321 (1976)). However, D. Ehrlich and colleagues have recently shown that the type I pol III is also required for cell viability (personal communication). In the closely related organism S. pyogenes, the minimal components for the elongation apparatus (type II pol III, β, τ, δ and δ′) have recently been expressed and used to reconstitute a minimal replicase (Bruck, I. and O'Donnell, M., J. Biol. Chem. 275:28971-28983 (2000); Klemperer, N, Zhang, D, Skangalis, M and O'Donnell, M. J. Biol. Chem. 275:26136-26143 (2000); PCT WO 01/09164).

TABLE 2
E. coli and Corresponding B. subtilis Replication Proteins

| | | | |
|---|---|---|---|
| | | | homology |
| E coli | | B. subtilis | with E. |
| gene | subunit/function | gene | coli |

Elongation Components

| | | | |
|---|---|---|---|
| dnaE | α/replicative polymerase (pol III | dnaE | strong |
| | type I) | | |
| | α + ε/replicative polymerase (pol III | polC | moderate |
| | type II) | | |
| dnaQ | ε/3′→5′ proofreading exonuclease | not known | ND |
| holE | θ/no known function | not known | ND^{b} |
| dnaX | γ, τ/ATPase that loads β₂ onto DNA | dnaX | strong |
| holA | δ/binds β, essential part of DnaX | yqeN | weak |
| | complex | | |
| hol1B | δ′/essential part of DnaX complex | holB | strong |
| holC | χ/nonessential-interacts with SSB | not known | ND |
| holD | ψ/nonessential-increases the affinity | not known | ND |
| | of DnaX for δ′ | | |
| dnaN | β/processivity factor | dnaN | strong |
| ssb | SSB/single stranded DNA binding | ssb | strong |
| | protein | | |
| dnaG | primase/RNA priming | dnaG | strong |

^{b}No homology to E. coli replication proteins detectable.

The genomic sequences of B. subtilis and other related organisms reveal the likely structural genes for the DNA polymerase III holoenzyme auxiliary subunits with the exception of θ, χ and ψ which are not highly conserved, even among very closely related bacteria. B. subtilis homologs of dnaX, holB (δ′), dnaN (β) and ssb are also apparent and documented within the B. subtilis genome project web site (available at the SubtiList website of the Pasteur Institute, Paris). The primase and SSB proteins are apparent from sequence examination and has been documented in the genomes of B. subtilis and other organisms.

Cytology of DNA Replication Apparatus—Recently developed improvements in technology has enabled visualization of the replication apparatus in bacteria (Lemon, K. and Grossman, A. (1998) Science 282:1516-1519; Gordon, G. S., Sitnikov, D., Webb, C. D., Teleman, A., Straight, A., Losick, R., Murray A. W., Wright, A., Cell 90:1113-1121 (1997); Newman, G. and Crooke, E., J. Bacteriol. 182:2604-2610 (2000)). Using C-terminally tagged B. subtilis Pol C (DNA polymerase III type II), DnaX (τ) and HolB (δ′), Grossman and colleagues have demonstrated that the replication apparatus is fixed with a central part of the cell and that the chromosome passes through it during replication (Lemon, K. and Grossman, A., Science 282:1516-1519 (1998); Lemon, K. and Grossman, A., Molecular Cell 6:1321-1330 (2000)). However, the origin appears to progress to the cell poles as it is replicated. Two gene products that appear to be associated with the initiation of chromosomal replication (B. subtilis DnaB and DnaI proteins) have been visualized to co-localize as foci predominantly associated with the edges of the nucleoid during the initiation of replication (Imai Y, Ogasawara N, Ishigo-Oka D, Kadoya R, Daito T, Moriya S., Mol. Microbiol. 36:1037-48 (2000)).

Bacteria and Antibacterial Drugs. Bacterial infections continue to represent a major worldwide health hazard (Overcoming Antimicrobial Resistance. World Health Organization, Report on Infectious Diseases (WHO/CDS/2000.2) (2000)). Infections range from the relatively innocuous, such as skin rashes and common ear infections in infants, to the very serious and potentially lethal infections in immune-compromised patients. Drug-resistant hospital-acquired and community infections are increasing. With the recent emergence of numerous, clinically important, drug-14resistant bacteria including Staphylococcus aureus, Streptococcus pneumoniae, Enterococcus faecalis, Mycobacterium tuberculosis, and Pseudomonas aeruginosa, an emergency is becoming apparent.

Classes of Antibacterials and Mechanism of Resistance. Antibacterials kill bacteria by interfering with major processes of cellular function that are essential for survival. The β-lactams (penicillins and cephalosporins) and the glycopeptides (vancomycin and teicoplanin) inhibit synthesis of the cell wall. Macrolides (erythromycin, clarithromycin, and azithromycin), clindamycin, chloramphenicol, aminoglycosides (streptomycin, gentamicin, and amikacin) and the tetracyclines inhibit protein synthesis. Also inhibiting protein synthesis is the newest class of antibacterials to be approved (linezolid) that are synthetic oxazolidinones. Rifampin inhibits RNA synthesis, the fluoroquinolones (such as ciprofloxacin) inhibit DNA synthesis indirectly by inhibiting the enzymes that maintain the topological state of DNA. Trimethoprim and the sulfonamides inhibit folate biosynthesis directly and DNA synthesis indirectly by depleting the pools of one of the required nucleotides (Chambers, H. F. and Sande, M. A., Antimicrobial Agents. Goodman & Gilman 's The Pharmacological Basis of Therapeutics, McGraw-Hill, New York (1996)).

Resistance to antibacterials can occur when the target of a drug mutates so that it can still function, but is no longer blocked by the drug, or by mutation of efflux pumps or modification enzymes to broaden their specificity to a new drug. Because of the growth advantage this gives to the resistant cell and its progeny in the presence of the antibacterial, the resistant organisms quickly take over a population of bacteria. Resistance developed in one cell can be transferred to other bacteria in the population since bacteria have mechanisms for directly exchanging genetic material. In a recent congressional report, the General Accounting Office (GAO) has summarized the current and future public health burden resulting from drug-resistant bacteria (Antimicrobial Resistance. General Accounting Office, GAO/RCED-99-132 (1999)). According to this report, the number of patients treated in a hospital setting for an infection with drug-resistant bacteria has doubled from 1994 to 1996 and again almost doubled from 1996 to 1997. Resistant strains can spread easily in environments such as hospitals or tertiary care facilities that have a sizeable population of immunosuppressed patients. The same GAO report also provides clear evidence that previously susceptible bacteria are increasingly becoming resistant and spreading around the world. Furthermore, the proportion of resistant bacteria within bacterial populations is on the rise. An especially frightening development is the appearance of bacterial strains that are resistant to all approved antibacterials. Recognizing the dramatic increase of drug-resistant bacteria, the Food and Drug Administration has recently issued a recommendation urging physicians to use antibacterials more judiciously and only when clinically necessary (FDA Advisory. Federal Register 65 (182), 56511-56518 (2000)).

Until recently, drug developers tended to respond to emerging, drug-resistant bacterial strains by simply modifying existing antibacterials. Indeed, most of the antibacterials in late-stage development are analogs of drugs already in clinical use. This strategy is becoming less effective since the basic mechanism for resistance to the antibacterial class is already widespread in nature. Further mutation of the resistant target to confer resistance on a new antibacterial of the same class can often occur by a single base change in the gene for the target. Thus, the utility of antibacterials presently in the development pipeline is expected to be finite and the health care industry is currently in severe need of novel antibacterials that are mechanistically distinct from existing drugs. Accordingly, there remains a need for a chromosomal replication system to discover novel therapeutic agents that inhibit the central DNA replication apparatus of bacteria in order to combat bacterial health hazards.

### SUMMARY OF THE INVENTION

The present invention relates to gene and amino acid sequences encoding DNA polymerase III holoenzyme δ subunits from bacterial genes and nucleic acid molecules, including those that encode such proteins and to antibodies raised against such proteins. The present invention also includes methods to obtain such proteins, nucleic acid molecules and antibodies.

The present invention provides methods for identifying a bacterial DNA polymerase holoenzyme δ subunit protein from at least one completely sequenced genome comprising providing a database of sequence information for a plurality of different organisms, setting an inclusion threshold to a level sufficient to minimize the number of required iterations, selecting the organisms to be searched wherein the selected organisms are candidate organisms, providing the amino acid sequence of the δ subunit protein from a reference organism, comparing the sequences of the candidate organisms and the reference organism, excluding known DnaX proteins and known δ′ proteins from further steps, selecting proteins comprising between 300 and 400 amino acids, selecting a delta candidate from a candidate organism comprising selecting sequence with the lowest E score from the candidate organism and optionally repeating steps the preceding three steps wherein the sequences of the candidate organisms are sequences with the lowest E score from the candidate organism, wherein a bacterial DNA polymerase holoenzyme δ subunit protein may be identified.

The present invention provides bacterial DNA polymerase III holoenzyme δ subunit proteins identified by the method. The method can be modified to obtain δ subunit proteins from organisms with only partially sequenced genomes.

The present invention also provides methods for identifying a bacterial DNA polymerase holoenzyme δ protein from at least one completely sequenced genome comprising providing a database of sequence information for a plurality of different organisms, setting the inclusion threshold to a level sufficient to minimize the number of required iterations, selecting the organisms to be searched, wherein the selected organisms are candidate organisms, providing the amino acid sequence of the δ subunit protein from a reference organism, comparing the sequences of the candidate organisms and the reference organism, excluding known DnaX proteins and known δ′ proteins from further steps, selecting proteins comprising at least three of the following six characteristics:

a. having at least five out of ten conserved residues from the conserved region 17—(L/I/V)XX(L/I/V)Y(L/I/V)(L/I/V)XGX(D/E)XX(L/W/V)(L/I/V)XXXXXX(L/I/V)—38;

b. having at least five out of eleven conserved residues from the conserved region 64—(L/I/V)(L/I/V)XXXX(A/S)XX(L/I/V)F(A/S)X(K/R)X(L/I/V)(L/I/V)(L/I/V)(L/I/V)—82;

c. having at least five out of ten conserved residues from the conserved region 97—(L/I/V)XX(L/I/V)(L/I/V)XXXXX(D/E)X(L/I/V)(L/I/V)(L/I/V)(L/I/V)XXXK(L/I/V)—117;

d. having at least nine out of eighteen conserved residues from the conserved region 154—(R/K)XXX(L/I/V)X(L/I/V)X(L/I/V)(D/E)X(D/E)(A/S)(L/I/V)XX(L/I/V)XXXXXXN(L/I/V) XX(L/I/V)XX(D/E)(L/I/V)X(R/K)(L/I/V)X(L/I/V)(L/I/V)—191;

e. having at least eleven out of twenty-three conserved residues from the conserved region 215—FX(L/I/V)X(D/E)(A/S)(L/I/V)(L/I/V)XG(R/K)XXX(A/S)(L/I/V)X(L/I/V)(L/I/V)XX(L/I/V) XXXGX(D/E)P(L/I/V)X(L/I/V)(L/I/V)XX(L/I/V)XXX(L/I/V)XX(L/I/V)XX(L/I/V)—260; and

f. having at least six out of twelve conserved residues from the conserved region 298—(L/I/V)XXX(L/I/V)XX(L/I/V)XXX(D/E)XX(L/I/V)(R/K)XXXXX(D/E)XXXX(L/I/V)(D/E) XX(L/I/V)(L/I/V)X(L/I/V)—331; and

retaining the sequences above the threshold, wherein the retained sequences are delta candidate sequences; and optionally repeating steps the preceding three steps wherein the sequences of the candidate organisms are delta candidate sequences, whereby a δ protein may be identified.

The present invention provides bacterial DNA polymerase III holoenzyme δ subunit proteins identified by the method. The method can be modified to obtain δ subunit proteins from organisms with only partially sequenced genomes.

The present invention also provides methods for identifying a δ protein from at least one target organism comprising identifying a δ protein from an evolutionarily related organism, and performing a search for a similar sequence in the sequence of the target organism.

The present invention further provides isolated bacterial DNA polymerase δ subunit proteins, wherein the δ subunit protein is not a member of the group consisting of an E. coli δ subunit protein, a Haemophilis influenzae δ subunit protein, and a Neisseria meningitides δ subunit protein.

The present invention further provides isolated bacterial DNA polymerase δ subunit proteins, wherein the δ subunit protein is not a member of the group consisting of a gamma division of proteobacteria δ subunit protein and a beta division of proteobacteria δ subunit protein.

The present invention further provides isolated bacterial DNA polymerase δ subunit proteins, wherein the δ subunit protein is a gram-positive (firmicute phylum) bacteria δ subunit protein, including S. pyogenes, δ subunit proteins, S. aureus δ subunit proteins, and S. pneumoniae δ subunit proteins.

The present invention further provides isolated bacterial DNA polymerase δ subunit proteins derived from thermophilic organisms, wherein the δ subunit protein is selected from the group consisting of Thermus Thermophilus, Aquifex aeolicus, and Thermatoga maritima δ subunit protein.

The present invention provides an antibody to a δ subunit protein of the present invention, which may be a polyclonal, monoclonal, chimeric, single chain, F_{ab} fragments, or F_{ab} expression library, and a method for producing anti-DNA polymerase III δ subunit antibodies comprising exposing an animal having immunocompetent cells to an immunogen comprising at least an antigentic portion of DNA polymerase III δ subunit.

The present invention provides a method for detecting DNA polymerase III δ subunit protein comprising, providing in any order, a sample suspected of containing DNA polymerase III, an antibody capable of specifically binding to at least a portion of the DNA polymerase III δ subunit protein, mixing the sample and the antibody under conditions wherein the antibody can bind to the DNA polymerase III; and detecting the binding.

The present invention also provides isolated nucleic acid molecules which encode a δ subunit protein of the present invention and their complements.

The present invention also provides arecombinant molecules and recombinant cells comprising at least a portion of a bacterial DNA polymerase III nucleic acid molecule according to the present invention.

Furthermore, the present invention provides a method for detection of nucleic acid molecules encoding at least a portion of DNA polymerase III δ subunit in a biological sample comprising the steps of hybridizing at least a portion of a holA nucleic acid molecule to nucleic acid material of a biological sample, thereby forming a hybridization complex, and detecting the hybridization complex, wherein the presence of the complex correlates with the presence of a polynucleotide encoding at least a portion of DNA polymerase III δ subunit in the biological sample.

The present invention also provides a method for detecting DNA polymerase III δ subunit expression, including expression of modified or mutated DNA polymerase III δ subunit proteins or gene sequences comprising the steps of providing a test sample suspected of containing DNA polymerase III δ subunit protein, as appropriate; and comparing test DNA polymerase III δ subunit with quantitated DNA polymerase III holoenzyme or holoenzyme subunit in a control to determine the relative concentration of the test DNA polymerase III δ subunit in the sample.

In addition, the present invention provides a method of screening for a compound that modulates the activity of a DNA polymerase III replicase comprising contacting an isolated replicase with at least one test compound under conditions permissive for replicase activity, assessing the activity of the replicase in the presence of the test compound; and comparing the activity of the replicase in the presence of the test compound with the activity of the replicase in the absence of the test compound, wherein a change in the activity of the replicase in the presence of the test compound is indicative of a compound that modulates the activity of the replicase and wherein said replicase comprises an isolated DNA polymerase III δ subunit protein.

The present invention describes a method of identifying compounds which modulate the activity of a DNA polymerase III replicase. This method is carried out by forming a reaction mixture that includes a primed DNA molecule, a DNA polymerase III replicase, a candidate compound, a dNTP or up to 4 different dNTPs, ATP, and optionally either a β subunit, a DnaX complex, or both the β subunit and the DnaX complex; subjecting the reaction mixture to conditions effective to achieve nucleic acid polymerization in the absence of the candidate compound; analyzing the reaction mixture for the presence or absence of nucleic acid polymerization extension products and comparing the activity of the replicase in the presence of the test compound with the activity of the replicase in the absence of the test compound. A change in the activity of the replicase in the presence of the test compound is indicative of a compound that modulates the activity of the replicase. The DnaX complex comprises a δ subunit protein of the present invention.

The present invention describes a method to identify candidate pharmaceuticals that modulate the activity of a DnaX complex and a β subunit in stimulating the DNA polymerase. The method includes contacting a primed DNA (which may be coated with SSB) with a DNA polymerase, a β subunit, and a DnaX complex (or subunit or subassembly of the DnaX complex) in the presence of the candidate pharmaceutical, and dNTPs (or modified dNTPs) to form a reaction mixture. The reaction mixture is subjected to conditions which, in the absence of the candidate pharmaceutical, would effect nucleic acid polymerization and the presence or absence of the extension product in the reaction mixture is analyzed. The nucleic acid polymerization in the presence of the test compound is compared with the nucleic acid polymerization in the absence of the test compound. A change in the nucleic acid polymerization in the presence of the test compound is indicative of a compound that modulates the activity of a DnaX complex and a β subunit. The DnaX complex comprises a δ subunit of the present invention.

The present invention describes a method to identify chemicals that modulate the ability of a β subunit and a DnaX complex (or a subunit or subassembly of the DnaX complex) to interact. This method includes contacting the β subunit with the DnaX complex (or subunit or subassembly of the DnaX complex) in the presence of ATP (or a suitable ATP analog, such as ATPγS, a non-hydrolyzable analog of ATP, that enables interaction of β with the DnaX complex (or subunit or subassembly of the DnaX complex) the candidate pharmaceutical to form a reaction mixture. The reaction mixture is subjected to conditions under which the DnaX complex (or the subunit or subassembly of the DnaX complex) and the β subunit would interact in the absence of the candidate pharmaceutical. The reaction mixture is then analyzed for interaction between the β subunit and the DnaX complex (or the subunit or subassembly of the DnaX complex). The extent of interaction in the presence of the test compound is compared with the extent of interaction in the absence of the test compound. A change in the interaction between the β subunit and the DnaX complex (or the subunit or subassembly of the DnaX complex) is indicitive of a compound that modulates the interaction. The DnaX complex comprises a δ protein of the present invention.

The present invention describes a method to identify chemicals that modulate the ability of a δ subunit and the δ′ and/or DnaX subunit to interact. This method includes contacting the δ subunit with the δ′ and/or δ′ plus DnaX subunit in the presence of the candidate pharmaceutical to form a reaction mixture. The reaction mixture is subjected to conditions under which the δ subunit and the δ′ and/or δ′ plus DnaX subunit would interact in the absence of the candidate pharmaceutical. The reaction mixture is then analyzed for interaction between the δ subunit and the δ′ and/or DnaX subunit. The extent of interaction in the presence of the test compound is compared with the extent of interaction in the absence of the test compound. A change in the interaction between the δ subunit and the δ′ and/or DnaX subunit is indicitive of a compound that modulates the interaction.

The present invention describes a method to identify chemicals that modulate the ability of a DnaX complex (or a subassembly of the DnaX complex) to assemble a β subunit onto a DNA molecule. This method involves contacting a circular primed DNA molecule (which may be coated with SSB) with the DnaX complex (or the subassembly thereof) and the β subunit in the presence of the candidate pharmaceutical, and ATP or DATP to form a reaction mixture. The reaction mixture is subjected to conditions under which the DnaX complex (or subassembly) assembles the β subunit on the DNA molecule absent the candidate pharmaceutical. The presence or absence of the β subunit on the DNA molecule in the reaction mixture is analyzed. The extent of assembly in the presence of the test compound is compared with the extent of assmbly in the absence of the test compound. A change in the amount of β subunit on the DNA molecule is indicative of a compound that modulates the ability of a DnaX complex (or a subassembly of the DnaX complex) to assemble a β subunit onto a DNA molecule. The DnaX complex comprises a delta protein of the present invention.

The present invention describes a method to identify chemicals that modulate the ability of a DnaX complex (or a subunit (s) of the DnaX complex) to disassemble a β subunit from a DNA molecule. This method comprises contacting a DNA molecule onto which the β subunit has been assembled in the presence of the candidate pharmaceutical, to form a reaction mixture. The reaction mixture is subjected to conditions under which the DnaX complex (or a subunit (s) or subassembly of the DnaX complex) disassembles the β subunit from the DNA molecule absent the candidate pharmaceutical. The presence or absence of the β subunit on the DNA molecule in the reaction mixture is analyzed. The extent of assembly in the presence of the test compound is compared with the extent of assembly in the absence of the test compound. A change in the amount of β subunit on the DNA molecule is indicative of a compound that modulates the ability of a DnaX complex (or a subassembly of the DnaX complex) to disassemble a β subunit onto a DNA molecule. The DnaX complex comprises a δ subunit of the present invention.

The present invention describes a method to identify chemicals that modulate the dATP/ATP binding activity of a DnaX complex or a DnaX complex subunit (e. g. DnaX subunit). This method includes contacting the DnaX complex (or the DnaX complex subunit) with dATP/ATP either in the presence or absence of a DNA molecule and/or the β subunit in the presence of the candidate pharmaceutical to form a reaction. The reaction mixture is subjected to conditions in which the DnaX complex (or the subunit of DnaX complex) interacts with dATP/ATP in the absence of the candidate pharmaceutical. The reaction is analyzed to determine if dATP/ATP is bound to the DnaX complex (or the subunit of DnaX complex) in the presence of the candidate pharmaceutical. The extent of binding in the presence of the test compound is compared with the extent of binding in the absence of the test compound. A change in the dATP/ATP binding is indicative of a compound that modulates the dATP/ATP binding activity of a DnaX complex or a DnaX complex subunit (e.g. DnaX subunit). The DnaX complex comprises a δ subunit of the present invention.

The present invention describes a method to identify chemicals that modulate the dATP/ATPase activity of a DnaX complex or a DnaX complex subunit (e.g., the DnaX subunit). This method involves contacting the DnaX complex (or the DnaX complex subunit) with dATP/ATP either in the presence or absence of a DNA molecule and/or a β subunit in the presence of the candidate pharmaceutical to form a reaction mixture. The reaction mixture is subjected to conditions in which the DnaX subunit (or complex) hydrolyzes dATP/ATP in the absence of the candidate pharmaceutical. The reaction is analyzed to determine if dATP/ATP was hydrolyzed. The extent of hyrdolysis in the presence of the test compound is compared with the extent of hydrolysis in the absence of the test compound. A change in the amount of dATP/ATP hydrolyzed is indicative of a compound that modulates the dATP/ATPase activity of a DnaX complex or a DnaX complex subunit (e. g., the DnaX subunit). The DnaX complex comprises a δ protein of the present invention.

The invention provides a further method for identifying chemicals that modulate the activity of a DNA polymerase comprising contacting a circular primed DNA molecule (may be coated with SSB) with a DnaX complex, β, subunit and an α subunit in the presence of the candidate pharmaceutical, and dNTPs (or modified dNTPs) to form a reaction mixture. The reaction mixture is subjected to conditions, which in the absence of the candidate pharmaceutical, affect nucleic acid polymerization, and the presence or absence of the extension product in the reaction mixture is analyzed. The activity of the replicase in the presence of the test compound is compared with the activity of the replicase in the absence of the test compound. A change in the activity of the replicase in the presence of the test compound is indicative of a compound that modulates the activity of the replicase. The DnaX complex comprises a δ protein of the present invention.

The present invention also includes a test kit to identify a compound capable of modulating DNA polymerase III replicase activity. Such a test kit includes an isolated δ subunit protein and a means for determining the extent of modulation of DNA replication activity in the presence of (i.e., effected by) a putative inhibitory compound.

The present invention also includes modulators and inhibitors isolated by such a method, and/or test kit, and their use to inhibit any replicase comprising a δ subunit that is susceptible to such an inhibitor. Modulators of bacterial DNA replication can also be used directly as to treat animals, plants and humans suspected of having a bacterial infection as long as such compounds are not harmful to the species being treated. Similarly, molecules that bind to any replicase comprising a δ subunit, incuding but not limited to antibodies and small molecules, can also be used to target cytotoxic, therapeutic or imaging entities to a site of infection.

The present invention also includes a method of synthesizing a DNA molecule comprising hybridizing a primer to a first DNA molecule; and incubating said DNA molecule in the presence of a thermostable DNA polymerase replicase and one or more dNTPs under conditions sufficient to synthesize a second DNA molecule complementary to all or a portion of said first DNA molecule, wherein said thermostable DNA polymerase replicase comprises an δ subunit protein selected from the group consisting of an Aquifex, Thermus and Thermatoga δ subunit protein.

The present invention also provides a method of amplifying a double-stranded DNA molecule comprising providing a first and second primer, wherein said first primer is complementary to a sequence at or near the 3′-termini of the first strand of said DNA molecule and said second primer is complementary to a sequence at or near the 3′-termini of the second strand of said DNA molecule, hybridizing said primer to said first strand and said second primer to said second strand in the presence of a thermostable DNA polymerase replicase, under conditions such that a third DNA molecule complementary to said first strand and a fourth DNA molecule complementary to said second strand are synthesized, denaturing said first and third strand, and said second and fourth strands; and repeating the steps one or more times; wherein said thermostable DNA polymerase is selected from the group consisting of an Aquifex, Thermus and Thermatoga δ subunit protein.

The present invention also provides method of amplifying nucleic acid sequences, the method comprising mixing a rolling circle replication primer with one or more amplification target circles, to produce a primer-amplification target circle mixture, and incubating the primer-amplification target circle mixture under conditions that promote hybridization between the amplification target circles and the rolling circle replication primer in the primer-amplification target circle mixture, wherein the amplification target circles each comprise a single-stranded, circular DNA molecule comprising a primer complement portion, and wherein the primer complement portion is complementary to the rolling circle replication primer; and mixing a thermostable DNA polymerase replicase with the primer-amplification target circle mixture, to produce a replicase-amplification target circle mixture, and incubating the replicase-amplification target circle mixture under conditions that promote replication of the amplification target circles, wherein replication of the amplification target circles results in the formation of tandem sequence DNA, and wherein said thermostable DNA polymerase replicase comprises an δ subunit protein selected from the group consisting of an Aquifex, Thermus and Thermatoga δ subunit protein.

The present invention further provides a method of synthesizing DNA which comprises utilizing one or more polypeptides, said one or more polypeptides comprising an amino acid sequence having at least 95% sequence identity to an δ subunit protein of th present invention.

### BRIEF DESCRIPTION OF THE FIGURES

FIG. 1 shows structural features of the DNA polymerase III holoenzyme.

FIG. 2A shows the annotated DNA polymerase III holoenzyme alpha subunit gene from Aquifex aeolicus.

FIG. 2B shows the annotated DNA polymerase III holoenzyme β subunit gene from Aquifex aeolicus.

FIG. 2C shows the annotated DNA polymerase III holoenzyme epsilon subunit gene from Aquifex aeolicus.

FIG. 2D shows the annotated DNA polymerase III holoenzyme gamma (DnaX) subunit gene from Aquifex aeolicus.

FIG. 2E shows the annotated DNA polymerase III holoenzyme SSB gene from Aquifex aeolicus.

FIG. 3A shows the annotated DNA polymerase III holoenzyme gamma/τ (DnaX) gene from Thermotoga maritima.

FIG. 3B shows the annotated DNA polymerase III holoenzyme β gene from Thermotoga maritima.

FIG. 3C shows the annotated DNA polymerase III holoenzyme epsilon gene from Thermotoga maritima.

FIG. 3D shows the annotated DNA polymerase III holoenzyme alpha gene for DNA polymerase III, type II from Thermotoga maritima.

FIG. 3E shows the annotated DNA polymerase III holoenzyme alpha gene for DNA polymerase III, type I from Thermotoga maritima.

FIG. 3F shows the annotated DNA polymerase III SSB gene from Thermotoga maritima.

FIGS. 4A-HHH shows delta sequences discovered from organisms with completely sequenced genomes and the DNA sequence of their structural gene (holA).

FIGS. 5A-G shows delta protein and DNA sequences for S. pyogenes, S. pneumoniae, S. aureus.

FIG. 6 shows the amino acid alignment of the newly identified δ proteins shown in FIGS. 4 and 5 with the established δ protein sequences of E. coli and T. thermophilus with the alignment performed using ClustalW 1.8.

FIGS. 7A-F shows δ protein and gene sequences identified from partially sequenced genomes.

FIG. 8 shows a listing of all of the recognized bacterial phyla and the first level of subclassifications of each pyla along with a listing of bacteria from which delta sequences have been identified.

FIG. 9 shows a phylogenic tree constructed by relating organisms by their evolutionary relationship determined by their rRNA sequences that allows evolutionarily close organisms to be identified for use in searching for new δ proteins.

FIGS. 10A-BB shows the additional δ proteins and their structural (holA) genes that were identified using phylogenetic considerations.

FIG. 11 shows a graphical depiction of the PCR product “PCR Aquifex NB delta” which will be used to express the tagged Aquifex aeolicus holA gene.

FIG. 12 shows the region of pA1-NB-KpnI that the PCR product ““PCR Aquifex NB delta” will be inserted into after digestion with appropriate restriction enzymes.

FIG. 13 shows a graphical depiction of the PCR product “PCR Aquifex native delta” which will be used to express the native Aquifex aeolicus holA gene.

FIG. 14 shows the reigion of pA1-NB-KpnI that the PCR product “PCR Aquifex native delta” will be inserted into after digestion with appropriate restriction enzymes.

FIG. 15 shows a graphical depiction of the PCR product “PCR NB Thermotoga D” which which will be used to express the tagged Thermotoga maritima holA gene.

FIG. 16 shows a graphical depiction of the PCR product “PCR native Thermatoga D” which which will be used to express the native Thermotoga maritima holA gene.

FIG. 17 shows the three dnaX like Thermotoga maritima as aligned using ClustalW.

FIG. 18 shows a graphical depiction of the PCR product “PCR AQ NB delta” which will be used to express the tagged Aquifex holB gene (AQ_{—}1526).

FIG. 19 shows the region of pA1-NB-Avr2 that PCR product “PCR AQ NB delta” will be inserted into following digestion with appropriate restriction enzymes.

FIG. 20 shows a graphical depiction of the PCR product “PCR of native Aquifex D” which will be used to express Aquifex δ′ subunit in an untagged form.

FIG. 21 shows a graphical depiction of the PCR product “PCR TM NB D′(0508)” which will be used to express the tagged T. maritima TM0508 gene.

FIG. 22 shows a graphical depiction of the PCR product “PCR TM native D′(0508)” which will be used to express the native T. maritima TM0508 gene.

FIG. 23 shows the region of pA1-CB-NcoI that “PCR TM native D′(0508)” will be inserted into following digestion with appropriate restriction enzymes.

FIG. 24 shows the region of pA1-TM[0508] containing the site in which the RcaI digested restriction site was spliced into the NcoI restriction site of pA1-CB-NcoI.

FIG. 25 shows a graphical description of the PCR product “PCR TM NB D′0771,” which will be used for the expression of tagged T. maritima protein TM0771.

FIG. 26 shows a graphical description of the PCR product “PCR TM native D′(0771)” which will be used for the expression of native T. maritima protein TM0771.

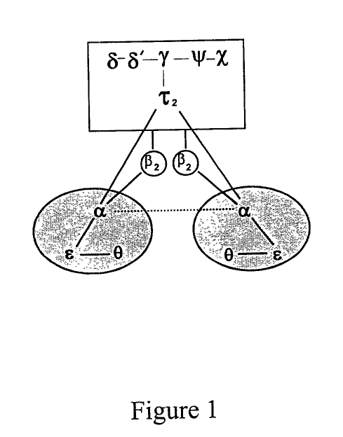

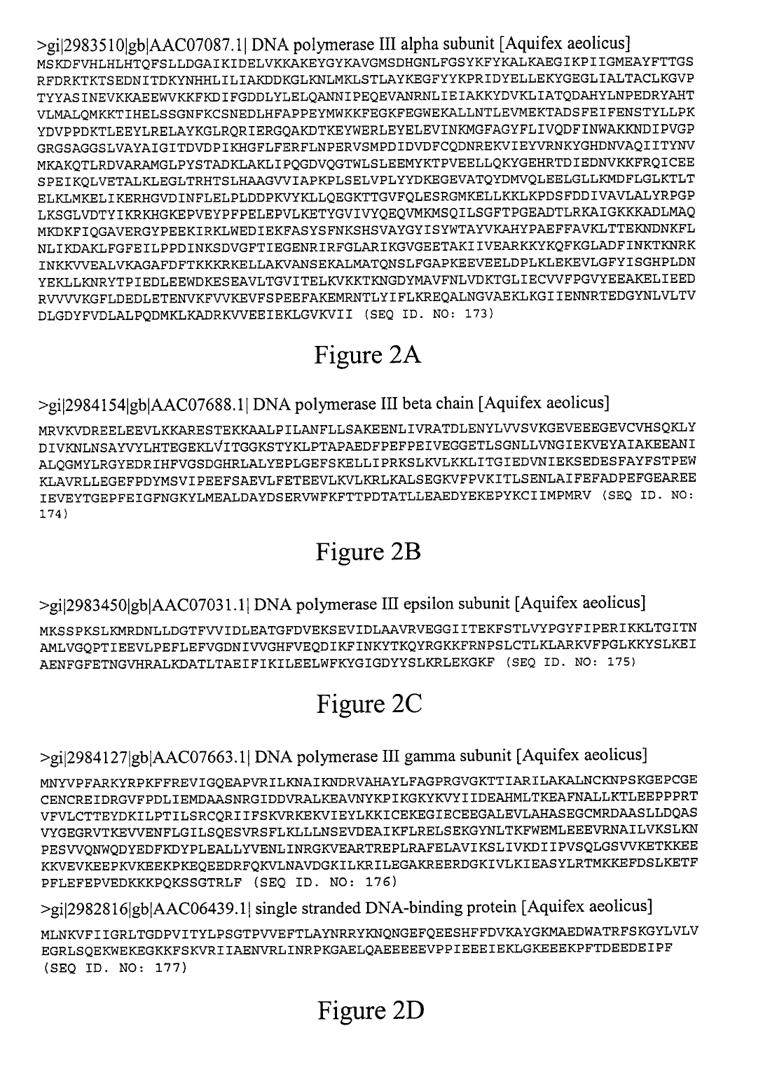

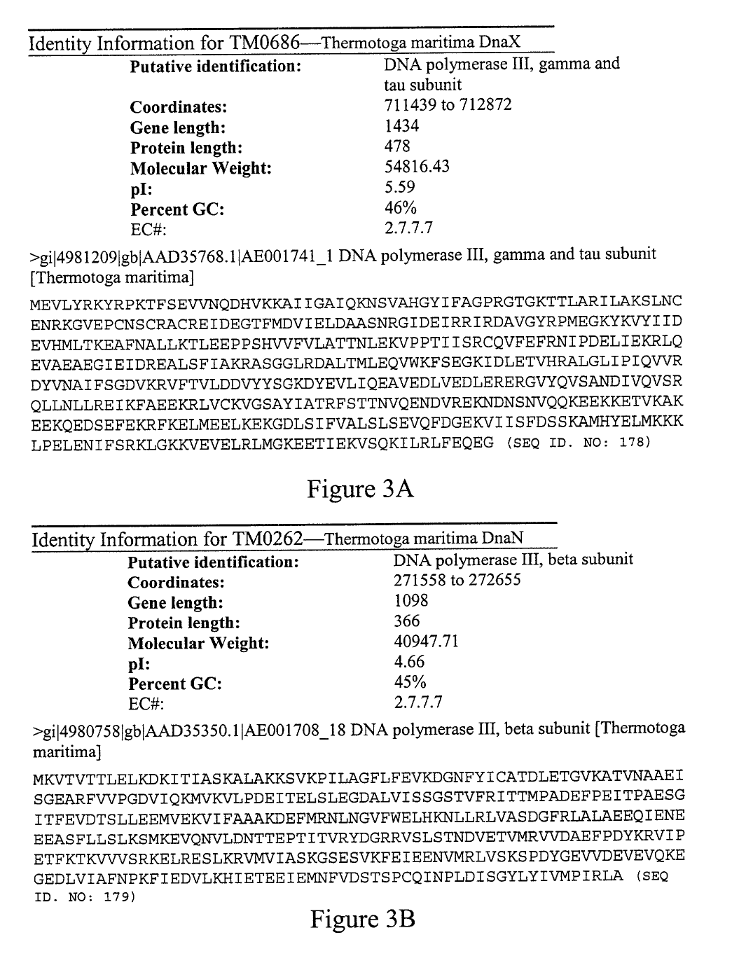

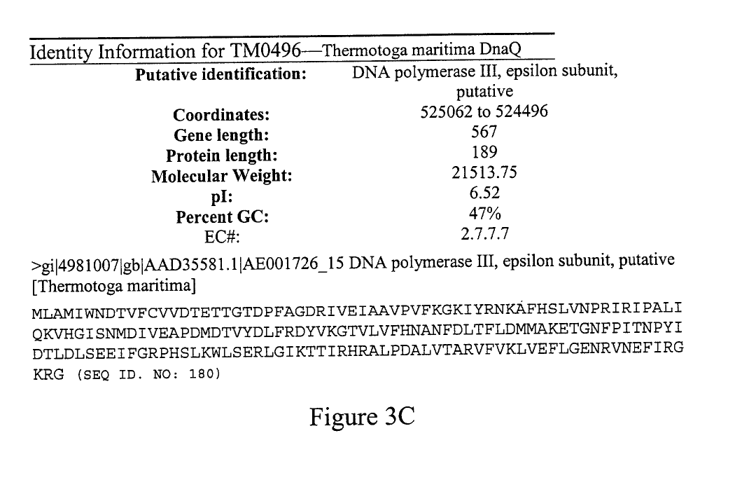

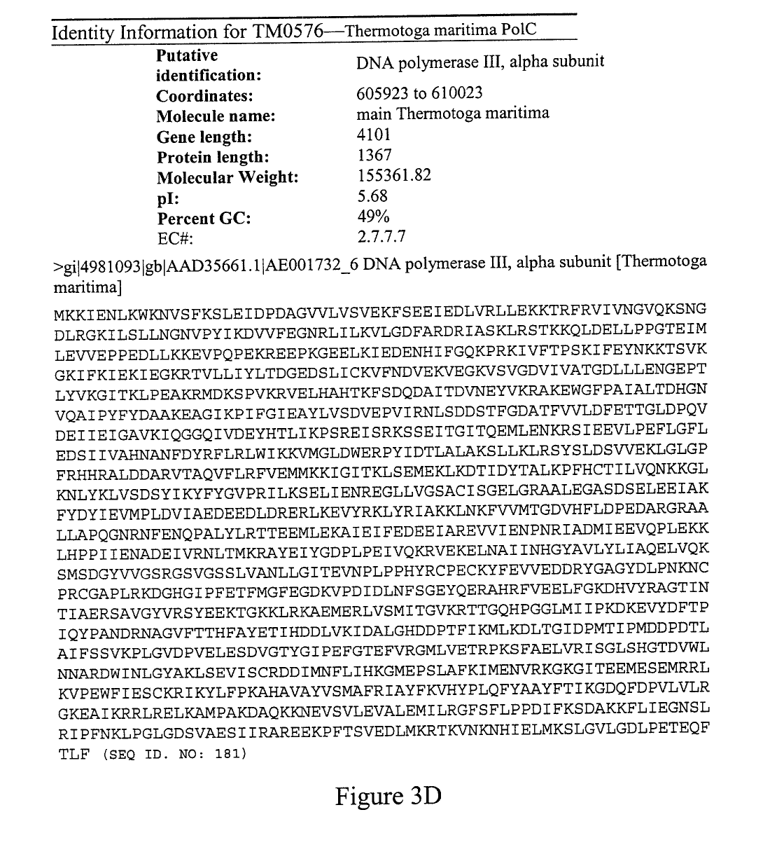

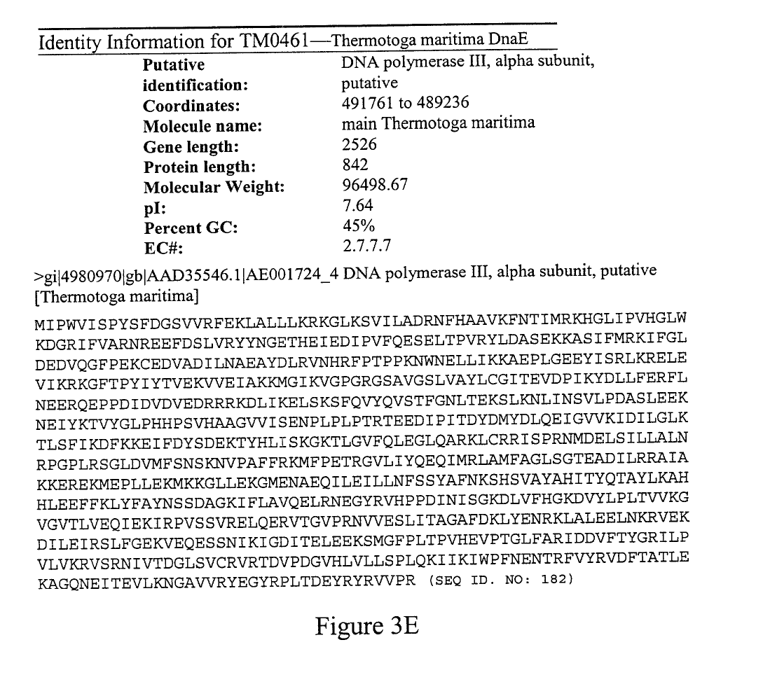

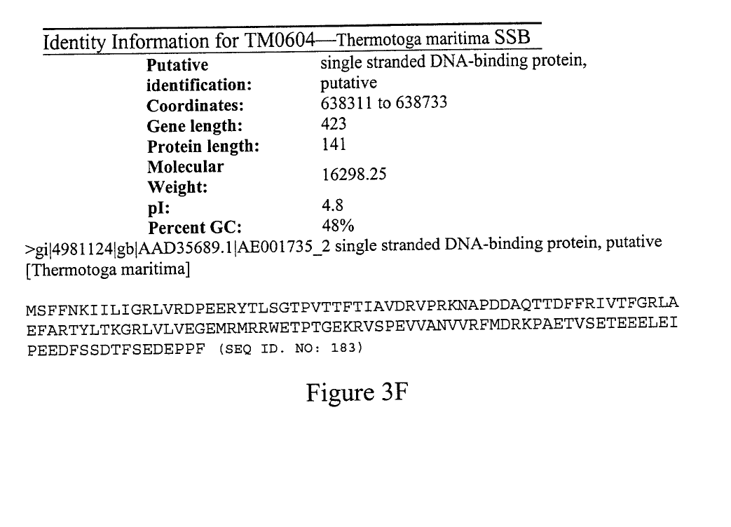

### DETAILED DESCRIPTION OF THE PREFERRED EMBODIMENTS

The present invention relates to structural gene and amino acid sequences encoding δ subunits of DNA polymerase III holoenzyme from a variety of bacteria. As used herein, the term “gene” refers to a nucleic acid (e.g., DNA) sequence that comprises coding sequences necessary for the production of a polypeptide or precursor (e.g., DNA polymerase III holoenzyme or holoenzyme subunit, as appropriate). The polypeptide can be encoded by a full length coding sequence or by any portion of the coding sequence so long as the desired activity or functional properties (e.g., enzymatic activity, ligand binding, binding of another DNA polymerase III holoenzyme subunit, processivity, etc.) of the full-length or fragment are retained. The term also encompasses the coding region of a structural gene and the including sequences located adjacent to the coding region on both the 5′ and 3′ ends for a distance of about 1 kb on either end such that the gene corresponds to the length of the full-length mRNA. The term “gene” encompasses both cDNA and genomic forms of a gene. A genomic form or clone of a gene contains the coding region interrupted with non-coding sequences termed “intervening regions” or “intervening sequences.” The mRNA functions during translation to specify the sequence or order of amino acids in a nascent polypeptide.

As used herein, the term “DNA polymerase III holoenzyme” refers to the entire DNA polymerase III entity (i.e., all of the polymerase subunits, as well as the other associated accessory proteins required for processive replication of a chromosome or genome), while “DNA polymerase III” is just the core [α, ε, θ] for type I DNA Polymerase IIIs (encoded by dnaE gene) or just an α catalytic subunit for type II DNA Polymerase IIIs (encoded by polC gene) that includes and epsilon-like sequence within the same polypeptide chain. “DNA polymerase III holoenzyme subunit” is used in reference to any of the subunit entities that comprise the DNA polymerase III holoenzyme. Thus, the term “DNA polymerase III” encompasses “DNA polymerase III holoenzyme subunits” and “DNA polymerase III subunits.” Subunits include, but are not limited to, DnaX or τ (and γ from some organisms, including, but not limited to, many proteobacteria and T. thermophilus), HolA or δ, HolB or δ′ proteins, DnaN or β proteins, DnaE or α subunit of DNA polymerase III—type I, PolC or α subunit of DNA polymerase III—type II and DnaQ or ε proteins. As used herein, “replicase” means an enzyme that duplicates a DNA polynucleotide sequence.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited proteins. It is to be noted that the term “a” or “an” entity refers to one or more of that entity; for example, a protein refers to one or more proteins or at least one protein. As such, the terms “a” (or “an”), “one or more” and “at least one” can be used interchangeably herein. It is also to be noted that the terms “comprising”, “including”, and “having” can be used interchangeably. Furthermore, a compound “selected from the group consisting of” refers to one or more of the compounds in the list that follows, including mixtures (i.e., combinations) of two or more of the compounds.

Methods of Identifying δ Subunit Proteins and Nucleic Acid Molecules of the Present Invention

One embodiment of the present invention is an isolated DNA polymerase III holoenzyme subunit δ protein. According to the present invention, an isolated, or biologically pure, protein, is a protein that has been removed from its natural environment. As such, “isolated” and “biologically pure” do not necessarily reflect the extent to which the protein has been purified. An isolated δ subunit protein of the present invention can be obtained from its natural source, can be produced using recombinant DNA technology or can be produced by chemical synthesis. As used herein, an isolated δ subunit protein can be a full-length protein or any homologue of such a protein.

The minimal size of a protein homolog of the present invention is a size sufficient to be encoded by a nucleic acid molecule capable of forming a stable hybrid with the complementary sequence of a nucleic acid molecule encoding the corresponding natural protein. As such, the size of the nucleic acid molecule encoding such a protein homolog is dependent on nucleic acid composition and percent homology between the nucleic acid molecule and complementary sequence as well as upon hybridization conditions per se (e.g., temperature, salt concentration, and formamide concentration). The minimal size of such nucleic acid molecules is typically at least about 12 to about 15 nucleotides in length if the nucleic acid molecules are GC-rich and at least about 15 to about 17 bases in length if they are AT-rich. As such, the minimal size of a nucleic acid molecule used to encode a protein homolog of the present invention is from about 12 to about 18 nucleotides in length. There is no limit on the maximal size of such a nucleic acid molecule in that the nucleic acid molecule can include a portion of a gene, an entire gene, or multiple genes, or portions thereof. Similarly, the minimal size of a DNA polymerase III subunit protein homolog of the present invention is from about 4 to about 6 amino acids in length, with preferred sizes depending on whether a full-length, multivalent (i.e., fusion protein having more than one domain each of which has a function), or functional portions of such proteins are desired. Polymerase protein homologs of the present invention preferably have activity corresponding to the natural subunit, such as being able to perform the activity of the subunit in a binding assay or DNA replication assay.

DNA polymerase III holoenzyme subunit δ protein homologs can be the result of natural allelic variation or natural mutation. The protein homologs of the present invention can also be produced using techniques known in the art including, but not limited to, direct modifications to the protein or modifications to the gene encoding the protein using, for example, classic or recombinant DNA techniques to effect random or targeted mutagenesis.

According the methods of the present invention, δ subunit proteins may be identified from any bacteria. Such identification of δ subunit proteins has previously not been possible due to the limited sequence relatedness of δ proteins. Prior to the present invention, δ proteins were known only for E. coli, and two related proteobacterial deltas—Haemophilis influenzae and Neisseria meningitides. A simple BLAST search of NCBI databases for δ proteins based on the known E. coli sequence returns results for only a small number of closely related organisms: Vibrio cholerae, Pasteurella multocida, Haemophilus influenzae, Buchnera sp. APS, Pseudomonas aeruginosa, Xylella fastidiosa, from the gamma proteobacteria division, and Neisseria meningitides from the beta division of proteobacteria. No sequences from the alpha, delta or epsilon divisions of proteobacteria are retrieved. The present invention provides methods, detailed below and and in the Examples section, that allow for the identification of δ protein subunits from bacteria from all known phyla.

The present invention also provides δ subunit proteins identified by the methods. In one embodiment, the present invention provides a method for identifying a bacterial δ protein from a sequenced genome.

In a preferred embodiment, the method for identifying a bacterial DNA polymerase holoenzyme δ subunit protein from at least one completely sequenced genome comprises providing a database of sequence information for a plurality of different organisms, setting an inclusion threshold to a level sufficient to minimize the number of required iterations, selecting the organisms to be searched, providing the amino acid sequence of the δ subunit protein from a reference organism, comparing the sequences of the selected organisms and the reference organism, excluding known DnaX proteins and known δ′ proteins from further steps, and repeating the cycle to permit identification of additional δs using the pattern of similarity of identified δ sequences in subsequent searches, selecting proteins comprising between about 300 and about 400 amino acids, wherein a bacterial DNA polymerase III holoenzyme δ subunit protein may be identified.

As used herein, setting the inclusion threshold to a level sufficient to minimize the number of iterations required refers to a threshold that permits a search to be found with a reasonable time and effort. An iteration is a repetition of the steps of excluding known DnaX proteins and known δ′ proteins from further steps, and selecting proteins comprising between about 300 and about 400 amino acids. A reasonable number of iterations will depend on the complexity of the database searched, the sequence relatedness of the δ sequences from the targeted organisms, among other factors. Under the conditions used in the present invention, less than about 20 iterations is a reasonable number. In preferred embodiments, less than about 15 iterations is a reasonable number. In more preferred embodiments, less than about 10 iterations is a reasonable number. In other preferred embodiments, less than about 5 iteration is a reasonable number.

As used herein, the inclusion threshold is also referred to as the E or “Expect” value limit. Potential ‘hits’ that have an E value higher than the inclusion threshold are excluded. In one preferred embodiment, the inclusion threshold is 0.002. In another preferred embodiment, the threshold is 0.001. (With the threshold set to 0.001, there is a 1 in 1000 chance for an inappropriate hit, based on the referenced stochastic model.)

Conserved regions are based on alignments of identified deltas from a number of organisms. After finding a number of candidate delta using this method, they were aligned using Clustal W (Thompson, J. D., Higgins, D. G., and Gibson, T. J., Nucleic Acids Res. 22:4673-4680 (1994)), and conserved regions were identified using methods known to those skilled in the art. Conserved amino acids within the conserved regions are shown in boldface letters.

These conserved regions are:

| | | |
|---|---|---|
| 1.) | 17--(L/I/V)XX(L/I/V)Y(L/I/V)(L/I/V)XGX(D/E)XX(L/I/V)(L/I/V)XXXXXX(L/I/V)--38 | (SEQ ID NO:194) |
| 2.) | 64--(L/I/V)(L/I/V)XXXX(A/S)XX(L/I/V)F(A/S)X(K/R)X(L/I/V)(L/I/V)(L/I/V)(L/I/V)--82 | (SEQ ID NO:195) |
| 3.) | 97--(L/I/V)XX(L/I/V)(L/I/V)XXXXX(D/E)X(L/I/V)(L/I/V)(L/I/V)(L/I/V)XXXK(L/I/V)--17 | (SEQ ID NO:196) |
| 4.) | 154--(R/K)XXX(L/I/V)X(L/I/V)X(L/I/V)(D/E)X(D/E)(A/S)(L/I/V)XX(L/I/V)XXXXXXN(L/I/V) | (SEQ ID NO:197) |
| | XX(L/I/V)XX(D/E)(L/I/V)X(R/K)(L/I/V)X(L/I/V)(L/I/V)--191 | |
| 5.) | 215--FX(L/I/V)X(D/E)(A/S)(L/I/V)(L/I/V)XG(R/K)XXX(A/S)(L/I/V)X(L/I/V)(L/I/V)XX | (SEQ ID NO:198) |
| | (L/I/V)XXXGXD/E)P(L/I/V)X(L/I/V)(L/I/V)XX(L/I/V)XXX(L/I/V)XX(L/I/V)XX(L/I/V)--260 | |
| 6.) | 298--(L/I/V)XXX(L/I/V)XX(L/I/V)XXX(D/E)XX(L/I/V)(R/K)XXXXX(D/E)XXXX(L/I/V)(D/E)XX | (SEQ ID NO:198) |
| | (L/I/V)(L/I/V)X(L/I/V)--331 | |

The present invention also provides a method for identifying a δ protein from at least one partially sequenced genome. The step of selecting organisms comprises selecting organisms in the database which are only partially sequenced.

The present invention also provides a method for identifying a δ protein from at least one target organism comprising using the method to identify a δ protein from an evolutionarily related organism, and performing a search for a similar sequence in the sequence of the target organism.

In another embodiment, the method comprises providing a database of sequence information of different organisms, setting the inclusion threshold to a level sufficient to minimize the number of required iterations, selecting the organism or organisms to be searched, entering the amino acid sequence of the δ subunit protein from a reference organism for comparison, comparing the sequences of the selected organisms and the reference organism, excluding known DnaX proteins and known δ′ proteins from further steps, selecting proteins comprising i) comprising at least three of the follwing six characterisics:

a. having at least five out of ten conserved residues fom the conserved region 17—(L/I/V)XX(L/I/V)Y(L/I/V)(L/I/V)XGX(D/E)XX(L/I/V)(L/I/V)XXXXXX(L/I/V)—38; (SEQ ID NO: 194)

b. having at least five out of eleven conserved residues fom the conserved region 64—(L/I/V)(L/I/V)XXXX(A/S)XX(L/I/V)F(A/S)X(K/R)X(L/I/V)(L/I/V)(L/I/V)(L/I/V)—82; (SEQ ID NO: 195)

c. having at least five out of ten conserved residues fom the conserved region 97—(L/I/V)XX(L/I/V)(L/I/V)XXXXX(D/E)X(L/I/V)(L/I/V)(L/I/V)(L/I/V)XXXK(L/I/V)—117; (SEQ ID NO: 196)

d. having at least nine out of eighteen conserved residues fom the conserved region

| | |
|---|---|
| 154--(R/K)XXX(L/I/V)X(L/I/V)X(L/I/V)(D/E)X(D/E)(A/S)(L/I/V)XX(L/I/V)XXXXXXN(L/I/V)XX | (SEQ ID NO:197) |
| (L/I/V)XX(D/E)(L/I/V)X(R/K)(L/I/V)X(L/I/V)(L/I/V)--191; | |

e. having at least eleven out of twenty-three conserved residues fom the conserved region 215—FX(L/I/V)X(D/E)(A/S)(L/I/V)(L/I/V) XG(R/K)XXX(A/S)(L/I/V)X(L/I/V)(L/I/V)XX(L/I/V)XXXGX(D/E)P(L/I/V)X(L/I/V)(L/I/V)XX(L/I/V)XXX(L/I/V)XX(L/I/V)XX(L/I/V)—260 (SEQ ID NO: 198); and

f. having at least six out of twelve conserved residues fom the conserved region 298—(L/I/V)XXX(L/I/V)XX(L/I/V)XXX(D/E)XX(L/I/V)V(R/K)XXXXX(D/E)XXXX(L/I/V)(D/E)XX(L/I/V)(L/I/V)X(L/I/V)—331 (SEQ ID NO: 199); and retaining the sequences above the threshold. The retained sequences are identified as δ subunit proteins.

An iteration is a repetition of the steps of excluding known DnaX proteins and known δ′ proteins from further steps, and selecting proteins having three of the six conserved region characteristic.

The present invention also provides methods to identify δ subunit proteins from genomes that are not completely sequenced, but are partially sequenced. This is accomplished, in the above method, by replacing the step of selecting the organism or organisms to be searched with the selection of organisms in the database which are partially sequenced. In databases that do not allow a search of only the partially sequenced genomes, it is permissible to search all bacteria which includes partially and completely sequenced organisms, or other suitable subset of organisms. Sequences already identified from completely sequenced organisms could be specifically excluded or, alternatively, one can just pick out the new δ sequences identified.

The present invention also provides methods for identifying δ proteins not already identified by the above methods. These methods are useful for identifying δ from already sequenced or partially sequenced genomes, but for which a δ protein has not been found, and for identifying δ from organisms as the sequences of these organisms become available. The method is based on phylogenetic relationships among bacteria. To identify δ from a bacterial species for which a δ protein has not been found, the search comprises searching the sequences for the new organism against the δ amino acid sequences from organisms that are evolutionarily close (i.e., members of the same class/phylum) to the target organism. That is, the δ protein from an evolutionarily close organism is used as the reference or query sequence. The reference δ sequence can be obtained by any of the methods of the present invention. If the initial search produces no hits, then searches should continue using δ amino acid sequences as query sequences that are gradually more evolutionarily distant from the target organism until δ has been identified, or until δ sequences from all known phyla have been exhausted. Illustrative examples of the use of these methods are described in the examples section.

δ Subunit Proteins

One embodiment of the present invention includes an isolated Aquifex, Bacillus, Buchnera, Borrelia, Campylobacter, Caulobacter, Chlamydia, Chlamydophila, Deinococcus, Haemophilus, Helicobacter, Lactococcus, Mycobacterium, Mesorhizobium, Mycoplasma, Neisseria, Pasteurella, Pseudomonas, Rickettsia, Synechocystis, Thermatoga, Treponema, Ureaplasma, Vibrio, Xylella, Mycoplasma, Staphylococcus, Streptococcus, Streptomyces, Streptococcus, Streptococcus, Thermus, Yersisna, Actinobacillus, Bordetealla, Bacillus, Burkholderia, Chlorobium, Chloroflexus, Clostridium, Corynebacterium, Cytopahaga, Dehalococcoides, Porphyromonas, Prochlorococcus, and Salmonella DNA polymerase III holoenzyme δ subunit protein.

One embodiment of the present invention includes an isolated Aquifex aeolicus, Bacillus halodurans, Bacillus subtilis, Buchnera, Borrelia burgdorferi, Campylobacter jejuni, Caulobacter crescentus, Chlamydia muridarum, Chlamydia trachomatis, Chlamydophila pneumonia, Deinococcus radiodurans, Haemophilus influenzae, Helicobacter pylori, Helicobacter pylori 26695, Helicobacter pylori J99, Lactococcus lactis, Mycobacterium laprae, Mycobacterium tuberculosis, Mesorhizobium loti, Mycoplasma genitalium, Mycoplasma pneumoniae, Neisseria meningitidis MC58, Neisseria meningitidis Z2491, Pasteurella multocida, Pseudomonas aeruginosa, Rickettsia prowazekii, Synechocystis, Thermatoga maritima, Treponema pallidum, Ureaplasma urealyticum, Vibrio cholerae, Xylella fastidiosa, Mycoplasma pulmonis, Staphylococcus aureus, Streptococcus agalactiae, Streptomyces coelicolor, Streptococcus pneumoniae, Streptococcus pyogenes, Thermus thermophilus, Yersisna pestis, Actinobacillus actinomycetemcomitans, Bordetealla pertussis, Bacillus anthracis, Burkholderia pseudomallei, Chlorobium tepidum, Chloroflexus aurantiacus, Clostridium difficile, Corynebacterium diphtheriae, Cytopahaga hutchinsonii, Dehalococcoides ethenogenes, Porphyromonas gingivalis, Prochlorococcus marinus, and Salmonella typhi DNA polymerase III holoenzyme δ subunit protein.

Also preferred is a delta protein comprising an amino acid sequence that is at least about 75%, more preferably at least about 80%, more preferably at least about 85%, more preferably at least about 90%, more preferably at least about 95%, and more preferably at least about 98% identical to amino acid sequence SEQ ID NO: 3, SEQ ID NO: 226, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 60, SEQ ID NO: 63, SEQ ID NO: 66, SEQ ID NO: 69, SEQ ID NO: 72, SEQ ID NO: 75, SEQ ID NO: 78, SEQ ID NO: 81, SEQ ID NO: 84, SEQ ID NO: 87, SEQ ID NO: 90, SEQ ID NO: 93, SEQ ID NO: 96, SEQ ID NO: 99, SEQ ID NO: 102, SEQ ID NO: 229, SEQ ID NO: 105, SEQ ID NO: 108, SEQ ID NO: 111, SEQ ID NO: 114, SEQ ID NO: 117, SEQ ID NO: 120, SEQ ID NO: 123, SEQ ID NO: 126, SEQ ID NO: 129, SEQ ID NO: 132, SEQ ID NO: 135, SEQ ID NO: 138, SEQ ID NO: 141, SEQ ID NO: 144, SEQ ID NO: 147, SEQ ID NO: 150, SEQ ID NO: 153, SEQ ID NO: 156, or a protein encoded by an allelic variant of a nucleic acid molecule encoding a protein comprising any of these sequences. Methods to determine percent identities between amino acid sequences and between nucleic acid sequences are known to those skilled in the art. Preferred methods to determine percent identities between sequences include computer programs such as GCG program (available from Genetics Computer Group, Madison, Wis.), the MacVectors program (available from the Eastman Kodak Company, New Haven, Conn.), the DNAsis™ program (available from Hitachi Software, San Bruno, Calif.) the Vector NTI Suite of Programs (available from Informax, Inc., North Bethesda, Md.) or the BLAST software available on the NCBI website.

In one embodiment, a preferred δ subunit protein comprises an amino acid sequence of at least about 5 amino acids, preferably at least about 50 amino acids, more preferably at least about 100 amino acids, more preferably at least about 200 amino acids, more preferably at least about 250 amino acids, more preferably at least about 275 amino acids, more preferably at least about 300 amino acids, more preferably at least about 350 amino acids, more preferably at least about 375 amino acids or the full-length δ protein, whichever is shorter. In another embodiment, preferred δ subunit proteins comprise full-length proteins, i.e., proteins encoded by full-length coding regions, or post-translationally modified proteins thereof, such as mature proteins from which initiating methionine and/or signal sequences or “pro” sequences have been removed.

A fragment of a δ subunit protein of the present invention preferably comprises at least about 5 amino acids, more preferably at least about 10 amino acids, more preferably at least about 15 amino acids, more preferably at least about 20 amino acids, more preferably at least about 25 amino acids, more preferably at least about 30 amino acids, more preferably at least about 35 amino acids, more preferably at least about 40 amino acids, more preferably at least about 45 amino acids, more preferably at least about 50 amino acids, more preferably at least about 55 amino acids, more preferably at least about 60 amino acids, more preferably at least about 65 amino acids, more preferably at least about 70 amino acids, more preferably at least about 75 amino acids, more preferably at least about 80 amino acids, more preferably at least about 85 amino acids, more preferably at least about 90 amino acids, more preferably at least about 95 amino acids, and even more preferably at least about 100 amino acids in length.

In one embodiment, a preferred isolated δ subunit protein of the present invention is a protein encoded by a nucleic acid molecule having the nucleic acid sequence SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 223, SEQ ID NO: 52, SEQ ID NO: 55, SEQ ID NO: 58, SEQ ID NO: 61, SEQ ID NO: 64, SEQ ID NO: 67, SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103, SEQ ID NO: 106, SEQ ID NO: 109, SEQ ID NO: 112, SEQ ID NO: 115, SEQ ID NO: 118, SEQ ID NO: 121, SEQ ID NO: 124, SEQ ID NO: 127, SEQ ID NO: 130, SEQ ID NO: 133, SEQ ID NO: 136, SEQ ID NO: 139, SEQ ID NO: 142, SEQ ID NO: 145, SEQ ID NO: 148, SEQ ID NO: 151, SEQ ID NO: 154; or by an allelic variant of a nucleic acid molecule having any of these sequences.

One embodiment of the present invention includes a non-native δ subunit protein encoded by a nucleic acid molecule that hybridizes under stringent hybridization conditions with a δ subunit gene, or holA gene. As used herein, stringent hybridization conditions refer to standard hybridization conditions under which nucleic acid molecules, including oligonucleotides, are used to identify molecules having similar nucleic acid sequences. Such standard conditions are disclosed, for example, in Sambrook et al., Molecular Cloning: A Laboratory Manual, Cold Spring Harbor Labs Press (1989). Sambrook et al., ibid., is incorporated by reference herein in its entirety. Stringent hybridization conditions typically permit isolation of nucleic acid molecules having at least about 70% nucleic acid sequence identity with the nucleic acid molecule being used to probe in the hybridization reaction. Formulae to calculate the appropriate hybridization and wash conditions to achieve hybridization permitting 30% or less mismatch of nucleotides are disclosed, for example, in Meinkoth, J. et al., Anal. Biochem. 138:267-284 (1984); Meinkoth, J. et al., ibid., is incorporated by reference herein in its entirety. In preferred embodiments, hybridization conditions will permit isolation of nucleic acid molecules having at least about 80% nucleic acid sequence identity with the nucleic acid molecule being used to probe. In more preferred embodiments, hybridization conditions will permit isolation of nucleic acid molecules having at least about 90% nucleic acid sequence identity with the nucleic acid molecule being used to probe. In more preferred embodiments, hybridization conditions will permit isolation of nucleic acid molecules having at least about 95% nucleic acid sequence identity with the nucleic acid molecule being used to probe.

A preferred δ subunit protein includes a protein encoded by a nucleic acid molecule which is at least about 50 nucleotides and which hybridizes under conditions which preferably allow about 20% base pair mismatch, more preferably under conditions which allow about 15% base pair mismatch, more preferably under conditions which allow about 10% base pair mismatch, more preferably under conditions which allow about 5% base pair mismatch, and even more preferably under conditions which allow about 2% base pair mismatch with a nucleic acid molecule selected from the group consisting of SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 223, SEQ ID NO: 52, SEQ ID NO: 55, SEQ ID NO: 58, SEQ ID NO: 61, SEQ ID NO: 64, SEQ ID NO: 67, SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103, SEQ ID NO: 106, SEQ ID NO: 109, SEQ ID NO: 112, SEQ ID NO: 115, SEQ ID NO: 118, SEQ ID NO: 121, SEQ ID NO: 124, SEQ ID NO: 127, SEQ ID NO: 130, SEQ ID NO: 133, SEQ ID NO: 136, SEQ ID NO: 139, SEQ ID NO: 142, SEQ ID NO: 145, SEQ ID NO: 148, SEQ ID NO: 151, SEQ ID NO: 154, or a complement of any of these nucleic acid molecules.

As used herein, a holA gene includes all nucleic acid sequences related to a natural holA gene such as regulatory regions that control production of the δ subunit protein encoded by that gene (such as, but not limited to, transcription, translation or post-translation control regions) as well as the coding region itself. In one embodiment, a holA gene includes the nucleic acid sequence SEQ ID NO: 1. Nucleic acid sequence SEQ ID NO: 1 represents the deduced sequence of a nucleic acid molecule denoted herein as nAeod, the deduction of which is disclosed in the Examples. It should be noted that since nucleic acid sequencing technology is not entirely error-free, SEQ ID NO: 1 (as well as other sequences presented herein), at best, represents an apparent nucleic acid sequence of the nucleic acid molecule encoding a Aquifex aeolicus δ subunit protein of the present invention.

In another embodiment, a holA gene can be an allelic variant that includes a similar but not identical sequence to SEQ ID NO: 1. An allelic variant of a holA gene including SEQ ID NO: 1 is a gene that occurs at essentially the same locus (or loci) in the genome as the gene including SEQ ID NO: 1, but which, due to natural variations caused by, for example, mutation or recombination, has a similar but not identical sequence. Allelic variants typically encode proteins having similar activity to that of the protein encoded by the gene to which they are being compared. Allelic variants can also comprise alterations in the 5′ or 3′ untranslated regions of the gene (e.g., in regulatory control regions). Allelic variants are well known to those skilled in the art and would be expected to be found within a given bacterium since the genome is diploid and/or among a population comprising two or more bacteria. For example, it is believed that H. pylori 26695 δ subunit nucleic acid molecule nHpyl2d, having nucleic acid sequence SEQ ID NO: 37 and to be described in more detail below, represents an allelic variant of H. pylori J99 δ subunit nucleic acid molecule nHpylJd, having nucleic acid sequence SEQ ID NO: 40.

Similarly, a Streptococcus pyogenes holA gene includes all nucleic acid sequences related to a natural Streptococcus pyogenes holA gene such as regulatory regions that control production of the Streptococcus pyogenes δ subunit protein encoded by that gene as well as the coding region itself. In one embodiment, an Streptococcus pyogenes holA gene includes the nucleic acid sequence SEQ ID NO: 103. Nucleic acid sequence SEQ ID NO: 103 represents the deduced sequence of a DNA nucleic acid molecule denoted herein as nSpyod, the production of which is disclosed in the Examples. In another embodiment, an Streptococcus pyogenes holA gene can be an allelic variant that includes a similar but not identical sequence to SEQ ID NO: 103.

Similarly, all other holA genes described herein include all nucleic acid sequences related to the natural holA gene such as regulatory regions that control production of the δ subunit protein encoded by that gene as well as the coding region itself.

An isolated δ subunit protein of the present invention can be obtained from its natural source, can be produced using recombinant DNA technology or can be produced by chemical synthesis. As used herein, an isolated δ subunit protein can be a full-length protein or any homologue of such a protein. Examples of δ subunit homologues include δ proteins in which amino acids have been deleted (e.g., a truncated version of the protein, such as a peptide or by a protein splicing reaction when an intein has been removed or two exteins are joined), inserted, inverted, substituted and/or derivatized (e.g., by glycosylation, phosphorylation, acetylation, methylation, myristylation, prenylation, palmitoylation, amidation and/or addition of glycerophosphatidyl inositol) such that the homologue includes at least one epitope capable of eliciting an immune response against a δ protein. That is, when the homologue is administered to an animal as an immunogen, using techniques known to those skilled in the art, the animal will produce a humoral and/or cellular immune response against at least one epitope of a δ protein. δ homologues can also be selected by their ability to selectively bind to immune serum. Methods to measure such activities are disclosed herein. δ homologues also include those proteins that are capable of performing the function of the native δ proteins in a functional assay.

A δ subunit protein of the present invention may be identified by its ability to perform the function of a δ subunit in a functional assay. The phrase “capable of performing the function of that subunit in a functional assay” means that the protein has at least about 10% of the activity of the natural protein subunit in the functional assay. In other preferred embodiments, has at least about 20% of the activity of the natural protein subunit in the functional assay. In other preferred embodiments, has at least about 30% of the activity of the natural protein subunit in the functional assay. In other preferred embodiments, has at least about 40% of the activity of the natural protein subunit in the functional assay. In other preferred embodiments, has at least about 50% of the activity of the natural protein subunit in the functional assay. In other preferred embodiments, the protein has at least about 60% of the activity of the natural protein subunit in the functional assay. In more preferred embodiments, the protein has at least about 70% of the activity of the natural protein subunit in the functional assay. In more preferred embodiments, the protein has at least about 80% of the activity of the natural protein subunit in the functional assay. In more preferred embodiments, the protein has at least about 90% of the activity of the natural protein subunit in the functional assay.

Examples of functional assays are described in detail below in the section entitled “Use of the proteins of the present invention” and include, but are not limited to, DNA replication assays, β clamp-loading or -unloading assays, ATP binding and hydrolysis assays, protein binding assays and the like.

An isolated protein of the present invention can be produced in a variety of ways, including recovering such a protein from a bacterium and producing such a protein recombinantly. One embodiment of the present invention is a method to produce an isolated protein of the present invention using recombinant DNA technology. Such a method includes the steps of (a) culturing a recombinant cell comprising a nucleic acid molecule encoding a protein of the present invention to produce the protein and (b) recovering the protein therefrom. Details on producing recombinant cells and culturing thereof are presented below. The phrase “recovering the protein” refers simply to collecting the whole fermentation medium containing the protein and need not imply additional steps of separation or purification. Proteins of the present invention can be purified using a variety of standard protein purification techniques.

Isolated proteins of the present invention are preferably retrieved in “substantially pure” form. As used herein, “substantially pure” refers to a purity that allows for the effective use of the protein in a DNA replication assay.

δ Subunit Nucleic Acid Molecules or holA Genes

Another embodiment of the present invention is an isolated nucleic acid molecule capable of hybridizing under stringent conditions with a gene encoding a δ subunit protein. Such a nucleic acid molecule is also referred to herein as a δ subunit nucleic acid molecule or a holA nucleic acid molecule. Particularly preferred is an isolated nucleic acid molecule that hybridizes under stringent conditions with a holA gene. The characteristics of such genes are disclosed herein. In accordance with the present invention, an isolated nucleic acid molecule is a nucleic acid molecule that has been removed from its natural milieu (i.e., that has been subject to human manipulation). As such, “isolated” does not reflect the extent to which the nucleic acid molecule has been purified. An isolated nucleic acid molecule can include DNA, RNA, or derivatives of either DNA or RNA.

As stated above, a holA gene includes all nucleic acid sequences related to a natural holA gene such as regulatory regions that control production of a δ subunit protein encoded by that gene (such as, but not limited to, transcriptional, translational or post-translational control regions) as well as the coding region itself. A nucleic acid molecule of the present invention can be an isolated holA nucleic acid molecule or a homologue thereof. A nucleic acid molecule of the present invention can include one or more regulatory regions, full-length or partial coding regions, or combinations thereof. The minimal size of a holA nucleic acid molecule of the present invention is the minimal size capable of forming a stable hybrid under stringent hybridization conditions with a corresponding natural gene. holA nucleic acid molecules can also include a nucleic acid molecule encoding a hybrid protein, a fusion protein, a multivalent protein or a truncation fragment.

An isolated nucleic acid molecule of the present invention can be obtained from its natural source either as an entire (i.e., complete) gene or a portion thereof capable of forming a stable hybrid with that gene. As used herein, the phrase “at least a portion of” an entity refers to an amount of the entity that is at least sufficient to have the functional aspects of that entity. For example, at least a portion of a nucleic acid sequence, as used herein, is an amount of a nucleic acid sequence capable of forming a stable hybrid with the corresponding gene under stringent hybridization conditions.

An isolated nucleic acid molecule of the present invention can also be produced using recombinant DNA technology (e.g., polymerase chain reaction (PCR) amplification, cloning, etc.) or chemical synthesis. Isolated holA nucleic acid molecules include natural nucleic acid molecules and homologues thereof, including, but not limited to, natural allelic variants and modified nucleic acid molecules in which nucleotides have been inserted, deleted, substituted, and/or inverted in such a manner that such modifications do not substantially interfere with the ability of the nucleic acid molecule to encode a δ subunit protein of the present invention or to form stable hybrids under stringent conditions with natural nucleic acid molecule isolates.

A holA nucleic acid molecule homologue can be produced using a number of methods known to those skilled in the art (see, for example, Sambrook et al., ibid.). For example, nucleic acid molecules can be modified using a variety of techniques including, but not limited to, classic mutagenesis techniques and recombinant DNA techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of nucleic acid molecules and combinations thereof. Nucleic acid molecule homologues can be selected from a mixture of modified nucleic acids by screening for the function of the protein encoded by the nucleic acid (e.g., the ability of a homologue to elicit an immune response against a δ subuit protein and/or to function in a DNA replication assay, or other functional assay, and/or by hybridization with isolated δ subunit-encoding nucleic acids under stringent conditions.

An isolated δ subunit nucleic acid molecule of the present invention can include a nucleic acid sequence that encodes at least one δ subunit protein of the present invention, examples of such proteins being disclosed herein. Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a a subunit protein.

One embodiment of the present invention is a holA nucleic acid molecule of the present invention that is capable of hybridizing under stringent conditions to a nucleic acid strand that encodes at least a portion of a δ subunit or a homologue thereof or to the complement of such a nucleic acid strand. A nucleic acid sequence complement of any nucleic acid sequence of the present invention refers to the nucleic acid sequence of the nucleic acid strand that is complementary to (i.e., can form a complete double helix with) the strand for which the sequence is cited. It is to be noted that a double-stranded nucleic acid molecule of the present invention for which a nucleic acid sequence has been determined for one strand, that is represented by a SEQ ID NO, also comprises a complementary strand having a sequence that is a complement of that SEQ ID NO. As such, nucleic acid molecules of the present invention, which can be either double-stranded or single-stranded, include those nucleic acid molecules that form stable hybrids under stringent hybridization conditions with either a given SEQ ID NO denoted herein and/or with the complement of that SEQ ID NO, which may or may not be denoted herein. Methods to deduce a complementary sequence are known to those skilled in the art. Preferred is a holA nucleic acid molecule that includes a nucleic acid sequence having at least about 65 percent, preferably at least about 70 percent, more preferably at least about 75 percent, more preferably at least about 80 percent, more preferably at least about 85 percent, more preferably at least about 90 percent and even more preferably at least about 95 percent homology with the corresponding region(s) of the nucleic acid sequence encoding at least a portion of a δ subunit protein. Particularly preferred is a δ subunit nucleic acid molecule capable of encoding at least a portion of a δ subunit protein that naturally is present in bacteria.

Preferred holA nucleic acid molecules include at least one of the following sequences: SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 223, SEQ ID NO: 52, SEQ ID NO: 55, SEQ ID NO: 58, SEQ ID NO: 61, SEQ ID NO: 64, SEQ ID NO: 67, SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103, SEQ ID NO: 106, SEQ ID NO: 109, SEQ ID NO: 112, SEQ ID NO: 115, SEQ ID NO: 118, SEQ ID NO: 121, SEQ ID NO: 124, SEQ ID NO: 127, SEQ ID NO: 130, SEQ ID NO: 133, SEQ ID NO: 136, SEQ ID NO: 139, SEQ ID NO: 142, SEQ ID NO: 145, SEQ ID NO: 148, SEQ ID NO: 151, SEQ ID NO: 154. Also preferred are allelic variants of such nucleic acid molecules.

Knowing a nucleic acid molecule of a δ subunit protein of the present invention allows one skilled in the art to make copies of that nucleic acid molecule as well as to obtain a nucleic acid molecule including additional portions of δ subunit protein-encoding genes (e.g., nucleic acid molecules that include the translation start site and/or transcription and/or translation control regions), and/or δ subunit nucleic acid molecule homologues. Knowing a portion of an amino acid sequence of a δ subunit protein of the present invention allows one skilled in the art to clone nucleic acid sequences encoding such a δ subunit protein. In addition, a desired δ subunit nucleic acid molecule can be obtained in a variety of ways including screening appropriate expression libraries with antibodies which bind to δ subunit proteins of the present invention; traditional cloning techniques using oligonucleotide probes of the present invention to screen appropriate libraries or DNA; and PCR amplification of appropriate libraries, or RNA or DNA using oligonucleotide primers of the present invention (genomic and/or cDNA libraries can be used). Illustrative examples are given in the Examples section.

The present invention also includes nucleic acid molecules that are oligonucleotides capable of hybridizing, under stringent conditions, with complementary regions of other, preferably longer, nucleic acid molecules of the present invention that encode at least a portion of a δ subunit protein. Oligonucleotides of the present invention can be RNA, DNA, or derivatives of either. The minimal size of such oligonucleotides is the size required to form a stable hybrid between a given oligonucleotide and the complementary sequence on another nucleic acid molecule of the present invention. Minimal size characteristics are disclosed herein. The size of the oligonucleotide must also be sufficient for the use of the oligonucleotide in accordance with the present invention. Oligonucleotides of the present invention can be used in a variety of applications including, but not limited to, as probes to identify additional nucleic acid molecules, as primers to amplify or extend nucleic acid molecules or in therapeutic applications to inhibit δ subunit production. Such therapeutic applications include the use of such oligonucleotides in, for example, antisense-, triplex formation-, ribozyme- and/or RNA drug-based technologies. The present invention, therefore, includes such oligonucleotides and methods to interfere with the production of δ subunit proteins by use of one or more of such technologies.

Natural Wild-Type Bacterial Cells and Recombinant Molecules and Cells

The present invention also includes a recombinant vector, which includes a holA nucleic acid molecule of the present invention inserted into any vector capable of delivering the nucleic acid molecule into a host cell. Such a vector contains heterologous nucleic acid sequences, that is nucleic acid sequences that are not naturally found adjacent to holA nucleic acid molecules of the present invention. The vector can be either RNA or DNA, either prokaryotic or eukaryotic, and typically is a virus or a plasmid. Recombinant vectors can be used in the cloning, sequencing, and/or otherwise manipulating of holA nucleic acid molecules of the present invention. One type of recombinant vector, herein referred to as a recombinant molecule and described in more detail below, can be used in the expression of nucleic acid molecules of the present invention. Preferred recombinant vectors are capable of replicating in the transformed cell. Preferred nucleic acid molecules to include in recombinant vectors of the present invention are disclosed herein.

As heretofore disclosed, one embodiment of the present invention is a method to produce a δ subunit protein of the present invention by culturing a cell capable of expressing the protein under conditions effective to produce the protein, and recovering the protein.

In a preferred embodiment, the cell to culture is a natural bacterial cell, and delta is isolated from these cells by resolution and reconstitution approaches where all of the required components of the DNA polymerase III holoenzyme are resolved and used to make an assay for one protein. That one protein, if present in limiting quantity, in the presence of saturating levels of the other proteins, permits DNA replication on long circlular DNA templates containing single primers to proceed at a level proportional to the amount of the limiting protein added. Such assays can be used to locate the protein during purification procedures and to assess it purity (ratio of activity/protein=specific activity). Examples of resolution and reconstitution are known in the art and are given in: Schekman, R., Weiner, A., and Kornberg, A., Science 186:987-993 (1974); and McHenry, C. S. and Kornberg, A., J. Biol Chem. 252:6478-6484 (1977).

In another embodiment, a preferred cell to culture is a recombinant cell that is capable of expressing the δ subunit protein, the recombinant cell being produced by transforming a host cell with one or more nucleic acid molecules of the present invention. Transformation of a nucleic acid molecule into a cell can be accomplished by any method by which a nucleic acid molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed nucleic acid molecules of the present invention can remain extrachromosomal or can integrate into one or more sites within a chromosome of the transformed (i.e., recombinant) cell in such a manner that their ability to be expressed is retained. Preferred nucleic acid molecules with which to transform a host cell are disclosed herein.

Suitable host cells to transform include any cell that can be transformed and that can express the introduced δ subunit protein. Such cells are, therefore, capable of producing δ subunit proteins of the present invention after being transformed with at least one nucleic acid molecule of the present invention. Host cells can be either untransformed cells or cells that are already transformed with at least one nucleic acid molecule. Suitable host cells of the present invention can include bacterial, fungal (including yeast), insect, animal and plant cells. Preferred host cells include bacterial cells, with E. coli and B. subtilis cells being particularly preferred. Alternative preferred host cells are untransformed (wild-type) bacterial cells producing cognate δ subunit proteins, including attenuated strains with reduced pathogenicity, as appropriate.

A recombinant cell is preferably produced by transforming a host cell with one or more recombinant molecules, each comprising one or more nucleic acid molecules of the present invention operatively linked to an expression vector containing one or more transcription control sequences. The phrase operatively linked refers to insertion of a nucleic acid molecule into an expression vector in a manner such that the molecule is able to be expressed when transformed into a host cell. As used herein, an expression vector is a DNA or RNA vector that is capable of transforming a host cell and of effecting expression of a specified nucleic acid molecule. Preferably, the expression vector is also capable of replicating within the host cell. Expression vectors can be either prokaryotic or eukaryotic, and are typically viruses or plasmids. Expression vectors of the present invention include any vectors that function (i.e., direct gene expression) in recombinant cells of the present invention, including in bacterial, fungal, insect, animal, and/or plant cells. As such, nucleic acid molecules of the present invention can be operatively linked to expression vectors containing regulatory sequences such as promoters, operators, repressors, enhancers, termination sequences, origins of replication, and other regulatory sequences that are compatible with the recombinant cell and that control the expression of nucleic acid molecules of the present invention. As used herein, a transcription control sequence includes a sequence which is capable of controlling the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to the art. Preferred transcription control sequences include those which function in bacterial, yeast, insect and mammalian cells, such as, but not limited to, tac, lac, pA1 (Kim, D. R. and McHenry, C. S., J. Biol. Chem. 271:20690-20698 (1996)) tzp, trc, oxy-pro, omp/1pp, rrnB, bacteriophage lambda (.lambda.) (such as .lambda.p.sub.L and .lambda.p.sub.R and fusions that include such promoters), bacteriophage T7, T7lac, bacteriophage T3, bacteriophage SP6, bacteriophage SP01, metallothionein, alpha mating factor, Pichia alcohol oxidase, alphavirus subgenomic promoters (such as Sindbis virus subgenomic promoters), baculovirus, Heliothis zea insect virus, vaccinia virus, herpesvirus, poxvirus, adenovirus, simian virus 40, retrovirus actin, retroviral long terminal repeat, Rous sarcoma virus, heat shock, phosphate and nitrate transcription control sequences as well as other sequences capable of controlling gene expression in prokaryotic or eukaryotic cells. Additional suitable transcription control sequences include tissue-specific promoters and enhancers as well as lymphokine-inducible promoters (e.g., promoters inducible by interferons or interleukins). Transcription control sequences of the present invention can also include naturally occurring transcription control sequences naturally associated with a DNA sequence encoding a δ subunit protein.

Expression vectors of the present invention may also contain secretory signals (i.e., signal segment nucleic acid sequences) to enable an expressed δ subunit protein to be secreted from the cell that produces the protein. Suitable signal segments include a δ subunit protein signal segment or any heterologous signal segment capable of directing the secretion of a δ subunit protein, including fusion proteins, of the present invention. Preferred signal segments include, but are not limited to, δ subunit, tissue plasminogen activator (t-PA), interferon, interleukin, growth hormone, histocompatibility and viral envelope glycoprotein signal segments.

Expression vectors of the present invention may also contain fusion sequences which lead to the expression of inserted nucleic acid molecules of the present invention as fusion proteins. Inclusion of a fusion sequence as part of a holA nucleic acid molecule of the present invention can enhance the stability during production, storage and/or use of the protein encoded by the nucleic acid molecule. Furthermore, a fusion segment can function as a tool to simplify purification of a δ subunit protein, such as to enable purification of the resultant fusion protein using affinity chromatography. A suitable fusion segment can be a domain of any size that has the desired function (e.g., increased stability and/or purification tool). It is within the scope of the present invention to use one or more fusion segments. Fusion segments can be joined to amino and/or carboxyl termini of a δ subunit protein. Linkages between fusion segments and δ subunit proteins can be constructed to be susceptible to cleavage to enable straightforward recovery of the δ subunit proteins. Fusion proteins are preferably produced by culturing a recombinant cell transformed with a fusion nucleic acid sequence that encodes a protein including the fusion segment attached to either the carboxyl and/or amino terminal end of a δ subunit protein.

A recombinant molecule of the present invention is a molecule that can include at least one of any nucleic acid molecule heretofore described operatively linked to at least one of any transcription control sequence capable of effectively regulating expression of the nucleic acid molecules in the cell to be transformed. A preferred recombinant molecule includes one or more nucleic acid molecules of the present invention, with those that encode one or more δ subunit proteins being particularly preferred. Preferred recombinant molecules of the present invention and their production are described in the Examples section. Similarly, a preferred recombinant cell includes one or more nucleic acid molecules of the present invention, with those that encode one or more δ subunit proteins. Preferred recombinant cells of the present invention include those disclosed in the Examples section.

It may be appreciated by one skilled in the art that use of recombinant DNA technologies can improve expression of transformed nucleic acid molecules by manipulating, for example, the number of copies of the nucleic acid molecules within a host cell, the efficiency with which those nucleic acid molecules are transcribed, the efficiency with which the resultant transcripts are translated, and the efficiency of post-translational modifications. Recombinant techniques useful for increasing the expression of nucleic acid molecules of the present invention include, but are not limited to, operatively linking nucleic acid molecules to high-copy number plasmids, integration of the nucleic acid molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of nucleic acid molecules of the present invention to correspond to the codon usage of the host cell, deletion of sequences that destabilize transcripts, and use of control signals that temporally separate recombinant cell growth from recombinant protein production during fermentation. The activity of an expressed recombinant protein of the present invention may be improved by fragmenting, modifying, or derivatizing the resultant protein.

In accordance with the present invention, recombinant cells can be used to produce δ subunit proteins of the present invention by culturing such cells under conditions effective to produce such a protein, and recovering the protein. Effective conditions to produce a protein include, but are not limited to, appropriate media, bioreactor, temperature, pH and oxygen conditions that permit protein production. An appropriate, or effective, medium refers to any medium in which a cell of the present invention, when cultured, is capable of producing a δ subunit protein. Such a medium is typically an aqueous medium comprising assimilable carbohydrate, nitrogen and phosphate sources, as well as appropriate salts, minerals, metals and other nutrients, such as vitamins. The medium may comprise complex, nutrients or may be a defined minimal medium.

Cells of the present invention can be cultured in conventional fermentation bioreactors, which include, but are not limited to, batch, fed-batch, cell recycle, and continuous fermentors. Culturing can also be conducted in shake flasks, test tubes, microtiter dishes, and petri plates. Culturing is carried out at a temperature, pH and oxygen content appropriate for the recombinant cell. Such culturing conditions are well within the expertise of one of ordinary skill in the art.

Depending on the vector and host system used for production, resultant δ subunit proteins may either remain within the recombinant cell; be secreted into the fermentation medium; be secreted into a space between two cellular membranes, such as the periplasmic space in E. coli; or be retained on the outer surface of a cell or viral membrane. Methods to purify such proteins are disclosed in the Examples section.

Antibodies

The present invention also includes isolated anti-δ subunit antibodies and their use. An anti-δ subunit antibody is an antibody capable of selectively binding to a δ subunit. Isolated antibodies are antibodies that have been removed from their natural milieu. The term “isolated” does not refer to the state of purity of such antibodies. As such, isolated antibodies can include anti-sera containing such antibodies, or antibodies that have been purified to varying degrees. As used herein, the term “selectively binds to” refers to the ability of such antibodies to preferentially bind to the protease against which the antibody was raised (i.e., to be able to distinguish that protease from unrelated components in a mixture). Binding affinities, commonly expressed as equilibrium association constants, typically range from about 10³ M⁻¹ to about 10¹² M⁻¹. Binding can be measured using a variety of methods known to those skilled in the art including immunoblot assays, immunoprecipitation assays, radioimmunoassays, enzyme immunoassays (e.g., ELISA), immunofluorescent antibody assays and immunoelectron microscopy; see, for example, Sambrook et al., ibid.

Antibodies of the present invention can be either polyclonal or monoclonal antibodies. Antibodies of the present invention include functional equivalents such as antibody fragments and genetically-engineered antibodies, including single chain antibodies, that are capable of selectively binding to at least one of the epitopes of the protein used to obtain the antibodies. Antibodies of the present invention also include chimeric antibodies that can bind to more than one epitope. Preferred antibodies are raised in response to proteins that are encoded, at least in part, by a δ subunit nucleic acid molecule of the present invention.

Anti-δ subunit antibodies of the present invention include antibodies raised in an animal administered a δ subunit. Anti-δ subunit antibodies of the present invention also include antibodies raised in an animal against one or more δ subunit proteins of the present invention that are then recovered from the cell using techniques known to those skilled in the art. Yet additional antibodies of the present invention are produced recombinantly using techniques as heretofore disclosed for δ subunit proteins of the present invention. Antibodies produced against defined proteins can be advantageous because such antibodies are not substantially contaminated with antibodies against other substances that might otherwise cause interference in a diagnostic assay or side effects if used in a therapeutic composition.

Anti-δ subunit antibodies of the present invention have a variety of uses that are within the scope of thus present invention. For example, such antibodies can be used in a composition of the present invention to passively immunize an animal in order to protect the animal from bacterial infection. Anti-δ subunit antibodies can also be used as tools to screen expression libraries and/or to recover desired proteins of the present invention from a mixture of proteins and other contaminants. Furthermore, antibodies of the present invention can be used to target cytotoxic, therapeutic or imaging agents to bacteria or bacteria-infected organs or tissues in order to kill bacteria, deliver therapeutic agents or localize imaging agents to bacteria-infected organs or tissues. Targeting can be accomplished by conjugating (i.e., stably joining) such antibodies to the cytotoxic, therapeutic or imaging agents using techniques known to those skilled in the art.

A preferred anti-δ subunit antibody of the present invention can selectively bind to, and preferentially reduce the DNA replication activity of a replicase comprising a δ subunit protein.

Use of the Proteins of the Present Invention

The present invention provides methods for the use of δ subunit proteins of the present invention in a reconstitution assay of DNA polymerase III holoenzyme (comprising α, β, DnaX, δ, and δ′ subunits). Such methods are described in detail in U.S. Provisional Patent application Serial Nos. 60/244,023, filed Oct. 27, 2000 and 60/290,725, filed May 14, 2001, incorporated by reference herein in their entirety.

The present invention also provides methods for the use of δ subunit proteins of the present invention derived from thermophilic bacteria for amplification of DNA, inlucluding polymerase chain reaction (PCR) and isothermal amplification, including, but not limited to, rolling circle amplification, for research (cloning and analytical) and for diagnostic purposes. Such methods are described in detail in WO 99/13060. Such δ subunit proteins include, but are not limited to those dervived from Aquifex, Thermus, and Thermatoga genuses. Specific examples are δ subunit protein from Aquifex aeolicus (SEQ ID NO: 3, SEQ ID NO: 226); Thermus thermophilus (SEQ ID NO: 108), and Thermatoga maratima (SEQ ID NO: 81).

In one embodiment, a thermostable polymerase is one that retains at least 50% of its activity upon incubation at 70° C. for 5 minutes in a solution containing 50 mM Tris-HCl (pH 7.5), 100 mM potassium glutamate, 20% glycerol and 1 mM dithiothreitol. In another embodiment, a thermostable polymerase is one that retains at least 50% of its activity upon incubation at 60° C. for 5 minutes in a solution containing 50 mM Tris-HCl (pH 7.5), 100 mM potassium glutamate, 20% glycerol and 1 mM dithiothreitol. In preferred embodiments, a thermostable polymerase is one that retains at least 50% of its activity upon incubation at 60° C. for 10 minutes in a solution containing 50 mM Tris-HCl (pH 7.5), 100 mM potassium glutamate, 20% glycerol and 1 mM dithiothreitol.

The present invention also includes the use of modulators of bacterial DNA replication that reduce the replication activity of a DNA polymerase III replicase comprising a δ subunit to reduce bacterial infection of animals, plants, humans and the surrounding environment. As used herein, modulators of bacterial DNA replication are compounds that interact with the replicase thereby altering the ability of replicase to replicate DNA. A preferred modulator inhibits replication.

The minimal assembly of the essential subunits of a bacterial replicase should permit rapid and processive synthesis of long stretches of DNA. In all bacterial replicases studied so far, the minimal functional holoenzyme consists of three components: 1) the polymerase core, 2) the β processivity factor-loading (or clamp-loading) complex and 3) the sliding clamp processivity factor, β. In E. coli, the minimal holoenzyme consists of the a subunit, DnaX-δδ′ clamp loading complex and the β subunit. The same components are minimally required for functional DNA Pol III holoenzyme from Gram-positive S. pyogenes and T. thermophilus. Given the generality of this requirement among distantly related organisms, it is expected that α, β, DnaX, δ, and δ′ will be sufficient for the assembly of a replicase for most organisms. As used herein, DnaX complex is the term used to describe an assembly of DnaX, delta and delta′.

Target indications may include arresting cell growth or causing cell death resulting in recovery from the bacterial infection in animal studies. A wide variety of assays for activity and binding agents are provided, including DNA synthesis, ATPase, clamp loading onto DNA, protein-protein binding assays, immunoassays, cell based assays, etc. The replication protein compositions, used to identify pharmacological agents, are in isolated, partially pure or pure form and are typically, but not necessarily, recombinantly produced. The replication protein may be part of a fusion product with another peptide or polypeptide (e. g., a polypeptide that is capable of providing or enhancing protein-protein binding, stability under assay conditions (e. g., a tag for detection or anchoring), etc.). The assay mixtures comprise a natural intracellular replication protein-binding target such as DNA, another protein, NTP, or dNTP. For binding assays, while native binding targets may be used, it is frequently preferred to use portions (e. g., peptides, nucleic acid fragments) thereof so long as the portion provides binding affinity and avidity to the subject replication protein conveniently measurable in the assay. The assay mixture also comprises a candidate pharmacological agent. Generally, a plurality of assay mixtures are run in parallel with different agent concentrations to obtain a differential response to the various concentrations. Typically, one of these concentrations serves as a negative control (i.e., at zero concentration or below the limits of assay detection). Additional controls are often present such as a positive control, a dose response curve, use of known inhibitors, use of control heterologous proteins, etc. Candidate agents encompass numerous chemical classes, though typically they are organic compounds; preferably they are small organic compounds and are obtained from a wide variety of sources, including libraries of synthetic or natural compounds. A variety of other reagents may also be included in the mixture. These include reagents like salts, buffers, neutral proteins (e.g., albumin, detergents, etc.) which may be used to facilitate optimal binding and/or reduce nonspecific or background intersactions, etc. Also, reagents that otherwise improve the efficiency of the assay (e.g., protease inhibitors, nuclease inhibitors, antimicrobial agents, etc.) may be used.

The invention provides replication protein specific assays and the binding agents including natural intracellular binding targets such as other replication proteins, etc., and methods of identifying and making such agents, and their use in a a variety of diagnostic and therapeutic applications, especially where disease is associated with excessive cell growth. Novel replication protein-specific antibodies and other natural intracellular binding agents identified with assays such as one- and two-hybrid screens, non-natural intracellular binding agents identified in screens of chemical libraries, etc.

Generally, replication protein-specificity of the binding agent is shown by equilibrium constants or values that approximate equilibrium constants such as the concentration that inhibits a reaction 50% (IC₅₀). Such agents are capable of selectively binding a replication protein (i.e., with an equilibrium association constant at least about 10⁷ M⁻¹, preferably, at least about 10⁸ M⁻¹, more preferably, at least about 10⁹ M⁻¹). A wide variety of cell-based and cell-free assays may be used to demonstrate replication protein-specific activity, binding, gel shift assays, immunoassays, etc.

The resultant mixture is incubated under conditions whereby, but for the presence of the candidate pharmacological agent, the replication protein specifically binds the cellular binding target, portion, or analog. The mixture of components can be added in any order that provides for the requisite bindings.

Incubations may be performed at any temperature which facilitates optimal binding, typically between 4° C. and 40° C., more commonly between 15° C. and 37° C. Incubation periods are likewise selected for optimal binding but also minimized to facilitate rapid, high-throughput screening, and are typically between 0.1 and 10 hours, preferably less than δ hours, more preferably less than 1 hour.

After incubation, the presence or absence of activity or specific binding between the replication protein and one or more binding targets is detected by any convenient way. For cell-free activity and binding type assays, a separation step may be used to separate the activity product or the bound from unbound components.

Separation may be effected by precipitation (e. g., immunoprecipitation or acid precipitation of a nucleic acid product), immobilization (e. g., on a solid substrate such as a microtiter plate), etc., followed by washing. Many assays that do not require separation are also possible such as use of europium conjugation in proximity assays, fluorescence increases upon double stranded DNA synthesis in the presence of dyes that increase fluorescence upon intercalating in double-stranded DNA, scintillation proximity assays or a detection system that is dependent on a reaction product or loss of substrate.

Detection may be effected in any convenient way. For cell-free activity and binding assays, one of the components usually comprises or is coupled to a label.

A wide variety of labels may be employed-essentially any label that provides for detection of DNA product, loss of DNA substrate, conversion of a nucleotide substrate, or bound protein is useful. The label may provide for direct detection such as radioactivity, fluorescence, luminescence, optical, or electron density, etc. or indirect detection such as an epitope tag, an enzyme, etc. The label may be appended to the protein (e.g., a phosphate group comprising a radioactive isotope of phosphorous), or incorporated into the DNA substrate or the protein structure (e.g., a methionine residue comprising a radioactive isotope of sulfur). A variety of methods may be used to detect the label depending on the nature of the label and other assay components. For example, the label may be detected bound to the solid substrate, or a portion of the bound complex containing the label may be separated from the solid substrate, and thereafter the label detected. Labels may be directly detected through optical or electron density, radioactive emissions, nonradiative energy transfer, fluorescence emission, etc. or indirectly detected with antibody conjugates, etc. For example, in the case of radioactive labels, emissions may be detected directly (e.g., with particle counters) or indirectly (e.g., with scintillation cocktails and counters).

The present invention provides methods by which replication proteins from bacteria are used to discover new pharmaceutical agents. The function of replication proteins is quantified in the presence of different chemical test compounds. A chemical compound that inhibits the function is a candidate antibiotic.

In some embodiments it is prefereable to use replication protein subunits derived from a single organism. In other embodiments, the various subunits may be derived from one or more organisms. Replication proteins from Gram positive bacteria and from Gram negative bacteria can be interchanged for one another in certain embodiments. Hence, they can function as mixtures. Suitable E. coli replication proteins are the subunits of its Pol III holoenzyme which are described in U.S. Pat. Nos. 5,583,026 and 5,668,004 to O'Donnell, which are hereby incorporated by reference.

Some other replication subunit proteins useful in the present invention are detailed in Table 3; “DnaX proteins identified from PSI-BLAST search of completely sequenced genomes;” Table 4, “δ′ proteins identified in PSI-BLAST searches of all finished genomes at NCBI;” Table 5, “DnaX proteins from S. pyogenes, S. pneumoniae and S. aureus;” Table 6, “δ′ proteins from S. pyogenes, S. pneumoniae and S. aureus;” Table 7, “δ proteins identified in PSI-BLAST searches of unfinished bacterial genomes at NCBI;” Table 8, “DnaX proteins corresponding to δ proteins identified from PSI-BLAST search of unfinished genomes at NCBI;” Table 9, “δ′ proteins corresponding to δ proteins identified from PSI-BLAST search of unfinished genomes at NCBI;” Table 10, “DnaX proteins corresponding to δ proteins identified in BLAST searches of phylogenically related organisms;” Table 11, “δ′ proteins corresponding to δ proteins identified in BLAST searches of phylogenically related organisms;” Table 12, “DnaE Proteins corresponding to δ proteins identified in PSI-BLAST searches of finished genomes at NCBI;” Table 13, “DnaE and PolC Proteins from S. pyogenes, S. pneumoniae and S. aureus;” Table 14, “DnaE and Pol C Proteins Corresponding to Additional δ Proteins Identified from PSI-BLAST search of Partially Sequenced Genomes;” Table 15, “DnaE and PolC proteins corresponding to δ proteins identified in BLAST searches of phylogenically related organisms;” Table 16, “β Proteins corresponding to δ identified from PSI-BLAST search of completely sequenced genomes;” Table 17, “β Proteins from S. pyogenes, S. pneumoniae and S. aureus;” and Table 18, “β Proteins Corresponding to δ Proteins Identified in BLAST Searches of Phylogenically Related Organisms.” Table 21, “β Proteins Corresponding to Additional δ Proteins Identified from PSI-BLAST Search of Partially Sequenced Genomes.” For those organisms not represented in the aforementioned Tables, the likely reason is that the relevant region of the genome has not yet been sequenced. Once sequenced, the proteins can be found using methods outlined herein. In each of the aforementioned Tables, NCBI refers to the NCBI nr database, a compilation of non-redundant sequences. The accession numbers reported are not NCBI database accession numbers per se, but reference the database that compiled the sequences.

Some of the information detailed in Tables 3-18 and 21 is known in the art. Some of this information was obtained using the methods detailed herein in the Examples section, particulary Examples 1-3 and 5. Other subunit proteins useful in the present invention are detailed in Figures. Still other subunit proteins are well known in the art.

The present invention, in one embodiment provides a method of screening for a compound or chemical that modulates the activity of a DNA polymerase III replicase comprising, a) contacting an isolated replicase with at least one test compound under conditions permissive for replicase activity, b) assessing the activity of the replicase in the presence of the test compound, and c) comparing the activity of the replicase in the presence of the test compound with the activity of the replicase in the absence of the test compound, wherein a change in the activity of the replicase in the presence of the test compound is indicative of a compound that modulates the activity of the replicase, wherein said replicase comprises an isolated DNA polymerase III δ subunit protein of the present invention.

The present invention describes a method of identifying compounds which modulate the activity of a DNA polymerase III replicase. This method is carried out by forming a reaction mixture that includes a primed DNA molecule, a DNA polymerase III replicase, a candidate compound, a dNTP or up to 4 different dNTPs, ATP, and optionally either a β subunit, a DnaX complex, or both the β subunit and the DnaX complex; subjecting the reaction mixture to conditions effective to achieve nucleic acid polymerization in the absence of the candidate compound; analyzing the reaction mixture for the presence or absence of nucleic acid polymerization extension products and comparing the activity of the replicase in the presence of the test compound with the activity of the replicase in the absence of the test compound. A change in the activity of the replicase in the presence of the test compound is indicative of a compound that modulates the activity of the replicase. The DnaX complex comprises a δ subunit protein of the present invention.

The present invention describes a method to identify candidate pharmaceuticals that modulate the activity of a DnaX complex and a β subunit in stimulating the DNA polymerase. The method includes contacting a primed DNA (which may be coated with SSB) with a DNA polymerase, a β subunit, and a DnaX complex (or subunit or subassembly of the DnaX complex) in the presence of the candidate pharmaceutical, and dNTPs (or modified dNTPs) to form a reaction mixture. The reaction mixture is subjected to conditions which, in the absence of the candidate pharmaceutical, would effect nucleic acid polymerization and the presence or absence of the extension product in the reaction mixture is analyzed. The nucleic acid polymerization in the presence of the test compound is compared with the nucleic acid polymerization in the absence of the test compound. A change in the nucleic acid polymerization in the presence of the test compound is indicative of a compound that modulates the activity of a DnaX complex and β subunit. The DnaX complex comprises a δ subunit of the present invention.

The present invention describes a method to identify chemicals that modulate the ability of a β subunit and a DnaX complex (or a subunit or subassembly of the DnaX complex) to interact. This method includes contacting the β subunit with the DnaX complex (or subunit or subassembly of the DnaX complex) in the presence of ATP (or a suitable ATP analog, such as ATPγS, a non-hydrolyzable analog of ATP, that enables interaction of β with the DnaX complex (or subunit or subassembly of the DnaX complex) the candidate pharmaceutical to form a reaction mixture. The reaction mixture is subjected to conditions under which the DnaX complex (or the subunit or subassembly of the DnaX complex) and the β subunit would interact in the absence of the candidate pharmaceutical. The reaction mixture is then analyzed for interaction between the β subunit and the DnaX complex (or the subunit or subassembly of the DnaX complex). The extent of interaction in the presence of the test compound is compared with the extent of interaction in the absence of the test compound. A change in the interaction between the β subunit and the DnaX complex (or the subunit or subassembly of the DnaX complex) is indicitive of a compound that modulates the interaction. The DnaX complex comprises a δ protein of the present invention.

The present invention describes a method to identify chemicals that modulate the ability of a δ subunit and the δ′ and/or DnaX subunit to interact. This method includes contacting the δ subunit with the δ′ and/or δ′ plus DnaX subunit in the presence of the candidate pharmaceutical to form a reaction mixture. The reaction mixture is subjected to conditions under which the δ subunit and the δ′ and/or δ′ plus DnaX subunit would interact in the absence of the candidate pharmaceutical. The reaction mixture is then analyzed for interaction between the δ subunit and the δ′ and/or DnaX subunit. The extent of interaction in the presence of the test compound is compared with the extent of interaction in the absence of the test compound. A change in the interaction between the δ subunit and the δ′ and/or DnaX subunit is indicitive of a compound that modulates the interaction.

The present invention describes a method to identify chemicals that modulate the ability of a DnaX complex (or a subassembly of the DnaX complex) to assemble a β subunit onto a DNA molecule. This method involves contacting a circular primed DNA molecule (which may be coated with SSB) with the DnaX complex (or the subassembly thereof) and the β subunit in the presence of the candidate pharmaceutical, and ATP or dATP to form a reaction mixture. The reaction mixture is subjected to conditions under which the DnaX complex (or subassembly) assembles the β subunit on the DNA molecule absent the candidate pharmaceutical. The presence or absence of the β subunit on the DNA molecule in the reaction mixture is analyzed. The extent of assembly in the presence of the test compound is compared with the extent of assmbly in the absence of the test compound. A change in the amount of β subunit on the DNA molecule is indicative of a compound that modulates the ability of a DnaX complex (or a subassembly of the DnaX complex) to assemble a β subunit onto a DNA molecule. The DnaX complex comprises a delta protein of the present invention.

The present invention describes a method to identify chemicals that modulate the ability of a DnaX complex (or a subunit (s) of the DnaX complex) to disassemble a β subunit from a DNA molecule. This method comprises contacting a DNA molecule onto which the β subunit has been assembled in the presence of the candidate pharmaceutical, to form a reaction mixture. The reaction mixture is subjected to conditions under which the DnaX complex (or a subunit (s) or subassembly of the DnaX complex) disassembles the β subunit from the DNA molecule absent the candidate pharmaceutical. The presence or absence of the β subunit on the DNA molecule in the reaction mixture is analyzed. The extent of assembly in the presence of the test compound is compared with the extent of assembly in the absence of the test compound. A change in the amount of β subunit on the DNA molecule is indicative of a compound that modulates the ability of a DnaX complex (or a subassembly of the DnaX complex) to disassemble a β subunit onto a DNA molecule. The DnaX complex comprises a δ subunit of the present invention.

The present invention describes a method to identify chemicals that modulate the dATP/ATP binding activity of a DnaX complex or a DnaX complex subunit (e. g. DnaX subunit). This method includes contacting the DnaX complex (or the DnaX complex subunit) with dATP/ATP either in the presence or absence of a DNA molecule and/or the β subunit in the presence of the candidate pharmaceutical to form a reaction. The reaction mixture is subjected to conditions in which the DnaX complex (or the subunit of DnaX complex) interacts with dATP/ATP in the absence of the candidate pharmaceutical. The reaction is analyzed to determine if dATP/ATP is bound to the DnaX complex (or the subunit of DnaX complex) in the presence of the candidate pharmaceutical. The extent of binding in the presence of the test compound is compared with the extent of binding in the absence of the test compound. A change in the dATP/ATP binding is indicative of a compound that modulates the dATP/ATP binding activity of a DnaX complex or a DnaX complex subunit (e.g. DnaX subunit). The DnaX complex comprises a δ subunit of the present invention.

The present invention describes a method to identify chemicals that modulate the dATP/ATPase activity of a DnaX complex or a DnaX complex subunit (e.g., the DnaX subunit). This method involves contacting the DnaX complex (or the DnaX complex subunit) with dATP/ATP either in the presence or absence of a DNA molecule and/or a β subunit in the presence of the candidate pharmaceutical to form a reaction mixture. The reaction mixture is subjected to conditions in which the DnaX subunit (or complex) hydrolyzes dATP/ATP in the absence of the candidate pharmaceutical. The reaction is analyzed to determine if dATP/ATP was hydrolyzed. The extent of hyrdolysis in the presence of the test compound is compared with the extent of hydrolysis in the absence of the test compound. A change in the amount of dATP/ATP hydrolyzed is indicative of a compound that modulates the dATP/ATPase activity of a DnaX complex or a DnaX complex subunit (e. g., the DnaX subunit). The DnaX complex comprises a δ protein of the present invention.

The invention provides a further method for identifying chemicals that modulate the activity of a DNA polymerase comprising contacting a circular primed DNA molecule (may be coated with SSB) with a DnaX complex, a β subunit and an α subunit in the presence of the candidate pharmaceutical, and dNTPs (or modified dNTPs) to form a reaction mixture. The reaction mixture is subjected to conditions, which in the absence of the candidate pharmaceutical, affect nucleic acid polymerization, and the presence or absence of the extension product in the reaction mixture is analyzed. The activity of the replicase in the presence of the test compound is compared with the activity of the replicase in the absence of the test compound. A change in the activity of the replicase in the presence of the test compound is indicative of a compound that modulates the activity of the replicase. The DnaX complex comprises a δ protein of the present invention.

The present invention also includes a test kit to identify a compound capable of modulating DNA polymerase III replicase activity. Such a test kit includes an isolated δ subunit protein and a means for determining the extent of modulation of DNA replication activity in the presence of (i.e., effected by) a putative inhibitory compound.

The present invention also includes modulators and inhibitors isolated by such a method, and/or test kit, and their use to inhibit any replicase comprising a δ subunit that is susceptible to such an inhibitor. Modulators of bacterial DNA replication can also be used directly as to treat animals, plants and humans suspected of having a bacterial infection as long as such compounds are not harmful to the species being treated. Similarly, molecules that bind to any replicase comprising a δ subunit, incuding but not limited to antibodies and small molecules, can also be used to target cytotoxic, therapeutic or imaging entities to a site of infection.

With the availability of the essential DNA polymerase III holoenzyme subunits purified by a reconstitution approach, replication methods of the invention are amenable to a high-throughput screen against the new target provided. This can be done by titrating each component until it is barely limiting so that modulation will be observed upon interaction with active compounds within small molecule libraries. Buffer conditions will be optimized to ensure stability of the enzyme mix, stored in a separate chilled container, during the multi-plate screening process. The time and temperature of the assay will also be reoptimized to ensure a stable linear response. Similarly, concentrations of all the relevant components of the assay will be optimized to ensure high sensitivity of the assay to perturbation by test compounds.

The methods are amenable to automated, cost-effective high-throughput screening of chemical libraries for lead compounds. Identified reagents find use in the pharmaceutical industries for animal and human trials as well as agricultural industries for plant pathogens; for example, the reagents may be derivatized and rescreened in in vitro and in vivo assays to optimize activity and minimize toxicity for pharmaceutical, animal care or agricultural product development.

### EXAMPLES

### Example 1

General Strategy

Identification of members of a related family of bacterial DnaX complex subunits (DnaX, δ′ and δ of DNA polymerase III holoenzymes from organisms whose genomes have been completely sequenced and deposited at the National Center for Biotechnology Information (NCBI) is possible using the computer program PSI-BLAST (Position-Specific Iterated BLAST) (Altschul, et al., Nucleic Acids Res. 25:3389-402 (1997)). PSI-BLAST (or ψ-BLAST) is a program in which a profile (or position specific scoring matrix, PSSM) is constructed from a multiple alignment of the highest scoring hits in an initial BLAST (Basic Local Alignment Search Tool) search (Altschul, et al., J. Mol. Biol. 215:403-410 (1990)). BLAST is a set of similarity search programs designed to explore all of the available sequence databases regardless of whether the query is protein or DNA.

The ψ-BLAST profile contruct from BLAST searches is used to perform a second (and successive) search and the results of each “iteration” used to refine the profile. This iterative searching strategy results in increased sensitivity. BLAST and PSI-BLAST can be accessed at the NCBI web site accessible by Microsoft Explorer, Netscape or other suitable web browser. Rules or criteria are presented to allow for the identification of bacterial DnaX, δ′ and δ.

The DnaX proteins from targeted organisms are first identified. Next, the δ′ subunits from those organisms are identified. δ′ subunits exhibit sequence similarity with DnaX, but lack certain conserved motifs found only in DnaX proteins. Finally, the δ subunits from the organisms are identified. δ subunits are less conserved but two procedures have been developed that permit their unambiguous identification.

### Example 2

Identification of DnaX Sequences from Completely Sequenced Genomes

Identification of DnaX proteins: The sensitivity of the initial BLAST search and ensuing iterations may be manipulated by settings found in the “options” and “format” portions of the search window. The settings are described in the steps listed below and were used in searches for DnaX, δ′, and δ.

Step 1: Search criteria should be set in “format” to these settings:

Set inclusion threshold (E or Expect value) to 0.001 or 0.002

Search criteria should be set in “option” to these settings:

Set matrix to Blosum62

Set gap cost to Existence: 11 Extension: 1

Set expect to 10

Information regarding the input parameters is available from the BLAST web page available throught NCBI. The statistical significance threshold for including a sequence in the model used by PSI-BLAST to create the PSSM on the next iteration.

For example, different substitution matrices are tailored to detecting similarities among sequences that are diverged by differing degrees. A single matrix may nevertheless be reasonably efficient over a relatively broad range of evolutionary change. Experimentation has shown that the BLOSUM-62 matrix is among the best for detecting most weak protein similarities. A detailed statistical theory for gapped alignments has not been developed, and the best gap costs to use with a given substitution matrix are determined empirically; however, it is suggested that an initial search start with Existence: 10, Extension: 1.

“Expect”, also referred to as an ‘E value’ or ‘E’ in this application is the statistical significance threshold for reporting matches against database sequences. When Expect is set to 10, 10 matches are expected to be found merely by chance, according to the stochastic model of Karlin and Altschul (1990). With Expect set to 0.001, there is a 1 in 1000 chance for an inappropriate hit, based on the referenced stochastic model. If the statistical significance ascribed to a match is greater than the Expect threshold, the match will not be reported. Lower Expect thresholds are more stringent, leading to fewer chance matches being reported. Increasing the threshold shows less stringent matches. Fractional values are acceptable.

Step 2: There are also settings in the “option” portion of the search window to limit the search to the genomes of specific organisms. In the window labeled “Limit by entrez query” the names of the organisms to which you are limiting your search must be typed or pasted. Entrez is a retrieval system for searching several linked databases, by entering specific organism here the search is limited to searching for protein sequences similar to the query sequence only from the specified organisms. The search was limited to only those organisms that have the genomic DNA completely sequenced and available at National Center for Biotechnology Information. The listing for these organisms can be found at the NCBI web site. The search for proteins in the examples described in this application was limited to the 34 organisms listed below.

Aquifex aeolicus[Organism] OR Bacillus halodurans[Organism] OR Bacillus subtilis[Organism] OR Buchnera[Organism] OR Borrelia burgdorferi[Organism] OR Campylobacter jejuni[Organism] OR Caulobacter crescentus[Organism] OR Chlamydia pneumoniae[Organism] OR Chlamydia muridarum[Organism] OR Chlamydia trachomatis[Organism] OR Deinococcus radiodurans[Organism] OR Escherichia coli[Organism] OR Escherichia coli K12[Organism] OR Escherichia coli O157[Organism] OR Haemophilus influenzae[Organism] OR Helicobacter pylori 26695[Organism] OR Helicobacter pylori J99[Organism] OR Lactococcus lactis[Organism] OR Mesorhizobium loti[Organism] OR Mycobacterium leprae[Organism] OR Mycobacterium tuberculosis[Organism] OR Mycoplasma genitalium[Organism] OR Mycoplasma pneumoniae[Organism] OR Neisseria meningitidis MC58[Organism] OR Neisseria meningitidis Z2491[Organism] OR Pasteurella multocida[Organism] OR Pseudomonas aeruginosa[Organism] OR Rickettsia prowazekii[Organism] OR Synechocystis[Organism] OR Thermotoga maritima[Organism] OR Treponema pallidum[Organism] OR Ureaplasma urealyticum[Organism] OR Vibrio cholerae[Organism] OR Xylella fastidiosa[Organism]

The format for entering organisms must be as shown. The entire name of the organism must be spelled out. Using the abbreviated form of a name (i.e. E. coli) is not permitted. The name can be followed by a strain designation (i.e. Neisseria meningitidis Z2491), but not by a designation for an entire species (i.e. Buchnera but not Buchnera sp.). Also, the name must be followed by a classification enclosed by brackets (i.e. [Organism] or [orgn]) with no spaces separating the name from the bracket.

Step 3: The amino acid sequence of a query protein must be entered into the search box. The initial BLAST search was against this initial amino acid query sequence. There is a window (search) within the initial search window to specify the query sequence. In this search, the amino acid sequence for E. coli DnaX (SEQ ID. NO. 200) was used (shown below).

MSYQVLARKWRPQTFADVVGQEHVLTALANGLSLGRIHHAYLFSGTRGVG
KTSIARLLAKGLNCETGITATPCGVCDNCREIEQGRFVDLIEIDAASRTK
VEDTRDLLDNVQYAPARGRFKVYLIDEVHMLSRHSFNALLKTLEEPEHVK
FLLATTDPQKLPVTILSRCLQFHLKALDVEQIRHQLEHILNEEHIAHEPR
ALQLLARAAEGSLRDALSLTDQAIASGDGQVSTQAVSAMLGTLDDDQALS
LVEAMVEANGERVMALINEAAARGIEWEALLVEMLGLLHRIAMVQLSPAA
LGNDMAAIELRMRELARTIPPTDIQLYYQTLLIGRKELPYAPDRRMGVEM
TLLRALAFHPRMPLPEPEVPRQSFAPVAPTAVMTPTQVPPQPQSAPQQAP
TVPLPETTSQVLAARQQLQRVQGATKAKKSEPAAATRARPVNNAALERLA
SVTDRVQARPVPSALEKAPAKKEAYRWKATTPVMQQKEVVATPKALKKAL
EHEKTPELAAKLAAEAIERDPWAAQVSQLSLPKLVEQVALNAWKEESDNA
VCLHLRSSQRHLNNRGAQQKLAEALSMLKGSTVELTIVEDDNPAVRTPLE
WRQAIYEEKLAQARESIIADNNIQTLRRFFDAELDEESIRPI

The PSI-BLAST search returned 143 hits. Hits are shown in descending similarity to the E. coli query sequence, and typically proteins similar to the E. coli query protein are among the first hits.

Step 4: Criteria used to select hits that are DnaX proteins.

ATP binding proteins share local similarities required for nucleotide binding and hydrolysis called the Walker A and Walker B motifs. The Walker A nucleotide binding fold consensus sequence is G(X)₄GK(T/S)(X)₆(I/V) (SEQ ID NO: 201) (where X is any amino acid) and the Walker B binding fold consensus sequence is (R/K)(X)₃G(X)₃L(hydrophobic)₄D (SEQ ID NO: 202) as described by Walker, J. E. et al., EMBO J. 1:945-951 (1982). When two amino acids are indicated in parentheses separated by a/mark, that indicates either amino acid may appear at that position. DnaX proteins as part of this group of ATP binding proteins contain the defined conserved residues of the Walker A motif. However, there are additional conserved residues that are used for verification of DnaX that are expanded for DnaX beyond the Walker A motif and this entire region will be referred to as the ‘DnaX Walker A’ sequence. Conserved residues in DnaX proteins that encompass the Walker B motif have also been expanded for identification and verification purposed and will be referred to as the ‘DnaX Walker B’. Conservation of these residues is criteria used for selection of DnaX proteins.

i. Select proteins that contain the required strictly conserved residues indicated for DnaX from the parameters listed below. Possible DnaX, δ′ and δ must be aligned with the corresponding E. coli protein (in a pairwise alignment) using CLUSTAL W (Thompson, J. D. et al., Nucleic Acids Res. 22:4673-4680 (1994)). In these searches CLUSTAL W was used as part of Vector NTI v. 5.2.1.3. The “slow pairwise alignment algorithm” was used with the residue substitution matrix set to “Blosum”, the gap opening penalty set to 10, and the gap extension penalty set to 0.1. Numbers indicate the N-terminal and C-terminal residues in E. coli numbering. When there is more than one residue in parenthesis and separated by a slash, any of these residues may correspond to a conserved residue. To be scored, a residue must align with the conserved E. coli residue at the position strictly according to E. coli numbering even though gaps may be inserted between defined positions for optimal alignment (in either the E. coli sequence or target sequence) by the CLUSTAL W program. Residues designated as X can be any amino acid and the number of X's indicate the number of amino acids between defined conserved residues (in the E. coli numbering system).

a. Conserved residues (indicated with boldface letters) encompassing the DnaX Walker A motif. Six of the eleven conserved residues must be present. These six conserved residues must include the K(T/S) residues at position 51 and 52: 39-HAYLFSGXXGXGK(T/S)-52 SEQ ID NO: 203)

b. Conserved residues between the DnaX Walker A and B motifs. Seven of the ten conserved residues must be present: 88-(L/I/V)D(L/I/V)(L/I/V)E(L/I/V)D(A/G)AS-97 (SEQ ID NO: 204)

c. Conserved residues encompassing the DnaX Walker B motif. Nine of the eleven conserved residues must be present. These nine conserved residues must include the D residue at position 126: 121-(K/R)(L/I/V)(Y/F)(L/I/V)(L/I/V)DEXHM(L/I/V)(S/T)-132 (SEQ ID NO: 205)

Some of these conserved DnaX residues have been reported in the scientific literature (Walker, J. R., et al., J. Bacteriol. 182:6106-6113 (2000); Neuwald, A. F., et al., Genome Res. 9:27-43 (1999))

The initial search (iteration) gave 143 total hits with 90 above threshold and 53 below threshold. DnaX from all genomes searched were contained within the first 40 hits. The remaining hits in the top forty contained duplicate sequences. The accession numbers and GI numbers for the sequences of the DnaX proteins identified are shown in Table 3.

Step 5. Perform additional iterations if DnaX from all target organisms were not identified.

i. Limit the hits for further searches (iterations) to sequences with E-values greater than threshold and sequences selected as DnaX proteins in step 4.

DnaX proteins identified in additional iterations must meet criteria outlined in 4i.

### Example 3

Identification of δ′ Proteins from Completely Sequenced Genomes

The search for δ′ was preformed using PSI-BLAST as in the search for DnaX proteins. DnaX and δ′ have regions of similarity; therefore, the DnaX proteins identified in the DnaX search were excluded.

Step 1. Set search criteria in PSI-BLAST as defined in step 1 of the DnaX search (Example 2).

Step 2. Limit the search by Entrez query to only those organisms that have the genomic DNA completely sequenced and available at National Center for Biotechnology Information. To exclude DnaX proteins as possible hits they must be exclude in the window labeled “Limit by entrez query” by listing the GenBank identification (GI) numbers following the list of organism the search is limited to as shown below. All duplicated proteins with different GI numbers shown in PSI-BLAST alignments must also be excluded. The search for proteins was limited to the organisms listed below, with DnaX proteins excluded by GI number as shown below.

Aquifex aeolicus[Organism] OR Bacillus halodurans[Organism] OR Bacillus subtilis[Organism] OR Buchnera[Organism] OR Borrelia burgdorferi[Organism] OR Campylobacter jejuni[Organism] OR Caulobacter crescentus[Organism] OR Chlamydia pneumoniae[Organism] OR Chlamydia muridarum[Organism] OR Chlamydia trachomatis[Organism] OR Deinococcus radiodurans[Organism] OR Escherichia coli[Organism] OR Escherichia coli K12[Organism] OR Escherichia coli O157[Organism] OR Haemophilus influenzae[Organism] OR Helicobacter pylori 26695[Organism] OR Helicobacter pylori J99[Organism] OR Lactococcus lactis[Organism] OR Mesorhizobium loti[Organism] OR Mycobacterium leprae[Organism] OR Mycobacterium tuberculosis[Organism] OR Mycoplasma genitalium[Organism] OR Mycoplasma pneumoniae[Organism] OR Neisseria meningitidis MC58[Organism] OR Neisseria meningitidis Z2491[Organism] OR Pasteurella multocida[Organism] OR Pseudomonas aeruginosa[Organism] OR Rickettsia prowazekii[Organism] OR Synechocystis[Organism] OR Thermotoga maritima[Organism] OR Treponema pallidum[Organism] OR Ureaplasma urealyticum[Organism] OR Vibrio cholerae[Organism] OR Xylella fastidiosa[Organism] NOT (7514776[gi] OR 98292[gi] OR 7463153[gi] OR 11346858[gi] OR 7468911[gi] OR 7471826[gi] OR 1786676[gi] OR 1075209[gi] OR 7464043[gi] OR 3150103[gi] OR 1293572[gi] OR7477980[gi] OR 1361493[gi] OR2146109[gi] OR 11261620[gi] OR 7467603[gi] OR 11348451[gi] OR OR 7469304[gi] OR 7462368[gi] OR 2583049[gi] OR 7521020[gi] OR 11356842[gi] OR 11261617[gi] OR 11261615[gi] OR 12720609[gi] OR 12513340[gi] OR 118808[gi] OR 1169397[gi] OR 10039144[gi] OR 10172646[gi] OR 118807[gi] OR 9789547[gi] OR 7468224[gi] OR 8978415[gi] OR 12725258[gi] OR 13357643[gi] OR 6014997[gi] OR 2494198[gi] OR 12045280[gi] OR 581255[gi] OR 13474589[gi] OR 13421402[gi] OR 7464040[gi] OR 11360902[gi] OR 13508357[gi] OR 13359980[gi] OR 67058[gi] OR 41291[gi] OR 43320[gi] OR 145297[gi] OR 1773152[gi] OR 1574159[gi] OR 9655520[gi] OR 9947490[gi] OR 9106885[gi] OR 7380298[gi] OR 580855[gi] OR 467409[gi] OR 580914[gi] OR 2632286[gi] OR 6968590[gi] OR 2984127[gi] OR 4981209[gi] OR 4155207[gi] OR2313841[gi] OR4376294[gi] OR 7189650[gi] OR 3328753[gi] OR 7190650[gi] OR 13093951[gi] OR 3323329[gi] OR 2688379[gi] OR 3861390[gi] OR 6899039[gi] OR 6460225[gi] OR 2960145[gi] OR 1673890[gi] OR 1352305[gi] OR 3845015[gi] OR 1652627[gi])

The format for listing organisms is as described for DnaX proteins. Proteins to be excluded are listed by GI numbers followed without spaces by brackets enclosing the term “gi”.

Step 3. The amino acid sequence of a query protein must be entered into the search box. In this search, the amino acid sequence for E. coli δ′ was used (shown below).

MRWYPWLRPDFEKLVASYQAGRGHHALLIQALPGMGDDALIYALSRYLLC
QQPQGHKSCGHCRGCQLMQAGTHPDYYTLAPEKGKNTLGVDAVREVTEKL
NEHARLGGAKVVWVTDAALLTDAAANALLKTLEEPPAETWFFLATREPER
LLATLRSRCRLHYLAPPPEQYAVTWLSREVTMSQDALLAALRLSAGSPGA
ALALFQGDNWQARETLCQALAYSVPSGDWYSLLAALNHEQAPARLHWLAT
LLMDALKRHHGAAQVTNVDVPGLVAELANHLSPSRLQAILGDVCHIREQL
MSVTGINRELLITDLLLRIEHYLQPGVVLPVPHL

The Psi-Blast search returned 42 hits listed in decreasing similarity, with 32 hits having E-values greater than threshold.

Step 4. Criteria used to select hits that are δ′.

i. Select only proteins that contain conserved residues for δ′ from the parameters listed in a. and b. below. Numbers indicate the N-terminal and C-terminal residues in E. coli δ′ numbering. To be scored, a residue must align with the conserved E. coli residue (in a pairwise alignment) at the position strictly according to E. coli numbering even though gaps may be inserted between defined positions for optimal alignment (in either the E. coli sequence or target sequence) by the CLUSTAL W program. Residues designated as X can be any amino acid.

a. δ′ molecules must contain eight of the twelve conserved residues in the conserved region

| | |
|---|---|
| 124--(A/S)AN(A/S)(L/I/V)(L/I/V)(K/R)X(L/I/V)E(E/D)PP--136. | (SEQ ID NO:207) |

b. δ′ molecules must contain four of the six conserved residues in the conserved region

151-(L/I/V)(L/I/V)XT(L/I/V)XSR-158 (SEQ ID NO: 208).

ii. Exclude proteins containing parameters outlined in 4ib in the search for DnaX proteins above (Example 2).

In the initial search (iteration) δ′ was identified for 29 of the organisms searched (1 hit was a duplicate). The δ′-subunits for four remaining organisms were noted as having E-values less than threshold (giving a total of 33 δ′ identified).

Some of these concerved residues have been reported (Neuwald, A. F., et al., Genome Res. 9:27-43 (1999))

Step 5. Perform additional iterations if δ′ from all target organisms were not identified.

i. Limit the hits for further searches (iterations) to sequences with E-values greater than threshold and sequences selected as δ′ proteins in step 4.

ii. δ′ proteins identified in additional iterations must meet criteria outlined in step 4i for identification of δ′.

iii. Repeat step 5 until δ′ from all specified organisms have been identified.

The second iteration gave 317 total hits and indicated that the δ′-subunits from the four organisms with E-values below threshold now contained E-values greater than threshold. The δ′ from the final (34 th) species was also identified above threshold. The accession numbers and GI numbers for these sequences are provided in Table 4.

### Example 4

Identification of δ Proteins from Completely Sequenced Genomes

As indicated above, δ proteins are not well conserved, and thus are not searchable through standard techniques. For example, a simple BLAST search of NCBI databases for δ proteins based on the known E. coli sequence returns results for only a small number of organisms: Vibrio cholerae, Pasteurella multocida, Haemophilus influenzae, Buchnera sp. APS, Pseudomonas aeruginosa, Xylella fastidiosa, from the gamma proteobacter division, and Neisseria meningitides Z2491 from the beta division of proteobacteria. No sequences from the alpha or epsilon divisions of proteobacteria are retrieved.

Two related methods were used to identify delta sequences using Psi-Blast. The first (method A) as described in our provisional application filed Jul. 14, 2000, uses ψ-Blast starting with the E. coli δ sequence (accession number P28630; GI number G1123452). Candidate identified sequences are restricted by elimination of DnaX sequences and δ sequences, by the requirement that candidate δ sequences be between 300 and 400 amino acids long and that they be the ‘best’ hit (lowest E score) from an organism after elimination of DnaX and δ sequences and proteins less than 300 amino acids or greater than 400. In the second method (method B), the sequences identified by method A were used to perform an alignment enabling identification of certain conserved residues present in all δ sequences. These conserved residues were included in the δ identification criteria as described below. This permitted elimination of the length criteria, permitting δ sequences to be identified that fell ourside of the 300-400 amino acid length or proteins resulting from genes that had errors in their reported sequences, resulting in a deduced protein sequence incorrectly documented as being shorter than 300 amino acids or longer than 400 amino acids.

Method A.

Step 1. Perform a ψ-blast search using the NCBI website and the NCBI nr database with the ‘E value’ threshold set to 0.001. The delta sequences from Vibrio cholerae, Pasteurella multocida, Haemophilus influenzae, Buchnera sp. APS, Pseudomonas aeruginosa, Xylella fastidiosa, and Neisseria meningitides are identified and with E values lower than the threshold value. If the E value was set at 0.002 instead of 0.001, deltas subunit proteins from the same seven organisms were still identified.

Step 2. Hits are examined and those for DnaX proteins (generally annotated as DnaX protein, X and/or y protein, etc. in their respective databases and found to give a better match (lower E score when searched against E. coli DnaX sequences than with δ sequence)) and δ (generally annotated as holB gene product, delta’ or DnaX-like protein, τ/γ-like protein, etc. in their respective databases and found to give a better match (lower E score when searched against E. coli δ′ sequences than with δ sequence)) were eliminated. Since these methods address bacterial δ sequences and their cognate replicases, sequences from organisms other than bacteria were ignored.

Step 3. The length of the encoded candidate δ proteins were examined and only those between 300 and 400 amino acids were further considered.

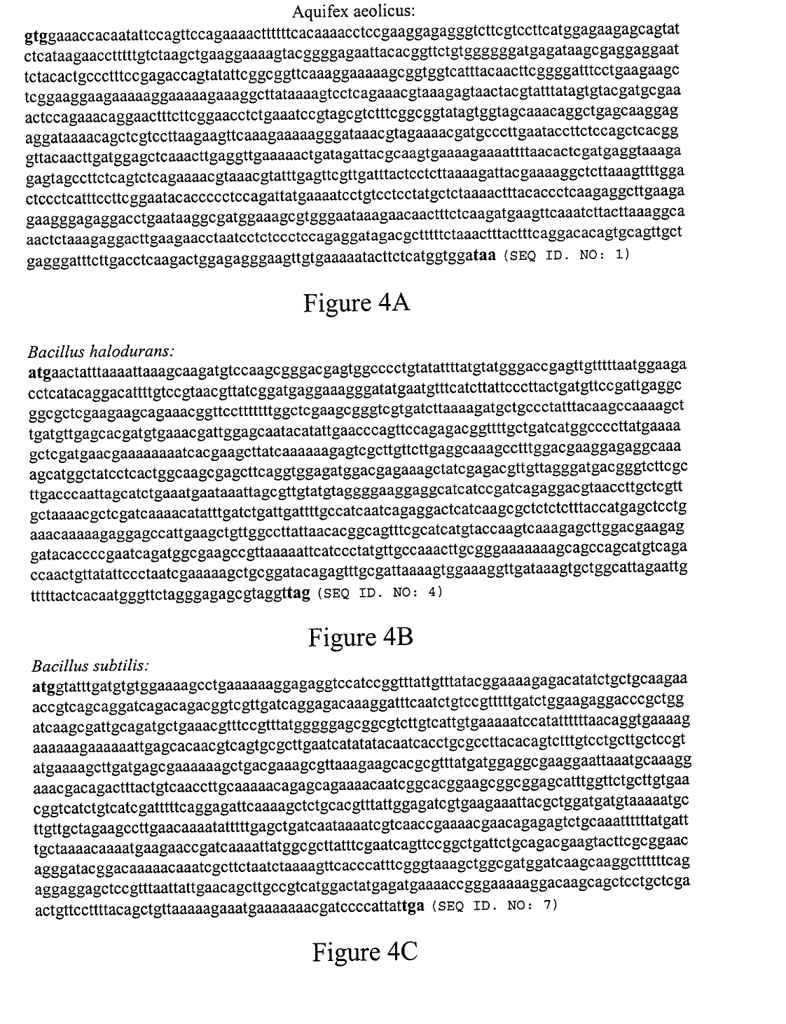

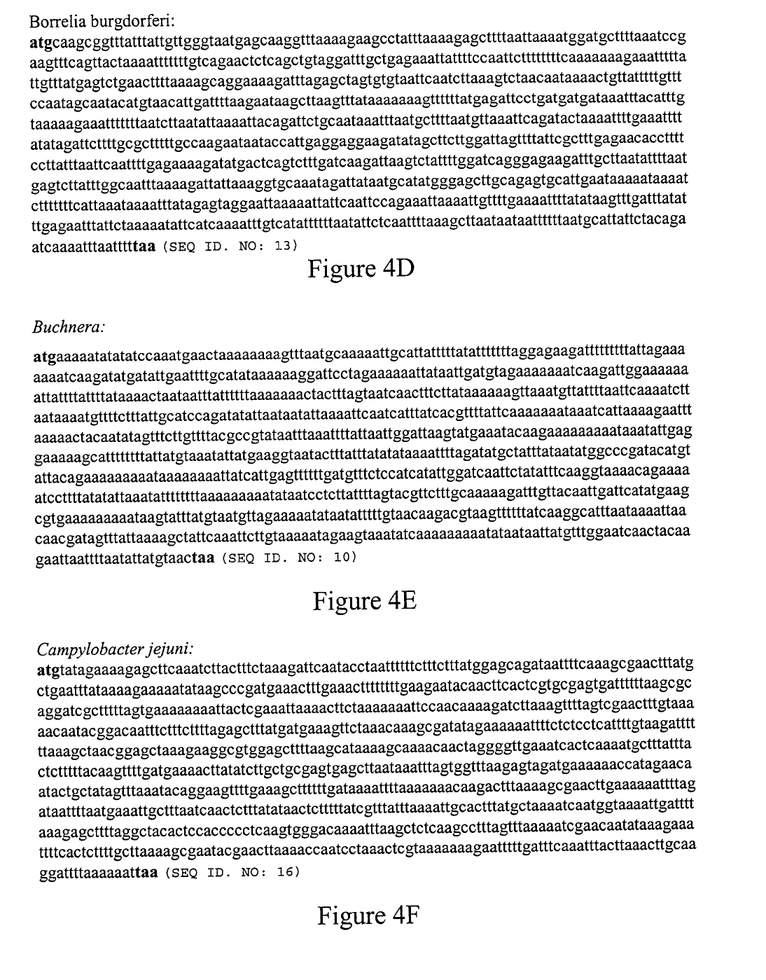

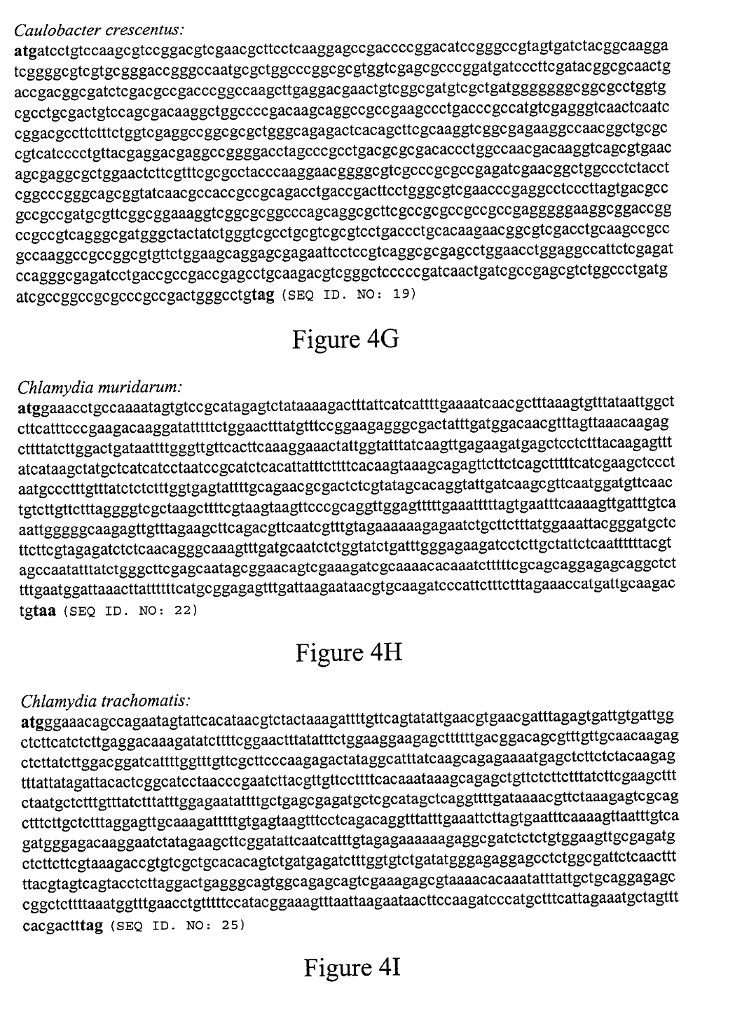

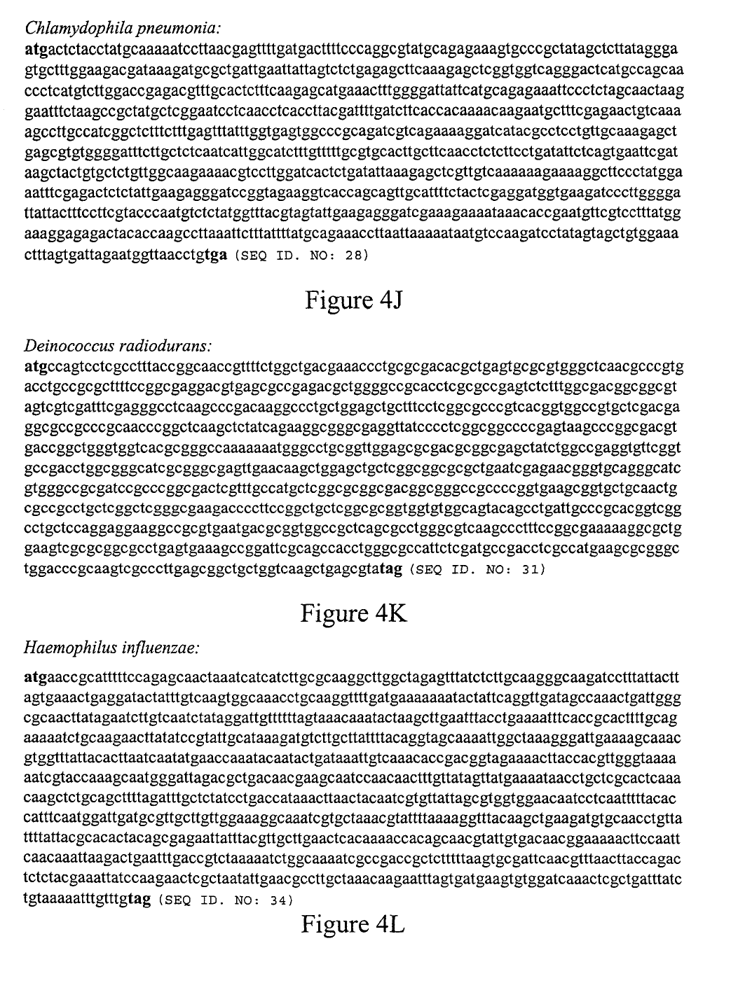

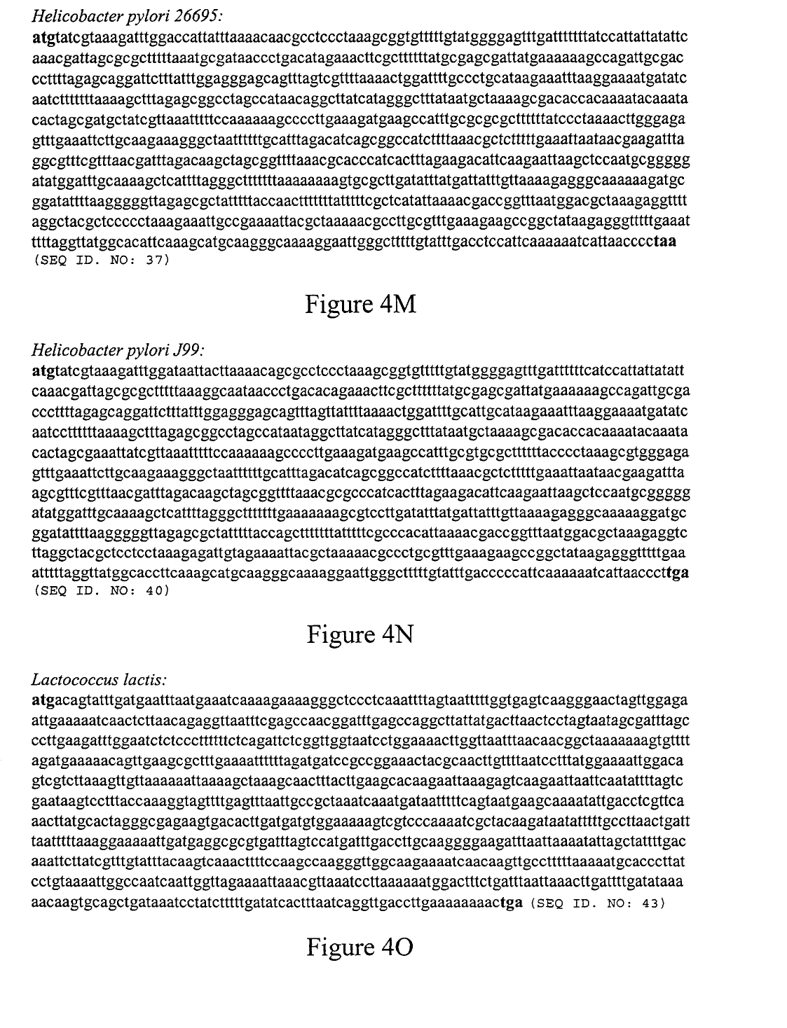

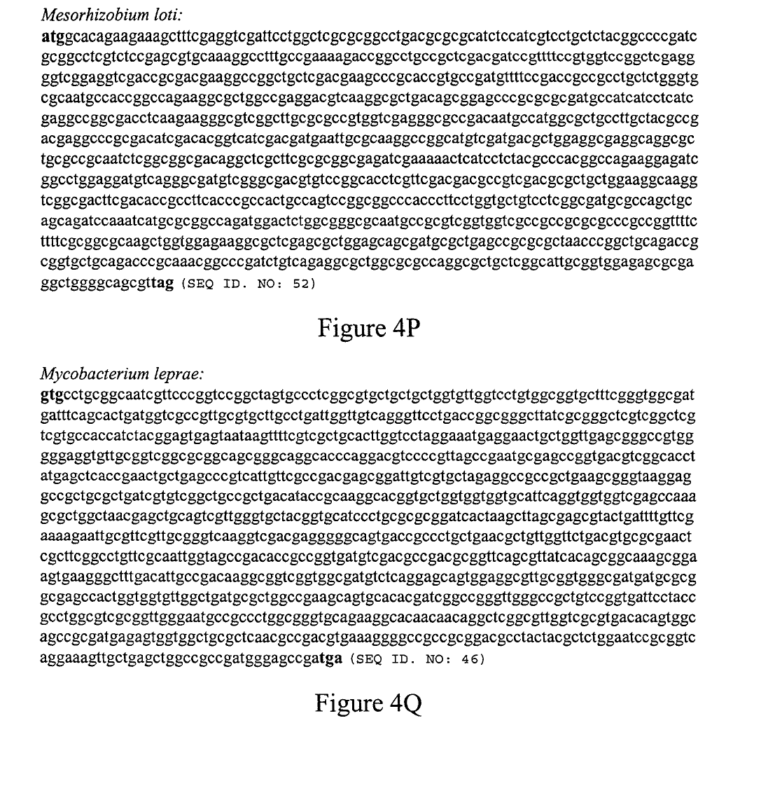

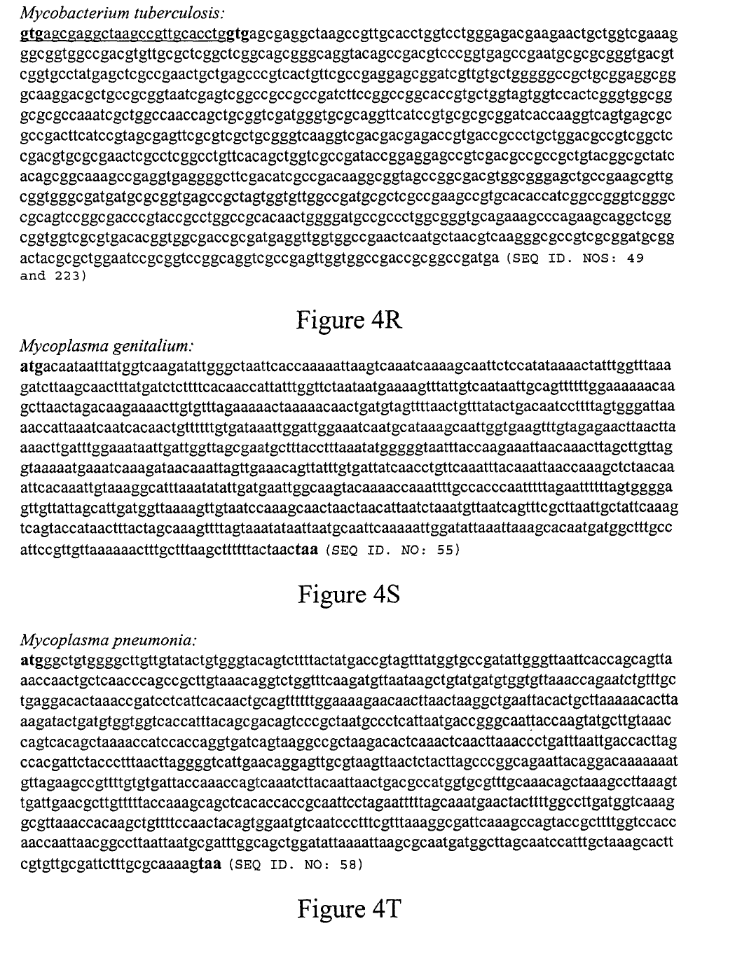

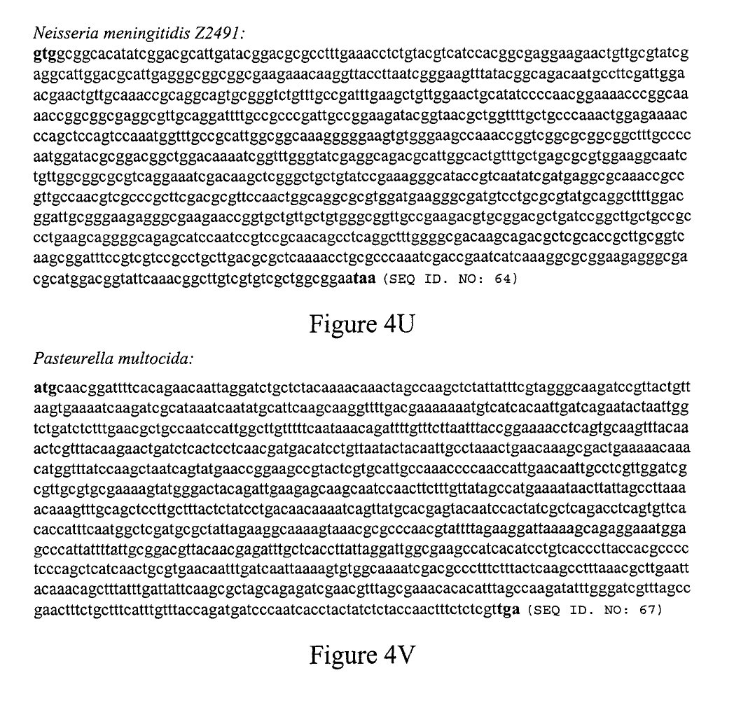

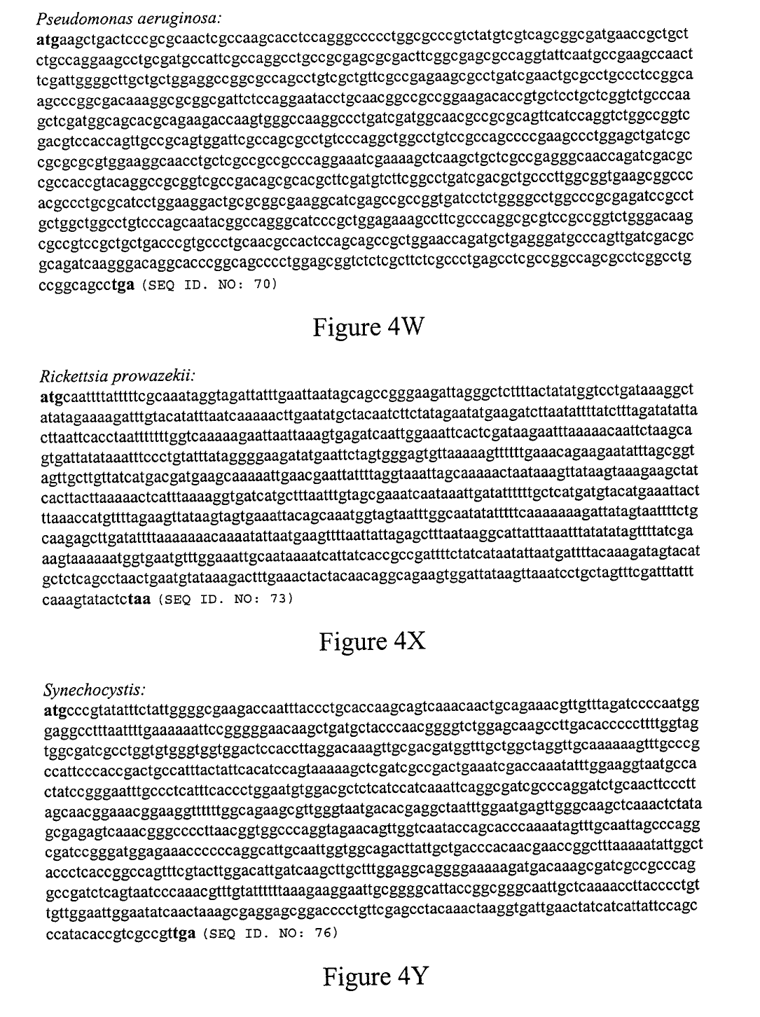

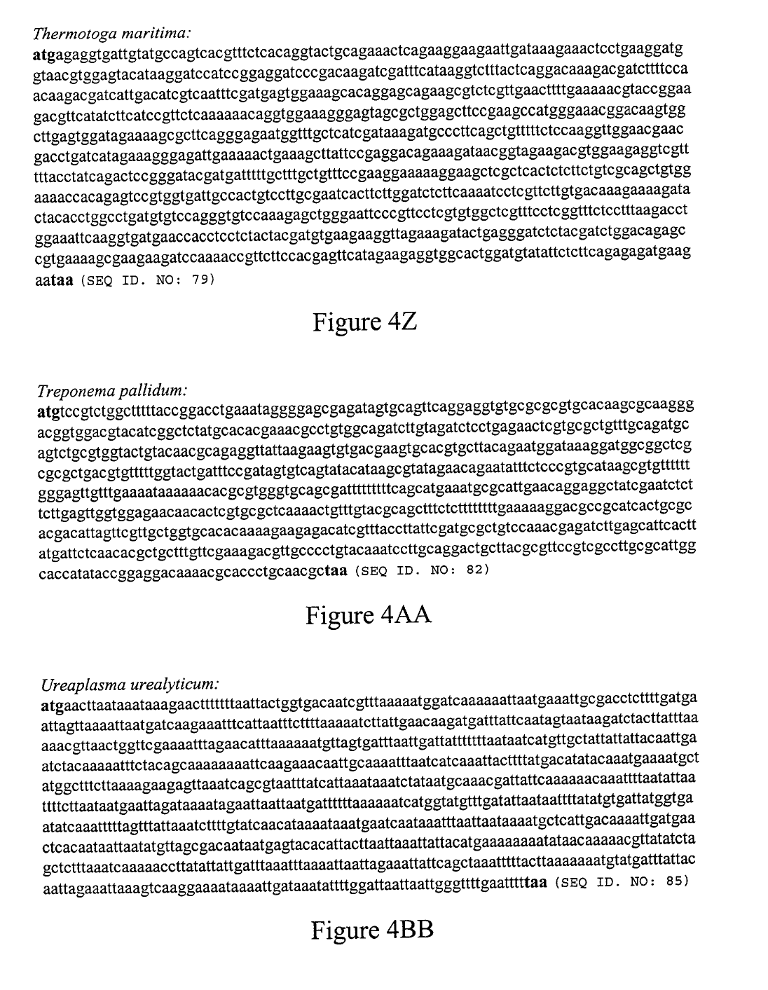

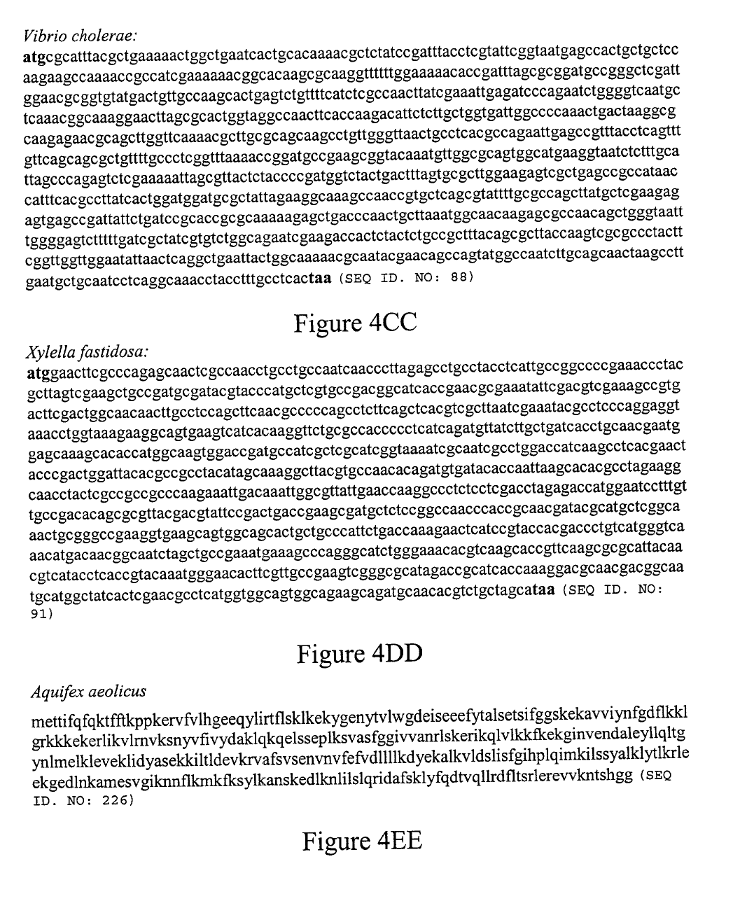

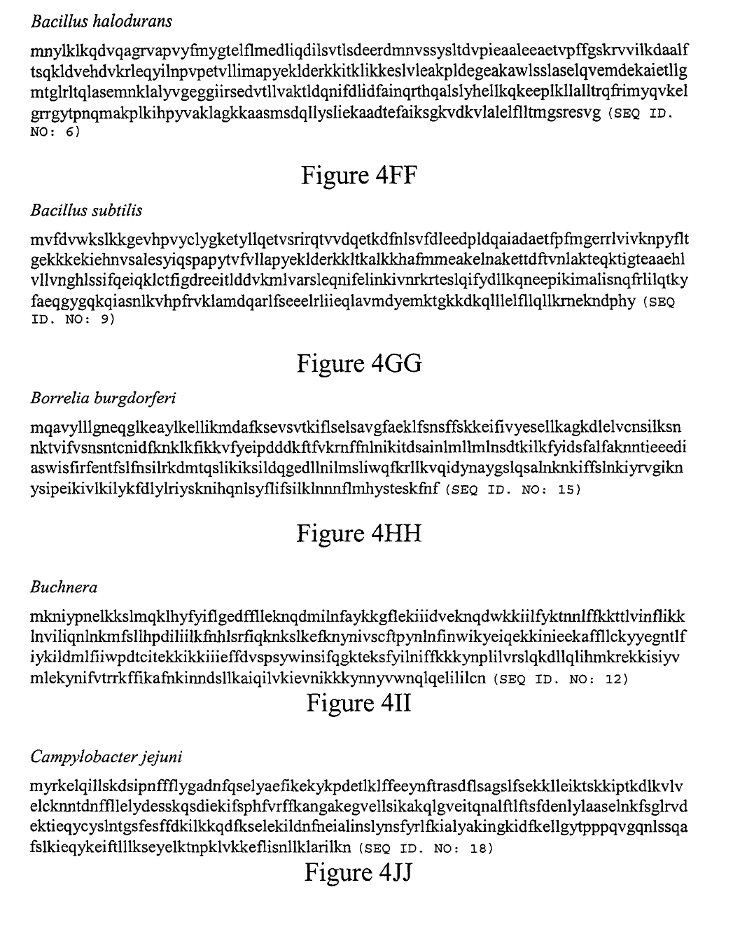

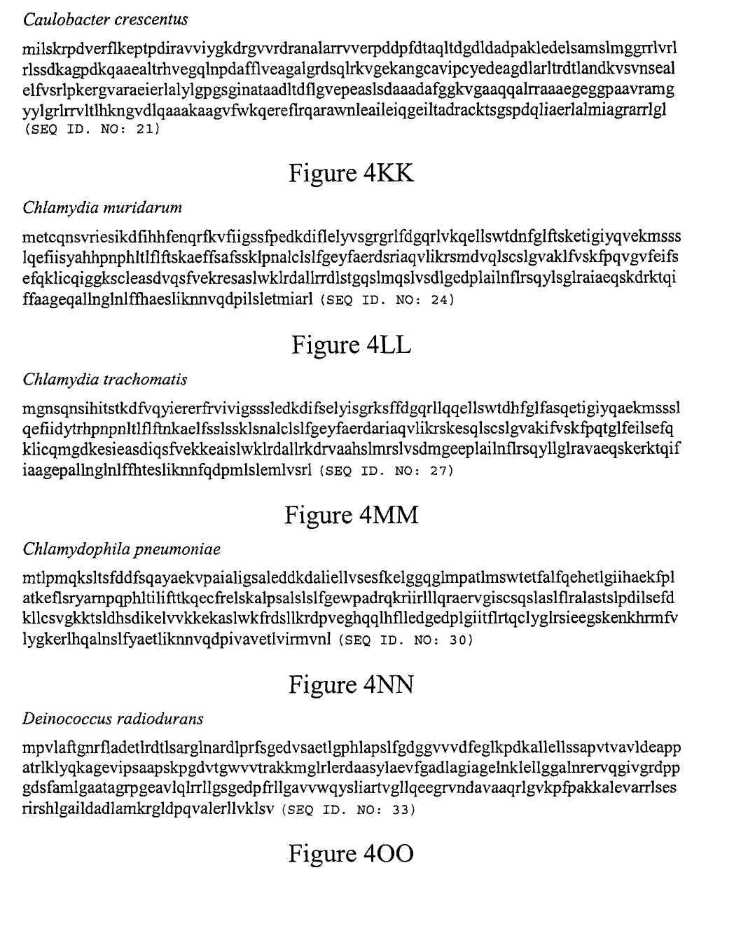

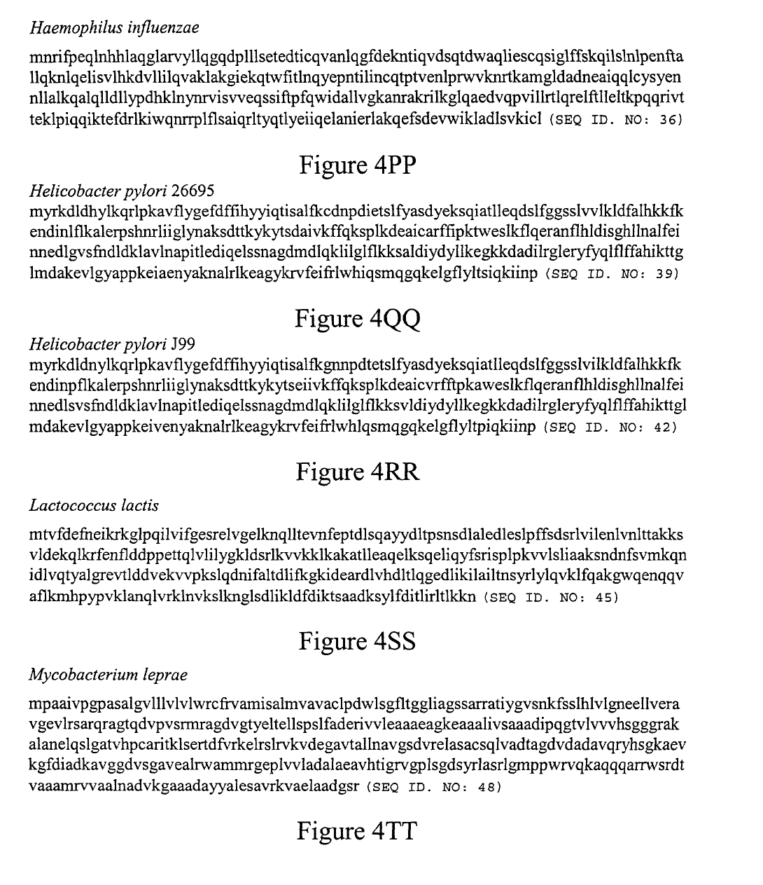

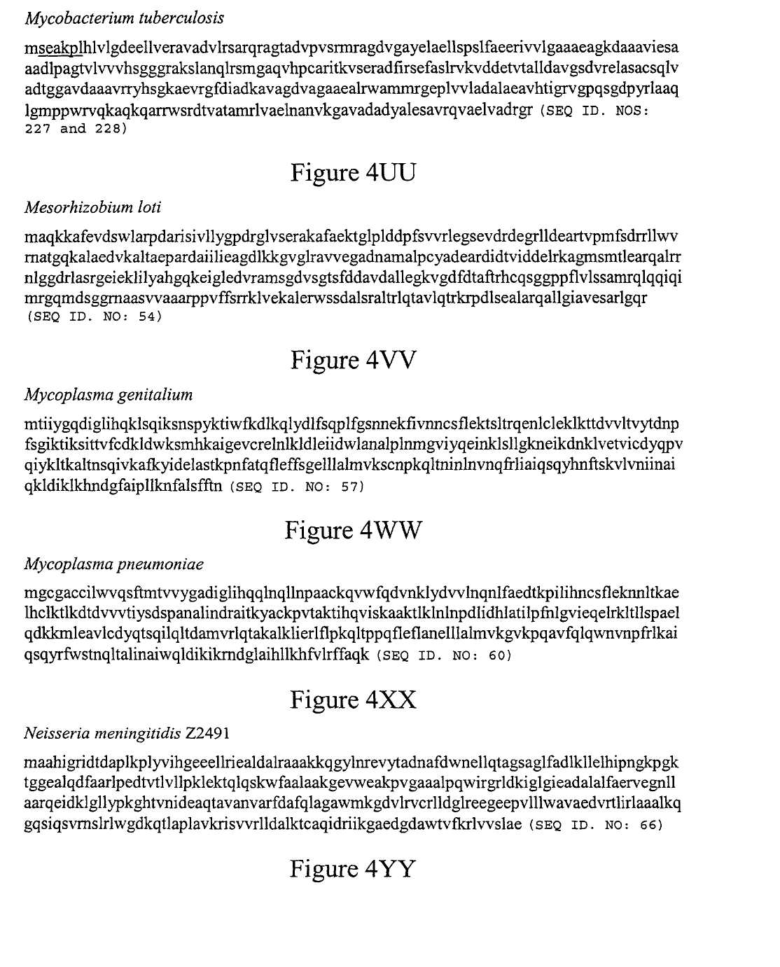

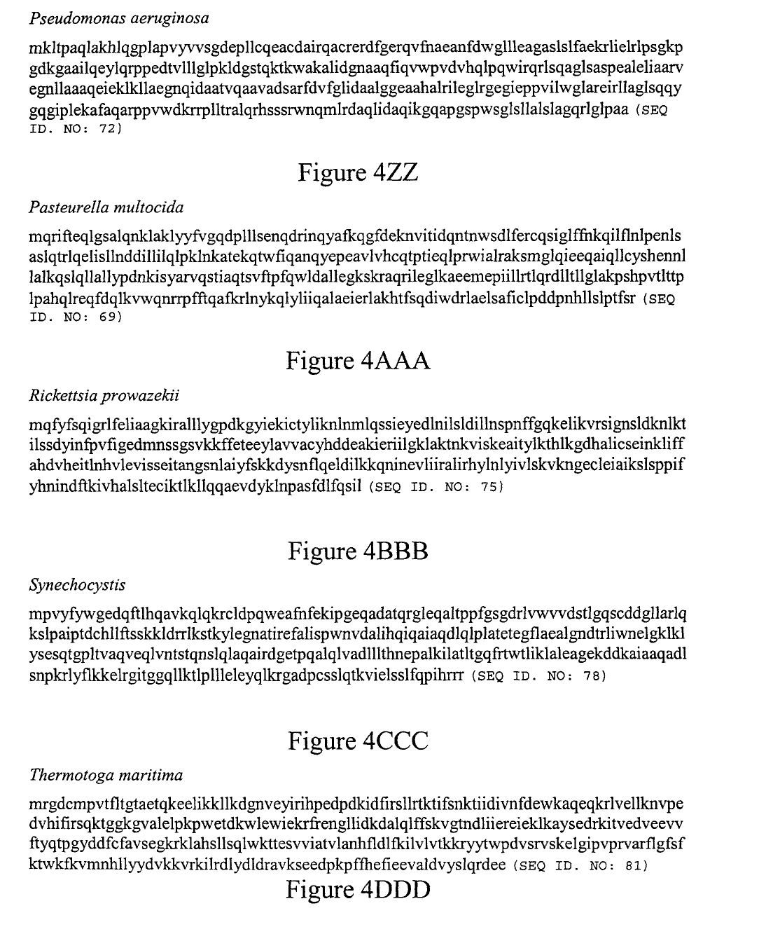

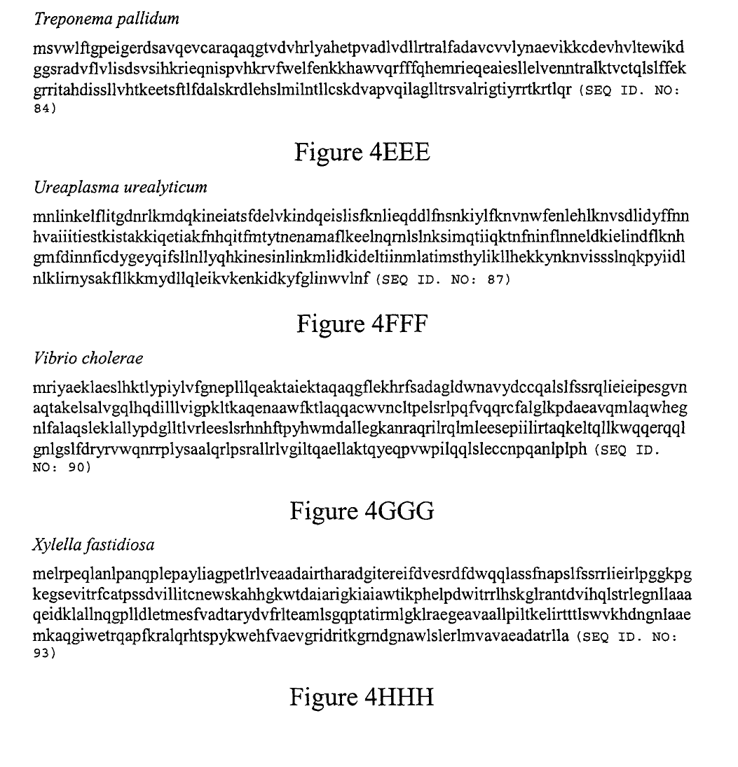

Step 4. Only the best hit from a given candidate organism was included (having excluded any additional DnaX and HolB sequences). From those organisms that have had their genomes completely sequences, the list of delta sequences shown in FIG. 4 was discovered.

Steps 2-4 can be repeated with additional iterations with the same setting until delta from the target organism(s) are identified.

Step 5. Although the identification of the δ sequences from the genomes that have presently been completely sequenced and are present in the NCBI nr database are unambiguous, additional criteria (outlined in the Jul. 14, 2000 provisional application) could be applied to confirm δ sequences if ambiguities exist. These include i) the ability to bind δ′ encoded by the holB gene of the same organism, ii) the ability to bind β encoded by the dnaN gene from the same organism and iii) the ability to reconstitute DNA polymerase III holoenzyme function when combined with the α subunit(s) of DNA polymerase III, the β processivity factor and the DnaX and δ′ components of the DnaX complex expressed from the corresponding structural genes for these proteins from the same organism. Also, candidate δ sequences can be aligned with other identified δ sequences using the Clustal W program, as performed in the Jul. 14, 2000 application, with visual inspection to insure that the overall pattern of conserved residues is mostly correct.

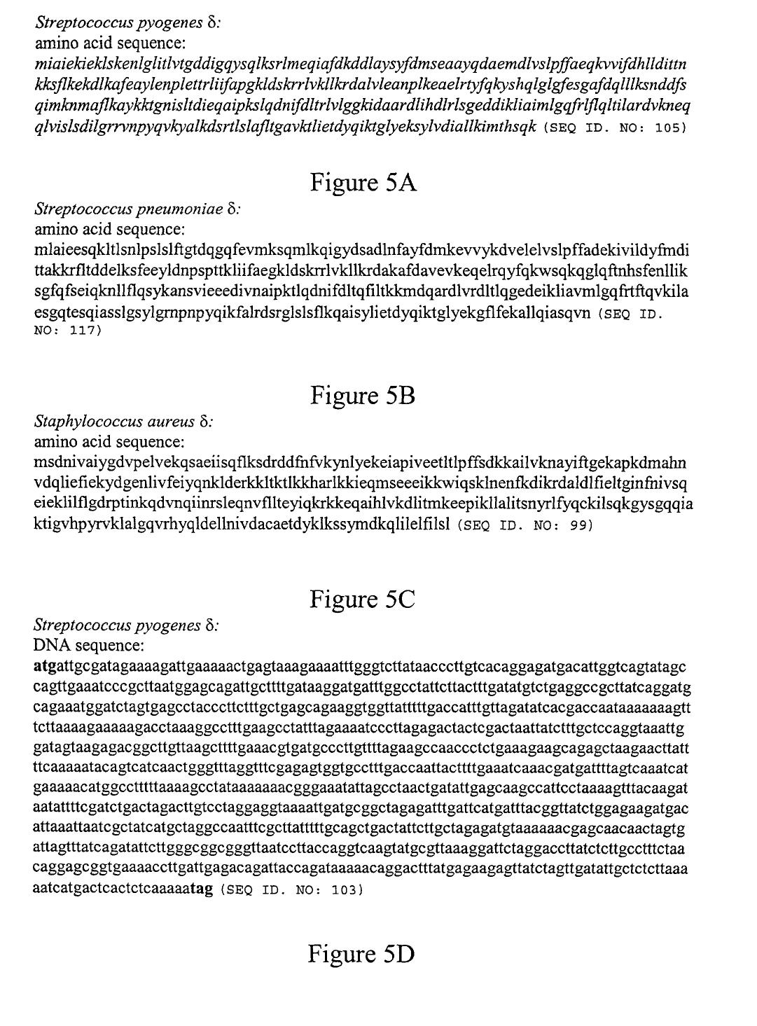

Having this more diverse group of δ sequences permitted the identification of sequences from additional organisms not accessible by Psi-Blast or the NCBI database (but could have been accessible using the same methods on locally run Psi-Blast searching local databases). Simple BLAST searches of reported ‘in progress’ or other remote databases were performed using tBLASTn and identified coding sequences for δ genes. The settings used in these tblastn BLAST (Gish, W. & States, D. J., Nature Genet. 3:266-272 (1993)) searches were as described below. This program compares a protein query sequence against a nucleotide database that has been translated in all six reading frames. Default parameters were used in each case. For example, searching the The University of Oklahoma Advanced Center for Genome Technology database of S. pyogenes sequences against the Bacillus subtilis δ protein sequence permitted identification of the sequences endoding S. pyogenes δ (FIG. 5). Search of the TIGR, the Institute for Genomic Research database against the Bacillus subtilis δ protein sequence permitted identification of the sequences of Streptococcus pneumoniae δ (FIG. 5). Search of the Sanger Centre database against the Bacillus subtilis δ protein sequence permitted identification of the sequences of Staphylococcus aureus δ (FIG. 5). The corresponding genes for DnaX and delta’ proteins from S. pyogenes, S. pneumoniae and S. aureus are shown in Table 6.

Method B. The search for δ was preformed using PSI-BLAST as in the search for DnaX and δ′. DnaX and δ′ may have regions of similarity with δ therefore the DnaX and δ′ proteins identified in the DnaX and δ′ search were excluded as possible hits.

Step 1. Set search criteria in PSI-BLAST as defined in step 1 of the DnaX search (Example 2).

Step 2. Limit the search by Entrez query to only those organisms that have the genomic DNA completely sequenced and available at NCBI. To exclude DnaX and δ′ proteins as possible hits they must be exclude in the window labeled “Limit by entrez query” by listing the GI numbers following the list of organism the search is limited to as shown below. All duplicated DnaX and δ′ proteins with different GI numbers shown in PSI-BLAST alignments must also be excluded. The search for proteins was limited to the organisms listed below, with DnaX and δ′ proteins excluded by GI number as shown below.

Aquifex aeolicus[Organism] OR Bacillus halodurans[Organism] OR Bacillus subtilis[Organism] OR Buchnera[Organism] OR Borrelia burgdorferi[Organism] OR Campylobacter jejuni[Organism] OR Caulobacter crescentus[Organism] OR Chlamydia pneumoniae[Organism] OR Chlamydia muridarum[Organism] OR Chlamydia trachomatis[Organism] OR Deinococcus radiodurans[Organism] OR Escherichia coli[Organism] OR Escherichia coli K12[Organism] OR Escherichia coli O157[Organism] OR Haemophilus influenzae[Organism] OR Helicobacter pylori 26695[Organism] OR Helicobacter pylori J99[Organism] OR Lactococcus lactis[Organism] OR Mesorhizobium loti[Organism] OR Mycobacterium leprae[Organism] OR Mycobacterium tuberculosis[Organism] OR Mycoplasma genitalium[Organism] OR Mycoplasma pneumoniae[Organism] OR Neisseria meningitidis MC58[Organism] OR Neisseria meningitidis Z2491[Organism] OR Pasteurella multocida[Organism] OR Pseudomonas aeruginosa[Organism] OR Rickettsia prowazekii[Organism] OR Synechocystis[Organism] OR Thermotoga maritima[Organism] OR Treponema pallidum[Organism] OR Ureaplasma urealyticum[Organism] OR Vibrio cholerae[Organism] OR Xylella fastidiosa[Organism] NOT (7514776[gi] OR98292[gi] OR 7463153[gi] OR 11346858[gi]OR 7468911[gi] OR 7471826[gi] OR 1786676[gi] OR 1075209[gi] OR 7464043[gi] OR 3150103[gi] OR 1293572[gi] OR 7477980[gi] OR 1361493[gi] OR 2146109[gi] OR 11261620[gi] OR 7467603[gi] OR 11348451[gi] OR 7469304[gi] OR 7462368[gi] OR 2583049[gi] OR 7521020[gi] OR 11356842[gi] OR 11261617[gi] OR 11261615[gi] OR 12720609[gi] OR 12513340[gi] OR 118808[gi] OR 1169397[gi] OR 10039144[gi] OR 10172646[gi] OR 118807[gi] OR 9789547[gi] OR 7468224[gi] OR 8978415[gi] OR 12725258[gi] OR 13357643[gi] OR 6014997[gi] OR 2494198[gi] OR 12045280[gi] OR 581255[gi] OR 13474589[gi] OR 13421402[gi] OR 7464040[gi] OR 11360902[gi] OR 13508357[gi] OR 13359980[gi] OR 67058[gi] OR 41291[gi] OR 43320[gi] OR 145297[gi] OR 1773152[gi] OR 1574159[gi] OR 9655520[gi] OR 9947490[gi] OR 9106885[gi] OR 7380298[gi] OR 580855[gi] OR 467409[gi] OR 580914[gi] OR 2632286[gi] OR 6968590[gi] OR 2984127[gi] OR 4981209[gi] OR 4155207[gi] OR 2313841[gi] OR 4376294[gi] OR 7189650[gi] OR 3328753[gi] OR 7190650[gi] OR 13093951[gi] OR 3323329[gi] OR 2688379[gi] OR 3861390[gi] OR 6899039[gi] OR 6460225[gi] OR 2960145[gi] OR 1673890[gi] OR 1352305[gi] OR 3845015[gi] OR 1652627[gi] OR 2506368[gi] OR 479843[gi] OR 3212391[gi] OR 145783[gi] OR 1651540[gi] OR 1787341[gi] OR 1074027[gi] OR 2506369[gi] OR 1573429[gi] OR 12722081[gi] OR 11280161[gi] OR 9656559[gi] OR 12644665[gi] OR 11348448[gi] OR 9949059[gi] OR 11280162[gi] OR 972778[gi] OR 11353892[gi] OR 7379683[gi] OR 11353126[gi] OR 7226000[gi] OR 11360901[gi] OR 9105556[gi] OR 7477702[gi] OR 2105050[gi] OR 3097239[gi] OR 13092554[gi] OR 12723272[gi] OR 10172656[gi] OR 13423258[gi] OR 6318311[gi] OR 13470653[gi] OR 586870[gi] OR 2126931[gi] OR 467421[gi] OR 2632298[gi] OR 7473469[gi] OR 6460143[gi] OR 7514459[gi] OR 2983903[gi] OR 7467604[gi] OR 3860738[gi] OR 7462369[gi] OR 4981299[gi] OR 11360903[gi] OR 7190502[gi] OR 7468912[gi] OR 3328592[gi] OR 7469488[gi] OR 1653573[gi] OR 13507746[gi] OR 2496262[gi] OR 2146108[gi] OR 1673807[gi] OR 7468225[gi] OR 4376545[gi] OR 7189405[gi] OR 8978646[gi] OR 7463400[gi] OR 2688709[gi] OR 13357575[gi] OR 11356841[gi] OR 7465204[gi] OR 4155746[gi] OR 7521018[gi] OR 3322812[gi] OR 7464041[gi] OR 2314393[gi] OR 11346661[gi] OR 6968051[gi])

Step 3. Search designated genomes against E. coli δ amino acid sequence (shown below). This amino acid sequence of E. coli δ (SEQ ID NO: 209 must be entered into the search box of the initial search window.

MIRLYPEQLRAQLNEGLRAAYLLLGNDPLLLQESQDAVRQVAAAQGFEEH
HTFSIDPNTDWNAIFSLCQAMSLFASRQTLLLLLPENGPNAAINEQLLTL
TGLLHDDLLLIVRGNKLSKAQENAAWFTALANRSVQVTCQTPEQAQLPRW
VAARAKQLNLELDDAANOVLCYCYEGNLLALAQALERLSLLWPDGKLTLP
RVEQAVNDAAHFTPFHWVDALLMGKSKRALHILQQLRLEGSEPVILLRTL
QRELLLLVNLKRQSAHTPLRALFDKHRVWQNRRGMMGEALNRLSQTQLRQ
AVQLLTRTELTLKQDYGQSVWAELEGLSLLLCHKPLADVFIDG

The PSI-BLAST search returned 13 hits listed in decreasing similarity, with 10 hits containing E-values greater than threshold and 3 hits with E-values less than threshold.

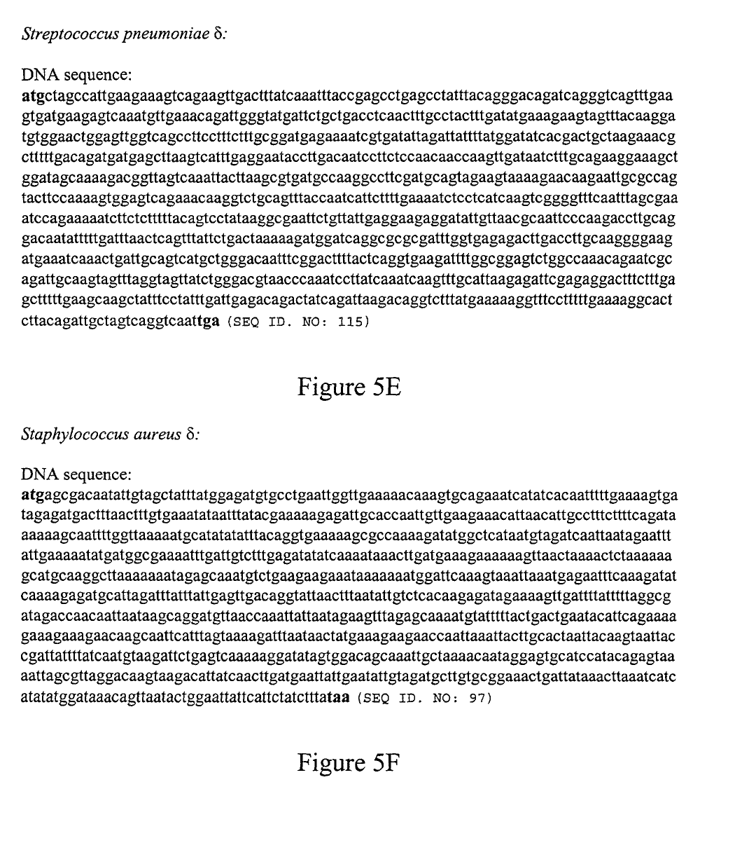

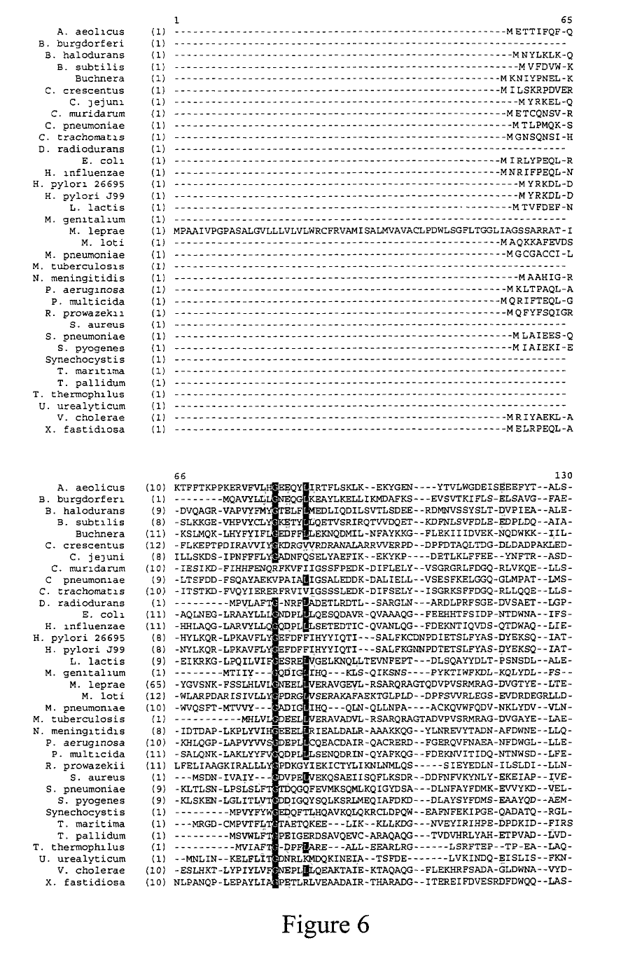

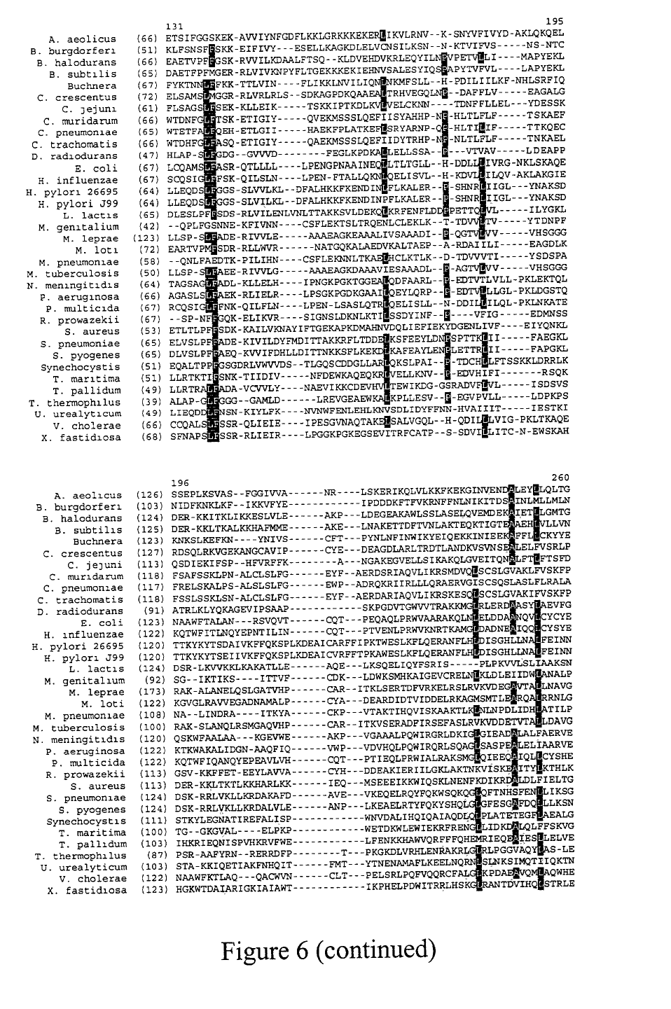

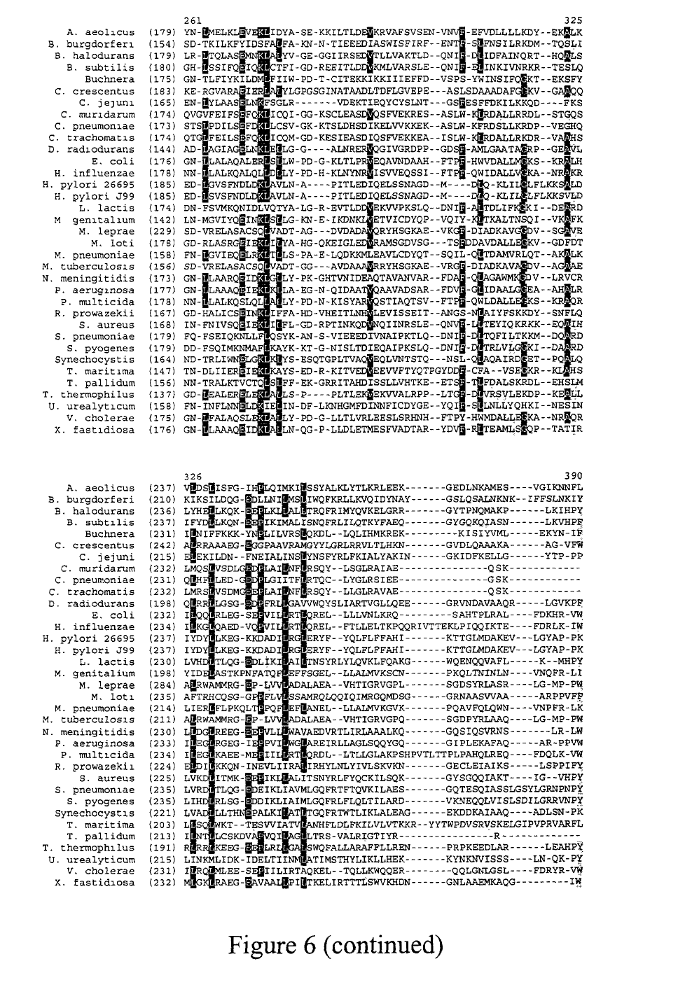

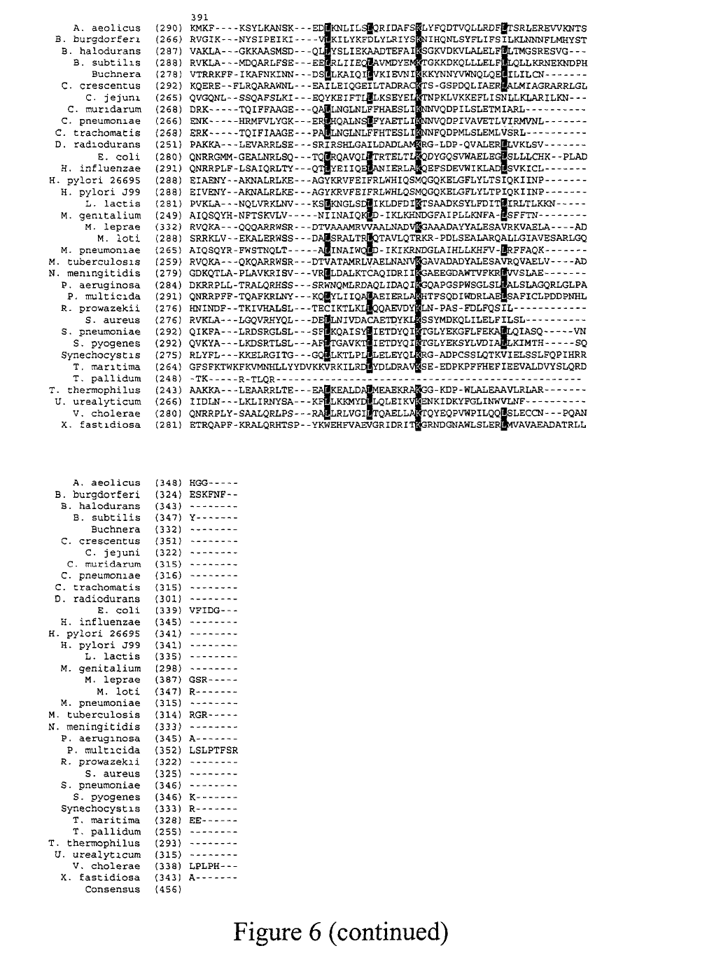

Step 4. Criteria to select hits that are δ proteins. Using the δ proteins identified in method A, the δ sequence from Thermus thermophilus (patent application Ser. No. 09/818,780) and δ subunit sequences from Streptococcus pyogenes, Streptococcus pneumoniae and Staphylococcus aureus (FIG. 5), alignments using Clustal W indicated six areas containing highly conserved amino acids. Conserved residues in these six regions provide additional sequence-based criteria for confirmation of δ proteins. The sequences selected represented the most conserved stretches greater than 17 amino acids in aligned deltas (FIG. 6)

i. Exclude DnaX and δ′ sequences that were identified in the DnaX and δ′ searches.

ii Identified δ subunits must meet the following criteria in at least 3 of the 6 conserved regions shown below. In conserved regions #1-6 the indicated number of conserved residues must be observed: #1, 5 out of possible 10, #2, 5 out of possible 11, #3, 5 out of possible 10, #4, 9 out of possible 18, #5, 11 out of possible 23, #6 6 out of possible 12 conserved residues. Pairwise alignments and selection must be performed as described in step 4i in Example 2.

| | | |
|---|---|---|
| 1.) | 17--(L/I/V)XX(L/I/V)Y(L/I/V)(L/I/V)XGX(D/E)XX(L/I/V)(L/I/V)XXXXXX(L/I/V)--38 | (SEQ ID NO:194) |
| 2.) | 64--(L/I/V)(L/I/V)XXXX(A/S)XX(L/I/V)F(A/S)X(K/R)X(L/I/V)(L/I/V)(L/I/V)(L/I/V)--82 | (SEQ ID NO:195) |
| 3.) | 97--(L/I/V)XX(L/I/V)(L/I/V)XXXXX(D/E)X(L/I/V)(L/I/V)(L/I/V)(L/I/V)XXXK(L/I/V)- | (SEQ ID NO:196) |
| | -117 | |
| 4.) | 154--(R/K)XXX(L/I/V)X(L/I/V)X(L/I/V)(D/E)X(D/E)(A/S)(L/I/V)XX(L/I/V)XXXXXXN(L/I/V) | (SEQ ID NO:197) |
| | XX(L/I/V)XX(D/E)(L/I/V)X(R/K)(L/I/V)X(L/I/V)(L/I/V)--191 | |
| 5.) | 215--FX(L/I/V)X(D/E)(A/S)(L/I/V)(L/I/V)XG(R/K)XXX(A/S)(L/I/V)X(L/I/V)(L/I/V)XX | (SEQ ID NO:198) |
| | (L/I/V)XXXGX(D/E)P(L/I/V)X(L/I/V)(L/I/V)XX(L/I/V)XXX(L/I/V)XX(L/I/V)XX(L/I/V)--260 | |
| 6.) | 298--(L/I/V)XXX(L/I/V)XX(L/I/V)XXX(D/E)XX(L/I/V)(R/K)XXXXX(D/E)XXXX(L/I/V)(D/E)XX | (SEQ ID NO:199) |
| | (L/I/V)(L/I/V)X(L/I/V)--331 | |

All of the hits (10) with E-values greater than threshold contained sequences that were likely δ proteins. These hits were marked for the second iteration.

Step 5. Additional iterations if δ from all target organism were not identified; that is, Step 4 is repeated until δ from all target organisms are identified.

i. Limit the hits for further searches (iterations) by sequences with E-values greater than threshold and sequences selected as δ in step 4.

ii. Possible δ proteins identified in additional iterations must meet criteria outlined in the δ search step 4 shown above.

iii. Repeat step 5 until δ from all specified organisms have been identified.

The second iteration returned 33 hits with 20 hits having E-values greater than threshold. Nine new hits were identified as δ subunits. These hits were selected as described above and marked for the third iteration. The third iteration returned 50 hits, containing δ from all except one of the target organisms. The fourth iteration contained 64 hits including the final δ. Conserved residues in the conserved regions for each δ is shown in Table 19.

### Example 5

Identification of DnaX. δ′ and δ from Partially Sequenced Genomes Using PSI-BLAST

Some protein sequences that are not from totally sequenced genomes but were identified as a protein from partially sequenced genomes have also been filed with NCBI. DnaX, δ′ and δ proteins from these partially sequenced genomes can be identified if they have been deposited with NCBI by using the criteria described above for identifying DnaX, δ′ and δ from fully sequenced genomes. The only exception with the search methods is that the search will not be limited by entrez query to only completely sequenced genomes, but will instead be limited by entrez query to all bacteria.

To search for DnaX proteins the term bacteria should be entered into the window labeled “Limit by entrez query” in the initial search window followed by exclusion of the DnaX and δ proteins identified in the search for DnaX and δ′ proteins from fully sequenced genomes as shown below.

bacteria [Organism] NOT (7514776[gi] OR 98292[gi] OR 7463153[gi] OR 11346858[gi] OR 7468911[gi] OR 7471826[gi] OR 1786676[gi] OR 1075209[gi] OR 7464043[gi] OR 3150103[gi] OR 1293572[gi] OR 7477980[gi] OR 1361493[gi] OR 2146109[gi] OR 11261620[gi] OR 7467603[gi] OR 11348451[gi] OR 7469304[gi] OR 7462368[gi] OR 2583049[gi] OR 7521020[gi] OR 11356842[gi] OR 11261617[gi] OR 11261615[gi] OR 12720609[gi] OR 12513340[gi] OR 118808[gi] OR 1169397[gi] OR 10039144[gi] OR 10172646[gi] OR 118807[gi] OR 9789547[gi] OR 7468224[gi] OR 8978415[gi] OR 12725258[gi] OR 13357643[gi] OR 6014997[gi] OR 2494198[gi] OR 12045280[gi] OR 581255[gi] OR 13474589[gi] OR 13421402[gi] OR 7464040[gi] OR 11360902[gi] OR 13508357[gi] OR 13359980[gi] OR 67058[gi] OR 41291[gi] OR 43320[gi] OR 145297[gi] OR 1773152[gi] OR 1574159[gi] OR 9655520[gi] OR 9947490[gi] OR 9106885[gi] OR 7380298[gi] OR 580855[gi] OR 467409[gi] OR 580914[gi] OR 2632286[gi] OR 6968590[gi] OR 2984127[gi] OR 4981209[gi] OR 4155207[gi] OR 2313841[gi] OR 4376294[gi] OR 7189650[gi] OR 3328753[gi] OR 7190650[gi] OR 13093951[gi] OR 3323329[gi] OR 2688379[gi] OR 3861390[gi] OR 6899039[gi] OR 6460225[gi] OR 2960145[gi] OR 1673890[gi] OR 1352305[gi] OR 3845015[gi] OR 1652627[gi] OR 2506368[gi] OR 479843[gi] OR 3212391[gi] OR 145783[gi] OR 1651540[gi] OR 1787341[gi] OR 1074027[gi] OR 2506369[gi] OR 1573429[gi] OR 12722081[gi] OR 11280161[gi] OR 9656559[gi] OR 12644665[gi] OR 11348448[gi] OR 9949059[gi] OR 11280162[gi] OR 972778[gi] OR 11353892[gi] OR 7379683[gi] OR 11353126[gi] OR 7226000[gi] OR 11360901[gi] OR 9105556[gi] OR 7477702[gi] OR 2105050[gi] OR 3097239[gi] OR 13092554[gi] OR 12723272[gi] OR 10172656[gi] OR 13423258[gi] OR 6318311[gi] OR 13470653[gi] OR 586870[gi] OR 2126931[gi] OR 467421[gi] OR 2632298[gi] OR 7473469[gi] OR 6460143[gi] OR 7514459[gi] OR 2983903[gi] OR 7467604[gi] OR 3860738[gi] OR 7462369[gi] OR 4981299[gi] OR 11360903[gi] OR 7190502[gi] OR 7468912[gi] OR 3328592[gi] OR 7469488[gi] OR 1653573[gi] OR 13507746[gi] OR 2496262[gi] OR 2146108[gi] OR 1673807[gi] OR 7468225[gi] OR 4376545[gi] OR 7189405[gi] OR 8978646[gi] OR 7463400[gi] OR 2688709[gi] OR 13357575[gi] OR 11356841[gi] OR 7465204[gi] OR 4155746[gi] OR 7521018[gi] OR 3322812[gi] OR 7464041[gi] OR 2314393[gi] OR 11346661[gi] OR 6968051[gi])

To be distinguished as DnaX, a protein must meet the criteria outlined above in step 4 of the search for DnaX from fully sequenced genomes (Example 2). Three additional DnaX proteins were identified after one iteration. The organisms from which the DnaX proteins were identified and that correspond to delta subunit proteins identified in an analogous manner (see below) were Mycoplasma pulmonis and Streptomyces coelicolor (Table 8).

This same procedure is used to identify δ′ proteins deposited with NCBI from partially sequenced genomes. To search for δ′ proteins the term bacteria should be entered into the window labeled “Limit by entrez query” in the initial search window followed by exclusion of the DnaX and δ′ proteins identified in the search for DnaX proteins and the search for δ′ proteins. δ′ proteins identified in the searches from both completely sequenced genomes and from genomes that have not been fully sequenced should be entered as shown below.

bacteria [Organism] NOT (7514776[gi] OR 98292[gi] OR 7463153[gi] OR 11346858[gi] OR 7468911[gi] OR 7471826[gi] OR 1786676[gi] OR 1075209[gi] OR 7464043[gi] OR 3150103[gi] OR 1293572[gi] OR 7477980[gi] OR 1361493[gi] OR 2146109[gi] OR 11261620[gi] OR 7467603[gi] OR 11348451[gi] OR 7469304[gi] OR 7462368[gi] OR 2583049[gi] OR 7521020[gi] OR 11356842[gi] OR 11261617[gi] OR 11261615[gi] OR 12720609[gi] OR 12513340[gi] OR 118808[gi] OR 1169397[gi] OR 10039144[gi] OR 10172646[gi] OR 118807[gi] OR 9789547[gi] OR 7468224[gi] OR 8978415[gi] OR 12725258[gi] OR 13357643[gi] OR 6014997[gi] OR 2494198[gi] OR 12045280[gi] OR 581255[gi] OR 13474589[gi] OR 13421402[gi] OR 7464040[gi] OR 11360902[gi] OR 13508357[gi] OR 13359980[gi] OR 67058[gi] OR 41291[gi] OR 43320[gi] OR 145297[gi] OR 1773152[gi] OR 1574159[gi] OR 9655520[gi] OR 9947490[gi] OR 9106885[gi] OR 7380298[gi] OR 580855[gi] OR 467409[gi] OR 580914[gi] OR 2632286[gi] OR 6968590[gi] OR 2984127[gi] OR 4981209[gi] OR 4155207[gi] OR 2313841[gi] OR 4376294[gi] OR 7189650[gi] OR 3328753[gi] OR 7190650[gi] OR 13093951[gi] OR 3323329[gi] OR 2688379[gi] OR 3861390[gi] OR 6899039[gi] OR 6460225[gi] OR 2960145[gi] OR 1673890[gi] OR 1352305[gi] OR 3845015[gi] OR 1652627[gi] OR 2506368[gi] OR 479843[gi] OR 3212391[gi] OR 145783[gi] OR 1651540[gi] OR 1787341[gi] OR 1074027[gi] OR 2506369[gi] OR 1573429[gi] OR 12722081[gi] OR 11280161[gi] OR 9656559[gi] OR 12644665[gi] OR 11348448[gi] OR 9949059[gi] OR 11280162[gi] OR 972778[gi] OR 11353892[gi] OR 7379683[gi] OR 11353126[gi] OR 7226000[gi] OR 11360901[gi] OR 9105556[gi] OR 7477702[gi] OR 2105050[gi] OR 3097239[gi] OR 13092554[gi] OR 12723272[gi] OR 10172656[gi] OR 13423258[gi] OR 6318311[gi] OR 13470653[gi] OR 586870[gi] OR 2126931[gi] OR 467421[gi] OR 2632298[gi] OR 7473469[gi] OR 6460143[gi] OR 7514459[gi] OR 2983903[gi] OR 7467604[gi] OR 3860738[gi] OR 7462369[gi] OR 4981299[gi] OR 11360903[gi] OR 7190502[gi] OR 7468912[gi] OR 3328592[gi] OR 7469488[gi] OR 1653573[gi] OR 13507746[gi] OR 2496262[gi] OR 2146108[gi] OR 1673807[gi] OR 7468225[gi] OR 4376545[gi] OR 7189405[gi] OR 8978646[gi] OR 7463400[gi] OR 2688709[gi] OR 13357575[gi] OR 11356841[gi] OR 7465204[gi] OR 4155746[gi] OR 7521018[gi] OR 3322812[gi] OR 7464041[gi] OR 2314393[gi] OR 11346661[gi] OR 6968051[gi] OR 1527142[gi] OR 13700368[gi] OR 5918469[gi] OR 14089462[gi])

To be distinguished as δ′, a protein must meet the criteria outlined above in step 4 of the search for δ′ from fully sequenced genomes.

After three iterations five additional δ′ proteins were identified from: Mycoplasma fermentans, Mycoplasma pulmonis, Plectonema boryanum, Streptomyces coelicolor.

This same procedure is used to identify δ proteins deposited with NCBI from partially sequenced genomes. To search for δ proteins the term bacteria should be entered into the window labeled “Limit by entrez query” in the initial search window followed by exclusion of the DnaX and δ′ proteins identified in the search for DnaX proteins and the search for δ′ proteins. δ′ proteins identified in the searches from both completely sequenced genomes and from genomes that have not been fully sequenced should be entered as shown below.

bacteria[Organism] NOT (7514776[gi] OR 98292[gi] OR 7463153[gi] OR 11346858[gi] OR 7468911[gi] OR 7471826[gi] OR 1786676[gi] OR 1075209[gi] OR 7464043[gi] OR 3150103[gi] OR 1293572[gi] OR 7477980[gi] OR 1361493[gi] OR 2146109[gi] OR 11261620[gi] OR 7467603[gi] OR 11348451[gi] OR 5918469[gi] OR 7469304[gi] OR 7462368[gi] OR 2583049[gi] OR 7521020[gi] OR 11356842[gi] OR 11261617[gi] OR 11261615[gi] OR 12720609[gi] OR 12513340[gi] OR 118808[gi] OR 1169397[gi] OR 10039144[gi] OR 10172646[gi] OR 118807[gi] OR 9789547[gi] OR 7468224[gi] OR 8978415[gi] OR 12725258[gi] OR 13357643[gi] OR 6014997[gi] OR 2494198[gi] OR 12045280[gi] OR 581255[gi] OR 13474589[gi] OR 13421402[gi] OR 7464040[gi] OR 11360902[gi] OR 13508357[gi] OR 13359980[gi] OR 67058[gi] OR 41291[gi] OR 43320[gi] OR 145297[gi] OR 1773152[gi] OR 1574159[gi] OR 9655520[gi] OR 9947490[gi] OR 9106885[gi] OR 7380298[gi] OR 580855[gi] OR 467409[gi] OR 580914[gi] OR 2632286[gi] OR 6968590[gi] OR 2984127[gi] OR 4981209[gi] OR 4155207[gi] OR 2313841[gi] OR 4376294[gi] OR 7189650[gi] OR 3328753[gi] OR 7190650[gi] OR 13093951[gi] OR 3323329[gi] OR 2688379[gi] OR 3861390[gi] OR 6899039[gi] OR 6460225[gi] OR 2960145[gi] OR 1673890[gi] OR 1352305[gi] OR 3845015[gi] OR 1652627[gi] OR 2506368[gi] OR 479843[gi] OR 3212391[gi] OR 145783[gi] OR 1651540[gi] OR 1787341[gi] OR 1074027[gi] OR 2506369[gi] OR 1573429[gi] OR 12722081[gi] OR 11280161[gi] OR 9656559[gi] OR 12644665[gi] OR 11348448[gi] OR 9949059[gi] OR 11280162[gi] OR 972778[gi] OR 11353892[gi] OR 7379683[gi] OR 11353126[gi] OR 7226000[gi] OR 11360901[gi] OR 9105556[gi] OR 7477702[gi] OR 2105050[gi] OR 3097239[gi] OR 13092554[gi] OR 12723272[gi] OR 10172656[gi] OR 13423258[gi] OR 6318311[gi] OR 13470653[gi] OR 586870[gi] OR 2126931[gi] OR 467421[gi] OR 2632298[gi] OR 7473469[gi] OR 6460143[gi] OR 7514459[gi] OR 2983903[gi] OR 7467604[gi] OR 3860738[gi] OR 7462369[gi] OR 4981299[gi] OR 11360903[gi] OR 7190502[gi] OR 7468912[gi] OR 3328592[gi] OR 7469488[gi] OR 1653573[gi] OR 13507746[gi] OR 2496262[gi] OR 2146108[gi] OR 1673807[gi] OR 7468225[gi] OR 4376545[gi] OR 7189405[gi] OR 8978646[gi] OR 7463400[gi] OR 2688709[gi] OR 13357575[gi] OR 11356841[gi] OR 7465204[gi] OR 4155746[gi] OR 7521018[gi] OR 3322812[gi] OR 7464041[gi] OR 2314393[gi] OR 11346661[gi] OR 6968051[gi] OR 1527142[gi] OR 13700368[gi] OR 5918469[gi] OR 14089462[gi] OR 4587466[gi] OR 14089466[gi] OR 1732451[gi] OR 13700374[gi] OR 7480627[gi])

To be distinguished as δ, a protein must meet the criteria outlined above in step 4 of the search for δ from fully sequenced genomes.

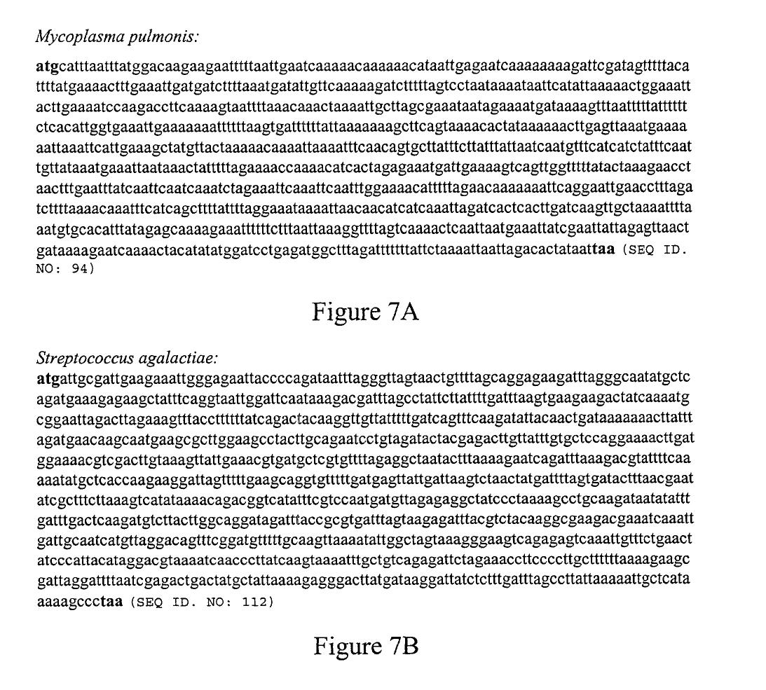

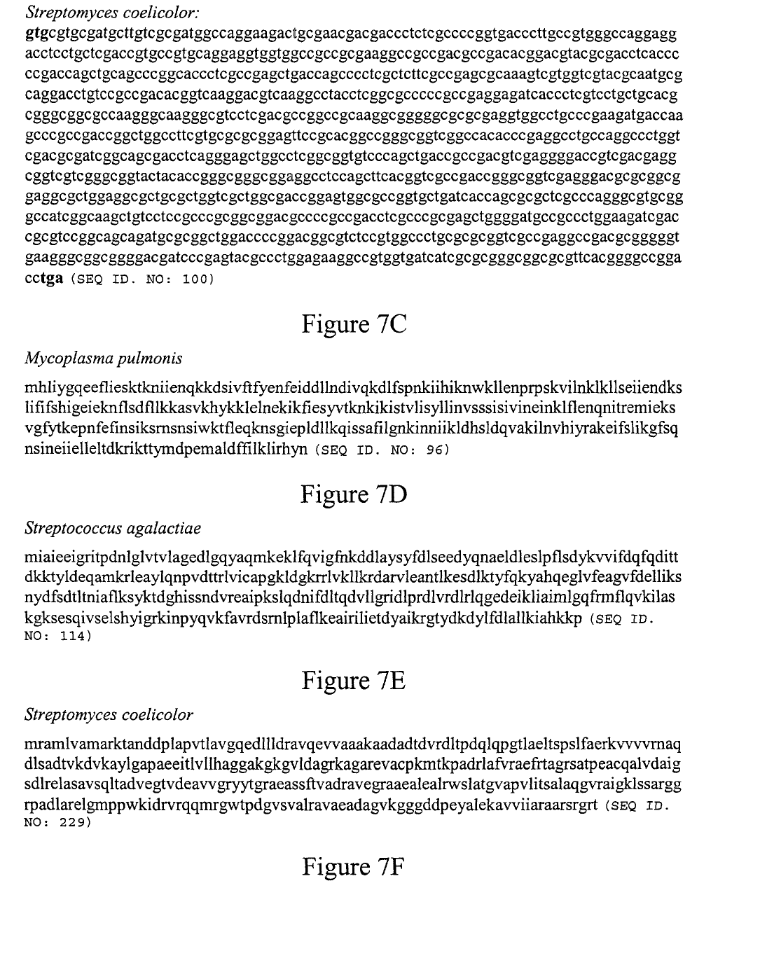

After four iterations four additional δ proteins were identified from: Mycoplasma pulmonis, Streptococcus agalactiae and Streptomyces coelicolor. The sequences of these δ proteins are shown in FIG. 7. The locations of the corresponding DnaX and δ genes from Mycoplasma pulmonis and Streptomyces coelicolor are shown in Tables 8 and 9, respectively.

### Example 6

Identification of Additional δ-Encoding Sequences Using BLAST Searches Initiated with Delta Sequences from Phylogenically Related Organisms

In this example, the broad group of delta sequences generated in the preceding sections (Examples 4 and 5) to enables, using phylogenic relationships, the identification of delta sequences from any organism, using the standard ‘BLAST’ program. This method would not have been possible in the beginning using only the few known delta sequences since delta sequences are not well-conserved. But, now that representatives in all major branches of the bacterial phylogenic tree have been identified, deltas can be identified from newly sequenced organisms by searching with a delta sequence from a closely related organism.

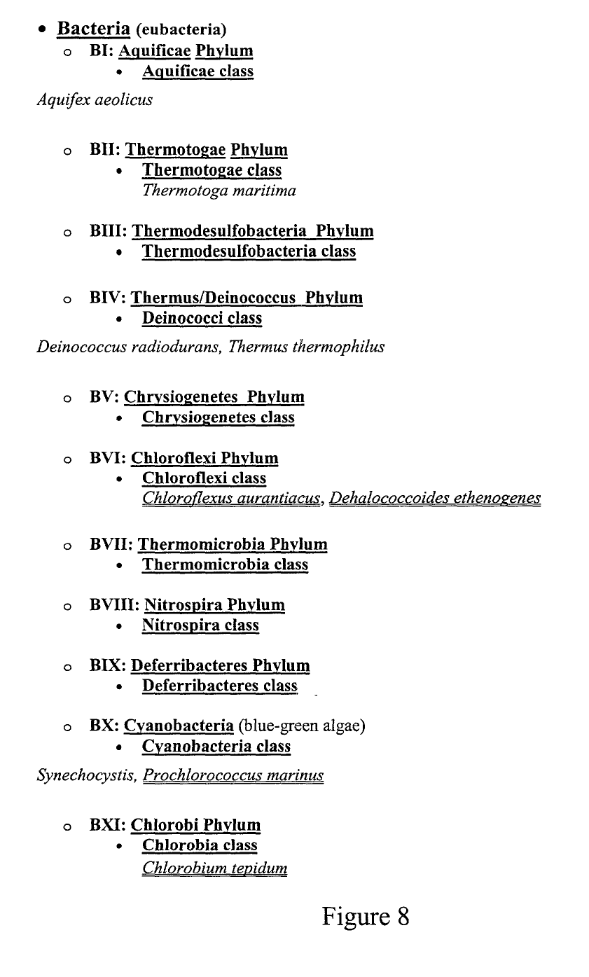

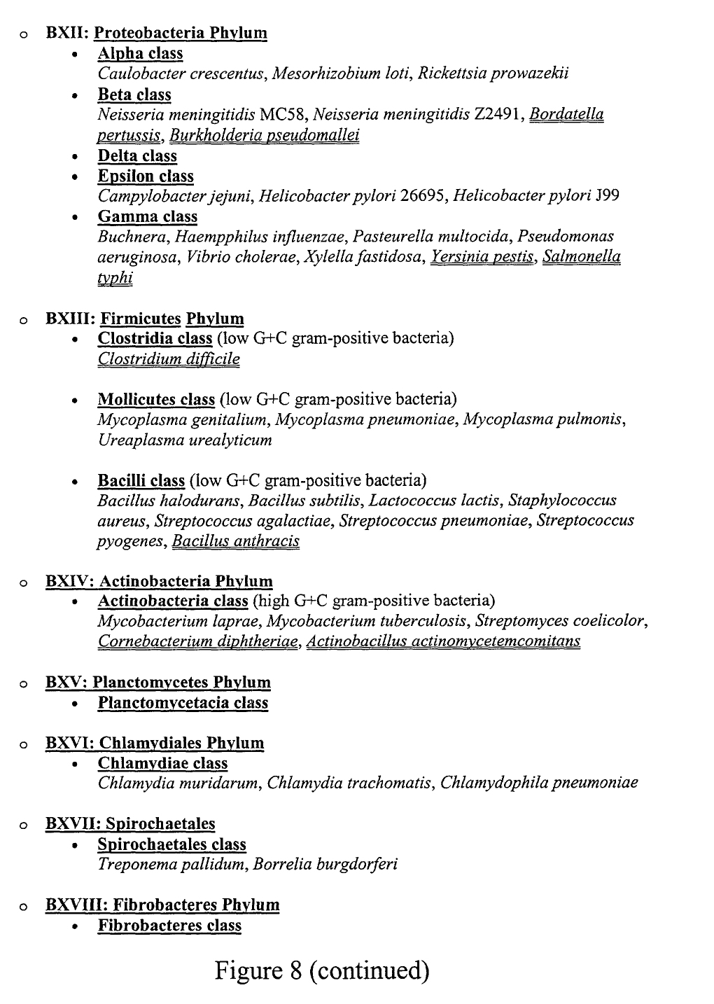

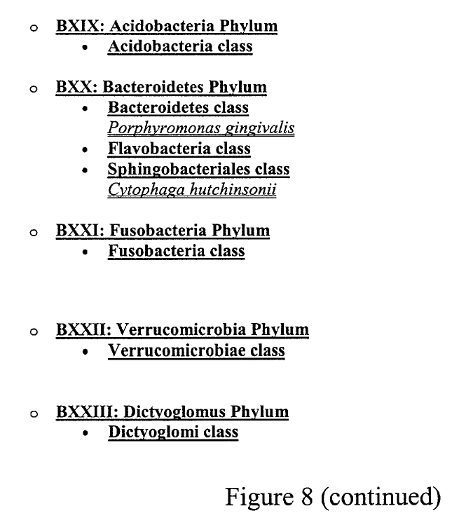

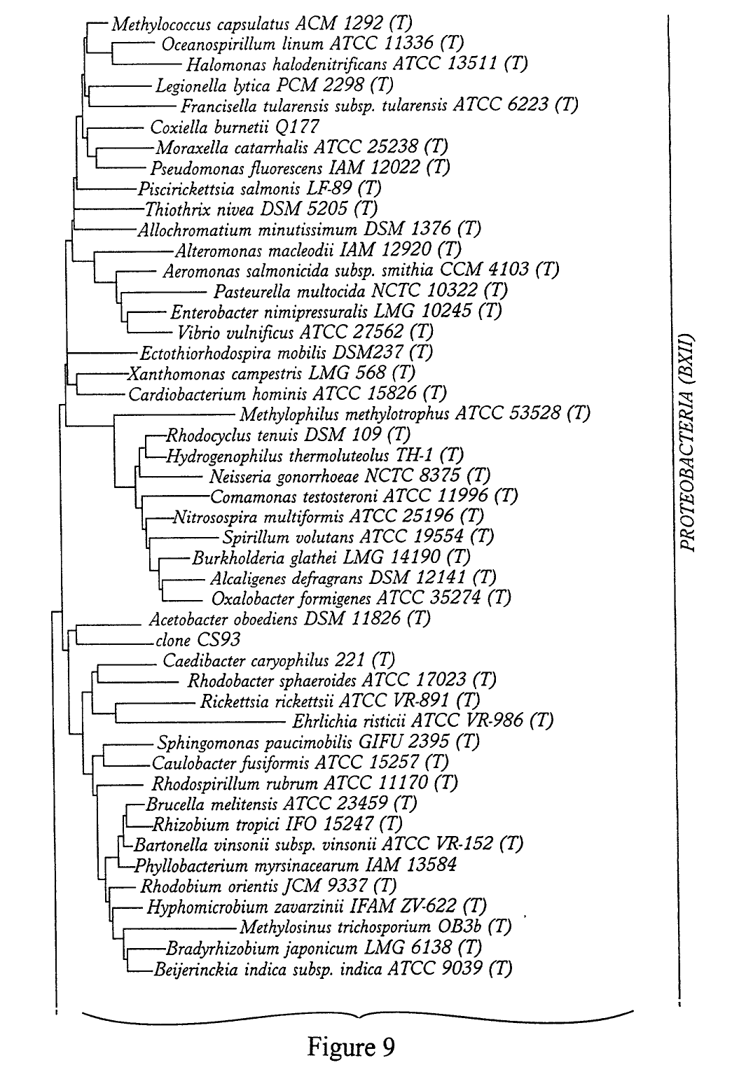

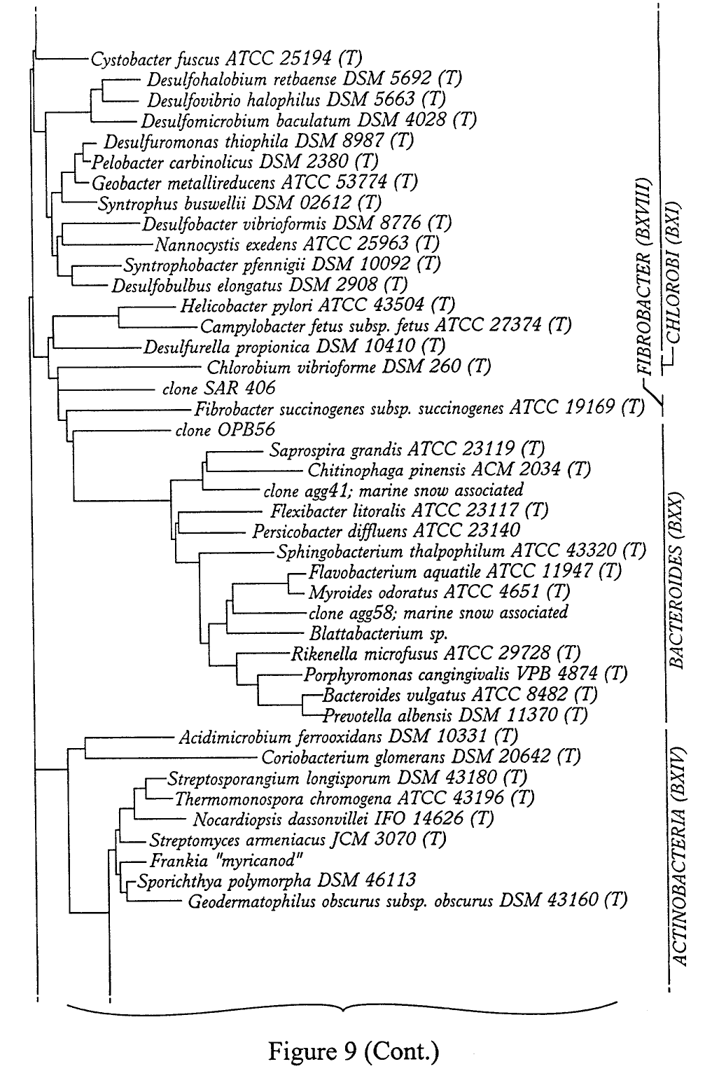

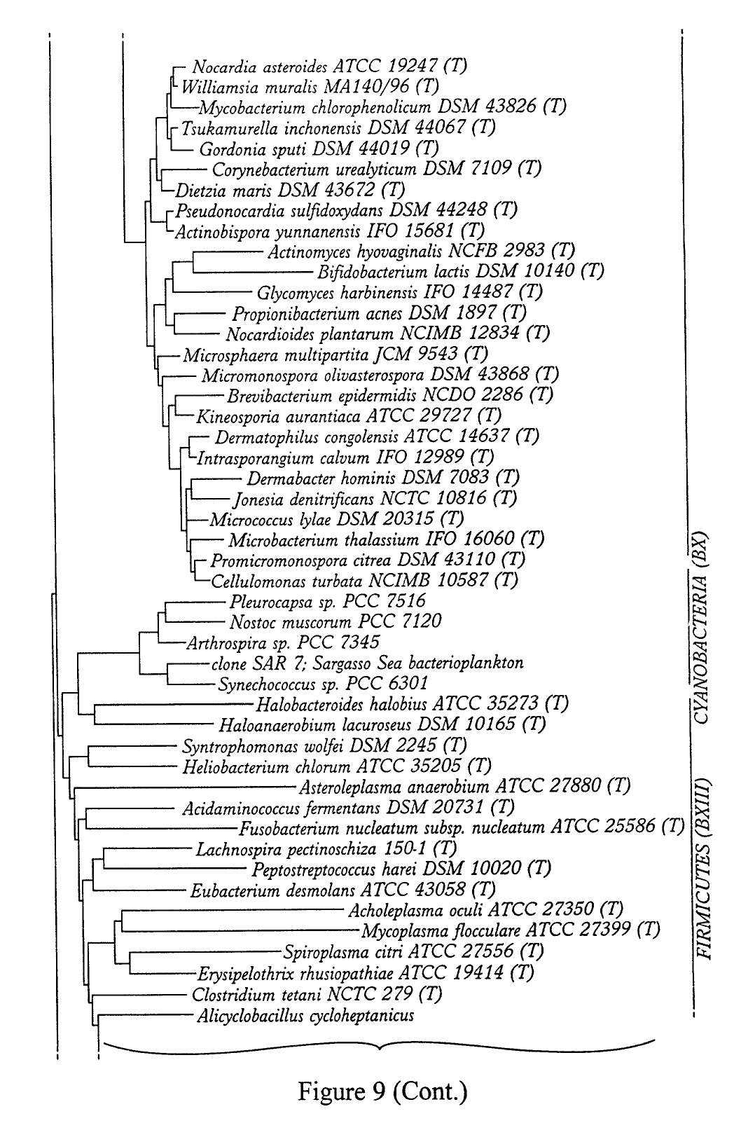

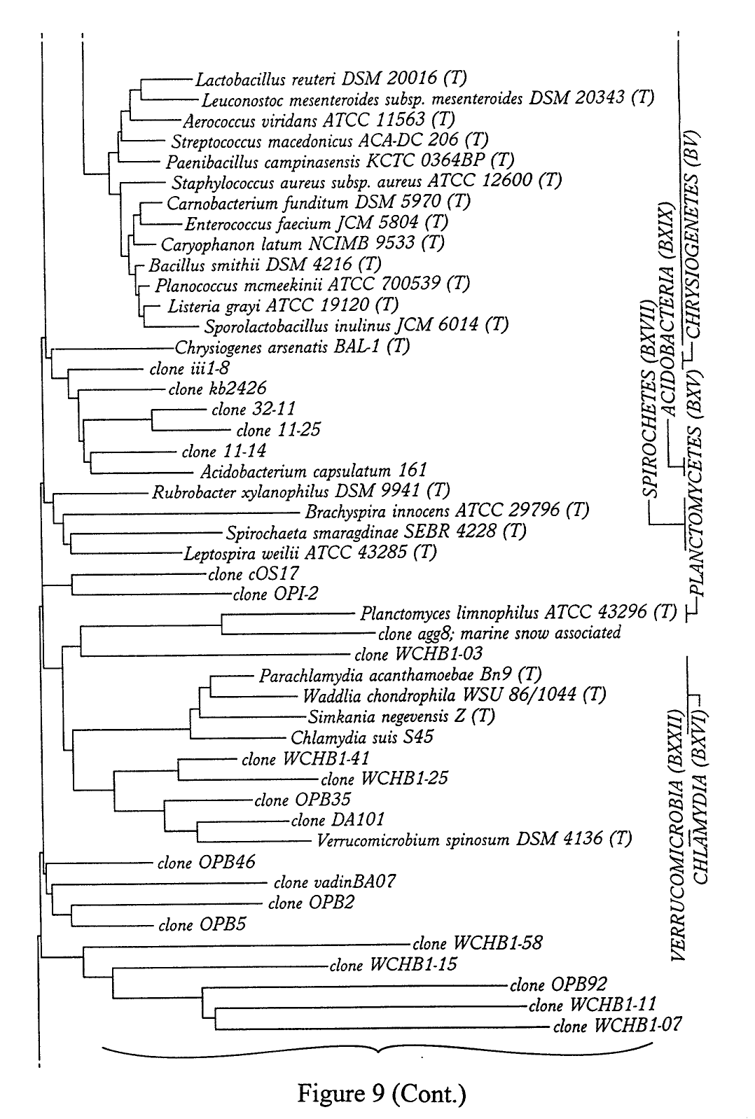

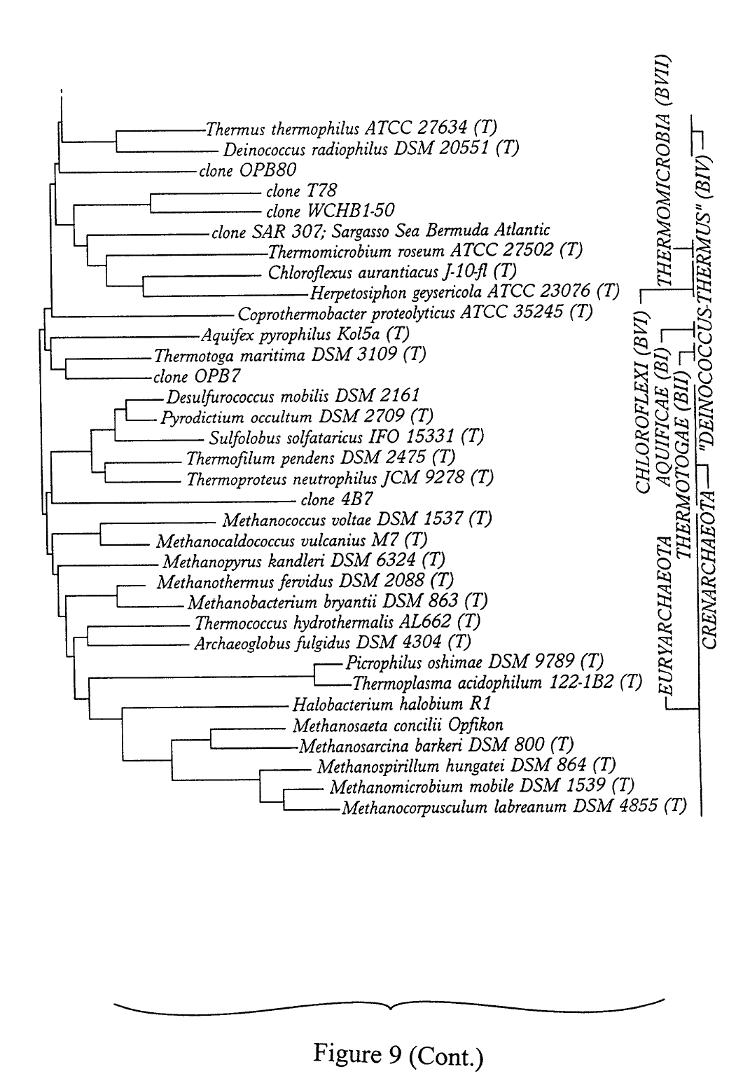

A phylogenic model exists relating organisms by their evolutionary relationship determined by their rRNA sequences (“Taxonomic Outline of the Procaryotic Genera”, Bergey's Manual® of Systemic Bacteriology, Second edition, Release 1.0, April 2001) and is shown in FIG. 8. This phylogenic model shows 23 different phyla and the classes within these phyla based on data available at the time of the release. A phylum is the first taxonomic level into which classes (the second taxonomic level) of bacteria are assigned by the relationship of their rRNA sequences. The phylogenic model was used to determine closely related organisms so that new delta sequences could be identified by blast searches. After use of psi-blast in examples 4 and 5, delta sequences remained unidentified in several phyla because there were no representatives of those phyla in the NCBI nr database used for Psi Blast searches. To identify 5 proteins from organism representative of other phyla of the phylogenic model additional searches are required. To aid in identifying other organisms the 2000 Poster Backbone Tree supplement for ASM 2000 poster presented by Michigan State University and The Ribosome Database Project was also used. This figure has been adapted and appears here as FIG. 9. This phylogenic tree allows evolutionarily close organisms to be identified for use in searching for new 5 proteins.

Databases were searched from sequencing projects underway at: The Sanger Centre, TIGR, the Institute for Genomic Research, The University of Oklahoma Advanced Center for Genome Technology and DOE-Joint Genome Institute. The searches were conducted using the tblastn BLAST search. The settings used in these tblastn BLAST (Gish, W. and States, D. J., Nature Genet. 3:266-272 (1993)) searches were as described below. This program compares a protein query sequence against a nucleotide database that has been translated in all six reading frames. The search parameters should be set at default, in these searches default parameter options were:

Expect set at 10

Matrix set at BLOSUM 62

HSP score set at “sump”

Filter type set at “seg”

Filter low complexity regions

Sort results by pvalue

Genetic code set at standard

To be designated as a δ, the protein (hits) must meet the requirements of step 4 of the PSI-BLAST δ search described above in Example 4, method B. To identify δ from new bacterial species the search should initially begin by searching against δ amino acid sequences from organisms that are evolutionarily close (i.e., members of the same class/phylum) to the target organism. If the initial search produces no hits, then searches should continue using δ amino acid sequences as query sequences that are gradually more evolutionarily distant from the target organism using FIG. 9 as a guide (picking an organism that is linked by the fewest branches to the target organism) until δ has been identified, or until δ sequences from all known phyla have been exhausted. The organisms whose δ amino acid sequences were used to search target databases to identify new δ proteins are shown in Table 20.

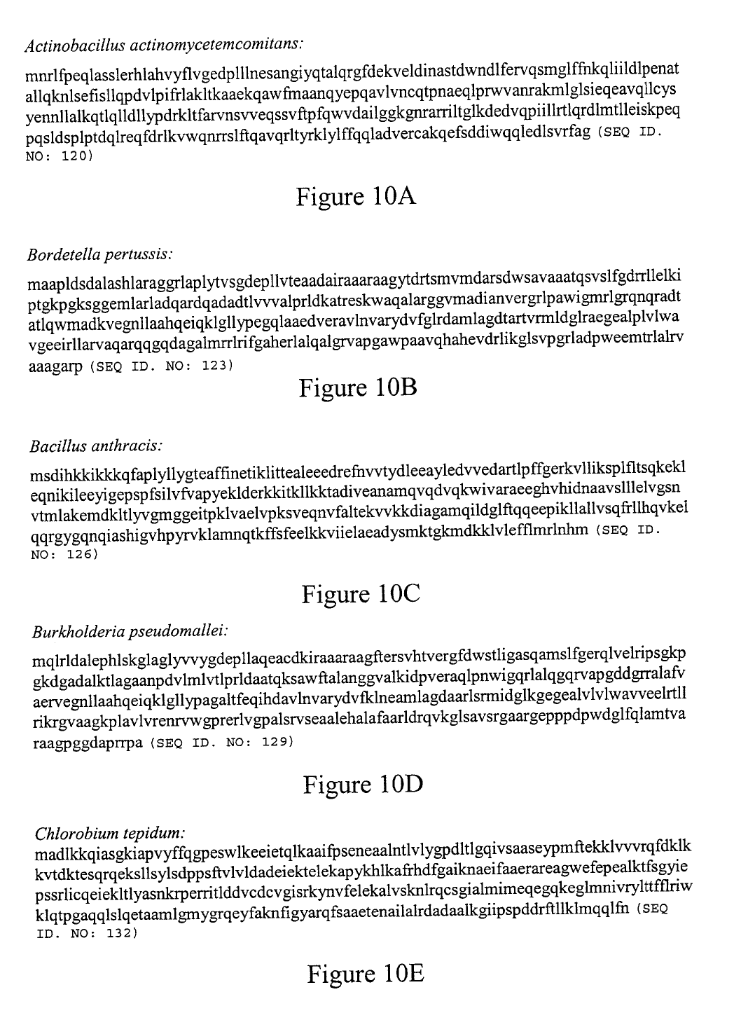

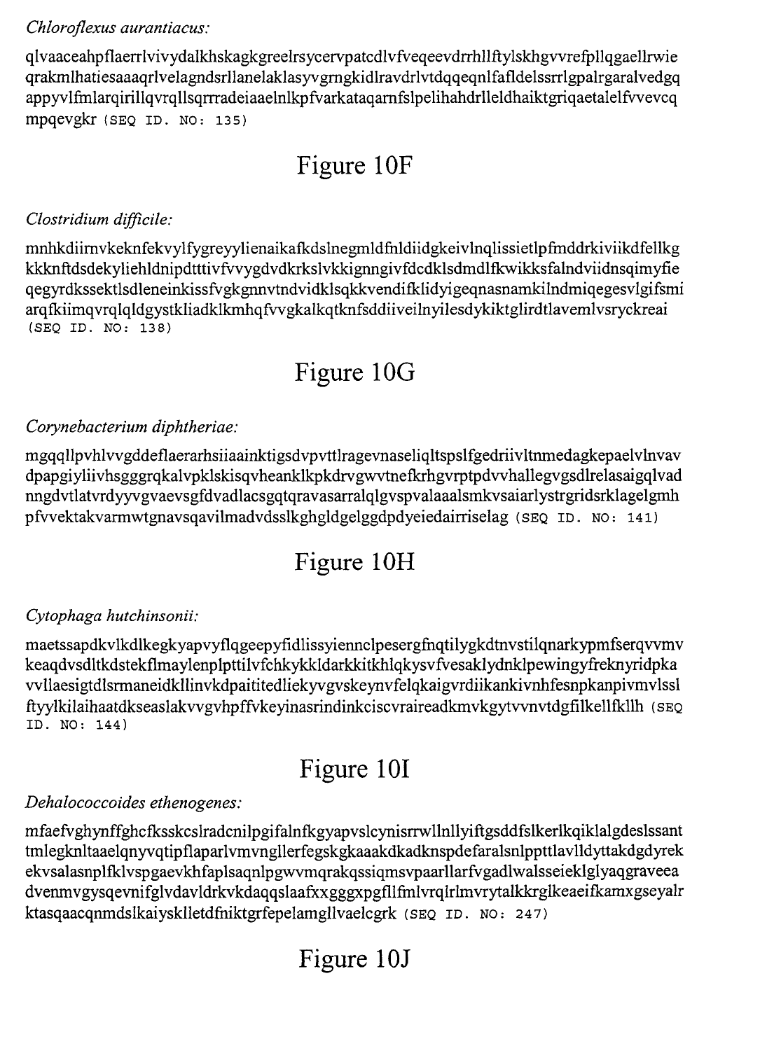

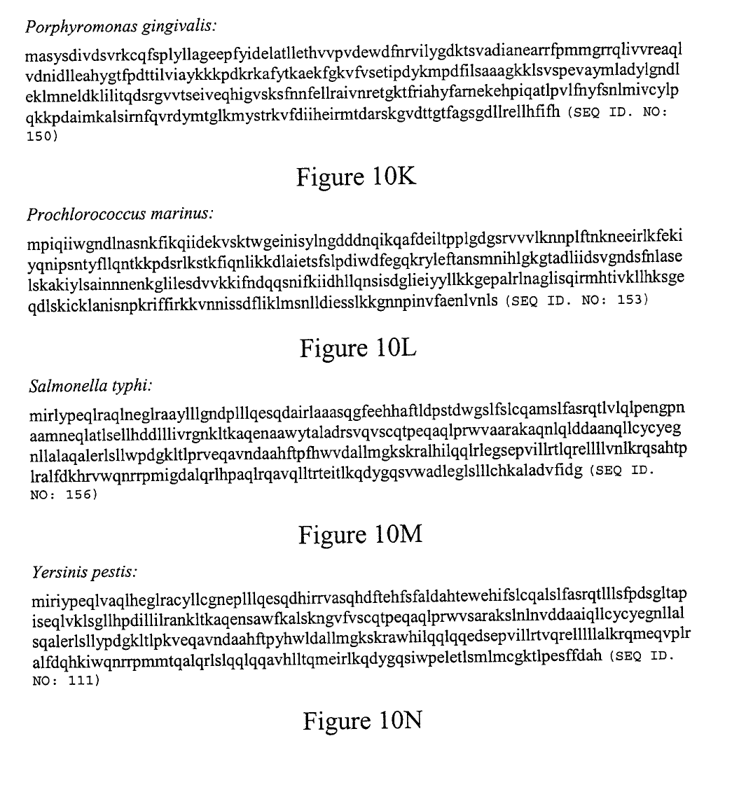

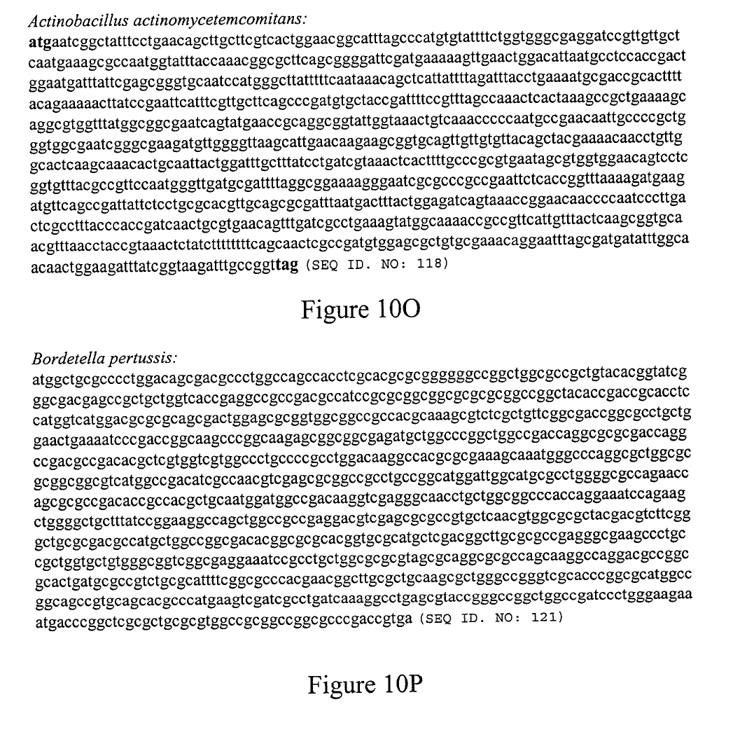

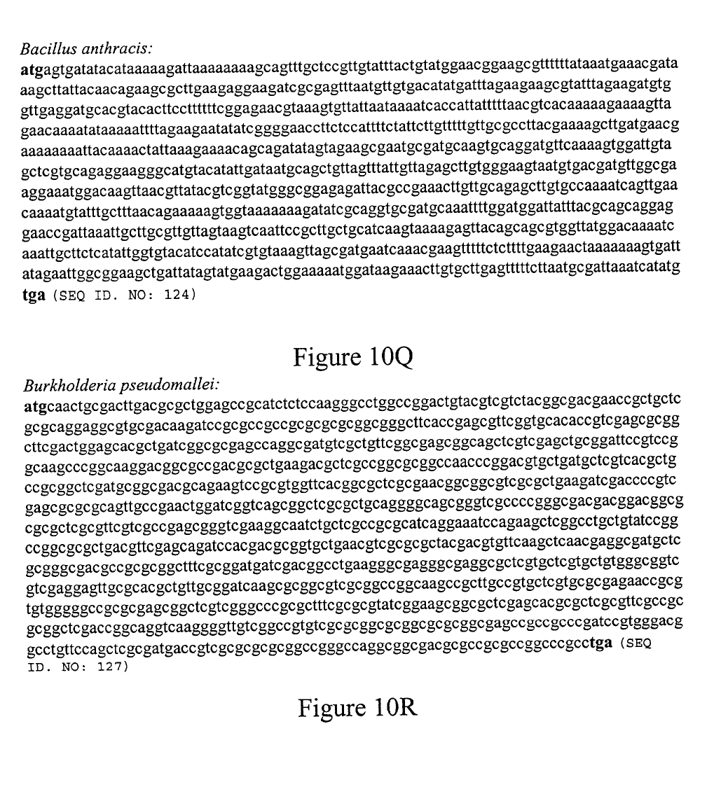

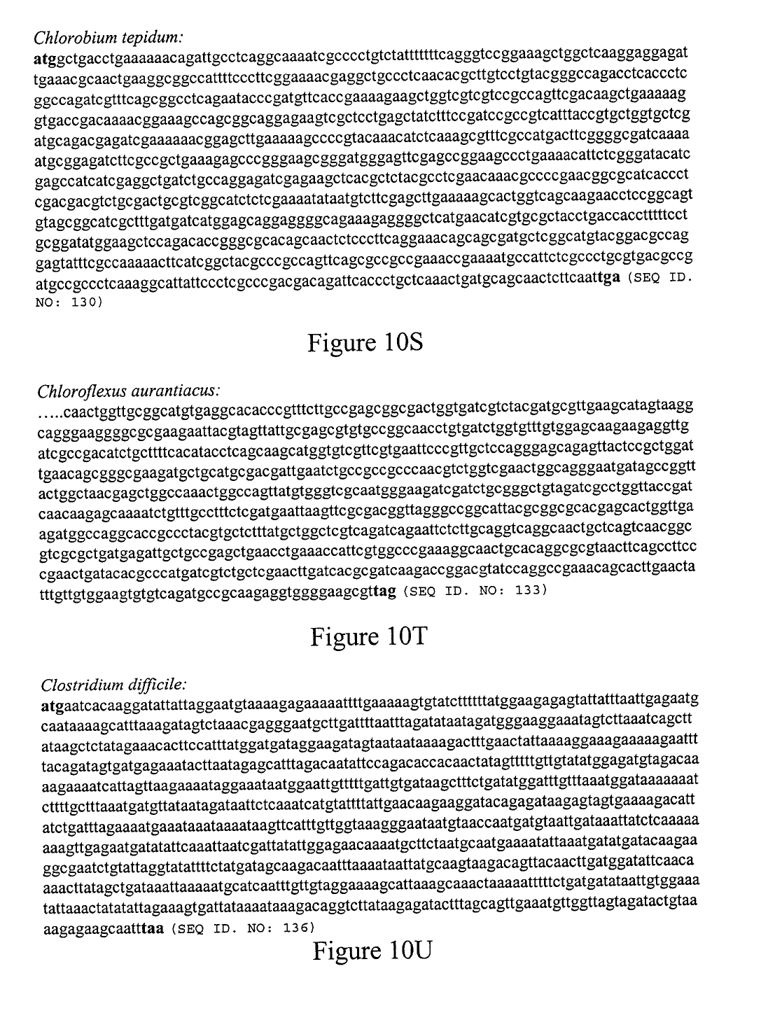

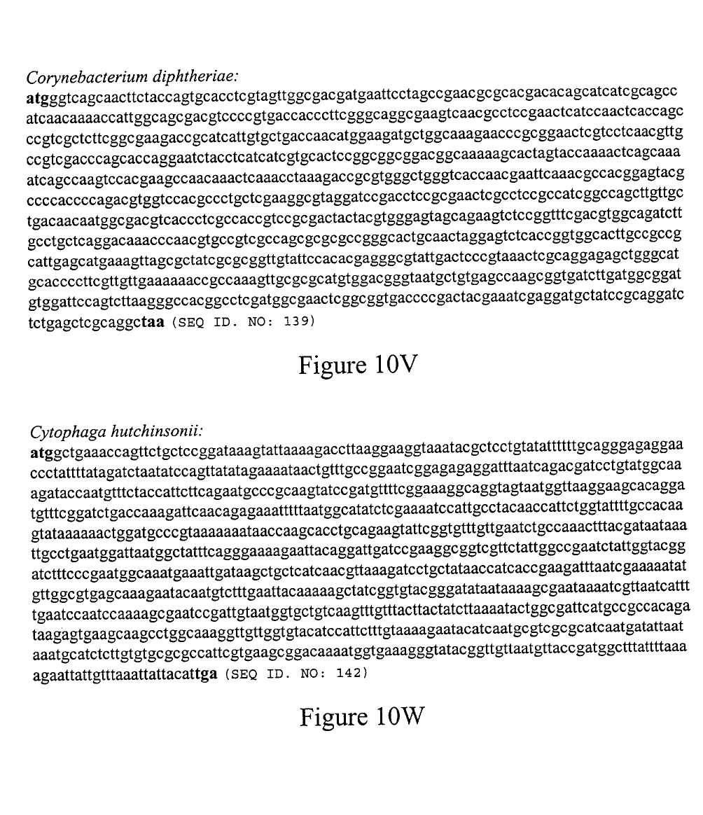

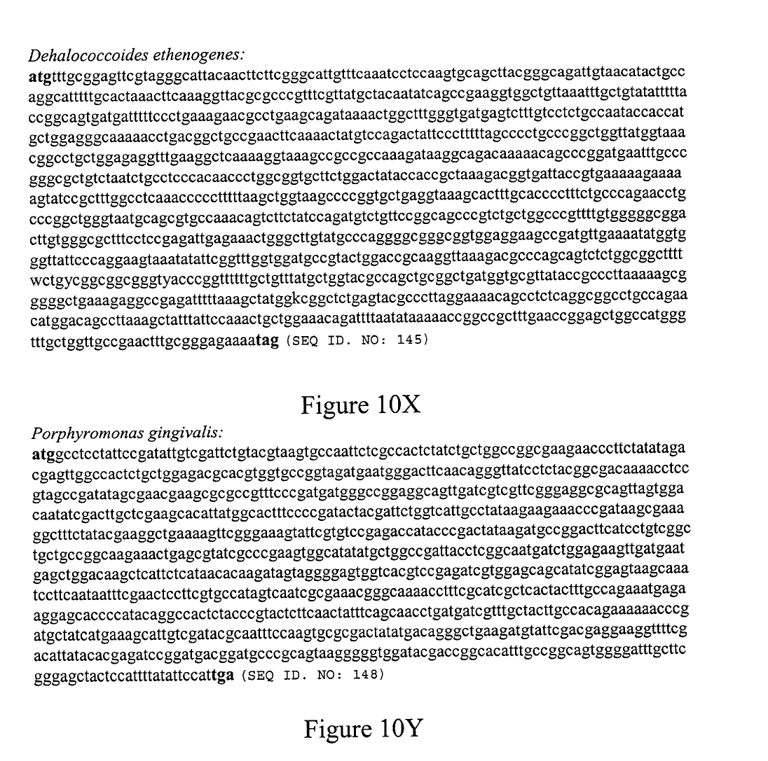

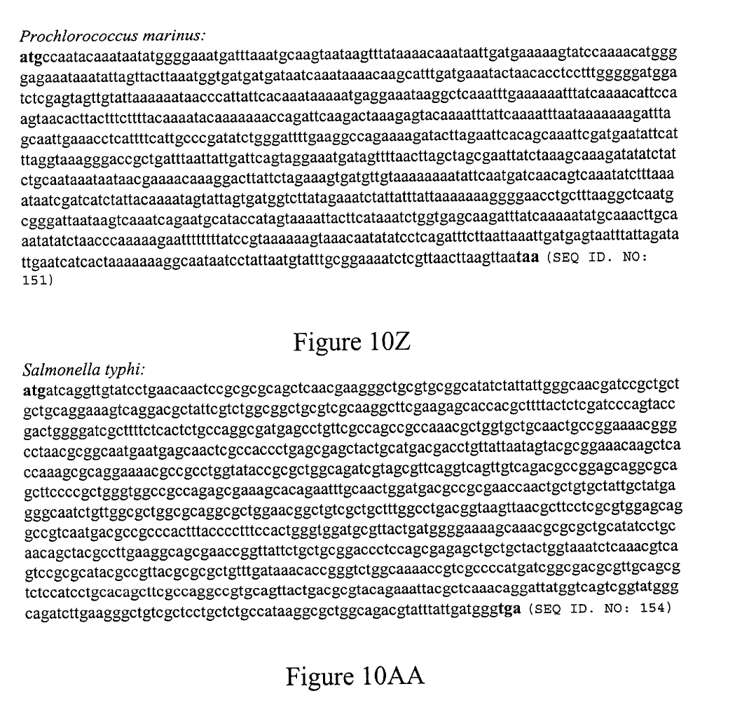

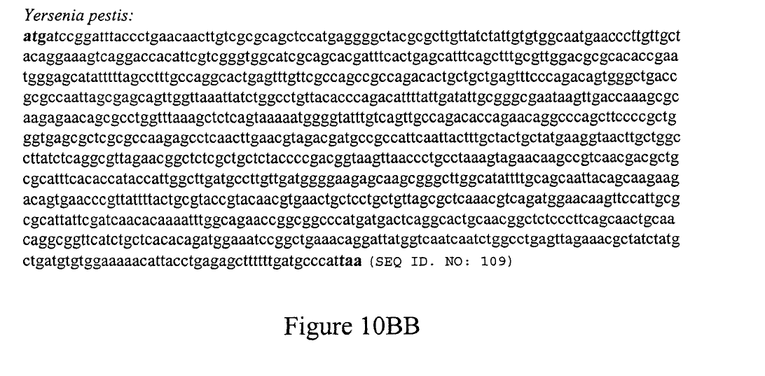

Fourteen additional δ proteins (FIG. 10) were identified representing organisms from various phyla. Bacillus anthracis, Bordatella pertussis, Burkholderia pseudomallei, Chlorobium tepidum, Clostridum difficile, Corynebacterium diphtheriae, Cytophaga hutchinsonii, Dehalococcoides ethenogenes, Prochlorococcus marinus, Porphyromonas gingivalis, Actinobacillus actinomycetemcomitans, Yersenia pestis and Salmonella typhi. A search of the database of Chloroflexus aurantiacus resulted in a protein meeting the requirements of step 4 of the PSI-BLAST δ search described above, however the DNA encoding this protein was located at the 5′ end of a contig and therefore as many as 60 amino acids at the N-terminus of this δ sequence may be missing. These organisms containing the 14 additional δ proteins identified have been added to the phylogenic classification shown as double underlined organisms in FIG. 8. This model may be used as a guide in identification of new δ proteins.

These results indicate that using the above outlined procedures, δ can be identified from any organism in which the region of the genome encompassing the gene encoding δ has been sequenced.

The databases containing genomic DNA sequences from the same fourteen organisms from which δ was identified was next searched against evolutionarily close DnaX and δ′ proteins using tblastn as described above for tblastn searches to identify δ. The DnaX and δ′ gene location or protein annotations are shown in (Table 10) and (Table 11).

As an alternative to performing the described searches in Examples 1-6 using the internet, sequence databases may be installed locally and searches may be conducted using Psi-Blast and Blast. BLAST and PSI-BLAST can be run locally and used to perform searches against local databases. BLAST binaries are provided for Macintosh, Win32 (PC), LINUX, Solaris, SGI, and HP UX systems. Standalone BLAST executables may be found on the NCBI anonymous FTP server under. Read the README file in this directory for more information. Information on setting up Standalone BLAST can also be found at the National Human Genome Research Institute web site.

### Example 7

Identification of the Structural Gene for the δ Subunit of DNA Polymerase III Holoenzyme (holA) from Sequenced Thermophilic Organisms, Thermotoga maritima and Aquifex aeolicus.

Using the described ψ-BLAST procedure, starting with a 0.001 expectation value for inclusion in the first and subsequent iterations, only holA genes closely related to E. coli were identified (Haemophilis influenzae or Neisseria meningitidis). These three sequences were included in a second iteration, permitting a search by only elements common between the family of three δs. This resulted in identification of a candidate from Aquifex aeolicus (hypothetical protein AQ_{—}1104; length=350 aa; score e=9×10⁻⁷) and candidates from B. subtilis and Deinococcus radiodurans. This procedure was repeated through three iterations until a Thermotoga maritima candidate was identified (TM0181; length=324 amino acids; score e=2×10⁻⁷).

From the The Institute for Genomic Research (TIGR) web site the following sequence for Thermotoga maritima protein TM0181 was downloaded:

mpvtfltgtaetqkeelikkllkdgnveyirihpedpdkidfirsllrtktifsnktiidivnfdewkaqeqkrlvellknvpedvhifir sqktggkgvalelpkpwetdkwlewiekrfrengllidkdalqlffskvgtndliiereieklkaysedrkitvedveevvftyqtpg yddfcfavsegkrklahsllsqlwkttesvviatvlanhfldlfkilvlvtkkryytwpdvsrvskelgipvprvarflgfsfktwkfk vmnhllyydvkkvrkilrdlydldravkseedpkpffhefieevaldvyslqrdee (SEQ ID NO: 210)

The gene sequenced, obtained from the same website was:

SEQ ID NO:211)
cactttgaaattgtttctcaaagcgtgttaaaatcaccATGagaggtgattgtatgccagtcacgtttctcacaggtactgcagaaactc
agaaggaagaattgataaagaaactcctgaaggatggtaacgtggagtacataaggatccatccggaggatcccgacaagatcgatttca
taaggtctttactcaggacaaagacgatcttttccaacaagacgatcattgacatcgtcaatttcgatgagtggaaagcacaggagcaga
agcgtctcgttgaacttttgaaaaacgtaccggaagacgttcatatcttcatccgttctcaaaaaacaggtggaaagggagtagcgctgg
agcttccgaagccatgggaaacggacaagtggcttgagtggatagaaaagcgcttcagggagaatggtttgctcatcgataaagatgccc
ttcagctgtttttctccaaggttggaacgaacgacctgatcatagaaagggagattgaaaaactgaaagcttattccgaggacagaaaga
taacggtagaagacgtggaagaggtcgtttttacctatcagactccgggatacgatgatttttgctttgctgtttccgaaggaaaaagga
agctcgctcactctcttctgtcgcagctgtggaaaaccacagagtccgtggtgattgccactgtccttgcgaatcacttcttggatctct
tcaaaatcctcgttcttgtgacaaagaaaagatactacacctggcctgatgtgtccagggtgtccaaagagctgggaattcccgttccag
atactgagggatctctacgatctggacagagccgtgaaaagcgaagaagatccaaaaccgttcttccacgagttcatagaagaggtggca
ctggatgtatattctcttcagagagatgaagaataactggtacagcctcgcggcccttctctcaaccatatacagcaggcacctcgatgt
ggaagcaagacctgtgaagtttgaggaaataaa

The stop codon (taa) and start (atg) specified in the sequence database are in lower case and underlined.

Subjecting the identified gene and flanking sequences to open reading frame analysis using the NCBI orf finder program (NCBI web site) revealed another in-frame possible start five codons upstream (ATG) in capitals and underlined in the sequence listed above. This alternative start would encode a protein with the penta-peptide sequence MRGDC on the amino-terminal end of the database listing from Thermotoga maritima protein TM0181. Other orf finder programs are widely available and well known in the art can be used to identify open reading frames, i.e., Vector NTI V.5.2.1.0, Informax, Inc. (Dec. 9, 1998).

From the NCBI Entrez website, the structural gene for hypothetical protein AQ_{—}1104 (SEQ ID NO: ) was downloaded and the corresponding protein sequence (SEQ ID NO: ) was verified:

(SEQ ID NO:212)
tggcttttacctctttgtagagttaagtattaaatttaatttaactgtggaaaccacaatattccagttccagaaaacttttttcacaaa
acctccgaaggagagggtcttcgtccttcatggagaagagcagtatctcataagaacctttttgtctaagctgaaggaaaagtacgggga
gaattacacggttctgtggggggatgagataagcgaggaggaattctacactgccctttccgagaccagtatattcggcggttcaaagga
aaaagcggtggtcatttacaacttcggggatttcctgaagaagctcggaaggaagaaaaaggaaaaagaaaggcttataaaagtcctcag
aaacgtaaagagtaactacgtatttatagtgtacgatgcgaaactccagaaacaggaactttcttcggaacctctgaaatccgtagcgtc
tttcggcggtatagtggtagcaaacaggctgagcaaggagaggataaaacagctcgtccttaagaagttcaaagaaaaagggataaacgt
agaaaacgatgcccttgaataccttctccagctcacgggttacaacttgatggagctcaaacttgaggttgaaaaactgatagattacgc
aagtgaaaagaaaattttaacactcgatgaggtaaagagagtagccttctcagtctcagaaaacgtaaacgtatttgagttcgttgattt
actcctcttaaaagattacgaaaaggctcttaaagttttggactccctcatttccttcggaatacaccccctccagattatgaaaatcct
gtcctcctatgctctaaaactttacaccctcaagaggcttgaagagaagggagaggacctgaataaggcgatggaaagcgtgggaataaa
gaacaactttctcaagatgaagttcaaatcttacttaaaggcaaactctaaagaggacttgaagaacctaatcctctccctccagaggat
agacgctttttctaaactttactttcaggacacagtgcagttgctgagggatttcttgacctcaagactggagagggaagttgtgaaaaa
tacttctcatggtggataatcttttttatgaagtttgcggtttgcgtttttcccggttctaactgcgattacgacacctactacgttata
agggacattcttgaaaaggacgtggaattc

The gtg start codon and the taa stop codon are shown as bold underlined text.

(SEQ ID NO:213)

mettifqfqktfftkppkervfvlhgeeqylirtflsklkekygenytvl
wgdeiseeefytalsetsifggskekavviynfgdflkklgrkkkekerl
ikvlrnvksnyvfivydaklqkqelsseplksvasfggivvanrlskeri
kqlvlkkfkekginvendaleyllqltgynlmelkleveklidyasekki
ltldevkrvafsvsenvnvfefvdllllkdyekalkvldslisfgihplq
imkilssyalklytlkrleekgedlnkamesvgiknnflkmkfksylkan
skedlknlilslqridafsklyfqdtvqllrdfltsrlerevvkntshgg

Alignment of the two newly identified δ's with the established δ sequences of E. coli and T. thermophilus and the candidate B. subtilis candidate (hypothetical protein YQEN) using ClustalW 1.8 at The Baylor College of Medicine Search Launcher web site (Aiyar, A., et al., Methods Mol. Biol. 132:221-241 (2000); Thompson, J. D., et al., Nucleic Acids Res. 25(24):4876-4882 (1997)) resulted in the the alignment shown in FIG. 6.

The Baylor College of Medicine Search Launcher is an on-going project to organize molecular biology-related search and analysis services available on the WWW by function by providing a single point-of-entry for related searches (e.g., a single page for launching protein sequence searches using standard parameters). It can be noted that the two newly identified Aquifex aeolicus and Thermotoga maritima candidate δs share the same pattern of similar residues as the other δ sequences. The ambiguity in the proper start for the Thermotoga maritima holA gene was not resolved by the alignment.

### Example 8

Expression of Aquifex aeolicus holA.

Aquifex aeolicus holA will be expressed to produce a native Aquifex aeolicus δ protein with an ATG start codon replacing the native gtg start and the natural termination codon and as a protein with an amino-terminal extension with a peptide sequence that is biotinylated in E. coli and a hexaHis sequence. This peptide (MAGCLNDIFEAQKIEWHHHHHHLVPRGSGGGG . . . ) (SEQ ID NO: 214) and vectors used for expressing proteins fused to it are described (Kim, D. R. and McHenry, C. S., ibid.; PCT WO99/13060). The tagged protein will first be expressed with the amino-terminal Biotin-hexaHis extension because of (i) the ease of monitoring and quantifying expression of tagged δ using conjugated streptavidin reagents in biotin blots (Kim, D. R. and McHenry, C. S., ibid.) and (ii) for ease in subsequent purifications aided by affinity chromatography on Ni⁺⁺-NTA and monomeric avidin columns. Purified tagged protein will be used for initial attempts to form reconstitution assays (see Example 16) and for an antigen for production of rabbit polyclonal antibodies that can be used to monitor the purification of native Aquifex aeolicus δ in the absence of a functional assay, if necessary. Polyclonal and monoclonal antibodies will be made by methods old and well known in the art. Total protein levels will be determined by the Bradford procedure (Bradford, M. M., Anal. Biochem. 72:248-254 (1976)) or other suitable method in this and other examples.

To express tagged Aquifex aeolicus δ, the sequence encoding the biotin/hexaHis tag will be fused with the second codon of Aquifex aeolicus holA. This will be accomplished by PCR, which will be used to amplify the Aquifex holA gene (AQ_{—}1104). The primers will be designed so that a PstI site will be added to the 5′ end of the gene and a HindIII site to the 3′ end of the gene.

The forward primer will be:

5′-gatcctgcagGAAACCACAATATTCCAGTTCC-3′ (SEQ ID NO: 157)

The complementary portion of this primer is shown as upper case and begins at codon #2 (the ATG start codon of the Aquifex holA gene will be excluded). The PstI site is shown in italics and underlined. This will place the open reading frame (ORF) of the Aquifex delta gene inframe with the N-terminal fusion peptide. There is a 4 base extension on the 5′ end of the primer to allow for efficient digestion by PstI.

The reverse primer will be:

(SEQ ID NO:158)

5′-gatcaagcttttaTTATCCACCATGAGAAGTATTTTTCAC-3′

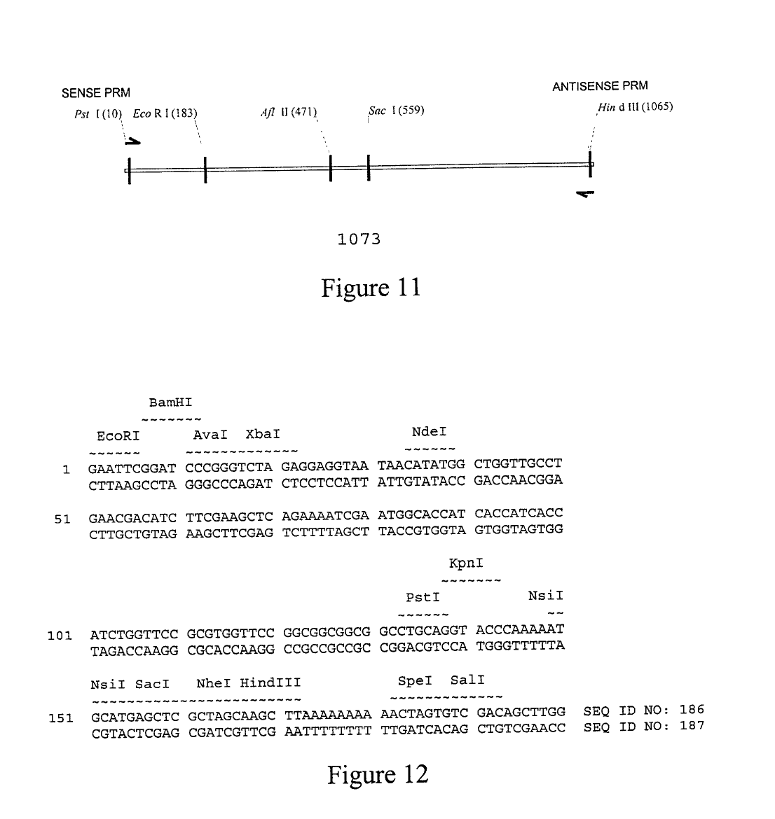

The complementary portion of the primer is shown as upper case. The HindIII restriction site is in italics and underlined and there is a 4 base extension to allow for efficient digestion by HindIII. The non-complementary portion contains an additional stop codon (tta) to enhance termination of translation. A graphical depiction of the PCR product is shown in FIG. 11. The PCR product will be digested with PstI and HindIII and inserted into the vector pA1-NB-KpnI digested with the same restriction enzymes. The region of pA1-NB-KpnI that the PCR product will be inserted into is shown in FIG. 12. This resulting plasmid (pA1-NB-AQD) will be transformed into E. coli DH5α. Positive isolates will be selected by ampicillin resistance and plasmids isolated from the positive isolates will be sequenced across the region contain the insert. Verification of expression will be accomplished by cell growth to OD 0.5 and induction by addition of 1 mM IPTG for three hours. Extracts of cells will be subjected to electrophoresis on SDS gels, transferred to membranes and detected with streptavidin conjugated reagents as taught in PCT WO 99/13060 and U.S. Provisional Patent Application No. 60/192,736, filed Jul. 14, 2000, the disclosure of which are hereby incorporated by reference.

In the above procedures and in the following examples, the following general molecular cloning procedures will be used: All intermediate plasmids will be transformed into E. coli DH5α. Amp^{R} colonies will be selected and the plasmids will be screened for gain or loss of the appropriate restriction sites. The sequences of inserted DNA will be confirmed by DNA sequencing. Ligations will be performed in the presence of T4 DNA ligase and ATP. Expression of proteins will be confirmed by transforming the plasmids into E. coli MGC1030 (mcrA, mcrB, lamBDA(−), (RRND-RRNE)1, lexA3) and AP1.L1 E. coli bacteria. The parent to the AP1.L1 bacterial strain was BLR bacterial strain [F-, ompT hsdSB(rB-mB-) gal dcm δ(srl-recA)306::Tn10 (Novagen). A T1 phage-resistant version of this BLR strain, is desginated AP1.L1. Single colonies (3 colonies from each transformation) of transformed cells selected for by ampicillin-resistance will be inoculated into 2 ml of 2×YT culture media containing 100 μg/ml ampicillin and grown overnight at 37° C. in a shaking incubator. In the morning, 0.5 ml of the turbid culture from the overnight growth will be inoculated into 1.5 ml of fresh 2×YT culture media. The cultures will be grown for 1 hour at 37° C. with shaking and expression will be induced by addition of isopropyl-β-D-thiogalactopyranoside (IPTG) to a final concentration of 1 mM. The cells will be harvested by centrifugation 3 hours post-induction. The cell pellets will be immediately resuspended in 1/10 culture volume of 2×Laemelli sample buffer (2× solution: 125 mM Tris-HCl (pH 6.8), 20% glycerol, 4% sodium dodecyl sulfate (SDS), 5% β-mercaptoethanol, and 0.005% bromophenol blue w/v), and sonicated to complete lysis of cells and to shear the DNA. The samples will be heated for 10 minutes at 90-100° C., and centrifuged to remove insoluble debris. An aliquot (3 μl) of each supernatant will be subjected to electrophoresis in a 4-20% SDS-polyacrylamide mini-gel (Novex, EC60255; 1 mm thick, with 15 wells/gel) in 25 mM in Tris base, 192 mM glycine, and 0.1% SDS. The resulting gels will be stained with Coomassie Brilliant Blue to visualize protein bands.

Proteins expressed as tagged proteins will receive d-biotin at the time of induction to a final concentration of 10 μM. The control culture received d-biotin only and will not be induced with IPTG. The expressed proteins will be analyzed using biotin blots. The total protein in each lysate will be transferred (blotted) from polyacrylamide gel to nitrocellulose membrane using a Novex transfer apparatus at 30 V constant voltage in 12 mM Tris base, 96 mM glycine, 0.01% SDS (w/v), and 20% methanol (v/v) for 60 minutes at room temperature. The membrane will be blocked in 0.2% Tween 20 (v/v)-TBS (TBST) (tris-buffered saline; 8 g/L NaCl, 0.2 g/L KCl, 3 g/L Tris-HCl (pH 7.4)) containing 5% non-fat dry milk (w/v) for 1 hour at room temperature. The blotted nitrocellulose will next be rinsed using TBST, and then incubated in 2 μg/ml alkaline phosphatase-conjugated streptavidin (Pierce Chemical Co.) in TBST for 1 hour at room temperature. Following extensive washing with TBST, the blot will be developed with BCIP/NBT (KPL #50-81-07; one component system). The endogenous E. coli biotin-carboxyl carrier protein (biotin-CCP, ca. 20 kDa) can be detectable in both induced and non-induced samples. The target protein expressed as an N-terminal tagged protein will be visualized in the induced samples only.

Aquifex aeolicus δ will be expressed as a native protein. The gene (AQ_{—}1104) will be amplified from Aquifex genomic DNA using primers designed to allow insertion of the gene into a vector in which the gene can be expressed in a native form.

The forward primer will be designed so that the start “ATG” codon will incorporated into an NdeI restriction site. The forward primer is shown below.

5′-gatccatATGGAAACCACAATATTCCAGTTCC-3′(SEQ ID NO: 159)

The complementary portion of this primer is shown as upper case, the NdeI site is in italics and underlined. There is a 4 base extension on the 5′ end of the primer to allow for efficient digestion by NdeI.

The reverse primer will contain a KpnI site and is shown below.

(SEQ ID NO:160)

gatcggtaccttaTTATCCACCATGAGAAGTATTTTTCAC-3′

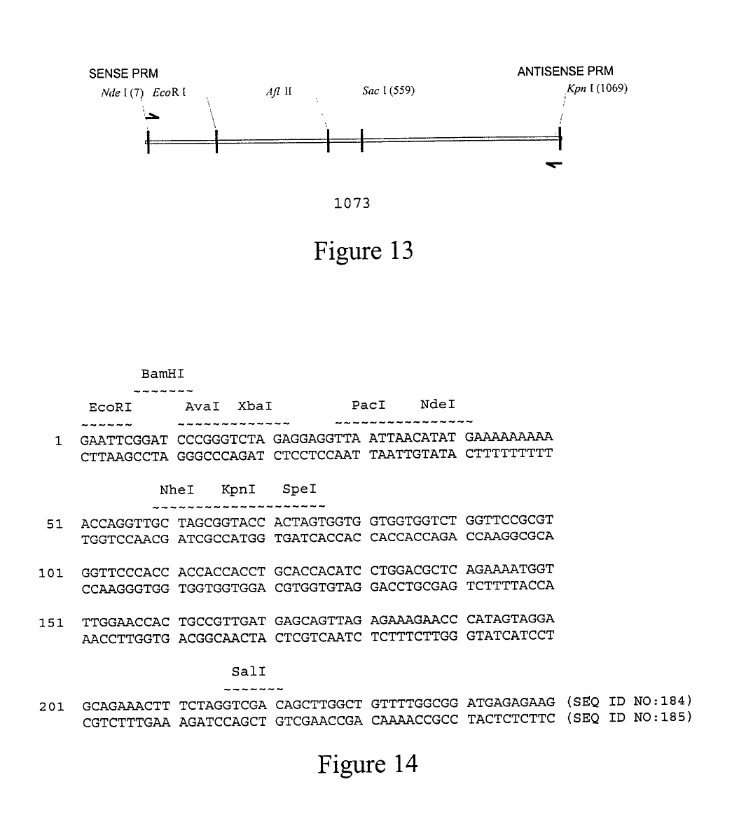

The complementary portion of the primer is shown as upper case. The KpnI restriction site is in italics and underlined and there is a 4 base extension to allow for efficient digestion by KpnI. The non-complementary portion also contains an additional stop codon (tta) to enhance termination of translation. A graphical depiction of the PCR product is shown in FIG. 13.

The PCR product will be digested with NdeI and KpnI and inserted into the vector pA1-CB-NdeI (74) digested with the same restriction enzymes. The region of pA1-CB-NdeI that the PCR product will be inserted into is shown in FIG. 14. There is a sequence encoding a C-terminal biotin/hexaHis fusion peptide located downstream of the KpnI site and thus downstream of the stop codon of the inserted Aquifex holA gene (AQ_{—}1104) and will not be expressed. The sequence of these plasmids will verified as described above and expressed in E. coli MGC 1030 and AP1.L1.

Verification of expression will be accomplished by examination of Coomassie-stained SDS gels from extracts of candidate cells for IPTG-expression of a band of the expected molecular weight. If that procedure fails, polyclonal antibodies will be raised against purified tagged Aquifex aeolicus δ and expressed proteins detected by Western Blots. Expression will also be verified and quantified by employing functional assays as described under Example 16.

### Example 9

Purification of Aquifex aeolicus δ

Tagged Aquifex aeolicus δ will be purified by ammonium sulfate fractionation, Ni⁺⁺-NTA chromatography, and, if needed, monomeric avidin chromatography. Extracts will be prepared by formation of spheroplasts and a gently heat-induced lysis in the presence of spermidine (PCT WO99/13060). Ammonium sulfate fractions (30, 40, 50, 60 70% saturation) will be made and the amount of tagged δ precipitated relative to total protein determined by biotin blots in the linear response range. The concentration of ammonium sulfate that results in precipitation of 80% of the tagged δ will be used in subsequent purifications. An ammonium sulfate pellet resulting from an optimized fractionation procedure will be dissolved in a suitable buffer and chromatographed on Ni⁺⁺-NTA columns by procedures similar to those described (PCT WO99/13060). The elution position of tagged δ will be determined from stained SDS gels and Biotin-blots. If protein is pure, it will be dialyzed and used to support antibody production and preliminary functional assays. If needed, tagged δ first will be further purified by monomeric avidin chromatography (PCT WO99/13060).

Native Aquifex aeolicus δ will be purified by ammonium sulfate fractionation and standard chromatographic procedures. Purification will await the assembly of a reconstituted reaction employing purified tagged Aquifex aeolicus pol III holoenzyme subunits α, β, δ, δ′, ε and DnaX (see Example 16). A functional assay is important for optimal production of fully active subunits free of tags that might partially impair reaction efficiency or weaken protein-protein interactions. Only with a functional assay is it apparent when a procedure leads to protein inactivation. A functional assay is also useful in monitoring the presence of the desired protein in column fractions in the presence of contaminants. Activity is quantified before and after each chromatographic step and only those procedures that result in retention of significant (generally >50%) activity with the loss of contaminating proteins are included in the final purification procedures.

Purification development is an empirical process. Optimizing an ammonium sulfate fractionation from lysates will be the first step, much as described for the tagged protein above, except that δ will be quantified by a functional assay. Then, a survey a variety of cation exchange, anion exchange, hydroxyapatite and hydrophobic interaction chromatographic resins will be used to determine buffer conditions required for binding of 5 activity and elution. For those resins that give the highest yields, gradient elution conditions will be optimized to give the optimal separation of δ from contaminants consistent with high recovery. Successive chromatographic procedures will be developed until δ is nearly homogeneous. The final purification step will likely be gel filtration since it permits removal of any aggregated protein and provides an efficient method to place the purified protein in an optimal storage buffer. Final recovered protein will be aliquoted and stored under optimal conditions, likely rapid freezing by plunging into liquid N₂ and storage at −80 ° C. until needed.

### Example 10

Expression of Thermotoga maritima holA

TM0181 will be expressed as a fusion protein, tagged on its N-terminus, much like described for Aquifex aeolicus holA under Example 8. Since there is ambiguity in the start site, the more upstream site, will be used to ensure all coding sequences are included. Specifically, PCR will be used to amplify the T. maritima holA gene (TM0181). The primers will be designed so that a KpnI site will be added to the 5′ end of the gene and a SpeI site to the 3′ end of the gene. The gene encoding the T. maritima holA gene contains an internal PstI site, which is normally used for inserting genes in frame with an N-terminal biotin/hexaHis fusion peptide. To overcome this problem the expression vector pA1-NB-KpnI contains a KpnI restriction site that partially overlaps the PstI site. Use of this restriction sites will result in an additional two amino acids (Val and Pro) being added in the final protein between the N-terminal biotin/hexaHis fusion peptide and the T maritima holA gene.

The forward primer will be:

5-′gatcggtacccAGAGGTGATTGTATGCCAGTC-3′ (SEQ ID NO: 161)

The complementary portion of this primer is shown as upper case and begins at codon #2 (the ATG start codon of the T. maritima holA gene will be excluded), the KpnI site is in italics and underlined. There is a 4 base extension on the 5′ end of the primer to allow for efficient digestion by KpnI. There is an additional nucleotide “c” in the non-complementary region of the primer to maintain an ORF between the biotin/hexaHis fusion peptide and the T. maritima holA gene.

The reverse primer is shown below.

5′-gatcactagtttaTTATTCTTCATCTCTCTGAAG-3′ (SEQ ID NO: 162)

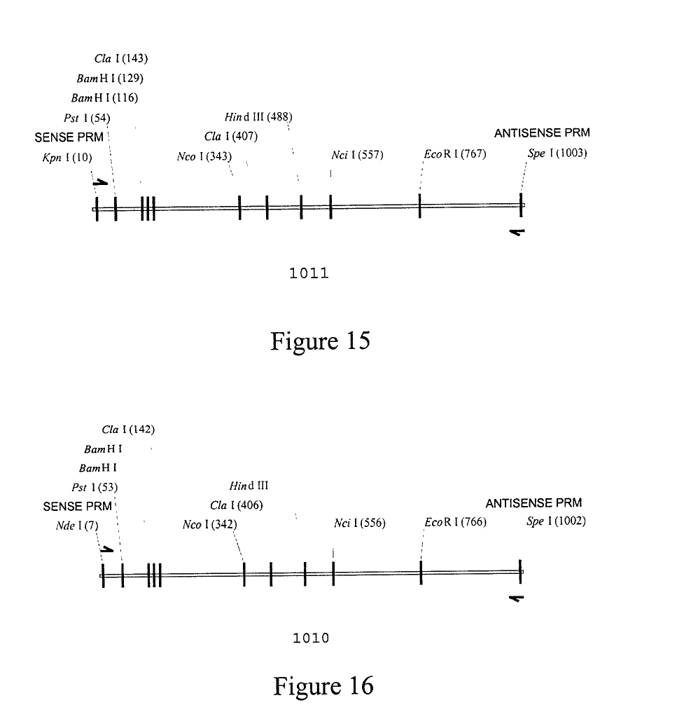

The complementary portion of the primer is shown as upper case. The SpeI restriction site is in italics and underlined and there is a 4 base extension to allow for efficient digestion by SpeI. The non-complementary portion also contains an additional stop codon (tta) to enhance termination of translation. A graphical depiction of the PCR product is shown in FIG. 15.

The PCR product will be digested with KpnI and SpeI and inserted into the vector pA1-NB-KpnI digested with the same restriction enzymes. The region of pA1-NB-KpnI that the PCR product will be inserted into is shown in FIG. 12. These plasmids (pA1-NB-TMD) will be transformed into DH5α bacteria and screened for ampicillin resistance, isolated from positive isolates and the sequence of the isolated plasmids will verified be as described above and expressed in E. coli MGC1030 and AP1.L1. Expressing cells will be verified as described under Example 8.

Antibody against purified tagged Thermotoga maritima δ (obtained from the procedures described under Example 8) will be used to immunoprecipitate δ from Thermotoga maritima extracts. The precipitated protein will be further purified by SDS polyacrylamide gel electrophoresis, transferred to a membrane and sequenced at its amino-terminus to determine which of the two alternative start sites are used. If this procedure fails, both possible δ proteins will be expressed to determine which works best in reconstituting a Thermotoga maritima DNA polymerase III holoenzyme.

Native (untagged) Thermotoga maritima δ will be expressed by amplification of the gene (TM0181) from T. maritima genomic DNA using primers designed to allow insertion of the gene into a vector in which the gene can be expressed in a native form.

The forward primer will be designed so that the start “ATG” codon will be incorporated into an NdeI restriction site. The forward primer is shown below.

5′-gatccatATGAGAGGTGATTGTATGCCAG-3′ (SEQ ID NO: 163)

The complementary portion of the primer is shown as upper case. The NdeI restriction site is in italics and underlined and there is a 4 base extension to allow for efficient digestion by NdeI.

The reverse primer is the same reverse primer used in construction of pA1-NB-TMD (SEQ ID NO:) and contains a SpeI restriction site and an addition stop codon adjacent to the native stop codon.

A graphical depiction of the PCR product is shown in FIG. 16. The PCR product will be digested with NdeI and SpeI and inserted into the vector pA1-CB-NdeI digested with the same restriction enzymes. The region of pA1-CB-NdeI that the PCR product will be inserted into is shown in FIG. 14. These plasmids (pA1-TMD) will be transformed into DH5α screened for ampicillin resistance, isolated from positive isolates and the sequence of the isolated plasmids will verified as described above and expressed in E. coli MGC1030 and AP1.L1. Expression will be verified as described under Example 8 for Aquifex aeolicus δ. The shorter version of this gene beginning at the second possible start site can also be inserted into expression vectors. This will be accomplished using the same strategy used to clone both tagged and untagged forms of the gene encoding the longer version of the Thermotoga maritima δ gene.

### Example 11

Purification of Thermotoga maritima δ

Tagged and native Thermotoga maritima δ will be purified by strategies similar to those described under Example 9. Antibodies against tagged protein will be prepared as described in Example 8.

### Example 12

Identification of the Structural Gene for the δ′ Subunit of DNA Polymerase III Holoenzyme (holB) from Sequenced Thermophilic Organisms, Thermotoga maritima and Aquifex aeolicus

All sequenced bacteria contain two genes that show significant homology to bacterial dnaX. The shorter of the two proteins that shows lesser homology is δ′ in E. coli (Carter, J. R., et al., (1993) ibid. ) and T. thermophilus. This principal was exploited to detect the structural genes for δ′ (holB) in Thermotoga maritima and Aquifex aeolicus. A blast search was conducted using E. coli dnaX and the default parameters of BLAST except the option of showing up to 250 matches was indicated. The Aquifex aeolicus dnaX gene was located (e=1×10⁻⁶⁵ ) and a second gene (aq_{—}1526; 312 amino acids; e=5×10⁻¹⁴) was located, providing a candidate Aquifex holB. The following protein sequence of Aquifex aeolicus hypothetical protein aq_{—}1526 (SEQ ID NO: 215) was downloaded from the NCBI Entrez website:

>gi|2983903|gb|AAC07454.1|hypothetical protein [Aquifex aeolicus]

| | |
|---|---|
| MEKVFLEKLQKTLHIPGGLLFYGKEGSGKTKTAFEFAKGILCKENVPWGCGSCPSCK | (SEQ ID NO:215) |
| HVNELEEAFFKGEIEDFKVYKDKDGKKHFVYLMGEHPDFVVIIPSGHYIKIEQIREVK | |
| NFAYVKPALSRRKVIIIDDAHAMTSQAANALLKVLEEPPADTTFILTTNRRSAILPTILS | |
| RTFQVEFKGFSVKEVMEIAKVDEEIAKLSGGSLKRAILLKENKDILNKVKEFLENEPL | |
| KVYKLASEFEKWEPEKQKLFLEIMEELVSQKLTEEKKDNYTYLLDTIRLFKDGLARG | |
| VNEPLWLFTLAVQAD | |

The following DNA sequence encoding aq_{—}1526 (SEQ ID NO: 216) was downloaded and verified using the NCBI orf finder program. (The start and stop codons are underlined):

(SEQ ID NO:216)

actctctttcagacttaaagaaaagatacgggaaattctcggcgagaaatttgagcatttaataaaagttttaggggatataatttccgataatacacttg
aaacacctttaataacactcggtgaaggtaaaaacctcaagaaaaagtttaacgaggaattcatagaaacccttgagaatgcttcctattcaaacgag
gaggccggaaaattaagggaatttttaaagacactttacgctttggcactctgtaatagtagggaagaaatcactctggacctttgtgtaatattactcc
actccgaacagcacgcaactttagaaaaacaactcctttctctcgtacaaccgctactaaaaattgataaaaaaccgaagaaagaactgaacctata
gtccgcgaagttgcaaaggttataaacgaaacctgtttaaattaaatttgtATGgaaaaagttttttggaaaaactccagaaaaccttgcacatacc
cggaggactccttttttacggcaaagaaggaagcggaaagacgaaaacagcttttgaatttgcaaaaggtattttatgtaaggaaaacgtaccgtgg
ggatgcggagttgtccctcctgcaaacacgtaaacgagctggaggaagccttctttaaaggagaaatagaagactttaaagtttataaagacaag
gacggtaaaaagcacttcgtttaccttatgggcgaacatcccgactttgtggtaataatcccgagcggacattacataaagatagaacagataaggg
aagttaagaactttgcctatgtgaagcccgcactaagcaggagaaaagtaattataatagacgacgcccacgcgatgacctctcaggcggcaaac
gctcttttaaaggtattggaagagccacctgcggacaccacctttatcttgaccacgaacaggcgttctgcaatcctgccgactatcctctccagaac
ttttcaagtggagttcaagggcttttcagtaaaagaggttatggaaatagcgaaagtagacgaggaaatagcgaaactctctggaggcagtctaaaa
agggctatcttactaaaggaaaacaaagatatcctaaacaaagtaaaggaattcttggaaaacgagccgttaaaagtttacaagcttgcaagtgaatt
cgaaaagtgggaacctgaaaagcaaaaactcttccttgaaattatggaagaattggtatctcaaaaattgaccgaagagaaaaaagacaattacac
ctaccttcttgatacgatcagactctttaaagacggactcgcaaggggtgtaaacgaacctctgtggctgtttacgttagccgttcaggcggatTA
Ataaaccgttattgattccgtaacatttaaaccttaatctaaattatgagagcctttgaaggaggtctggtatggaaaatttgaagattagatatatagat
acgaggaagataggaaccgtgagcggtgtaaaagttccggtaaaaaagggctacaaaatagtcgtggaagacgag

For Thermotoga maritima, the primary homology was again found to the annotated dnax (TM0686; 478 amino acids; e=2×10⁻⁶⁴) however, two protein of secondary homology were identified: TM0508 (599 residues; e=1×10⁻⁴) and TM0771 (documented as γ-subunit related in the Thermotoga maritima TIGR database) (312 residues; e=6×10⁻⁴) The finding of three dnaX-like genes in one bacterium is unprecedented. Documented δ sequences fall in the 300-400 amino acid range, suggesting that TM0771 was the most likely Thermotoga maritima holB candidate. The following protein and DNA sequences were downloaded from the NCBI website; the start and stop codons (underlined below) of each orf was verified and the corresponding start and stop sites underlined within the DNA sequence:

| | |
|---|---|
| TM0508: | (SEQ ID NO:217) |

MSISEKLPLSELLRPKDFEDFVGQDHIFGNKGILRRTLKTGNMFSSILYGPPGSGKTSVF
SLLKRYFNGEVVYLSSTVHGVSEIKNVLKRGEQLRKYGKKLLLFLDEIHRLNKNQQ
MVLVSHVERGDIVLVATTTENPSFVIVPALLSRCRILYFKKLSDEDLMKILKKATEVL
NIDLEESVEKAIVRHSEGDARKLLNTLEIVHQAFKNKRVTLEDLETLLGNVSGYTKES
HYDFASAFIKSMRGSDPNAAVYYLVKMIEMGEDPRFIARRMIIFASEDVGLADPNAL
HIAVSTSIAVEHVGLPECLMNLVECAVYLSLAPKSNSVYLAMKKVQELPVEDVPLFL
RNPVTEEMKKRGYGEGYLYPHDFGGFVKTNYLPEKLKNEVIFQPKRVGFEEELFERL
RKLWPEKYGGESMAEVRKELEYKGKKIRIVKGDITREEVDAIVNAANEYLKHGGGV
AGAIVRAGGSVIQEESDRIVQERGRVPTGEAVVTSAGKLKAKYVIHTVGPVWRGGS
HGEDELLYKAVYNALLRAHELKLKSISMPAISTGIFGFPKERAVGIFSKAIRDFIDQHP
DTTLEEIRICNIDEETTKIFEEKFSV

(SEQ ID NO:218)

caagacaggatccgtacggtgaagcgggcgtggtggccatcttcactgaaaggatgctcaggggagaggaagttcacataftcggtgatggaga
gtacgtcagagactacgtttacgttgatgacgtggtcagagcgaatcttctcgccatggagaaaggcgacaacgaagtcttcaacataggaacgg
gaaggggcacaaccgtgaaccagctcttcaaactgctgaaagagatcactggctacgataaagagcctgtctataaaccaccgcgtaagggcga
cgtgagaaagagcattctcgattacacgaaggcgaaggaaaagctcggctgggaacccaaagtttcccttgaggagggattgaagctcaccgT
TGagtatttcagaaaaacccttgagtgaactgctcaggccgaaagacttcgaggatttcgtcggtcaggatcatatattcggtaataaagggattct
ccggcgaactttaaagacaggcaacatgttctcttctatcctctatggaccaccggggtctggtaagacctcggttttttcactgctgaaaaggtatttc
aacggcgaggtagtttatctgagctccaccgttcacggggtttctgaaataaagaacgttctcaaaagaggagaacagttgagaaaatatggaaaa
aaattgcttctctttctcgatgaaatacaccgcctgaacaaaaaccagcagatggttctggtttcccatgttgaacgaggagacatcgtactggttgcg
acaacaactgaaaatccgagttttgtcatcgtacctgcacttctctcaaggtgcaggattctttatttcaaaaactctctgacgaggacctgatgaaga
tcttgaaaaaagcaacagaggttctcaacatcgatctggaagaatcggtagagaaagccatagtaaggcattccgaaggagacgccagaaaactc
ttgaacaccctggagatcgtccaccaggcgttcaaaaacaagagagtaactcttgaggacctggaaactctgctgggaaacgtgagcgggtacac
gaaggaatcacactacgatttcgcttcggccttcataaagagtatgagaggtagcgatccaaacgccgctgtttactacctcgtcaaaatgatagag
atgggagaagacccgcgattcatagcacgaagaatgatcatattcgccagcgaagacgttggactcgccgacccaaacgctctacatatcgccgt
ttcgacctccatcgctgtcgaacacgtgggacttcccgaatgtttgatgaaccttgtagagtgtgccgtcatctttccctcgctcccaagagcaactct
gtttatctcgcaatgaaaaaagtccaggaacttcctgtggaggacgtaccgctttttctgagaaatcccgtcaccgaagagatgaaaaagcgtggat
acggagaagggtatctttatccccatgatttcggtggtttcgtgaaaacgaactatcttccagaaaagttgaaaaatgaggtcatcttccagccaaag
agagtaggttttgaggaagaactctttgaaaggctcagaaaactctggcccgagaagtacgggggtgaaagtatggccgaagtgagaaaagaac
tggaatacaaagggaaaaagatcagaatcgtaaagggagacatcacaagagaagaagtggacgccatagtgaacgcggcgaacgaatatctga
aacacggaggaggagtagcgggagcgatcgtgagagcaggtggaagcgttatccaggaagaaagcgacaggatcgttcaagaacgaggaag
agtcccgacaggtgaagcggtggtaaccagcgctggaaagctgaaggcaaaatacgtgatccacaccgtggggcccgtttggagaggaggca
gtcatggagaggacgaacttctctacaaagcagtttacaacgctcttcttcgagctcacgaactgaaattgaagagcatctcaatgcctgctatcagt
acaggaatattcggatttccgaaagaaagagcggtgggaatcttttcaaaagcaataagagatttcatcgatcaacatcctgataccactctggaag
agatccgcatatgcaacatagatgaggaaacaacgaagattttcgaagaaaagttcagcgttTGAtgaaaccaaaagaaggggcaaacgccc
cttcttttttaagacctctgaggaagtattgaatcaactcaaacgagttcttggacctcttccgcaacagatttccatacctgaaaggaactattgatatttt
catcataccacaaaaactaaaaaagtaaagggggccaagggccccctttatttccatcagaattcttcaggcatgctcggtgtttctttcttctcctcgg
gtttttcaacgatgagcacttccgttgtcaggagcattccagcgatggatgcagcaftctgcagggcgctccttgtaacctttgctgggtcaatgattc
ctctttcaaacatgttgcagtattctcctctgagtgcgtcgaatccataggctggatcgtcattcgaaagtatcttctcaatgatcaccgctccgtcgtat
cccgcgttttcagcaatctgcttaataggggctga

| | |
|---|---|
| TM0771: | (SEQ ID NO:219) |

mndlirkyakdqletlkriieksegisilingedlsyprevslelpeyvekfppkasdvleidpegenigiddirtikdflnyspelytr
kyvivhdcermtqqaanaflkaleeppeyavivlntrrwhyllptiksrvfrvvvnvpkefrdlvkekigdlweelpllerdfktale
ayklgaeklsglmeslkvletekllkkvlskglegylacrellerfskveskeffalfdqvtntitgkdaflliqrltriilhentwesved
qksvsfldsilrvkianlnnkltlmnilaihrerkrgvnaws

(SEQ ID NO:220)

ggaagatagctccgctccgcccaaacgtccggttgctcgttcagaatgtggtacagtatctcaagaccgtggtttgaagtacctacctcgtaagtgtc
gggaaaaacgagggctatccggagggaaatttcctctggatctttcacaacgctgttgatttctcttcctatgtatctacttggttttctaacccagggaa
gtgtttcattcaagaattttagaatcatcctttcacctcagttctgattttatcattggttgttataattagaaggcgaggtgagatcATGaacgatttgat
cagaaagtacgctaaagatcaactggaaactttgaaaaggatcatagaaaagtctgaaggaatatccatcctcataaatggagaagatctctcgtat
ccgagagaagtatcccttgaacttcccgagtacgtggagaaatttcccccgaaggcctcggatgttctggagatagatcccgagggggagaacat
aggcatagacgacatcagaacgataaaggacttcctgaactacagccccgagctctacacgagaaagtacgtgatagtccacgactgtgaaaga
atgacccagcaggcggcgaacgcgtttctgaaggcccttgaagaaccaccagaatacgctgtgatcgttctgaacactcgccgctggcattatcta
ctgccgacgataaagagccgagtgttcagagtggttgtgaacgttccaaaggagttcagagatctcgtgaaagagaaaataggagatctctggga
ggaacttccacttcttgagagagacttcaaaacggctctcgaagcctacaaacttggtgcggaaaaactttctggattgatggaaagtctcaaagtttt
ggagacggaaaaactcttgaaaaaggtcctttcaaaaggcctcgaaggttatctcgcatgtagggagctcctggagagattttcaaaggtggaatc
gaaggaattctttgcgctttttgatcaggtgactaacacgataacaggaaaagacgcgtttcttttgatccagagactgacaagaatcattctccacga
aaacacatgggaaagcgttgaagatcaaaaaagcgtgtctttcctcgattcaattctcagggtgaagatagcgaatctgaacaacaaactcactctg
atgaacatcctcgcgatacacagagagagaaagagaggtgtcaacgcttggagcTGAaggcaagggcagtgggagtggagataataccaa
agggtaagataatttactactctgtacccaacggtgatgagtacgagagaggagacctcgttctggtactgggagattttggacttgaagtgggaaa
ggttctgatacctcccagagaggtggtcatagacgaggtggggtacgaactcaaggccgtgatcagaaagttgacggatgaagatctggaacaat
acaggaagaatgtagaggatgcgttgaaggccttccagatatgtaagcagaaaataaaagaacacggtcttcccatgaagcttctctacgccaagt
acaccttcgatagaactaggctcatctttttcttcagtgcggaaggaagggttgatttcagagagcttgtaagagacctcgcaaagatcttcaaaaca
aggatagagttgaggcaagttggtgtgagagatgagatgaagttctttggaggccttggattgtgcggacttccgacatgctgttccacatttctcag
agagttcaccagcgtcacactgaagcacgcaaagaagcagcagatgatgatcaatccagcgaagatatccggaccatgcagaaggcttctgtgc
tgtcttacctacgaatacgatttctatgaaaaggagctggagggaattcctgacgaagggagcacgatcacctacgaaggaaagcgctacaaagt
cgtgaacgtgaacgtgtttctcagaacggtgactctcttctcggaaca

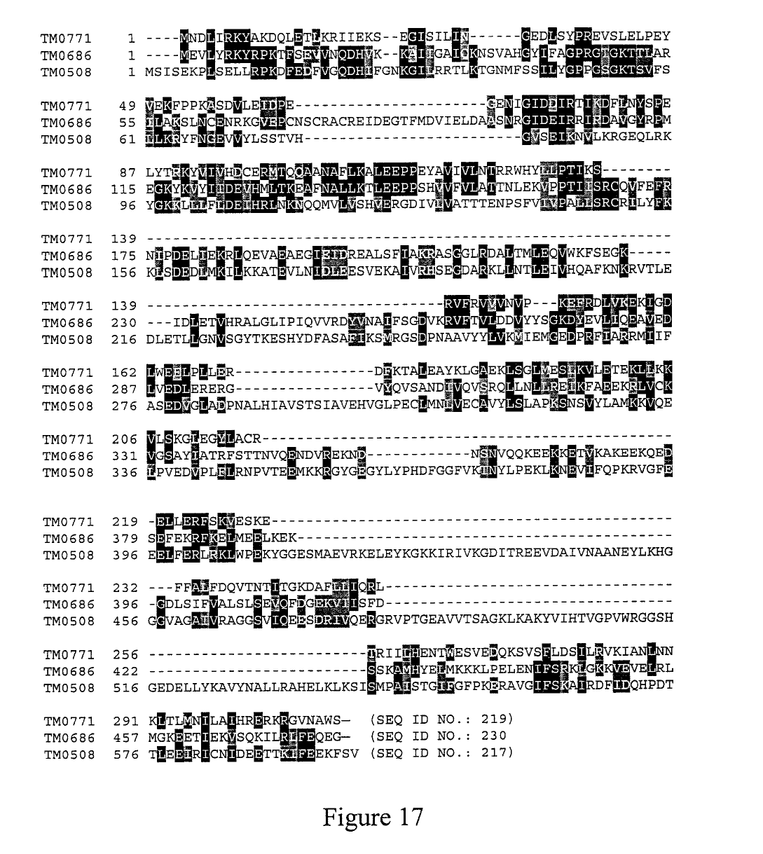

Rather than just relying on the homology score, the three dnaX like Thermotoga maritima proteins were aligned using Clustal so that their similarities and differences could be more closely scrutinized as shown in FIG. 17.

Both TM0686 and TM0508 appear, by sequence, to be functional ATPases. Both have perfect agreement with the Walker A motif (GXGKT(T/S) (SEQ ID NO: 221) found in all functional DnaX molecules, but lacking from δ's. Both have functional “DEAD” boxes of the type found in functional DnaX (DEhH, where h is a bulky hydrophobic residue), and both have an SRC sequence. TM0771 lacks a functional ATPase, and a reduced “DEAD-box” homology, a property shared with δ′ sequences. TM0771 and TM0686 share significant similarity through an 11-residue stretch of unknown function that is shared by other DnaX and δ′ proteins. TM0686 exhibits a perfect match to the NALLKTLEEPP (SEQ ID NO: 222) sequence found in both E. coli DnaX and δ′. SRV is found in TM0771 in place of the conserved SRC found in TM0686 and TM0508. These data suggest strongly that these three Thermotoga maritima proteins are part of a β processivity factor assembly apparatus and that TM0771 is the best holB candidate. TM0686 (annotated in the TIGR database as the Thermotoga maritima γ and τ subunits) and TM0508 (annotated as a hypothetical protein) are both likely functional ATPases in the DnaX complex. Perhaps TM0686 encodes the γ-homologue and the longer TM0508 the τ-homologue in Thermotoga maritima. Alternatively, with two distinct DNA polymerase III molecules in Thermotoga maritima, TM0508 and TM0686 may serve different polymerase IIIs. All candidates will be expressed and employ them to reconstitute a Thermotoga maritima processive DNA polymerase III holoenzyme.

### Example 13

Expression of Aquifex aeolicus holB

PCR will be used to amplify the Aquifex holB gene (AQ_{—}1526). The primers will be designed so that a PstI site will be added to the 5′ end of the gene and a KpnI site to the 3′ end of the gene.

The forward primer will be:

5′-gatcctgcagGAAAAAGTTTTTTTGGAAAAACTCC-3′ (SEQ ID NO: 164)

The complementary portion of this primer is shown as upper case and begins at codon #2 (the ATG start codon of the Aquifex holB gene will be excluded). The PstI site is shown in italics and underlined. This will place the open reading frame (ORF) of the Aquifex holB gene in frame with the N-terminal fusion peptide. There is a 4 base extension on the 5′ end of the primer to allow for efficient digestion by PstI.

The reverse primer will be:

5′-gatcggtaccttaTTAATCCGCCTGAACGGCTAACG-3′ (SEQ ID NO: 165)

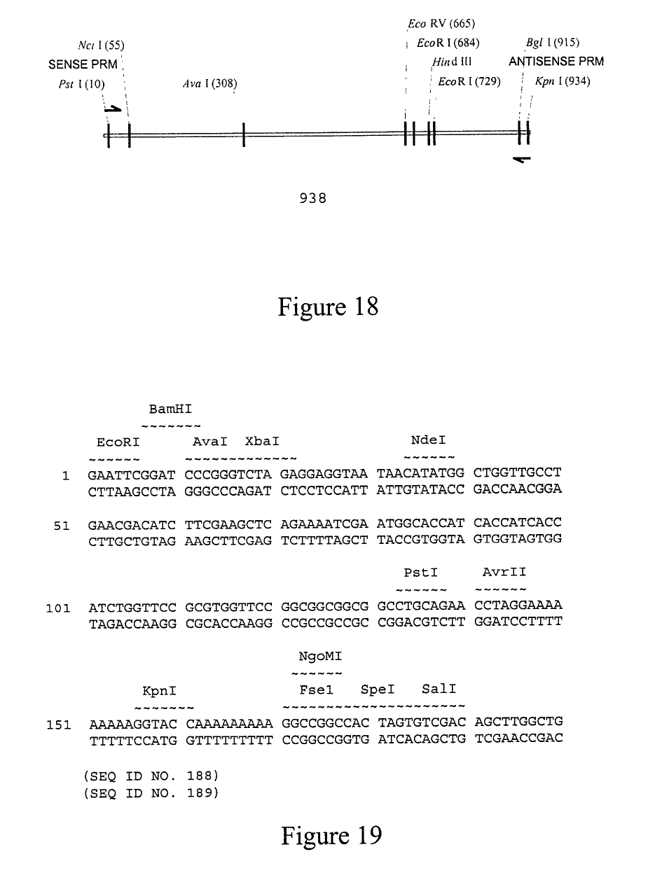

The complementary portion of the primer is shown as upper case. The KpnI restriction site is in italics and underlined and there is a 4 base extension to allow for efficient digestion by KpnI. The non-complementary portion also contains an additional stop codon (tta) to enhance termination of translation. A graphical depiction of the PCR product is shown in FIG. 18.

The PCR product will be digested with PstI and KpnI and inserted into the vector pA1-NB-Avr2 (74) digested with the same restriction enzymes. The region of pA1-NB-Avr2 that the PCR product will be inserted into is shown in FIG. 19.

These plasmids (pA1-NB-AQD′) will be transformed into DH5α bacteria and screened for ampicillin resistance, isolated from positive isolates and the sequence of the isolated plasmids will verified as described above and expressed in E. coli MGC1030 and AP1.L1.

To express the Aquifex δ subunit in an untagged form, the gene (AQ_{—}1526) will be amplified from Aquifex genomic DNA using primers designed to allow insertion of the gene into a vector in which the gene can be expressed in a native form.

The forward primer will be designed so that the start “ATG” codon will incorporated into an NdeI restriction site. The forward primer is shown below.

5′-gatccatATGGAAAAAGTTTTTTTGGAAAAACTCC-3′ (SEQ ID NO: 166)

The complementary portion of the primer is shown as upper case. The NdeI restriction site is in italics and underlined and there is a 4 base extension to allow for efficient digestion by NdeI.

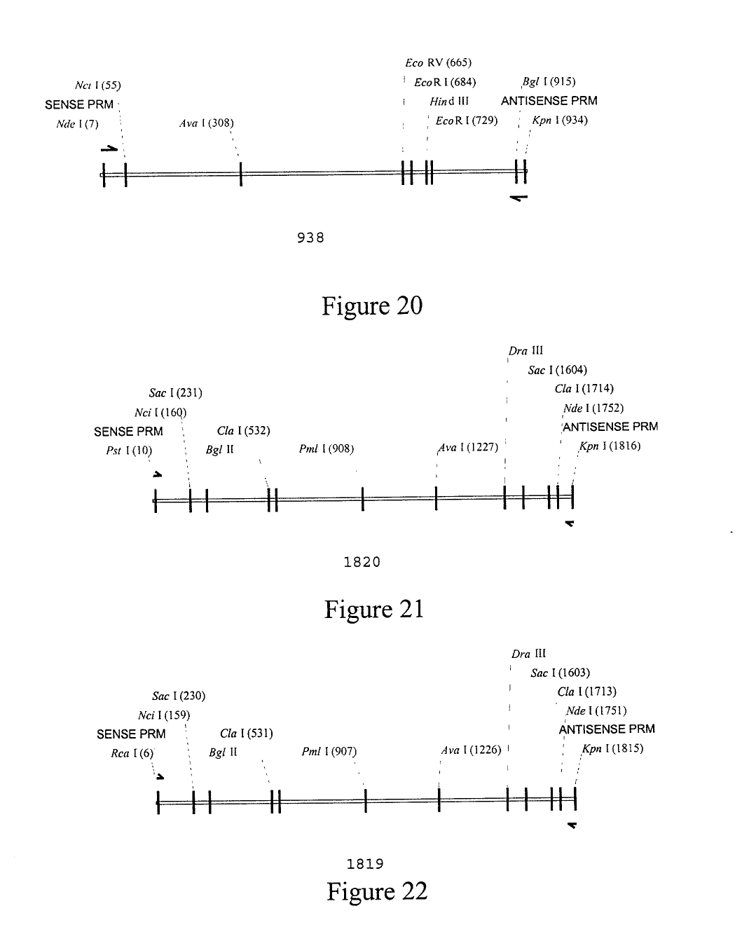

The reverse primer is the same reverse primer used in construction of pA1-NB-AQD′ (SEQ ID NO: 165) and contains a KpnI restriction site and an addition stop codon adjacent to the native stop codon. A graphical depiction of the PCR product is shown in FIG. 20.

The PCR product will be digested with NdeI and KpnI and inserted into the vector pA1-CB-NdeI digested with the same restriction enzymes. The region of pA1-CB-NdeI that the PCR product will be inserted into is shown in FIG. 14. These plasmids (pA1-AQD′) will be transformed into DH5α screened screened for ampicillin resistance, isolated from positive isolates and the sequence of the isolated plasmids will verified as described above and expressed in E. coli MGC1030 and AP1L1.

### Example 14

Purification of Aquifex aeolicus δ′

Aquifex aeolicus δ′, both in tagged and native (untagged) form will be purified using the approaches and strategies outlined in Example 9.

### Example 15

Expression and Purification of Aquifex aeolicus Replication Proteins: α, ε, β, DnaX and SSB

Native and tagged Aquifex aeolicus α, ε, β, DnaX and SSB will be expressed and purified using the general strategies described in the preceding examples and in references (Kim, D. R. and McHenry, C. S., ibid.; Dallmann, H. G. and McHenry, C. S., J Biol Chem 270:29563-29569 (1995); Dallmann, H. G., et al., J Biol Chem 270:29555-29562 (1995); PCT WO99/13060). α will be purified using a gap-filling assay on nuclease-activated DNA to monitor its presence, yield and specific activity. ε will be purified using its intrinsic nuclease activity in the presence and absence of α to monitor its purification (Maki and Kornberg, (1987) ibid.; Griep, M., et al., Biochemistry 29:9006-9014 (1990); Reems, J., et al., J. Biol. Chem. 266:4878-4882 (1991)). β and DnaX will initially be purified as tagged proteins and once a reconstitution assay is developed, they will be purified in native form. SSB will be expressed in native form and purified by established procedures (Griep, M. and McHenry, C., J. Biol. Chem. 264:11294-11301 (1989); Lohman, T. M., et al., Biochemistry 25:21 (1986)).

### Example 16

Development of Reconstitution Assays for Subunits of the Aquifex aeolicus DNA Polymerase III Holoenzyme

For both E. coli and T. thermophilus, a combination of the α, DnaX, β, δ and δ′ subunits results in reconstitution of a minimally processive replicase capable of replicating a long single-stranded template, such as primed M13Gori DNA (Kornberg, and Baker, ibid.; PCT WO99/13060). The ε subunit of the E. coli DNA polymerase III core stabilizes and stimulates the α subunits polymerization activity; thus, it too will be included in the anticipated replication reaction, as convenient. Once the Aquifex aeolicus replication subunits are available in tagged form, they will be mixed together with primed M13Gori DNA primed with an annealed DNA oligonucleotide primer or primed in a separate reaction by the action of E. coli SSB and E. coli DnaG primase. dNTPs, ATP and Mg⁺⁺ will be included at the experimentally determined optimal concentrations (initially 1 mM ATP, 10 mM Mg⁺⁺, 150 μM dCTP, dATP, dGTP and 50 μM [³H] TTP (ca. 400 cpm/pmol)). Reactions will be conducted at the experimentally-determined optimum temperature (starting at 65° C.) and performed for a time when the amount of DNA synthesis is proportional to time (initially 5 min.). Reactions will be compared with Tth proteins as a control. If roughly the same level of synthesis is observed with the Aquifex aeolicus proteins as the tagged Tth control proteins, optimization of the reaction will follow.

If poor synthesis is observed with approximately the same level of tagged Aquifex aeolicus proteins as used in the Tth control reaction, levels of individual tagged proteins (or groups of proteins) will be increased in an attempt to optimize synthesis. Higher levels of a protein or proteins may be required if missing proteins are required for optimal function of the complex or if tags interfere with critical protein-protein interactions. If increasing levels of tagged proteins does not result in reconstitution of an efficient reaction, ammonium sulfate cuts for individual subunits (or groups of subunits) for the corresponding tagged proteins will be substituted to determine which protein(s) are defective in tagged form. The ammonium sulfate concentration required to yield a precipitate optimized for protein recovery without inclusion of unnecessary contaminants will be determined using antibodies for quantitative Western Blots in advance of the availability of a functional assay. If an ammonium sulfate fraction of an untagged protein is found to be more active than a tagged purified protein, it will be substituted for the tagged protein in the reconstituted reaction. If problems remain, individual Aquifex aeolicus proteins will be expressed at decreased (15° C.) or increased (43° C.) temperatures for varying times or in the presence of co-overprodued chaperones (GroEL.GroES) and/or (DnaK/DnaJ/GrpE) to try to increase the relative levels of properly folded active proteins.

Once an optimal reaction is reconstituted, in the presence of individual limiting subunits, the level of the other subunits required to give an optimal reaction with minimal background will be determined. Whether the synthesis obtained in individual reactions is proportional to the level of the limiting protein added will then be determined. This linear response is necessary to use the assay as a tool for quantifying the level of the limiting protein.

Once the Aquifex aeolicus reconstitution reaction is established as a quantitative tool, it can be used to monitor and quantify the purification of individual native untagged Aquifex aeolicus replication proteins. Varying quantities of protein fractions containing unknown levels of a specific Aquifex aeolicus subunit will be added to reactions containing optimal levels of the other subunits. Values obtained where at least two levels of protein give proportional responses will be used for quantitation. The level of a given protein required to give one pmole of DNA synthesis (total nucleotide incorporated) in one minute under optimal reaction conditions as one unit will be expressed. As individual untagged proteins become available in pure form, they will be substituted for the untagged protein in the reconstitution assay. This will require reoptimization of the assay in terms of levels of the added untagged protein. If the untagged protein binds more tightly to the other components than its tagged counterpart, lower levels might be required to achieve optimal synthesis.

### Example 17

Tests of Interactions of Aquifex aeolicus δ and δ′ Subunits

In addition to the requirement for candidate Aquifex aeolicus δ′ and δ to reconstitute the Aquifex aeolicus replicase in the presence of the required components, being able to demonstrate function and implied utility in reconstituted replication reactions by showing these protein interact with each other and other components of the Aquifex aeolicus replicase is anticipated.

It is anticipated that expressed, purified Aquifex aeolicus δ and δ′ will interact with each other. Demonstration of an interaction will demonstrate that each participate in the same functional unit, providing evidence that each was appropriately identified and that a functional protein is expressed. Levels of δ and δ′ sufficient for detection upon a ten-fold dilution will be mixed and subjected to gel filtration on a Sepharose 12 column. Protein will be detected by either Coomassie-staining of SDS polyacrylamide gels of individual eluted fractions or by Western-blots of gels blotted to membranes. Individual δ and δ′ will be subjected to gel filtration under identical conditions. An interaction is indicated by a change in elution position of both δ and δ′ to a position to a higher molecular weight with both proteins present in roughly stoichiometric ratios in the eluted fractions. If an interaction is not observed, the concentration of δ and δ′ will be increased up to 10 μM.

If an interaction is not observed, it may be due to a requirement for DnaX for a tight complex to form. If an interaction is not observed between δ and δ′ alone, DnaX will be included in the mixture subjected to gel filtration and gels analyzed to see if δ and δ′ shift to a higher molecular weight and co-elute with DnaX.

It is also expected that Aquifex aeolicus δ will interact with Aquifex aeolicus β. δ and β will be mixed and subjected to gel filtration as described above in this section for δ and δ′. If an interaction is not observed, Aquifex aeolicus β will be mixed with δ-δ′ and DnaX in the presence of ATP to test an interaction. This is the complex that is expected to be functional in the assembly of β onto primed DNA (Naktinis, V., et al., ibid.).

### Example 18

Reconstitution of Aquifex aeolicus DNA Polymerase III Holoenzyme

Once all Aquifex aeolicus replication proteins are available in purified form they will be combined and used for purposes of (i) synthesis of DNA at high temperatures (temperatures at or above 60° C.), (ii) synthesis of high molecular weight DNA in applications requiring either a single high temperature or thermal cycling, (iii) processive synthesis of high molecular weight DNA (processive indicates incorporation of greater than 1000 nucleotides per association-elongation-polymerase dissociation event), (iv) rapid synthesis of DNA at rates greater than 200 nucleotides/second, (v) highly accurate DNA synthesis resulting from inclusion of the c subunit, and (vi) for processive reactions in the presence of expressed Aquifex aeolicus SSB or E. coli SSB.

### Example 19

Expression of Thermotoga maritima Protein TM0508 (Hypothetical Second DnaX Protein)

PCR will be used to amplify the T. maritima gene (TM0508). The primers will be designed so that a PstI site will be added to the 5′ end of the gene and a KpnI site to the 3′ end of the gene.

The forward primer will be:

5′-gatcctgcagAGTATTTCAGAAAAACCCTTG-3′ (SEQ ID NO: 167)

The complementary portion of this primer is shown as upper case and begins at codon #2 (the ATG (TTG) start codon of the T. maritima gene (TM0508) will be excluded). The PstI site is shown in italics and underlined. This will place the open reading frame of the T. maritima gene (TM0508) inframe with the N-terminal fusion peptide. There is a 4 base extension on the 5′ end of the primer to allow for efficient digestion by PstI.

The reverse primer will be:

5′-gatcggtacctaaTCAAACGCTGAACTTTTCTTCG-3′ (SEQ ID NO: 168)

The complementary portion of the primer is shown as upper case. The KpnI restriction site is in italics and underlined and there is a 4 base extension to allow for efficient digestion by KpnI. The non-complementary portion also contains an additional stop codon (tta) to enhance termination of translation, A graphical depiction of the PCR product is shown in FIG. 21.

The PCR product will be digested with PstI and KpnI and inserted into the vector pA1-NB-Avr2 (74) digested with the same restriction enzymes. The region of pA1-NB-Avr2 that the PCR product will be inserted into is shown in FIG. 19. These plasmids (pA1-NB-TM[0508]) will be transformed into DH5α bacteria and screened for ampicillin resistance, isolated from positive isolates and the sequence of the isolated plasmids will verified as described above and express in E. coli MGC1030 and AP1.L1.

To express the T. maritima subunit [TM0508] in an untagged form, the gene (TM0508) will be amplified from T maritima genomic DNA using primers designed to allow insertion of the gene into a vector in which the gene can be expressed in a native form. The TM0508 gene contains internal NdeI sites which will not allow the use of the pA1-CB-NdeI expression vector. This problem was circumvented by using an expression vector (pA1-CB-NcoI) which contained an NcoI restriction site in place of the NdeI restriction site. The NdeI restriction site contained the “ATG” used as the start codon at the end of the restriction site (catatg). The NcoI restriction site however, contains the “ATG” (c/catgg) internally. The first two codons of the TM0508 gene are “atgagt”, which code for Met and Ser. Thus, this restriction site could not be use as part of the forward primer and allow both insertion into the NcoI restriction site of pA1-CB-NcoI and retain both codons encoding Met and Ser. This problem was overcome by adding the RcaI restriction site (t/catga) to the forward primer. This restriction site allows the PCR product cleaved with RcaI to be spliced into pA1-CB-NcoI cleaved with NcoI. This will destroy both restriction sites but will allow the TM0508 gene to be inserted into the pA1-CB-NcoI expression vector. The forward primer is shown below.

5′-gatctcATGAGTATTTCAGAAAAACCC-3′ (SEQ ID NO: 169)

The complementary portion of the primer is shown as upper case. The RcaI restriction site is in italics and underlined and there is a 4 base extension to allow for efficient digestion by RcaI.

The reverse primer is the same reverse primer used in construction of pA1-NB-TM[0508] (SEQ ID NO: 168) and contains a KpnI restriction site and an addition stop codon adjacent to the native stop codon. A graphical depiction of the PCR product is shown in FIG. 22.

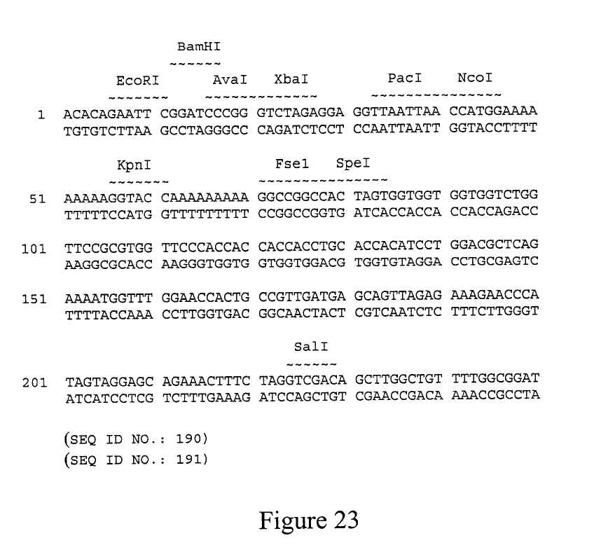

The PCR product will be digested with RcaI and KpnI and inserted into the vector pA1-CB-NcoI digested with the same restriction enzymes. The region of pA1-CB-NcoI that the PCR product will be inserted into is shown in FIG. 23.

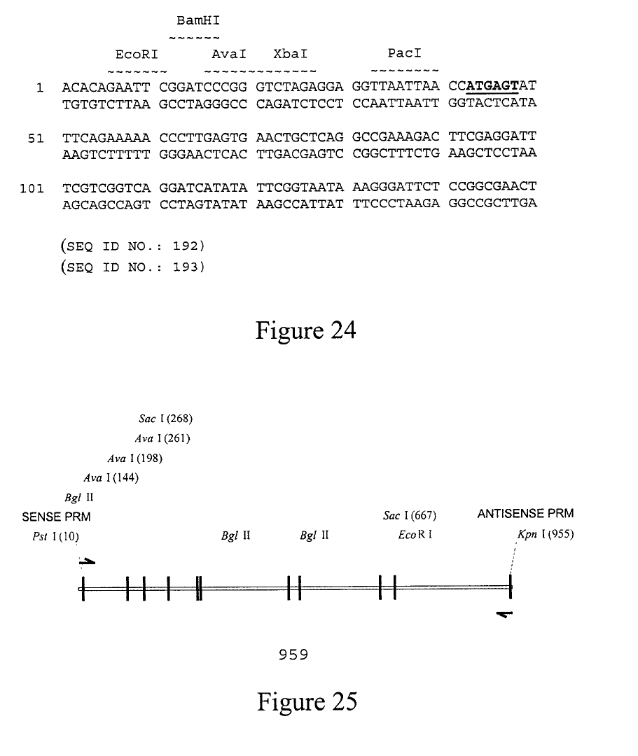

As described in the preceeding paragraphs, insertion of the RcaI/KpnI digested PCR product into the NcoI/KpnI digested pA1-CB-NcoI (74) resulted in the plasmid pA1-TM[0508]. The region of pA1-TM[0508] containing the site in which the RcaI digested restriction site was spliced into the NcoI restriction site of pA1-CB-NcoI is shown in FIG. 24.

The first two codons of the TM0508 gene in pA1-TM[0508] are shown in bold underlined text in FIG. 24. Both the NcoI and the RcaI restrictions no longer exist in this plasmid. These plasmids (pA1-TM[0508]) will be transformed into DH5α bacteria and screened for ampicillin resistance, isolated from positive isolates and the sequence of the isolated plasmids will verified as described above and express in E. coli MGC1030 and AP1.L1.

Note: This ‘second’ DnaX-like protein from Thermotoga maritima does not meet the full consensus sequence criteria for a DnaX protein as specified in this application, but is sufficiently DnaX-like that it will be expressed and tested in reconstituted Thermotoga DNA polymerase III holoenzyme reactions as described to test whether it constitutes an additional required or adjunct protein not found in other bacterial replicases to date.

### Example 20

Expression of Thermotoga maritima Protein TM0771 (Hypothetical HolB (δ′))

Primers will be designed so that a PstI site will be added to the 5′ end of the gene and a KpnI site to the 3′ end of the gene.

The forward primer will be:

5′-gatcctgcagAACGATTTGATCAGAAAGTACGC-3′ (SEQ ID NO: 170)

The complementary portion of this primer is shown as upper case and begins at codon #2 (the ATG start codon of the T. maritima δ′ gene (TM0771)will be excluded). The PstI site is shown in italics and underlined. This will place the open reading frame of the T. maritima δ′ gene (TM0771) in frame with the N-terminal fusion peptide. There is a 4 base extension on the 5′ end of the primer to allow for efficient digestion by PstI.

The reverse primer will be:

5′-gatcggtaccttaTCAGCTCCAAGCGTTGACACC-3′ (SEQ ID NO: 171)

The complementary portion of the primer is shown as upper case. The KpnI restriction site is in italics and underlined and there is a 4 base extension to allow for efficient digestion by KpnI. The non-complementary portion also contains an additional stop codon (tta) to enhance termination of translation. A graphical depiction of the PCR product is shown in FIG. 25.

The PCR product will be digested with PstI and KpnI and inserted into the vector pA1-NB-Avr2 digested with the same restriction enzymes. The region of pA1-NB-Avr2 that the PCR product will be inserted into is shown in FIG. 19. These plasmids (pA1-NB-TMD′[0771]) will be transformed into DH5α bacteria and screened for ampicillin resistance, isolated from positive isolates and the sequence of the isolated plasmids will verified as described above and expressed in E. coli MGC1030 and AP1.L1.

To express the T. maritima δ′ subunit [0771] in an untagged form, the gene (TM0771) will be amplified from T. maritima genomic DNA using primers designed to allow insertion of the gene into a vector in which the gene can be expressed in a native form.

The forward primer will be designed so that the start “ATG” codon will incorporated into an NdeI restriction site. The forward primer is shown below.

5′-gatccatATGAACGATTTGATCAGAAAGTACGC-3′ (SEQ ID NO: 172)

The complementary portion of the primer is shown as upper case. The NdeI restriction site is in italics and underlined and there is a 4 base extension to allow for efficient digestion by NdeI.

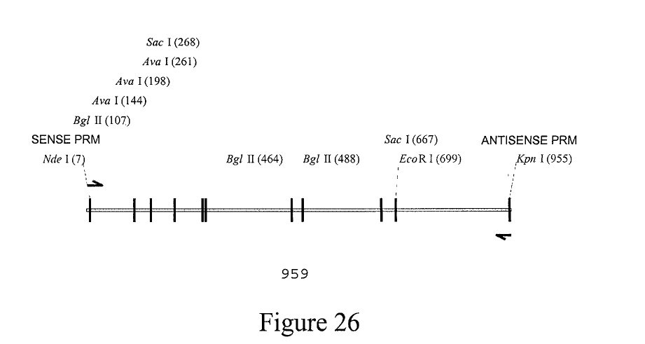

The reverse primer is the same reverse primer used in construction of pA1-NB-TMD′[0771] (SEQ ID NO: 171) and contains a KpnI restriction site and an addition stop codon adjacent to the native stop codon. A graphical depiction of the PCR product is shown in FIG. 26.

The PCR product will be digested with NdeI and KpnI and inserted into the vector pA1-CB-NdeI digested with the same restriction enzymes. The region of pA1-CB-NdeI that the PCR product will be inserted into is shown in FIG. 14. These plasmids (pA1-TMD′[0771]) will be transformed into DH5α screened screened for ampicillin resistance, isolated from positive isolates and the sequence of the isolated plasmids will verified as described above and expressed in E. coli MGC1030 and AP1.L1.

### Example 21

Purification of Thermotoga maritima Protein TM0508 (Hypothetical Second DnaX Protein)

Thermotoga maritima TM0508, both in tagged and native (untagged) form will be purified using the approaches and strategies outlined in Example 9. Being able to monitor the purification of the native protein will be dependent on its requirement for reconstitution of a Thermotoga maritima replicase.

### Example 22

Purification of Thermotoga maritima Protein TM0771 (Hypothetical HolB (δ′))

Thermotoga maritima δ′, both in tagged and native (untagged) form will be purified using the approaches and strategies outlined in Example 3 except the reconstitution assays described under Example 18 will be employed.

### Example 23

Expression of Components of the Thermotoga maritima DNA Polymerase III Holoenzyme: α Subunit of DNA polymerase III Type 1, α Subunit of DNA Polymerase III Type II, ε, β, DnaX and SSB.

Native and tagged Thermotoga maritima α both type I and type II, TM 0461 and TM 0576, respectively), ε (TM0496), β (TM0262), DnaX (TM 0686) and SSB (TM 0604) will be expressed and purified using the general strategies described in the preceding examples and in references (Kim, D. R. and McHenry, C. S., ibid.); Dallmann, H. G. and McHenry, C. S., J Biol Chem 270:29563-29569 (1995); Dallmann, H. G., et al., J Biol Chem 270:29555-29562 (1995); PCT WO99/13060; PCT WO9953074). Both α subunits (from type I and II DNA polymerase IIIs) will be purified using a gap-filling assay on nuclease-activated DNA to monitor its presence, yield and specific activity. ε will be purified using its intrinsic nuclease activity in the presence and absence of αfrom type I DNA polymerase III) to monitor its purification (Maki and Kornberg (I1987) ibid.; Griep, M., et al., (1 990) ibid.; Reems, J., et al., ibid.). β and DnaX will initially be purified as tagged proteins and once a reconstitution assay is developed, they will be purified in native form. SSB will be expressed in native form and purified by established procedures (general SSB refs).

### Example 24

Development of Reconstitution Assays for Subunits of the Thermotoga maritima DNA Polymerase III Holoenzyme

Reconstitution assays for components of the Thermotoga maritima replicase will be developed using the general procedures and strategies described for Aquifex aeolicus in Example 16 except all tests will be done with either the type I α subunit (plus ε when convenient) or type II α or both a: subunits in combination. Experiments will also be performed with the annotated Thermotoga maritima DnaX (TM0686) or putative second DnaX (TM 0508) alone or in combination.

### Example 25

Tests of Interactions of Thermotoga maritima δ and δ′ Subunits

Experiments to test the identity and function of purified Thermotoga maritima δ and δ′ will be as described in Example 17 except for those experiment involving DnaX, either TM0686 or the second putative DnaX (TM 0508) will be included alone or in combination.

### Example 26

Reconstitution of Thermotoga maritima DNA Polymerase III Holoenzyme

The Thermotoga maritima DNA polymerase III holoenzyme will be reconstituted using the components identified in the preceding examples as being necessary or stimulatory for the purposed described under Example 18 for the Aquifex aeolicus DNA polymerase III holoenzyme.

### Example 27

Extension of the Procedures Reported in Examples 1-26 to the Detection of Subunits of the DNA Polymerase III Holoenzyme from all other Bacteria

The general procedures described in the introduction and the preceding examples should be generally applicable to any bacteria that is sequenced. Particularly, our procedures for finding the δ component, δ′ components and additional DnaX-like components should permit these components to be identified and expressed. These components, together with the more easily-identified α, ε, β and primary DnaX proteins should reconstitute DNA polymerase III holoenzyme for use in synthesis of DNA as described under Example 18 plus any additional applications that involves synthesis of DNA or discovering inhibitors of that process.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifiations of the described modes for carrying out the invention which are obvious to those skilled in the relevant arts are intended to be within the scope of the following claims.

### Example 28

Expression of DNA Polymerase III Holoenzyme Subunits from Bacteria from any Phyla, Purification, Reconstitution of Holoenzyme, and Test of Function

The subunits of DNA polymerase III holoenzyme identified in this application or holoenzyme subunits identified from bacteria sequenced in the future using approaches described in this application will be expressed, purified and used to reconstitute active DNA polymerase III holoenzyme by methods and using assays described for Thermotoga maritima and Aquifex aeolicus in this application and for Yersinia pestis, Pseudomonas aeruginosa, Streptococcus pyogenes, Thermus thermophilus and other organisms in the referenced patents and patent applications. The authenticity of the identified δ subunits in this application can be further verified by their requirment to reconstitute DNA polymerase III holoenzyme activity and/or by their interaction with β or δ′ subunits from the cognate organism or other bacteria. DNA polymerase III holoenzyme reconstitutions assays can take place using subunits from the cognate organism or different organisms.

TABLE 3
DnaX proteins identified from PSI-BLAST search of completely
sequenced genomes

| | Accession | GenBank gi | |
|---|---|---|---|
| Organism | number | number | Database |
| Aquifex aeolicus | A70460, | 7514776, 2984127 | NCBI+¹ |
| | AAC07663.1 | | |
| Bacillus halodurans | BAB03753.1 | 10172646 | NCBI+ |
| Bacillus subtilis | S13786, | 98292, 467409, | NCBI+ |
| | BAA05255.1, | 580914, 2632286, | |
| | CAA34877.1, | 118807 | |
| | CAB11795.1, | | |
| | P09122 | | |
| Buchnera | BAB13178.1 | 10039144 | NCBI+ |
| Borrelia burgdorferi | D70157, | 7463153, 2688379 | NCBI+ |
| | AAC66831.1 | | |
| Campylobacter jejuni | G81320, | 11346858, 6968590 | NCBI+ |
| | CAB73411.1 | | |
| Caulobacter crescentus | AAK22254.1, | 13421402, 1293572 | NCBI+ |
| | AAB61695.1 | | |
| Chlamydia muridarum | D81682, | 11360902, 7190650 | NCBI+ |
| | AAF39442.1 | | |
| Chlamydia trachomatis | A71528, | 7468911, 3328753 | NCBI+ |
| | AAC67929.1 | | |
| Chlamydophila | C72127, | 7468224, 4376294, | NCBI+ |
| pneumonia | AAD18193.1, | 7189650, 8978415 | |
| | AAF38540.1, | | |
| | BAA98252.1 | | |
| Deinococcus | C75278, | 7471826, 6460225 | NCBI+ |
| radiodurans | AAF11953.1 | | |
| Haemophilus influenzae | P43746, F6411, | 1169397, 1075209, | NCBI+ |
| | AAC22882.1 | 1574159 | |
| Helicobacter pylori J99 | A71906, | 7464040, 4155207 | NCBI+ |
| | AAD06231.1 | | |
| Helicobacter pylori | E64609, | 7464043, 2313841 | NCBI+ |
| 26695 | AAD07767.1 | | |
| Lactococcus lactis | AAK06292.1 | 12725258 | NCBI+ |
| Mesorhizobium loti | NP_106158.1 | 13474589 | NCBI+ |
| Mycobacterium leprae | CAA19155.1, | 3150103, 13093951 | NCBI+ |
| | CAC31851.1 | | |
| Mycobacterium | O69688, B70796, | 6014997, 7477980, | NCBI+ |
| tuberculosis | CAA18043.1 | 2960145 | |
| Mycoplasma genitalium | NP_073091.1, | 12045280, | NCBI+ |
| | P47659, D64246, | 1352305, 1361493, | |
| | AAC71649.1 | 3845015 | |
| Mycoplasma | NP_110307.1, | 13508357, | NCBI+ |
| pneumoniae | P75177, S73550, | 2494198, 2146109, | |
| | AAB95872.1 | 1673890 | |
| Neisseria meningitidis | D81860, | 11261620, 7380298 | NCBI+ |
| Z2491 | CAB84884.1, | | |
| Pasteurella multocida | AAK02448 | 12720609 | NCBI+ |
| Pseudomonas | A83455, AE004581 | 11348451, 9947490 | NCBI+ |
| aeruginosa | | | |
| Rickettsia prowazekii | A71649, | 7467603, 3861390 | NCBI+ |
| | CAA15289.1 | | |
| Synechocystis | S77213, | 7469304, 1652627 | NCBI+² |
| | BAA17547.1 | | |
| Thermotoga maritima | D72344, | 7462368, 4981209 | NCBI+ |
| | AAD35768.1 | | |
| Treponema pallidum | G71254, | 7521020, 3323329 | NCBI+ |
| | AAC65953.1 | | |
| Ureaplasma urealyticum | NP_077917.1, | 13357643, | NCBI+ |
| | F82935, AAF30492.1 | 11356842, 6899039 | |
| Vibrio cholerae | H82246, | 11261617, 9655520 | NCBI+ |
| | AAF94213.1 | | |
| Xylella fastidiosa | B82635, | 11261615, 9106885 | NCBI+ |
| | AAF84614.1 | | |

¹A plus symbol next to the database name indicates that the protein is annotated as a DnaX or tau/gamma protein. A minus symbol next to the database indicates that the protein is not annotated.
²The mature form of DnaX from Synechocystis is 685 amino acids in length, but the native protein before the intein is removed is 1116 amino acids in length. The entries in NCBI indicate the longer unprocessed form of Synechocystis DnaX.

TABLE 4
δ′ proteins identified in PSI-BLAST searches of all finished
genomes at NCBI

| | Original | | |
|---|---|---|---|
| | accession | GenBank gi | |
| Organism | number³ | number⁴ | Database |
| Aquifex aeolicus | D70432 | 7514459 | NCBI⁵ −⁶ |
| Bacillus halodurans | BAB03763 | 10172656 | NCBI+ |
| Bacillus subtilis | S66061 | 2126931 | NCBI+ |
| Buchnera | P57435 | 11132951 | NCBI+ |
| Borrelia burgdorferi | AAC67118 | 2688709 | NCBI− |
| Campylobacter jejuni | CAB75220 | 6968051 | NCBI− |
| Caulobacter crescentus | AAK23798 | 13423258 | NCBI+ |
| Chlamydia muridarum | F81700 | 11360903 | NCBI− |
| Chlamydia trachomatis | D71546 | 7468912 | NCBI− |
| Chlamydophila pneumonia | E72098 | 7468225 | NCBI− |
| Deinococcus radiodurans | F75287 | 7473469 | NCBI− |
| Haemophilus influenzae | P43748 | 2506369 | NCBI+ |
| Helicobacter pylori J99 | G64673 | 7464041 | NCBI+ |
| Helicobacter pylori 26695 | AAD06725 | 4155746 | NCBI+ |
| Lactococcus lactis | AAK04497 | 12723272 | NCBI+ |
| Mesorhizobium loti | NP_102222 | 13470653 | NCBI+ |
| Mycobacterium leprae | CAA18816 | 3097239 | NCBI− |
| Mycobacterium tuberculosis | E70563 | 7477702 | NCBI− |
| Mycoplasma genitalium | NP_072667 | 12044857 | NCBI− |
| Mycoplasma pneumoniae | P75105 | 1673807 | NCBI+ |
| Neisseria meningitidis Z2491 | C81945 | 11353892 | NCBI+ |
| Pasteurella multocida | AAK03758 | 12722081 | NCBI+ |
| Pseudomonas aeruginosa | D83275 | 11348448 | NCBI+ |
| Rickettsia prowazekii | H71727 | 7467604 | NCBI+ |
| Synechocystis | S76227 | 7469488 | NCBI− |
| Thermotoga maritima | A72337 | 7462369 | NCBI− |
| Treponema pallidum | AAC65508 | 3322812 | NCBI− |
| Ureaplasma urealyticum | G82944 | 11356841 | NCBI+⁷ |
| Vibrio cholerae | H82127 | 11280161 | NCBI+ |
| Xylella fastidiosa | C82777 | 11360901 | NCBI+ |
| ³An accession number applies to the complete record and is usually a combination of letter(s) and numbers. Accession numbers do not change, even if information in the record is changed at the author's request. Records from different databases of reference sequences have different accession number formats. | | | |
| ⁴GenInfo Identifier (GI) sequence identification numbers are assigned consecutively by NCBI to each sequence it processes for nucleotide and amino acid sequences. If a sequence changes in any way, a new GI number will be assigned. A separate GI number is also assigned to each protein translation within a nucleotide sequence record, and a new GI is assigned if the protein translation changes in any way. Unlike accession numbers, GI numbers allow all sequences to be | | | |
| #maintained and therefore appears to be a better recording method. | | | |

⁵GenBank is the NIH genetic sequence database, an annotated collection of all publicly available DNA sequences. GenBank (at NCBI), together with the DNA DataBank of Japan (DDBJ) and the European Molecular Biology Laboratory (EMBL) comprise the International Nucleotide Sequence Database Collaboration. These three organizations exchange data on a daily basis.
⁶A plus symbol next to the database name indicates that the protein is annotated as a δ′ or probable δ′ protein. A minus symbol next to the database indicates that the protein is not annotated as a δ′ protein or is annotated as a tau/gama like protein. The nucleotide and amino acid sequences of proteins with a minus symbol are shown below the table.
⁷Ureaplasma urealyticum and Xylella fastidiosa are annotated as δ instead of δ′.

TABLE 5
DnaX Proteins from S. pyogenes, S. pneumoniae and S. aureus

| | Accession | GenBank gi | |
|---|---|---|---|
| | number/gene | number/reading | |
| Organism | location | frame | Database |
| Streptococcus | AAF98348 | 9789547 | NCBI + |
| pyogenes⁸ | contig1, position | | |
| | 1130862-1132529 at | | |
| | Univ. of Okla. | | |
| Streptococcus | contig 3836, position | +1 | TIGR − |
| pneumoniae | 705181-706833 | | |
| Staphylococcus | BAB41666 | 13700368 | NCBI + |
| aureus | | | |

⁸The protein sequenc of Streptococcus pyogenes DnaX protein was initially submitted to GenBank by Bruck, I. and O'Donnell, M. and Rockefeller University/HHMI, 1230 York Ave, New York, NY 10021, USA on Jun. 21, 2000 and published Aug. 10, 2000.

TABLE 6
δ′ Proteins from S. pyogenes, S. pneumoniae and S. aureus

| | Accession | GenBank gi | |
|---|---|---|---|
| | number/gene | number/reading | |
| Organism | location | frame | Database |
| Streptococcus | AAF98347 | 11992993 | NCBI + |
| pyogenes⁹ | | | |
| Streptococcus | contig 3836, position | +3 | TIGR − |
| pneumoniae | 781821-782675 | | |
| Staphylococcus | BAB41672 | 13700374 | NCBI + |
| aureus | | | |
| ⁹The protein sequenc of Streptococcus pyogenes δ′ protein was initially submitted to GenBank by Bruck, I. and O'Donnell, M. and Rockefeller University/HHMI, 1230 York Ave, New York, NY 10021, USA on Jun. 21, 2000 and published Aug. 10, 2000. This was the wrong sequence. The incorrect sequence was replaced by the correct sequence on Dec. 27, 2000. The corrected sequence did not contain the C-terminal Glu reisdue. | | | |
| #However, in the PCT #WO 01/09164 A2 the correct sequence is shown in its entirety. Another Streptococcus pyogenes δ′ protein from the streptococcus pyogenes M1 GAS strain was submitted to GenBank on Apr. 10, 2001 and published on Jun. 1, 2001 by Streptoccus pyogenes Genome Sequencing Project at the University of Oklahoma. | | | |

TABLE 7
δ proteins identified in PSI-BLAST searches of unfinished
bacterial genomes at NCBI

| | Accession | GenBank gi | |
|---|---|---|---|
| Organism | number | number | Database |
| Mycoplasma pulmonis | CAC13625 | 14089876 | NCBI |
| Streptococcus agalactiae | CAA72898 | 2765186 | NCBI |
| Streptomyces coelicolor | CAB66242 | 6714670 | NCBI |

TABLE 8
DnaX proteins corresponding to δ proteins identified from PSI-
BLAST search of unfinished genomes at NCBI

| | Accession | GenBank gi | |
|---|---|---|---|
| Organism | number | number | Database |
| Mycoplasma pulmonis | CAC13222 | 14089462 | NCBI + |
| Streptococcus agalactiae | Not found | | NCBI |
| Streptomyces coelicolor | CAB56347 | 5918469 | NCBI + |

TABLE 9
δ′ Proteins corresponding to δ proteins identified from PSI-
BLAST search of unfinished genomes at NCBI

| | Accession | GenBank gi | |
|---|---|---|---|
| Organism | number | number | Database |
| Mycoplasma pulmonas | CAC13226 | 14089466 | NCBI − |
| Streptococcus agalactiae | Not found | | NCBI |
| Streptomyces coelicolor | T36661 | 7480627 | NCBI + |

TABLE 10
DnaX proteins corresponding to δ proteins identified in BLAST
searches of phylogenically related organisms

| | | Reading | |
|---|---|---|---|
| Organism | Gene Location or Protein | Frame | Database |
| Actinobacillus | Contig 106, positions 9219-11261 | +3 | Univ. of |
| actinomycetemcomitans | | | Oklahoma − |
| Bacillus anthracis | Contig 4047, positions 9897-11585 | +3 | TIGR |
| Bordatella pertussis | Genomic positions 1938958-1940010 | +1 | Sanger − |
| Burkholderia | Genomic positions 113078-114112 | +2 | Sanger − |
| pseudomallei | | | |
| Chlorobium tepidum | contig 3499, position 798393-800252 | −2 | TIGR − |
| Chloroflexus | Contig 1016, positions 682-2133 | −3 | DOE-Joint |
| aurantiacus | | | Genome |
| | | | Institute − |
| Clostridum difficile | Contig 907 positions 3472-4407 | +1 | Sanger − |
| Corynebacterium | Genomic positions 221399-223522 | +2 | Sanger − |
| diphtheriae | | | |
| Cytophaga hutchinsonii | Contig 123, positions 1345-3077 | −3 | DOE-Joint |
| | | | Genome |
| | | | Institute |
| Dehalococcoides | Contig 6145, positions 43320-44988 | +3 | TIGR |
| ethenogenes | | | |
| Prochlorococcus | Contig 26, positions 834903-836642 | −1 | DOE-Joint |
| marinus | | | Genome |
| | | | Institute |
| Porphyromonas | Genomic positions 1503207-1505012 | TIGR | TIGR |
| gingivalis | | locus | |
| | | PG1418 | |
| Salmonella typhi | genomic position 533757-535685 | +3 | Sanger − |
| Yersenia pestis | genomic position 3478956-3480929 | −1 | Sanger − |

TABLE 11
δ′ proteins corresponding to δ proteins identified in BLAST
searches of phylogenically related organisms

| | Gene Location or | Reading | |
|---|---|---|---|
| Organism | Protein | Frame | Database |
| Actinobacillus | Contig 73, positions 16499-17482 | +2 | Univ. of |
| actinomycetemcomitans | | | Oklahoma − |
| Bacillus anthracis | Contig 3937, positions 5108-6091 | +2 | TIGR |
| Bordatella pertussis | Genomic positions 1938958-1940010 | +1 | Sanger − |
| Burkholderia | Genomic positions 113078-114112 | +2 | Sanger − |
| pseudomallei | | | |
| Chlorobium tepidum | Contig 3499, positions | +2 | TIGR |
| | 1065254-1066426 | | |
| Chloroflexus aurantiacus | Contig 1088, positions 13027-14049 | −1 | DOE-Joint |
| | | | Genome |
| | | | Institute |
| Clostridum difficile | Contig 907 positions 3472-4407 | +1 | Sanger − |
| Corynebacterium | Genomic positions 304135-305310 | +1 | Sanger − |
| diphtheriae | | | |
| Cytophaga hutchinsonii | Contig 154, positions 10459-11586 | +1 | DOE-Joint |
| | | | Genome |
| | | | Institute |
| Dehalococcoides | Contig 6152, positions 30780-31799 | +2 | TIGR |
| ethenogenes | | | |
| Prochlorococcus | Contig 26, positions 641321-642280 | +2 | DOE-Joint |
| marinus | | | Genome |
| | | | Institute |
| Porphyromonas | Genomic positions 988645-989892 | TIGR | TIGR |
| gingivalis | | locus | |
| | | PG0932 | |
| Salmonella typhi | Genomic positions 1193926-1194930 | +1 | Sanger |
| Yersinia pestis | Genomic position 1829514-1830536 | +3 | Sanger |

TABLE 12
DnaE and Pol C Proteins corresponding to δ proteins
identified in PSI-BLAST searches of finished genomes at NCBI

| | | Accession | GenBank | |
|---|---|---|---|---|
| Organism | Protein | number | gi number | Database |
| Aquifex aeolicus | DnaE | O67125 | 6014991 | NCBI−¹⁰ |
| Bacillus halodurans | DnaE | Q9K838 | 14194680 | NCBI + |
| Bacillus subtilis | DnaE | O34623 | 6166145 | NCBI + |
| Buchnera | DnaE | P57332 | 11132275 | NCBI + |
| Borrelia burgdorferi | DnaE | B70172 | 7434830 | NCBI − |
| Campylobacter jejuni | DnaE | Q9PPI9 | 14194692 | NCBI − |
| Caulobacter crescentus | DnaE | AAK- | 13423381 | NCBI + |
| | | 23901 | | |
| Chlamydia muridarum | DnaE | Q9PJJ7 | 14194688 | NCBI − |
| Chlamydia trachomatis | DnaE | O84549 | 14194664 | NCBI − |
| Chlamydophila | DnaE | Q9Z7N8 | 14194705 | NCBI − |
| pneumonia | | | | |
| Deinococcus | DnaE | Q9RX08 | 14194695 | NCBI − |
| radiodurans | | | | |
| Haemophilus influenzae | DnaE | P43743 | 1169392 | NCBI + |
| Helicobacter pylori J99 | DnaE | Q9ZJF9 | 11132624 | NCBI + |
| Helicobacter pylori | DnaE | P56157 | 2494191 | NCBI + |
| 26695 | | | | |
| Lactococcus lactis | DnaE | Q9C170 | 14194667 | NCBI + |
| Mesorhizobium loti | DnaE | NP_{—} | 13471011 | NCBI + |
| | | 102580 | | |
| Mycobacterium leprae | DnaE | Q9X7F0 | 14194699 | NCBI − |
| Mycobacterium | DnaE | Q10779 | 1706493 | NCBI − |
| tuberculosis | | | | |
| Mycoplasma genitalium | DnaE | NP_{—} | 12045116 | NCBI − |
| | | 072927 | | |
| Mycoplasma | DnaE | NP_{—} | 13508117 | NCBI + |
| pneumoniae | | 110066 | | |
| Neisseria meningitidis | DnaE | Q9JXZ2 | 14194679 | NCBI + |
| MC58 | | | | |
| Neisseria meningitidis | DnaE | Q9JVX8, | 14194676, | NCBI + |
| Z2491 | | Q9JVX8 | 14194676 | |
| Pasteurella multocida | DnaE | Q9CPK3 | 14194670 | NCBI + |
| Pseudomonas | DnaE | Q9HXZ1 | 14194674 | NCBI + |
| aeruginosa | | | | |
| Rickettsia prowazekii | DnaE | O05974 | 6226600 | NCBI + |
| Synechocystis | DnaE | A59016 | 7434836 | NCBI − |
| Thermotoga maritima | DnaE | Q9ZHG4 | 7227889 | NCBI − |
| Treponema pallidum | DnaE | O83675 | 6014994 | NCBI − |
| Ureaplasma urealyticum | DnaE | NP_078251 | 13357977 | NCBI + |
| Vibrio cholerae | DnaE | G82100, | 11261593, | NCBI + |
| | | P52022 | 14195659 | |
| Xylella fastidiosa | DnaE | C82834, | 11261591, | NCBI + |
| | | Q9PGU4 | 14194684 | |
| Bacillus subtilis | PolC | AAA22666 | 143342 | NCBI− |
| Bacillus halodurans | PolC | Q9KA72 | 13959348 | NCBI + |
| Lactococcus lactis | PolC | AAK06222 | 12725180 | NCBI + |
| Mycoplasma genitalium | PolC | NP_072691 | 12044881 | NCBI + |
| Mycoplasma | PolC | NP_109722 | 13507773 | NCBI − |
| pneumoniae | | | | |
| Thermotoga maritima | PolC | Q9ZHF6 | 6225287 | NCBI − |
| Ureaplasma urealyticum | PolC | NP_078211 | 13357937 | NCBI + |

¹⁰A plus symbol next to the database name indicates that the protein is annotated asa DnaE/PolC protein. A minus symbol next to the database indicates that the protein is not annotated

TABLE 13
DnaE and PolC proteins from S. pyogenes, S. pneumoniae and S.
aureus

| | | Accession | GenBank gi | |
|---|---|---|---|---|
| | | number/ | number/reading | |
| Organism | protein | gene location | frame | Database |
| Staphylococcus | DnaE | BAB42792 | 13701498 | NCBI +¹¹ |
| aureus | | | | |
| Streptococcus | DnaE | AAF98350 | 9789551 | NCBI + |
| pyogenes | | | | |
| Streptococcus | DnaE | Contig 3836, | +3 | TIGR − |
| pneumoniae | | positions 740775- | | |
| | | 743876 | | |
| Staphylococcus | PolC | Q53665 | 13959683 | NCBI + |
| aureus | | | | |
| Streptococcus | PolC | AAF98345 | 9789541 | NCBI + |
| pyogenes | | | | |
| Streptococcus | PolC | Contig 3836, | +1 | TIGR − |
| pneumoniae | | positions 144313- | | |
| | | 148704 | | |

¹¹A plus symbol next to the database name indicates that the protein is annotated as a DnaE/PolC protein. A minus symbol next to the database indicates that the protein is not annotated

TABLE 14
DnaE and Pol C Proteins Corresponding to Additional δ
Proteins Identified from PSI-BLAST search of
Partially Sequenced Genomes

| | | | GenBank gi | |
|---|---|---|---|---|
| Organism | protein | Accession number | number | Database |
| Mycoplasma | Dna E | CAC13892 | 14090134 | NCBI +¹² |
| pulmonis | | | | |
| Streptococcus | Dna E | Not found | | |
| agalactiae | | | | |
| Streptomyces | Dna E | Q53665, CAC36747 | 14194704, | NCBI + |
| coelicolor | | | 13620707 | |
| Mycoplasma | PolC | CAC13848 | 14090090 | NCBI + |
| pulmonis | | | | |
| Streptococcus | PolC | Not found | | |
| agalactiae | | | | |
| Streptomyces | PolC | Not found | | |
| coelicolor | | | | |

¹²A plus symbol next to the database name indicates that the protein is annotated as a DnaE/PolC protein. A minus symbol next to the database indicates that the protein is not annotated

TABLE 15
DnaE and PolC proteins corresponding to δ proteins identified
in BLAST searches of phylogenically related organisms

| | | Gene Location or | Reading | |
|---|---|---|---|---|
| Organism | Protein | Protein | Frame | Database |
| Actinobacillus | DnaE | Contig 264, position | −1 | Univ. of |
| actinomycetemcomitans | | 9232-12705 | | Oklahoma −¹³ |
| Bacillus anthracis | DnaE | Contig 3830, positions | −2 | TIGR |
| | | 46303-49629 | | |
| Bordatella pertussis | DnaE | Genomic position | −3 | Sanger − |
| | | 2465147-2468635 | | |
| Burkholderia | DnaE | Contig 181 positions | −1 | Sanger − |
| pseudomallei | | 61605-65129 | | |
| Chlorobium tepidum | DnaE | Contig 3499, positions | +1 | TIGR − |
| | | 354713-358282 | | |
| Chloroflexus | DnaE | Partial sequence | | DOE-Joint |
| aurantiacus | | corresponding to | | Genome |
| | | different regions of | | Institute − |
| | | DnaE found in | | |
| | | Contigs 918, 349, 261, | | |
| | | 1137 | | |
| Clostridum difficile | DnaE | Contig 4 positions | −3 | Sanger − |
| | | 65634-69203 | | |
| Corynebacterium | DnaE | Genomic positions | −1 | Sanger − |
| diphtheriae | | 1609896-1606336 | | |
| Cytophaga hutchinsonii | DnaE | Contig 152 positions | −1 | DOE-Joint |
| | | 55726-59310; Gene 48 | | Genome |
| | | | | Institute + |
| Dehalococcoides | DnaE | Contig 6150, positions | +1 | TIGR − |
| ethenogenes | | 75823-79336 | | |
| Prochlorococcus | DnaE | Contig 24, positions | +3 | DOE-Joint |
| marinus | | 147546-151043; | | Genome |
| | | gene105 | | Institute + |
| Porphyromonas | DnaE | TIGR gene PG0035 | | TIGR + |
| gingivalis | | | | |
| Salmonella typhi | DnaE | Genomic positions | +3 | Sanger − |
| | | 266430-269909 | | |
| Yersinia pestis | DnaE | Genomic positions | +1 | Sanger − |
| | | 1200879-1204361 | | |
| Bacillus anthracis | PolC | Contig 3835, positions | −1 | TIGR |
| | | 27724-32019 | | |
| Clostridum difficile | PolC | Contig 792, positions | +2 | Sanger − |
| | | 15028-19326 | | |

¹³A plus symbol next to the database name indicates that the protein is annotated as a DnaE/PolC protein. A minus symbol next to the database indicates that the protein is not annotated.

TABLE 16
β Proteins corresponding to δ identified from PSI-BLAST search
of completely sequenced genomes

| Organism | Accession number | GenBank gi number | Database |
|---|---|---|---|
| Aquifex aeolicus | O67725, C70462, | 3913514, 7434840, | NCBI +¹⁴ |
| | AAC07688.1 | 2984154 | |
| Bacillus halodurans | Q9RCA1, BAA82686.1, | 13959350, 5672648, | NCBI + |
| | BAB03721.1 | 10172614 | |
| Bacillus subtilis | P05649, B22930, | 118797, 80346, | NCBI + |
| | CAA26218.1, BAA05238.1, | 40015, 467392, | |
| | CAB11778.1 | 2632269 | |
| Buchnera | P57127, BAB12739.1, | 11132241, 10038704, | NCBI + |
| | AAG33956.1, P29439, | 11320921, 232008, | |
| | JC1159, AAA73150.1, | 94507, 144150, | |
| | AAC38107.1, AAG33955.1, | 2827015, 11320919, | |
| | AAK21713.1, | 13398088, 13398084, | |
| | AAK21711.1, AAK21716.1, | 1339809513398078, | |
| | AAG33944.1, AAK21701.1, | 11320897, 13398063, | |
| | AAK21702.1, AAK21703.1, | 13398065, 13398067, | |
| | AAK21704.1, AAK21705.1, | 13398069, 13398072, | |
| | AAK21706.1, AAK21707.1, | 13398074, 13398076, | |
| | AAK21709.1, AAK21710.1, | 13398080, 13398082, | |
| | AAK21712.1, AAK21714.1, | 13398086, 13398090, | |
| | AAK21715.1, AAK21717.1, | 13398093, 13398097, | |
| | AAK21718.1, AAK21720.1, | 13398099, 13398103, | |
| | AAK21721.1, AAK21719.1, | 13398105, 13398101, | |
| | AAG33948.1, AAG33945.1, | 11320905, 11320899, | |
| | AAG33947.1, AAG33949.1, | 11320903, 11320907, | |
| | AAG33943.1, AAG33951.1, | 11320895, 11320911, | |
| | AAG33954.1, AAG33942.1, | 11320917, 11320893. | |
| | AAF19207.1, AAF19206.1, | 6606556, 6606554, | |
| | AAF19208.1, AAG33950.1, | 6606558, 11320909, | |
| | AAG33953.1, AAG33952.1, | 11320915, 11320913, | |
| | AAG33946.1, AAF19209.1 | 11320901, 6606560 | |
| Borrelia burgdorferi | E70154, AAB91514.1, | 7463155, 2688357, | NCBI + |
| | P33761, S34948, AAA58942.1 | 461953, 479619, | |
| | | 454040 | |
| Campylobacter jejuni | E81415, CAB72495.1 | 11346582, 6967507 | NCBI + |
| Caulobacter crescentus | P48198, AAB51448.1, | 1352300, 1049324, | NCBI + |
| | AAK22143.1 | 13421269 | |
| Chlamydia muridarum | Q9PKW4, C81713, | 14194691, 11261632, | NCBI + |
| | AAF39208.1 | 7190388 | |
| Chlamydia trachomatis | O84078, E71559, | 14194661, 7468908, | NCBI + |
| | AAC67666.1 | 3328470 | |
| Chlamydophila | Q9Z8K0, BAA98549.1, | 14194707, 8978712, | NCBI + |
| pneumonia | H72090, AAD18487.1, | 7434845, 4376617, | |
| | F81578, AAF38262.1 | 11261630, 7189343 | |
| Deinococcus | D75571, AAF09595.1 | 7471825, 6457660 | NCBI + |
| radiodurans | | | |
| Haemophilus influenzae | P43744, A64107, | 1169393, 1074026, | NCBI + |
| | AAC22654.1 | 1574022 | |
| Helicobacter pylori J99 | Q9ZLX4, A71931, | 11132657, 7464044, | NCBI + |
| | AAD06030.1 | 4154988 | |
| Helicobacter pylori | O25242, D64582, | 3913505, 7464039, | NCBI + |
| 26695 | AAD07565.1 | 2313610 | |
| Lactococcus lactis | AAK04100, O54376, T30306, | 12722837, 3913511, | NCBI + |
| | AAC12964.1 | 7474141, 2909714 | |
| Mesorhizobium loti | NP_106216, BAB52002.1 | 13474647, 14025402 | NCBI + |
| Mycobacterium leprae | P46387, T10002, | 1169395, 7434842, | NCBI + |
| | AAB53142.1, CAA94709.1, | 886326, 1262353, | |
| | CAC29510.1 | 13092414 | |
| Mycobacterium | Q50790, F70850, | 3913519, 7434846, | NCBI + |
| tuberculosis | CAA16239.1, AAK44225.1, | 3261513, 13879043, | |
| | AAG00841.1 | 9845532 | |
| Mycoplasma genitalium | NP_072661.1, P47247, | 12044851, 6226601, | NCBI + |
| | AAC71217.1 | 3844620 | |
| Mycoplasma | NP_109689, Q50313, S62836, | 13507740, 2494195, | NCBI + |
| pneumoniae | S73479, AAG34739.1 | 2127610, 2146107, | |
| | | 11379480 | |
| Neisseria meningitidis | C81030, AAF42232.1 | 11353125, 7227158 | NCBI + |
| MD58 | | | |
| Neisseria meningitidis | A81974, A81974, | 11353891, 11353891, | NCBI + |
| Z2491 | CAB83846.1 | 7379292 | |
| Pasteurella multocida | AAK03244.1 | 12721508 | NCBI + |
| Pseudomonas | Q917C4, F83644, | 13959347, 11348446, | NCBI + |
| aeruginosa | AAG03392.1 | 9945820 | |
| Rickettsia prowazekii | Q9ZDB3, B71700, | 6225282, 7434844, | NCBI + |
| | CAA14876.1 | 3860976 | |
| Synechocystis | P72856, S74720, BAA16871.1 | 2494196, 7469303, | NCBI + |
| | | 1651945 | |
| Thermotoga maritima | E72400, AAD35350.1 | 7434843, 4980758 | NCBI + |
| Treponema pallidum | O83048, A71378, | 4033379, 7434841, | NCBI + |
| | AAC65000.1 | 3322257 | |
| Ureaplasma urealyticum | NP_077909, C82937, | 13357635, 11356839, | NCBI + |
| | AAF30484.1 | 6899030 | |
| Vibrio cholerae | Q9KVX5, F82376, | 14547987, 11354872, | NCBI + |
| | AAF93191.1 | 9654404 | |
| Xylella fastidiosa | H82859, AAF82815.1 | 11360900, 9104762 | NCBI + |

¹⁴A plus symbol next to the database name indicates that the protein is annotated as β protein. A minus symbol next to the database indicates that the protein is not annotated.

TABLE 17
β Proteins from S. pyogenes, S. pneumoniae and S. aureus

| | Accession | GenBank gi | |
|---|---|---|---|
| | number/gene | number/reading | |
| Organism | location | frame | Database |
| Streptococcus | AAK33147 | 13621327 | NCBI +¹⁵ |
| pyogenes | | | |
| Streptococcus | O06672 | 3913504 | NCBI + |
| pneumoniae | | | |
| Staphylococcus | S54708 | 1084187 | NCBI + |
| aureus | | | |

¹⁵A plus symbol next to the database name indicates that the protein is annotated as β protein. A minus symbol next to the database indicates that the protein is not annotated.

TABLE 18
β Proteins Corresponding to δ Proteins Identified in BLAST
Searches of Phylogenically Related Organisms

| | | Reading | |
|---|---|---|---|
| Organism | Gene Location or Protein | Frame | Database |
| Actinobacillus | Contig 213, position 10095-11192 | +3 | Univ. of |
| actinomycetemcomitans | | | Oklahoma −¹⁶ |
| Bacillus anthracis | Contig 3902, positions 32884-34024 | +1 | TIGR − |
| Bordatella pertussis | Contig 142 positions 503449-504558 | −1 | Sanger − |
| Burkholderia | Contig 298 positions 65824-66927 | −1 | Sanger − |
| pseudomallei | | | |
| Chlorobium tepidum | Contig 3499, positions 1708292-1707174 | −2 | TIGR − |
| Chloroflexus | Contig 1129, positions 766-1900 | +1 | DOE-Joint |
| aurantiacus | | | Genome |
| | | | Institute − |
| Clostridum difficile | Contig 871, positions 1457-2665 | +2 | Sanger − |
| Corynebacterium | Genomic positions 2316-3503 | +3 | Sanger − |
| diphtheriae | | | |
| Cytophaga hutchinsonii | Contig 132 positions 47216-48340; | −2 | DOE-Joint |
| | Gene 21 | | Genome |
| | | | Institute + |
| Dehalococcoides | Contig 6139, positions 20994-22129 | −1 | TIGR − |
| ethenogenes | | | |
| Prochlorococcus | Contig 26, positions 767092-768248; | −3 | DOE-Joint |
| marinus | gene 877 | | Genome |
| | | | Institute + |
| Porphyromonas | Contig GPG.con, positions | −1 | TIGR − |
| gingivalis | 1953200-1952090; TIGR gene | | |
| | PG1853 | | |
| Salmonella typhi | Genomic positions 3806530-3807632 | +1 | Sanger − |
| Yersinia pestis | Genomic positions 4617236-4618336 | −2 | Sanger − |

¹⁶A plus symbol next to the database name indicates that the protein is annotated as β protein. A minus symbol next to the database indicates that the protein is not annotated.

TABLE 19

| | conserved regions | | | | | | |
|---|---|---|---|---|---|---|---|
| | 17-38 | 64-82 | 97-117 | 154-191 | 215-260 | 298-331 | |
| | number of conserved aa per region | | | | | | regions meeting |
| Organism | 10 | 11 | 10 | 18 | 23 | 12 | criteria |
| Aquifex aeolicus | 7 | 5 | 7 | 15 | 16 | 7 | 6 |
| Bacillus halodurans | 7 | 7 | 8 | 12 | 15 | 10 | 6 |
| Bacillus subtilis | 9 | 6 | 7 | 10 | 16 | 11 | 6 |
| Buchnera sp. | 8 | 8 | 7 | 11 | 14 | 10 | 6 |
| Borrelia burgdorferi | 8 | 7 | 5 | 8 | 17 | 10 | 5 |
| Campylobacter jejuni | 5 | 8 | 5 | 13 | 11 | 7 | 6 |
| Caulobacter crescentus | 6 | 7 | 6 | 10 | 9 | 7 | 5 |
| Chlamydophila pneumoniae | 2 | 7 | 4 | 9 | 14 | 6 | 4 |
| Chlamydia muridarum | 2 | 5 | 5 | 10 | 13 | 6 | 5 |
| Chlamydia trachomatis | 4 | 7 | 4 | 9 | 15 | 6 | 4 |
| Deinococcus radiodurans | 4 | 7 | 5 | 15 | 15 | 11 | 5 |
| Escherichia coli | 9 | 9 | 9 | 15 | 22 | 10 | 6 |
| Haemophilus influenzae | 10 | 9 | 10 | 14 | 19 | 9 | 6 |
| Helicobacter pylori 26695 | 5 | 7 | 4 | 9 | 12 | 3 | 4 |
| Helicobacter pylori J99 | 5 | 7 | 4 | 9 | 11 | 3 | 4 |
| Lactococcus lactis | 8 | 8 | 7 | 5 | 17 | 11 | 5 |
| Mesorhizobium loti | 7 | 7 | 8 | 8 | 12 | 7 | 5 |
| Mycobacterium leprae | 8 | 9 | 4 | 10 | 14 | 11 | 5 |
| Mycobacterium tuberculosis | 7 | 9 | 4 | 10 | 13 | 9 | 5 |
| Mycoplasma genitalium | 7 | 2 | 5 | 15 | 9 | 8 | 4 |
| Mycoplasma pneumoniae | 7 | 6 | 6 | 15 | 14 | 9 | 6 |
| Neisseria meningitidis Z2491 | 9 | 9 | 7 | 16 | 17 | 9 | 6 |
| Pasteurella multocida | 8 | 8 | 10 | 14 | 20 | 8 | 6 |
| Pseudomonas aeruginosa | 9 | 10 | 9 | 14 | 20 | 7 | 6 |
| Rickettsia prowazekii | 6 | 7 | 6 | 11 | 10 | 5 | 4 |
| Streptococcus pneumoniae | 5 | 8 | 7 | 4 | 15 | 10 | 5 |
| Streptococcus pyogenes | 7 | 6 | 7 | 6 | 16 | 12 | 5 |
| Staphylococcus aureus | 5 | 3 | 3 | 12 | 16 | 5 | 3 |
| Streptomyces coelicolor | 8 | 10 | 6 | 7 | 17 | 12 | 5 |
| Synechocystis | 6 | 5 | 7 | 11 | 14 | 11 | 6 |
| Thermotoga maritima | 4 | 8 | 6 | 13 | 14 | 9 | 5 |
| Thermus thermophilus | 5 | 5 | 6 | 13 | 17 | 11 | 6 |
| Treponema pallidum | 5 | 8 | 5 | 10 | 16 | — | 5 |
| Ureaplasma urealyticum | 5 | 4 | 6 | 12 | 15 | 9 | 5 |
| Vibrio cholerae | 10 | 9 | 10 | 16 | 17 | 7 | 6 |
| Xylella fastidiosa | 8 | 9 | 6 | 13 | 15 | 8 | 6 |

TABLE 20

| | | | δ sequences |
|---|---|---|---|
| | Sequencing | δ sequences producing | producing no |
| Organism | Center | hits^{a} | hits |
| Actinobacillus | The University | Escherichia coli, | Dehalococcoides |
| actinomycetemcomitans | of Oklahoma | Neisseria meningitidis | ethenogenes, |
| | Advanced Center | | Campylobacter |
| | for Genome | | jejuni, |
| | Technology | | Caulobacter |
| | | | crescentus, |
| | | | Ureaplasma |
| | | | urealyticum, |
| | | | Chlamydia |
| | | | trachomatis, |
| | | | Synechocystis, |
| | | | Escherichia coli |
| Bacillus anthracis | TIGR, the | Bacillus subtilis, | Escherichia coli |
| | Institute for | Lactococcus lactis | |
| | Genomic | | |
| | Research | | |
| Bordatella | The Sanger | Neisseria meningitidis, | |
| pertussis | Centre | Escherichia coli | |
| Burkholderia | The Sanger | Neisseria meningitidis, | |
| pseudomallei | Centre | Escherichia coli, | |
| | | Bordatella pertussis | |
| Chlorobium | TIGR, the | Cytophaga hutchinsonii, | Chlamydia |
| tepidum | Institute for | Porphyromonas | muridarum, |
| | Genomic | gingivalis | Chlamydia |
| | Research | | trachomatis, |
| | | | Chlamydophila |
| | | | pneumonia, |
| | | | Synechocystis, |
| | | | Ureaplasma |
| | | | urealyticum, |
| | | | Mycobacterium |
| | | | leprae, |
| | | | Escherichia coli |
| Chloroflexus | DOE-Joint | Dehalococcoides | Treponema |
| anrantiacus | Genome Institute | ethenogenes, | pallidum, |
| | | Escherichia coli | Helicobacter |
| | | | pylori |
| Clostridum | The Sanger | Lactococcus lactis, | Escherichia coli |
| difficile | Centre | Streptococcus pyogenes | |
| Corynebacterium | The Sanger | Mycobacterium leprae, | Escherichia coli |
| diphtheriae | Centre | Streptomyces coelicolor | |
| Cytophaga | DOE-Joint | Neisseria meningitidis, | Escherichia coli |
| hutchinsonii | Genome Institute | Bordatella pertussis | |
| Dehalococcoides | TIGR, the | Thermus thermophilus, | |
| ethenogenes | Institute for | Deinococcus | |
| | Genomic | radiodurans, | |
| | Research | Escherichia coli | |
| Prochlorococcus | DOE-Joint | Synechocystis | Escherichia coli |
| marinus | Genome Institute | | |
| Porphyromonas | TIGR, the | Cytophaga hutchinsonii, | Escherichia coli, |
| gingivalis | Institute for | | Neisseria |
| | Genomic | | meningitidis |
| | Research | | |
| Yersinia pestis | The Sanger | Escherichia coli | |
| | Centre | | |
| Salmonella thphi | The Sanger | Escherichia coli | |
| | Centre | | |

^{a}A hit is defined as a sequence identified as delta using the criteria described in step 4 of the ‘psi-blast’ delta search

TABLE 21
β Proteins Corresponding to Additional δ Proteins Identified
from PSI-BLAST Search of Partially Sequenced Genomes

| | | GenBank gi | |
|---|---|---|---|
| Organism | Accession number | number | Database |
| Mycoplasma pulmonis | CAC13175 | 14089415 | NCBI +¹⁷ |
| Streptococcus agalactiae | Not found | | |
| Streptomyces coelicolor. | P27903 | 118804 | NCBI + |

¹⁷A plus symbol next to the database name indicates that the protein is annotated as β protein. A minus symbol next to the database indicates that the protein is not annotated.

What is claimed is:

1. A method for identifying a bacterial DNA polymerase holoenzyme δ subunit protein from at least one completely sequenced genome comprising:
a) providing a database of sequence information for a plurality of different organisms;
b) setting an inclusion threshold to a level sufficient to minimize the number of required iterations;
c) selecting the organisms to be searched wherein the selected organisms are candidate organisms;
d) providing the amino acid sequence of the δ subunit protein from a reference organism;
e) comparing the sequences of the candidate organisms and the reference organism;
f) excluding known DnaX proteins and known δ′ proteins from further steps;
g) selecting proteins comprising between 300 and 400 amino acids;
h) selecting a delta candidate from a candidate organism comprising selecting sequence with the lowest E score from the candidate organism; and
i) optionally repeating steps e)-g) wherein the sequences of the candidate organisms are sequences selected in step h, wherein a bacterial DNA polymerase holoenzyme δ subunit protein may be identified.

2. The method of claim 1, wherein the inclusion threshold is 0.001.

3. The method of claim 1, wherein the inclusion threshold is 0.002.

4. A bacterial DNA polymerase III holoenzyme δ subunit protein identified by the method of claim 1.

5. A method for identifying a δ protein from at least one partially sequenced genome comprising identifying a δ protein according the method of claim 1, wherein the selecting of step c) comprises selecting all organisms in the database for search which are not completely sequenced.

6. A method for identifying a bacterial DNA polymerase holoenzyme δ protein from at least one completely sequenced genome comprising:
a) providing a database of sequence information for a plurality of different organisms;
b) setting the inclusion threshold to a level sufficient to minimize the number of required iterations;
c) selecting the organisms to be searched, wherein the selected organisms are candidate organisms;
d) providing the amino acid sequence of the δ subunit protein from a reference organism;
e) comparing the sequences of the candidate organisms and the reference organism;
f) excluding known DnaX proteins and known δ′ proteins from further steps;
g) selecting proteins comprising at least three of the following six characteristics:
a. having at least five out of ten conserved residues from the conserved region 17—(L/I/V)XX(L/I/V)Y(L/I/V)(L/I/V)XGX(D/E)XX (L/I/V)(L/I/V)XXXXXX(L/I/V)—38;
b. having at least five out of eleven conserved residues from the conserved region 64—(L/I/V)(L/I/V)XXXX(A/S)XX(L/I/V)F(A/S)X (K/R)X(L/I/V)(L/I/V)(L/I/V)(L/I/V)—82;
c. having at least five out of ten conserved residues from the conserved region 97—(L/I/V)XX(L/I/V)(L/I/V)XXXXX(D/E)X(L/I/V) (L/I/V)(L/I/V)(L/I/V)XXXK(L/I/V)—117;
d. having at least nine out of eighteen conserved residues from the conserved region 154—(R/K)XXX(L/I/V)X(L/I/V)X(L/I/V) (D/E)X(D/E)(A/S)(L/I/V)XX(L/I/V)XXXXXXN(L/I/V)XX(L/I/V)XX(D/E)(L/I/V)X(R/K)(L/I/V) X(L/I/V)(L/I/V)—191;
e. having at least eleven out of twenty-three conserved residues from the conserved region 215—FX(L/I/V)X(D/E)(A/S)(L/I/V)(L/I/V) XG(R/K)XXX(A/S)(L/I/V)X(L/I/V)(L/I/V)XX(L/I/V)XXXGX(D/E)P(L/I/V)X(L/I/V)(L/I/V) XX(L/I/V)XXX(L/I/V)XX(L/I/V)XX(L/I/V)—260; and
f. having at least six out of twelve conserved residues from the conserved region 298—(L/I/V)XXX(L/I/V)XX(L/I/V)XX(D/E)XX (L/I/V)(R/K)XXXXX(D/E)XXXX(L/I/V)(D/E)XX(L/I/V)(L/I/V)X(L/I/V)—331; and
h) retaining the sequences above the threshold, wherein the retained sequences are delta candidate sequences; and
j) optionally repeating steps e)-g) wherein the sequences of the candidate organisms are delta candidate sequences, whereby a δ protein may be identified.

7. The method of claim B, wherein the plurality of different organisms comprises a plurality of evolutionarily distant organisms.

8. The method of claim 6, wherein the inclusion threshold is 0.001.

9. The method of claim 6, wherein the inclusion threshold is 0.002.

10. A bacterial DNA polymerase III holoenzyme δ subunit protein identified by the method of claim 6.

11. A method for identifying a δ protein from at least one partially sequenced genome comprising identifying a δ protein according the method of claim 6, wherein the selecting of step c) comprises selecting organisms in the database which are not completely sequenced.

12. A method for identifying a δ protein from at least one target organism comprising identifying a δ protein from an evolutionarily related organism, and performing a search for a similar sequence in the sequence of the target organism.

13. An isolated bacterial DNA polymerase δ subunit protein, wherein the δ subunit protein is not a member of the group consisting of an E. coli δ subunit protein, a Haemophilis influenzae δ subunit protein, and a Neisseria meningitides δ subunit protein.

14. An isolated bacterial DNA polymerase δ subunit protein, wherein the δ subunit protein is not a member of the group consisting of a gamma division of proteobacteria δ subunit protein and a beta division of proteobacteria δ subunit protein.

15. An isolated bacterial DNA polymerase δ subunit protein, wherein the δ subunit protein is a gram-positive bacteria δ subunit protein.

16. An isolated bacterial DNA polymerase δ subunit protein of claim 15, wherein the δ subunit protein is a member of the group consisting of an S. pyogenes, δ subunit protein, an S. aureus δ subunit protein, and an S. pneumoniae δ subunit protein.

17. An isolated bacterial DNA polymerase δ subunit protein, wherein the δ subunit protein is selected from the group consisting of Bacillus subtilis, Aquifex aeolicus, and Thermatoga maritima δ subunit protein.

18. The isolated bacterial DNA polymerase δ subunit protein of claim 17, wherein the protein comprises an amino acid sequence selected from the group consisting of
a) an amino acid sequence selected from the group consisting of SEQ ID NO: 3, SEQ ID NO: 226, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 227, SEQ ID NO: 228, SEQ ID NO: 54, SEQ ID NO: 57, SEQ ID NO: 60, SEQ ID NO: 63, SEQ ID NO: 66, SEQ ID NO: 69, SEQ ID NO: 72, SEQ ID NO: 75, SEQ ID NO: 78, SEQ ID NO: 81, SEQ ID NO: 84, SEQ ID NO: 87, SEQ ID NO: 90, SEQ ID NO: 93, SEQ ID NO: 96, SEQ ID NO: 99, SEQ ID NO: 102, SEQ ID NO: 229, SEQ ID NO: 105, SEQ ID NO: 108, SEQ ID NO: 111, SEQ ID NO: 114, SEQ ID NO: 117, SEQ ID NO: 120, SEQ ID NO: 123, SEQ ID NO: 126, SEQ ID NO: 129, SEQ ID NO: 132, SEQ ID NO: 135, SEQ ID NO: 138, SEQ ID NO: 141, SEQ ID NO: 144, SEQ ID NO: 147, SEQ ID NO: 150, SEQ ID NO: 153, and SEQ ID NO: 156; and
b) an amino acid sequence selected from the group consisting of an amino acid sequence having at least 95% sequence identity to an amino acid sequence of a).

19. An antibody, wherein the antibody is capable of specifically binding to at least one antigenic determinant on the protein encoded by an amino acid sequence according to claim 16.

20. The antibody of claim 19, wherein the antibody type is selected from the group consisting of polyclonal, monoclonal, chimeric, single chain, F_{ab} fragments, and an F_{ab} expression library.

21. A method for producing anti-DNA polymerase III δ subunit antibodies comprising exposing an animal having immunocompetent cells to an immunogen comprising at least an antigentic portion of DNA polymerase III δ subunit.

22. The method of claim 21, further comprising the step of harvesting the antibodies.

23. The method of claim 21, further comprising fusing the immunocompetent cells with an immortal cell line under conditions such that a hybridoma is produced.

24. A method for detecting DNA polymerase III δ subunit protein comprising, a) providing in any order, a sample suspected of containing DNA polymerase III, an antibody capable of specifically binding to at least a portion of the DNA polymerase III δ subunit protein;
b) mixing the sample and the antibody under conditions wherein the antibody can bind to the DNA polymerase III; and
c) detecting the binding.

25. An isolated nucleic acid molecule selected from the group consisting of:
a) an isolated bacterial nucleic acid molecule comprising a nucleic acid sequence encoding a protein selected from the group consisting of:
i) a protein having an amino acid sequence selected from the group consisting of SEQ ID NO: 3, SEQ ID NO: 226, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 227, SEQ ID NO: 228, SEQ ID NO: 54, SEQ ID NO: 57, SEQ ID NO: 60, SEQ ID NO: 63, SEQ ID NO: 66, SEQ ID NO: 69, SEQ ID NO: 72, SEQ ID NO: 75, SEQ ID NO: 78, SEQ ID NO: 81, SEQ ID NO: 84, SEQ ID NO: 87, SEQ ID NO: 90, SEQ ID NO: 93, SEQ ID NO: 96, SEQ ID NO: 99, SEQ ID NO: 102, SEQ ID NO: 229, SEQ ID NO: 105, SEQ ID NO: 108, SEQ ID NO: 111, SEQ ID NO: 114, SEQ ID NO: 117, SEQ ID NO: 120, SEQ ID NO: 123, SEQ ID NO: 126, SEQ ID NO: 129, SEQ ID NO: 132, SEQ ID NO: 135, SEQ ID NO: 138, SEQ ID NO: 141, SEQ ID NO: 144, SEQ ID NO: 147, SEQ ID NO: 150, SEQ ID NO: 153, and SEQ ID NO: 156; and
ii) a protein selected from the group consisting of a protein having at least 95% sequence identity to an amino acid sequence of i); and
b) an isolated bacterial nucleic acid molecule which is fully complementary to any said nucleic acid molecule recited in a).

26. The nucleic acid molecule of claim 25, wherein said nucleic acid molecule is selected from the group consisting of Aquifex, Bacillus, Buchnera, Borrelia, Campylobacter, Caulobacter, Chlamydia, Chlamydia, Chlamydophila, Deinococcus, Haemphilus, Helicobacter, Lactococcus, Mycobacterium, Mesorhizobium, Mycoplasma, Neisseria, Pasteurella, Pseudomonas, Rickettsia, Synechocystis, Thermatoga, Treponema, Ureaplasma, Vibrio, Xylella, Mycoplasma, Staphylococcus, Streptococcus, Streptomyces, Streptococcus, Streptococcus, Thermus, Yersinia, Actinobacillus, Bordetealla, Bacillus, Burkholderia, Chlorobium, Chloroflexus, Clostridium, Corynebacterium, Cytopahaga, Dehalococcoides, Porphyromonas, Prochlorococcus, and Salmonella nucleic acid molecules.

27. The nucleic acid molecule of claim 25, wherein said nucleic acid molecule is selected from the group consisting of Aquifex aeolicus, Bacillus halodurans, Bacillus subtilis, Buchnera, Borrelia burgdorferi, Campylobacter jejuni, Caulobacter crescentus, Chlamydia muridarum, Chlamydia trachomatis, Chlamydophila pneumonia, Deinococcus radiodurans, Haemophilus influenzae, Helicobacter pylori 26695, Helicobacter pylori J99, Lactococcus lactis, Mycobacterium laprae, Mesorhizobium loti, Mycoplasma genitalium, Mycoplasma pneumoniae, Pasteurella multocida, Pseudomonas aeruginosa, Rickettsia prowazekii, Synechocystis, Thermatoga maritima, Treponema pallidum, Ureaplasma urealyticum, Vibrio cholerae, Xylella fastidiosa, Mycoplasma pulmonis, Staphylococcus aureus, Streptococcus agalactiae, Streptomyces coelicolor, Streptococcus pneumoniae, Streptococcus pyogenes, Thermus thermophilus, Yersinia pestis, Actinobacillus actinomycetemcomitans, Bordetealla pertussis, Bacillus anthracis, Burkholderia pseudomallei, Chlorobium tepidum, Chloroflexus aurantiacus, Clostridium difficile, Corynebacterium diphtheriae, Cytopahaga hutchinsonii, Dehalococcoides ethenogenes, Porphyromonas gingivalis, Prochlorococcus marinus, and Salmonella typhi nucleic acid molecules.

28. The nucleic acid molecule of claim 25, wherein said nucleic acid molecule comprises a nucleic acid molecule selected from the group consisting of SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 223, SEQ ID NO: 52, SEQ ID NO: 55, SEQ ID NO: 58, SEQ ID NO: 61, SEQ ID NO: 64, SEQ ID NO: 67, SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103, SEQ ID NO: 106, SEQ ID NO: 109, SEQ ID NO: 112, SEQ ID NO: 115, SEQ ID NO: 118, SEQ ID NO: 121, SEQ ID NO: 124, SEQ ID NO: 127, SEQ ID NO: 130, SEQ ID NO: 133, SEQ ID NO: 136, SEQ ID NO: 139, SEQ ID NO: 142, SEQ ID NO: 145, SEQ ID NO: 148, SEQ ID NO: 151, and SEQ ID NO: 154.

29. A recombinant molecule comprising at least a portion of a bacterial DNA polymerase III holA nucleic acid molecule according to claim 25, 26, 27, or 28.

30. A recombinant cells comprising at least a portion of a bacterial DNA polymerase III holA nucleic acid molecule according to claim 25, 26, 27 or 28.

31. A method for detection of nucleic acid molecules encoding at least a portion of DNA polymerase III δ subunit in a biological sample comprising the steps of:
a) hybridizing at least a portion of a holA nucleic acid molecule to nucleic acid material of a biological sample, thereby forming a hybridization complex, and
b) detecting the hybridization complex, wherein the presence of the complex correlates with the presence of a polynucleotide encoding at least a portion of DNA polymerase III δ subunit in the biological sample.

32. The method of claim 31, wherein the holA nucleic acid molecule is a holA nucleic acid molecule according to claim 25, 26, 27, or 28.

33. A method for detecting DNA polymerase III δ subunit expression, including expression of modified or mutated DNA polymerase III δ subunit proteins or gene sequences comprising the steps of
a) providing a test sample suspected of containing DNA polymerase III δ subunit protein, as appropriate; and
b) comparing test DNA polymerase III δ subunit with quantitated DNA polymerase III holoenzyme or holoenzyme subunit in a control to determine the relative concentration of the test DNA polymerase III δ subunit in the sample.

34. A method of screening for a compound that modulates the activity of a DNA polymerase III replicase, said method comprising:
a) contacting an isolated replicase with at least one test compound under conditions permissive for replicase activity;
b) assessing the activity of the replicase in the presence of the test compound; and
c) comparing the activity of the replicase in the presence of the test compound with the activity of the replicase in the absence of the test compound, wherein a change in the activity of the replicase in the presence of the test compound is indicative of a compound that modulates the activity of the replicase,
wherein said replicase comprises an isolated DNA polymerase III δ subunit protein.

35. The method of claim 34, wherein said DNA polymerase III δ subunit protein is encoded by a nucleic acid molecule selected from the group consisting of
a) SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 223, SEQ ID NO: 52, SEQ ID NO: 55, SEQ ID NO: 58, SEQ ID NO: 61, SEQ ID NO: 64, SEQ ID NO: 67, SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103, SEQ ID NO: 106, SEQ ID NO: 109, SEQ ID NO: 112, SEQ ID NO: 115, SEQ ID NO: 118, SEQ ID NO: 121, SEQ ID NO: 124, SEQ ID NO: 127, SEQ ID NO: 130, SEQ ID NO: 133, SEQ ID NO: 136, SEQ ID NO: 139, SEQ ID NO: 142, SEQ ID NO: 145, SEQ ID NO: 148, SEQ ID NO: 151, and SEQ ID NO: 154; and
b) a protein comprising a homologue of a protein of a), wherein said homologue encodes a protein containing one or more amino acid deletions, substitutions, or insertions, and wherein said protein performs the function of a natural δ subunit protein in a bacterial replication assay; and
c) an isolated bacterial nucleic acid molecule which is fully complementary to any said nucleic acid molecule recited in a).

36. A compound that modulates the activity of a DNA polymerase III replicase identified by the method of claim 34 or 35.

37. A method of identifying compounds which modulate the activity of a DNA polymerase III replicase comprising
a) forming a reaction mixture that includes a primed DNA molecule, a DNA polymerase α subunit, a candidate compound, a dNTP, and optionally, a member of the group consisting of a β subunit, a τ complex, and both the β subunit and the τ complex to form a replicase;
b) subjecting the reaction mixture to conditions effective to achieve nucleic acid polymerization in the absence of the candidate compound: and
c) comparing the activity of the replicase in the presence of the test compound with the activity of the replicase in the absence of the test compound, wherein a change in the activity of the replicase in the presence of the test compound is indicative of a compound that modulates the activity of the replicase,
wherein said replicase comprises a DNA polymerase III δ subunit protein.

38. The method of claim 37, wherein said DNA polymerase III δ subunit protein is encoded by a nucleic acid molecule selected from the group consisting of
a) SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 223, SEQ ID NO: 52, SEQ ID NO: 55, SEQ ID NO: 58, SEQ ID NO: 61, SEQ ID NO: 64, SEQ ID NO: 67, SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103, SEQ ID NO: 106, SEQ ID NO: 109, SEQ ID NO: 112, SEQ ID NO: 115, SEQ ID NO: 118, SEQ ID NO: 121, SEQ ID NO: 124, SEQ ID NO: 127, SEQ ID NO: 130, SEQ ID NO: 133, SEQ ID NO: 136, SEQ ID NO: 139, SEQ ID NO: 142, SEQ ID NO: 145, SEQ ID NO: 148, SEQ ID NO: 151, and SEQ ID NO: 154; and
b) a protein comprising a homologue of a protein of a), wherein said homologue encodes a protein containing one or more amino acid deletions, substitutions, or insertions, and wherein said protein performs the function of a natural δ subunit protein in a bacterial replication assay; and
c) an isolated bacterial nucleic acid molecule which is fully complementary to any said nucleic acid molecule recited in a).

39. A compound that modulates the activity of a DNA polymerase III replicase identified by the method of claim 37 or 38.

40. A method of identifying compounds that modulate the activity of a DnaX complex and a β subunit in stimulating a DNA polymerase replicase comprising
a) contacting a primed DNA (which may be coated with SSB) with a DNA polymerase replicase, a β subunit, and a τ complex (or subunit or subassembly of the DnaX complex) in the presence of the candidate pharmaceutical, and dNTPs (or modified dNTPs) to form a reaction mixture
b) subjecting the reaction mixture to conditions effective to achieve nucleic acid polymerization in the absence of the candidate compound; and
c) comparing the nucleic acid polymerization in the presence of the test compound with the nucleic acid polymerization in the absence of the test compound, wherein a change in the nucleic acid polymerization in the presence of the test compound is indicative of a compound that modulates the activity of a DnaX complex and a β subunit,
wherein said τ complex (or subunit or subassembly of the τ complex) comprises a DNA polymerase III δ subunit protein.

41. The method of claim 40, wherein said DNA polymerase III δ subunit protein is encoded by a nucleic acid molecule selected from the group consisting of
a) SEQ ID NO: 1, SEQ ID NO: 4, SEQ ID NO: 7, SEQ ID NO:l0, SEQ ID NO: 13, SEQ ID NO: 16, SEQ ID NO: 19, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 223, SEQ ID NO: 52, SEQ ID NO: 55, SEQ ID NO: 58, SEQ ID NO: 61, SEQ ID NO: 64, SEQ ID NO: 67, SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103, SEQ ID NO: 106, SEQ ID NO: 109, SEQ ID NO: 112, SEQ ID NO: 115, SEQ ID NO: 118, SEQ ID NO: 121, SEQ ID NO: 124, SEQ ID NO: 127, SEQ ID NO: 130, SEQ ID NO: 133, SEQ ID NO: 136, SEQ ID NO: 139, SEQ ID NO: 142, SEQ ID NO: 145, SEQ ID NO: 148, SEQ ID NO: 151, and SEQ ID NO: 154; and
b) a protein comprising a homologue of a protein of a), wherein said homologue encodes a protein containing one or more amino acid deletions, substitutions, or insertions, and wherein said protein performs the function of a natural δ subunit protein in a bacterial replication assay; and
c) an isolated bacterial nucleic acid molecule which is fully complementary to any said nucleic acid molecule recited in a).

42. A compound that modulates the activity of a DNA polymerase III replicase identified by the method of claim 40 or 41.

43. A method to identify compounds that modulate the ability of a β subunit and a DnaX complex (or a subunit or subassembly of the DnaX complex) to interact comprising
a) contacting the β subunit with the DnaX complex (or subunit or subassembly of the DnaX complex) in the presence of the compounds to form a reaction mixture
b) subjecting the reaction mixture to conditions under which the DnaX complex (or the subunit or subassembly of the DnaX complex) and the β subunit would interact in the absence of the compound; and
c) comparing the extent of interaction in the presence of the test compound with the extent of interaction in the absence of the test compound, wherein a change in the interaction between the β subunit and the DnaX complex (or the subunit or subassembly of the DnaX complex) is indicative of a compound that modulates the interaction,
wherein said DnaX complex (or subunit or subassembly of the DnaX complex) comprises a DNA polymerase III δ subunit protein.

44. The method of claim 43, wherein said DNA polymerase III δ subunit protein is encoded by a nucleic acid molecule selected from the group consisting of
a) SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 223, SEQ ID NO: 52, SEQ ID NO: 55, SEQ ID NO: 58, SEQ ID NO: 61, SEQ ID NO: 64, SEQ ID NO: 67, SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103, SEQ ID NO: 106, SEQ ID NO: 109, SEQ ID NO: 112, SEQ ID NO: 115, SEQ ID NO: 118, SEQ ID NO: 121, SEQ ID NO: 124, SEQ ID NO: 127, SEQ ID NO: 130, SEQ ID NO: 133, SEQ ID NO: 136, SEQ ID NO: 139, SEQ ID NO: 142, SEQ ID NO: 145, SEQ ID NO: 148, SEQ ID NO: 151, and SEQ ID NO: 154; and
b) a protein comprising a homologue of a protein of a), wherein said homologue encodes a protein containing one or more amino acid deletions, substitutions, or insertions, and wherein said protein performs the function of a natural δ subunit protein in a bacterial replication assay; and
c) an isolated bacterial nucleic acid molecule which is fully complementary to any said nucleic acid molecule recited in a).

45. A compound that modulates the activity of a DNA polymerase III replicase identified by the method of claim 43 or 44.

46. A method to identify compounds that modulate the ability of a DnaX complex (or a subassembly of the DnaX complex) to assemble a β subunit onto a DNA molecule comprising
a) contacting a circular primed DNA molecule (which may be coated with SSB) with the DnaX complex (or the subassembly thereof) and the β subunit in the presence of the compound, and ATP or dATP to form a reaction mixture
b) subjecting the reaction mixture to conditions under which the DnaX complex (or subassembly) assembles the β subunit on the DNA molecule absent the compound; and
c) comparing extent of assembly in the presence of the test with the extent of assembly in the absence of the test compound, wherein a change in the amount of β subunit on the DNA molecule is indicative of a compound that modulates the ability of a DnaX complex (or a subassembly of the DnaX complex) to assemble a β subunit onto a DNA molecule,
wherein the DnaX complex (or a subassembly of the DnaX complex) comprises a DNA polymerase III δ subunit protein.

47. The method of claim 46, wherein said DNA polymerase III δ subunit protein is encoded by a nucleic acid molecule selected from the group consisting of
a) SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 223, SEQ ID NO: 52, SEQ ID NO: 55, SEQ ID NO: 58, SEQ ID NO: 61, SEQ ID NO: 64, SEQ ID NO: 67, SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103, SEQ ID NO: 106, SEQ ID NO: 109, SEQ ID NO: 112, SEQ ID NO: 115, SEQ ID NO: 118, SEQ ID NO: 121, SEQ ID NO: 124, SEQ ID NO: 127, SEQ ID NO: 130, SEQ ID NO: 133, SEQ ID NO: 136, SEQ ID NO: 139, SEQ ID NO: 142, SEQ ID NO: 145, SEQ ID NO: 148, SEQ ID NO: 151, and SEQ ID NO: 154; and
b) a protein comprising a homologue of a protein of a), wherein said homologue encodes a protein containing one or more amino acid deletions, substitutions, or insertions, and wherein said protein performs the function of a natural δ subunit protein in a bacterial replication assay; and
c) an isolated bacterial nucleic acid molecule which is fully complementary to any said nucleic acid molecule recited in a).

48. A compound that modulates the activity of a DNA polymerase III replicase identified by the method of claim 46 or 47.

49. A method to identify compounds that modulate the ability of a DnaX complex (or a subunit(s) of the DnaX complex) to disassemble a β subunit from a DNA molecule comprising
a) contacting a DNA molecule onto which the β subunit has been assembled in the presence of the compound, to form a reaction mixture;
b) subjecting the reaction mixture to conditions under which the DnaX complex (or a subunit(s) or subassembly of the DnaX complex) disassembles the β subunit from the DNA molecule absent the compound; and
c) comparing the extent of assembly in the presence of the test compound with the extent of assembly in the absence of the test compound, wherein a change in the amount of β subunit on the DNA molecule is indicative of a compound that modulates the ability of a DnaX complex (or a subassembly of the DnaX complex) to disassemble a β subunit onto a DNA molecule,
wherein the DnaX complex (or a subassembly of the DnaX complex) comprises a DNA polymerase III δ subunit protein.

50. The method of claim 49, wherein said DNA polymerase III δ subunit protein is encoded by a nucleic acid molecule selected from the group consisting of
a) SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 223, SEQ ID NO: 52, SEQ ID NO: 55, SEQ ID NO: 58, SEQ ID NO: 61, SEQ ID NO: 64, SEQ ID NO: 67, SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103, SEQ ID NO: 106, SEQ ID NO: 109, SEQ ID NO: 112, SEQ ID NO: 115, SEQ ID NO: 118, SEQ ID NO: 121, SEQ ID NO: 124, SEQ ID NO: 127, SEQ ID NO: 130, SEQ ID NO: 133, SEQ ID NO: 136, SEQ ID NO: 139, SEQ ID NO: 142, SEQ ID NO: 145, SEQ ID NO: 148, SEQ ID NO: 151, and SEQ ID NO: 154; and
b) a protein comprising a homologue of a protein of a), wherein said homologue encodes a protein containing one or more amino acid deletions, substitutions, or insertions, and wherein said protein performs the function of a natural δ subunit protein in a bacterial replication assay; and
c) an isolated bacterial nucleic acid molecule which is fully complementary to any said nucleic acid molecule recited in a).

51. A compound that modulates the activity of a DNA polymerase III replicase identified by the method of claim 49 or 50.

52. A method to identify compounds that modulate the dATP/ATP binding activity of a DnaX complex or a DnaX complex subunit (e.g. τ subunit) comprising
a) contacting the DnaX complex (or the DnaX complex subunit) with dATP/ATP either in the presence or absence of a DNA molecule and/or the β subunit in the presence of the compound to form a reaction mixture;
b) subjecting the reaction mixture to conditions in which the DnaX complex (or the subunit of DnaX complex) interacts with dATP/ATP in the absence of the compound; and
c) comparing the extent of binding in the presence of the test compound with the extent of binding in the absence of the test compound, wherein a change in the dATP/ATP binding is indicative of a compound that modulates the dATP/ATP binding activity of a DnaX complex or a DnaX complex subunit (e.g. τ subunit),
wherein the DnaX complex (or the subunit of DnaX complex) comprises a DNA polymerase III δ subunit protein.

53. The method of claim 52, wherein said DNA polymerase III δ subunit protein is encoded by a nucleic acid molecule selected from the group consisting of
a) SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 223, SEQ ID NO: 52, SEQ ID NO: 55, SEQ ID NO: 58, SEQ ID NO: 61, SEQ ID NO: 64, SEQ ID NO: 67, SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103, SEQ ID NO: 106, SEQ ID NO: 109, SEQ ID NO: 112, SEQ ID NO: 115, SEQ ID NO: 118, SEQ ID NO: 121, SEQ ID NO: 124, SEQ ID NO: 127, SEQ ID NO: 130, SEQ ID NO: 133, SEQ ID NO: 136, SEQ ID NO: 139, SEQ ID NO: 142, SEQ ID NO: 145, SEQ ID NO: 148, SEQ ID NO: 151, and SEQ ID NO: 154; and
b) a protein comprising a homologue of a protein of a), wherein said homologue encodes a protein containing one or more amino acid deletions, substitutions, or insertions, and wherein said protein performs the function of a natural δ subunit protein in a bacterial replication assay; and
c) an isolated bacterial nucleic acid molecule which is fully complementary to any said nucleic acid molecule recited in a).

54. An compound that modulates the activity of a DNA polymerase III replicase identified by the method of claim 52 or 53.

55. A method to identify compound that modulate the dATP/ATPase activity of a DnaX complex or a DnaX complex subunit (e.g., the τ subunit) comprising
a) contacting the DnaX complex (or the DnaX complex subunit) with dATP/ATP either in the presence or absence of a DNA molecule and/or a β subunit in the presence of the compound to form a reaction mixture;
b) subjecting the reaction mixture to conditions in which the DnaX subunit (or complex) hydrolyzes dATP/ATP in the absence of the compound; and
c) comparing the extent of hydrolysis in the presence of the test compound with the extent of hydrolysis in the absence of the test compound, wherein a change in the amount of dATP/ATP hydrolyzed is indicative of a compound that modulates the dATP/ATPase activity of a DnaX complex or a DnaX complex subunit (e.g., the τ subunit)
wherein the DnaX complex (or subunit) comprises a DNA polymerase III δ subunit protein.

56. The method of claim 55, wherein said DNA polymerase III δ subunit protein is encoded by a nucleic acid molecule selected from the group consisting of
a) SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 223, SEQ ID NO: 52, SEQ ID NO: 55, SEQ ID NO: 58, SEQ ID NO: 61, SEQ ID NO: 64, SEQ ID NO: 67, SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103, SEQ ID NO: 106, SEQ ID NO: 109, SEQ ID NO: 112, SEQ ID NO: 115, SEQ ID NO: 118, SEQ ID NO: 121, SEQ ID NO: 124, SEQ ID NO: 127, SEQ ID NO: 130, SEQ ID NO: 133, SEQ ID NO: 136, SEQ ID NO: 139, SEQ ID NO: 142, SEQ ID NO: 145, SEQ ID NO: 148, SEQ ID NO: 151, and SEQ ID NO: 154; and
b) a protein comprising a homologue of a protein of a), wherein said homologue encodes a protein containing one or more amino acid deletions, substitutions, or insertions, and wherein said protein performs the function of a natural δ subunit protein in a bacterial replication assay; and
c) an isolated bacterial nucleic acid molecule which is fully complementary to any said nucleic acid molecule recited in a).

57. An compound that modulates the activity of a DNA polymerase III replicase identified by the method of claim 55 or 56.

58. A method for identifying compound that modulate the activity of a DNA polymerase replicase comprising
a) contacting a circular primed DNA molecule, optionally coated with SSB, with a DnaX complex, a β subunit and an α subunit in the presence of the compound, and dNTPs (or modified dNTPs) to form a reaction mixture;
b) subjecting the reaction mixture to conditions, which in the absence of the compound, affect nucleic acid polymerization; and
c) comparing the nucleic acid polymerization in the presence of the test compound with the nucleic acid polymerization in the absence of the test compound, wherein a change in the activity of the replicase in the presence of the test compound is indicative of a compound that modulates the activity of the replicase,
wherein the DnaX complex comprises a DNA polymerase III δ subunit protein.

59. The method of claim 58, wherein said DNA polymerase III δ subunit protein is encoded by a nucleic acid molecule selected from the group consisting of
a) SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 223, SEQ ID NO: 52, SEQ ID NO: 55, SEQ ID NO: 58, SEQ ID NO: 61, SEQ ID NO: 64, SEQ ID NO: 67, SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103, SEQ ID NO: 106, SEQ ID NO: 109, SEQ ID NO: 112, SEQ ID NO: 115, SEQ ID NO: 118, SEQ ID NO: 121, SEQ ID NO: 124, SEQ ID NO: 127, SEQ ID NO: 130, SEQ ID NO: 133, SEQ ID NO: 136, SEQ ID NO: 139, SEQ ID NO: 142, SEQ ID NO: 145, SEQ ID NO: 148, SEQ ID NO: 151, and SEQ ID NO: 154; and
b) a protein comprising a homologue of a protein of a), wherein said homologue encodes a protein containing one or more amino acid deletions, substitutions, or insertions, and wherein said protein performs the function of a natural δ subunit protein in a bacterial replication assay; and
c) an isolated bacterial nucleic acid molecule which is fully complementary to any said nucleic acid molecule recited in a).

60. A compound that modulates the activity of a DNA polymerase III replicase identified by the method of claim 58 or 59.

61. A method to identify compound that modulate the ability of a δ subunit and the δ′ and/or DnaX subunit to interact comprising
a) contacting the δ subunit with the δ′ and/or δ′ plus DnaX subunit in the presence of the compound to form a reaction mixture
b) subjecting the reaction mixture to conditions under which the δ subunit and the δ′ and/or δ′ plus DnaX subunit would interact in the absence of the compound
c) comparing the extent of interaction in the presence of the test compound with the extent of interaction in the absence of the test compound, wherein a change in the interaction between the δ subunit and the δ′ and/or DnaX subunit is indicative of a compound that modulates the interaction,
wherein the DnaX complex comprises a DNA polymerase III δ subunit protein.

62. The method of claim 61, wherein said DNA polymerase III δ subunit protein is encoded by a nucleic acid molecule selected from the group consisting of
a) SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 223, SEQ ID NO: 52, SEQ ID NO: 55, SEQ ID NO: 58, SEQ ID NO: 61, SEQ ID NO: 64, SEQ ID NO: 67, SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103, SEQ ID NO: 106, SEQ ID NO: 109, SEQ ID NO: 112, SEQ ID NO: 115, SEQ ID NO: 118, SEQ ID NO: 121, SEQ ID NO: 124, SEQ ID NO: 127, SEQ ID NO: 130, SEQ ID NO: 133, SEQ ID NO: 136, SEQ ID NO: 139, SEQ ID NO: 142, SEQ ID NO: 145, SEQ ID NO: 148, SEQ ID NO: 151, and SEQ ID NO: 154; and
b) a protein comprising a homologue of a protein of a), wherein said homologue encodes a protein containing one or more amino acid deletions, substitutions, or insertions, and wherein said protein performs the function of a natural δ subunit protein in a bacterial replication assay; and
c) an isolated bacterial nucleic acid molecule which is fully complementary to any said nucleic acid molecule recited in a).

63. A compound that modulates the activity of a DNA polymerase III replicase identified by the method of claim 61 or 62.

64. A method of synthesizing a DNA molecule comprising
a) hybridizing a primer to a first DNA molecule; and
b) incubating said DNA molecule in the presence of a thermostable DNA polymerase replicase and one or more dNTPs under conditions sufficient to synthesize a second DNA molecule complementary to all or a portion of said first DNA molecule;
wherein said thermostable DNA polymerase replicase comprises an δ subunit protein selected from the group consisting of an Aquifex, Thermus and Thermatoga δ subunit protein.

65. The method of claim 66, wherein said thermostable DNA polymerase replicase comprises an δ subunit protein selected from the group consisting of an Aquifex aeolicus, Thermus thermophilus, and Thermatoga maratima δ subunit protein.

67. The method of claim 65, wherein said thermostable DNA polymerase replicase comprises a DNA polymerase δ subunit protein selected from the group consisting of
i) a protein selected from the group consisting of SEQ ID NO: 3, SEQ ID NO: 226, SEQ ID NO: 81, and SEQ ID NO: 108; and;
ii) and a protein selected from the group consisting of a protein having 95% sequence identity to an amino acid sequence of i).

68. A method of amplifying a double-stranded DNA molecule comprising:
a) providing a first and second primer, wherein said first primer is complementary to a sequence at or near the 3′-termini of the first strand of said DNA molecule and said second primer is complementary to a sequence at or near the 3′-termini of the second strand of said DNA molecule;
b) hybridizing said primer to said first strand and said second primer to said second strand in the presence of a thermostable DNA polymerase replicase, under conditions such that a third DNA molecule complementary to said first strand and a fourth DNA molecule complementary to said second strand are synthesized;
c) denaturing said first and third strand, and said second and fourth strands; and
d) repeating steps a) to c) one or more times; wherein:
said thermostable DNA polymerase is selected from the group consisting of an Aquifex, Thermus and Thermatoga δ subunit protein.

67. The method of claim X, wherein said thermostable DNA polymerase replicase comprises an δ subunit protein selected from the group consisting of an Aquifex aeolicus, Thermus thermophilus, and Thermatoga maratima δ subunit protein.

69. The method of claim 68, wherein said thermostable DNA polymerase replicase comprises a DNA polymerase δ subunit protein selected from the group consisting of
i) a protein selected from the group consisting of SEQ ID NO: 3, SEQ ID NO: 226, SEQ ID NO: 81, and SEQ ID NO: 108; and;
ii) and a protein selected from the group consisting of a protein having 95% sequence identity to an amino acid sequence of i).

70. A method of amplifying nucleic acid sequences, the method comprising,
a) mixing a rolling circle replication primer with one or more amplification target circles, to produce a primer-amplification target circle mixture, and incubating the primer-amplification target circle mixture under conditions that promote hybridization between the amplification target circles and the rolling circle replication primer in the primer-amplification target circle mixture,
wherein the amplification target circles each comprise a single-stranded, circular DNA molecule comprising a primer complement portion, and wherein the primer complement portion is complementary to the rolling circle replication primer, and
b) mixing a thermostable DNA polymerase replicase with the primer-amplification target circle mixture, to produce a replicase-amplification target circle mixture, and incubating the replicase-amplification target circle mixture under conditions that promote replication of the amplification target circles,
wherein replication of the amplification target circles results in the formation of tandem sequence DNA,
and wherein said thermostable DNA polymerase replicase comprises an δ subunit protein selected from the group consisting of an Aquifex, Thermus and Thermatoga δ subunit protein.

71. The method of claim 70, wherein said thermostable DNA polymerase replicase comprises an δ subunit protein selected from the group consisting of an Aquifex aeolicus, Thermus thermophilus, and Thermatoga maratima δ subunit protein.

72. The method of claim 71, wherein said thermostable DNA polymerase replicase comprises a DNA polymerase δ subunit protein selected from the group consisting of
i) a protein selected from the group consisting of SEQ ID NO: 3, SEQ ID NO: 226, SEQ ID NO: 81, and SEQ ID NO: 108; and;
ii) and a protein selected from the group consisting of a protein having 95% sequence identity to an amino acid sequence of i).

73. The method of claim 70, wherein said DNA polymerase replicase comprises a homolog of E. coli DnaB helicase.

74. A method of synthesizing DNA which comprises utilizing one or more polypeptides, said one or more polypeptides comprising an amino acid sequence having at least 95% sequence identity to an amino acid sequence from the group consisting of SEQ ID NO: 3, SEQ ID NO: 226, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 227, SEQ ID NO: 228, SEQ ID NO: 54, SEQ ID NO: 57, SEQ ID NO: 60, SEQ ID NO: 63, SEQ ID NO: 66, SEQ ID NO: 69, SEQ ID NO: 72, SEQ ID NO: 75, SEQ ID NO: 78, SEQ ID NO: 81, SEQ ID NO: 84, SEQ ID NO: 87, SEQ ID NO: 90, SEQ ID NO: 93, SEQ ID NO: 96, SEQ ID NO: 99, SEQ ID NO: 102, SEQ ID NO: 229, SEQ ID NO: 105, SEQ ID NO: 108, SEQ ID NO: 111, SEQ ID NO: 114, SEQ ID NO: 117, SEQ ID NO: 120, SEQ ID NO: 123, SEQ ID NO: 126, SEQ ID NO: 129, SEQ ID NO: 132, SEQ ID NO: 135, SEQ ID NO: 138, SEQ ID NO: 141, SEQ ID NO: 144, SEQ ID NO: 147, SEQ ID NO: 150, SEQ ID NO: 153, and SEQ ID NO: 156.

75. The method of claim 74 further comprising providing in any order: a reaction mixture comprising components comprising template, and nucleotides, and incubating said reaction mixture for a length of time and at a temperature sufficient to obtain DNA synthesis.

76. An isolated bacterial DNA polymerase δ subunit protein, wherein the δ subunit protein is a phylum Firmicutes δ subunit protein.
